SEQUENCE LISTING (156 sequences)

SEQ ID NO: 1 - protein, 96 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 2 - protein, 119 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 3 - protein, 117 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 4 - DNA, 1520 bp, Homo sapiens
atgaggcctctgcccagcgggaggaggaagacccgaggcatctccctaggactcttcgccctctgcctggccgcagcccgctgtctgcagagtcagggtgtgtccctatacattcctcaggccaccatcaatgccactgtcaaagaagacatcctgctctcagttgagtactcctgtcatggagtgcccaccatcgaatggacatattcatccaattggggaacgcagaagatcgtggagtggaaaccagggactcaggccaacatctctcaaagccacaaggacagagtctgcacctttgacaacggctccatccagctcttcagcgtgggagtgagggattccggctactatgtcatcaccgtgacggagcgcctggggagcagccagtttggcaccatcgtgctgcacgtctctgagatcctctatgaagacctgcactttgtcgctgtcatccttgcttttctcgctgctgtggccgcagtattaatcagcctcatgtgggtttgtaataagtgtgcatataaatttcagaggaagagaagacacaaactcaaagaaagcacaactgaggagattgagctggaagatgttgagtgttagccaaggctgggcctgactgcattcctacctcaagaggaaaccattctccaacaaaaagagcaagcacagctattatacccattgtgtgtggtcctgttgcagcccgctcctaacaggacagtgggagattaacaacattgactgcatggagttgaggactgtggatgggacaaagctagtattaggactcgcgctaagttcaaggagaagagtgattgaggctttgaaccaggagcttcgcttggctgcagcatcagggccgtgctgacacataaccaatggcagtcggaggaaccaggctctgggccaggacagtttccagtgctgtgaagaaacgaggtgagcagtatccaaggggctcaaggataatctgctgatttctttctcgtctttcacacagagaggccaccagcaggaaagcagtgggagttgggtagctgctggggccagcatgtcctcttccacactacctgcctttcagaactctgctcttccgcttgtcccgaagtcagggctggcaatcttcataatcaaaggggagttggaaaagaacacccactaagaggcctccatggcagatgtcaattaaattgcccccccccccaaatctcatgacagtttattcacatcagtagtgtgggaagttacaatgttttttttaaaaaaagcttctttccttggctttccatttctttgaaaaaagggtttcttttgaatttttaaagctctgccatactgaacattcctgtggaaaggtttaaaatgcagagcctgaggtttttgcttttcagaaaaataaaaatcatagagattagctagtgtaaatgttaagctataaattatgtttcaaactgtgaaaaaaaaaagttttaaagaatgaatgaaataaaacctgaaaataaagccctggtcctttg

SEQ ID NO: 5 - DNA, 1650 bp, Homo sapiens
atgaggcctctgcccagcgggaggaggaagacccgaggcatctccctaggactcttcgccctctgcctggccgcagcccgctgtctgcagagtcagggtgtgtccctatacattcctcaggccaccatcaatgccactgtcaaagaagacatcctgctctcagttgagtactcctgtcatggagtgcccaccatcgaatggacatattcatccaattggggaacgcagaagatcgtggagtggaaaccagggactcaggccaacatctctcaaagccacaaggacagagtctgcacctttgacaacggctccatccagctcttcagcgtgggagtgagggattccggctactatgtcatcaccgtgacggagcgcctggggagcagccagtttggcaccatcgtgctgcacgtctctgagatcctctatgaagacctgcactttgtcgctgtcatccttgcttttctcgctgctgtggccgcagtattaatcagcctcatgtgggtttgtaataagtgtgcatataaatttcagaggaagagaagacacaaactcaaaggtaaccccctgggccttgtgataatccatgagtggttttgaggccagaggtggcatgtgttatcttgttctcacgaagaaagcactatgtggcttgatttccaactcattgacttgtctttgctttacagaaagcacaactgaggagattgagctggaagatgttgagtgttagccaaggctgggcctgactgcattcctacctcaagaggaaaccattctccaacaaaaagagcaagcacagctattatacccattgtgtgtggtcctgttgcagcccgctcctaacaggacagtgggagattaacaacattgactgcatggagttgaggactgtggatgggacaaagctagtattaggactcgcgctaagttcaaggagaagagtgattgaggctttgaaccaggagcttcgcttggctgcagcatcagggccgtgctgacacataaccaatggcagtcggaggaaccaggctctgggccaggacagtttccagtgctgtgaagaaacgaggtgagcagtatccaaggggctcaaggataatctgctgatttctttctcgtctttcacacagagaggccaccagcaggaaagcagtgggagttgggtagctgctggggccagcatgtcctcttccacactacctgcctttcagaactctgctcttccgcttgtcccgaagtcagggctggcaatcttcataatcaaaggggagttggaaaagaacacccactaagaggcctccatggcagatgtcaattaaattgcccccccccccaaatctcatgacagtttattcacatcagtagtgtgggaagttacaatgttttttttaaaaaaagcttctttccttggctttccatttctttgaaaaaagggtttcttttgaatttttaaagctctgccatactgaacattcctgtggaaaggtttaaaatgcagagcctgaggtttttgcttttcagaaaaataaaaatcatagagattagctagtgtaaatgttaagctataaattatgtttcaaactgtgaaaaaaaaaagttttaaagaatgaatgaaataaaacctgaaaataaagccctggtcctttg

SEQ ID NO: 6 - protein, 200 aa, Homo sapiens
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVEC

SEQ ID NO: 7 - protein, 199 aa, Homo sapiens
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKGNPLGLVIIHEWF

SEQ ID NO: 8 - protein, 352 aa, Artificial Sequence
  Fusion protein mouse VSTM5-ECD fused to mouse IgG2a Fc
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 9 - protein, 456 aa, Artificial Sequence [sequence omitted]

SEQ ID NO: 10 - protein, 389 aa, Artificial Sequence
  Fusion protein human VSTM5-ECD fused to mouse IgG2a Fc
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 11 - protein, 199 aa, Homo sapiens
MRPLRCGERTQGIPLGLLAFWVTAARCLQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHFVAVFFALLAAVAVVLISLMWVCNQCAYKFQRKRRYKLKESTTEEIEMKEVEC

SEQ ID NO: 12 - protein, 18 aa, Homo sapiens
TIEWTYSSNWGTQKIVEW

SEQ ID NO: 13 - protein, 19 aa, Homo sapiens
TIEWTYSSNWGTQKIVEWK

SEQ ID NO: 14 - protein, 19 aa, Homo sapiens
PTIEWTYSSNWGTQKIVEW

SEQ ID NO: 15 - protein, 20 aa, Homo sapiens
PTIEWTYSSNWGTQKIVEWK

SEQ ID NO: 16 - protein, 28 aa, Homo sapiens
GYYVITVTERLGSSQFGTIVLHVSEILY

SEQ ID NO: 17 - protein, 23 aa, Homo sapiens
GYYVITVTERLGSSQFGTIVLHV

SEQ ID NO: 18 - protein, 27 aa, Homo sapiens
GYYVITVTERLGSSQFGTIVLHVSEIL

SEQ ID NO: 19 - protein, 12 aa, Homo sapiens
RVCTFDNGSIQL

SEQ ID NO: 20 - protein, 17 aa, Homo sapiens
RVCTFDNGSIQLFSVGV

SEQ ID NO: 21 - protein, 12 aa, Homo sapiens
VCTFDNGSIQLF

SEQ ID NO: 22 - protein, 107 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 23 - protein, 106 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 24 - protein, 105 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 25 - protein, 108 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 26 - protein, 109 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 27 - protein, 110 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 28 - protein, 111 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 29 - protein, 112 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 30 - protein, 113 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 31 - protein, 114 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 32 - protein, 115 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 33 - protein, 116 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 34 - protein, 118 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 35 - protein, 120 aa, Homo sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 36 - protein, 107 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 37 - protein, 108 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 38 - protein, 109 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 39 - protein, 110 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 40 - protein, 111 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 41 - protein, 112 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 42 - protein, 113 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 43 - protein, 114 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 44 - protein, 115 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 45 - protein, 116 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 46 - protein, 117 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 47 - protein, 118 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 48 - protein, 119 aa, Homo sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 49 - protein, 106 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 50 - protein, 107 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 51 - protein, 108 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 52 - protein, 109 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 53 - protein, 110 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 54 - protein, 111 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 55 - protein, 112 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEIL

SEQ ID NO: 56 - protein, 113 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILY

SEQ ID NO: 57 - protein, 114 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYE

SEQ ID NO: 58 - protein, 115 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYED

SEQ ID NO: 59 - protein, 116 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDL

SEQ ID NO: 60 - protein, 117 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLH

SEQ ID NO: 61 - protein, 118 aa, Homo sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHF

SEQ ID NO: 62 - protein, 96 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 63 - protein, 97 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 64 - protein, 98 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 65 - protein, 99 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 66 - protein, 100 aa, Homo sapiens
QATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 67 - protein, 95 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 68 - protein, 96 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 69 - protein, 97 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 70 - protein, 98 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 71 - protein, 99 aa, Homo sapiens
ATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 72 - protein, 94 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 73 - protein, 95 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 74 - protein, 97 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 75 - protein, 98 aa, Homo sapiens
TINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 76 - protein, 93 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 77 - protein, 94 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 78 - protein, 95 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 79 - protein, 96 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 80 - protein, 97 aa, Homo sapiens
INATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 81 - protein, 92 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIV

SEQ ID NO: 82 - protein, 93 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVL

SEQ ID NO: 83 - protein, 94 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 84 - protein, 95 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 85 - protein, 96 aa, Homo sapiens
NATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 86 - protein, 103 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 87 - protein, 104 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 88 - protein, 105 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 89 - protein, 106 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 90 - protein, 107 aa, Homo sapiens
SLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 91 - protein, 102 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 92 - protein, 103 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 93 - protein, 104 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 94 - protein, 105 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 95 - protein, 106 aa, Homo sapiens
LYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 96 - protein, 101 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 97 - protein, 102 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 98 - protein, 103 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 99 - protein, 104 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 100 - protein, 105 aa, Homo sapiens
YIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 101 - protein, 100 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 102 - protein, 101 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 103 - protein, 102 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 104 - protein, 103 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 105 - protein, 104 aa, Homo sapiens
IPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 106 - protein, 99 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLH

SEQ ID NO: 107 - protein, 100 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHV

SEQ ID NO: 108 - protein, 101 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVS

SEQ ID NO: 109 - protein, 102 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSE

SEQ ID NO: 110 - protein, 103 aa, Homo sapiens
PQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEI

SEQ ID NO: 111 - protein, 379 aa, Artificial Sequence
  Fusion protein human VSTM5-ECD fused to human IgG1 Fc C220S
MRPLPSGRRKTRGISLGLFALCLAAARCLQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 233 aa, Mus Musculus
EPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 113 - protein, 232 aa, Homo sapiens
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 232 aa, Homo sapiens
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 217 aa, Homo sapiens
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 217 aa, Mus Musculus
APNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 117 - protein, 2 aa, Artificial sequence
  Synthetic polypeptide linker
GS

SEQ ID NO: 118 - protein, 4 aa, Artificial sequence
  Synthetic polypeptide linker
GSGS

SEQ ID NO: 119 - protein, 2 aa, Artificial sequence
  Synthetic polypeptide linker
AS

SEQ ID NO: 120 - protein, 4 aa, Artificial sequence
  Synthetic polypeptide linker
GGGS

SEQ ID NO: 121 - protein, 5 aa, Artificial sequence
  Synthetic polypeptide linker
GGGGS

SEQ ID NO: 122 - protein, 10 aa, Artificial sequence
  Synthetic polypeptide linker
GGGGSGGGGS

SEQ ID NO: 123 - protein, 15 aa, Artificial sequence
  Synthetic polypeptide linker
GGGGSGGGGSGGGGS

SEQ ID NO: 124 - protein, 20 aa, Artificial sequence
  Synthetic polypeptide linker
GGGGSGGGGSGGGGSGGGGS

SEQ ID NO: 125 - protein, 12 aa, Artificial sequence
  Synthetic polypeptide linker
AGAAAKGAAAKA

SEQ ID NO: 126 - protein, 17 aa, Artificial sequence
  Synthetic polypeptide linker
AGAAAKGAAAKGAAAKA

SEQ ID NO: 127 - protein, 22 aa, Artificial sequence
  Synthetic polypeptide linker
AGAAAKGAAAKGAAAKGAAAKA

SEQ ID NO: 128 - protein, 27 aa, Artificial sequence
  Synthetic polypeptide linker
AGAAAKGAAAKGAAAKGAAAKGAAAKA

SEQ ID NO: 129 - protein, 7 aa, Artificial sequence
  Synthetic polypeptide linker
AGAAAKA

SEQ ID NO: 130 - protein, 351 aa, Artificial Sequence
  Fusion protein human VSTM5-ECD fused to human IgG1 Fc
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 361 aa, Artificial Sequence
  Fusion protein human VSTM5-ECD fused to mouse IgG2a Fc
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDGSENLYFQGSGEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 132 - protein, 172 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVEC

SEQ ID NO: 133 - protein, 428 aa, Artificial Sequence
  Fusion protein human VSTM5-ECD fused to EGFP
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHFVAVILAFLAAVAAVLISLMWVCNKCAYKFQRKRRHKLKESTTEEIELEDVECRILQSTVPRARDPPVATMVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 134 - protein, 337 aa, Artificial Sequence
  Fusion protein human VSTM5-ECD fused to human IgG1 Fc C220S
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 347 aa, Artificial Sequence
  Fusion protein human VSTM5-ECD fused to human IgG1 Fc C220S (G4S)x2 linker
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTGGGGSGGGGSEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 338 aa, Artificial Sequence
  Fusion protein mouse VSTM5-ECD fused to mouse IgG2a Fc
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 137 - protein, 348 aa, Artificial Sequence
  Fusion protein mouse VSTM5-ECD fused to mouse IgG2a Fc (G4S)x2 linker
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTGGGGSGGGGSEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 138 - protein, 11 aa, Artificial Sequence
  synthetic polypeptide linker
GSENLYFQGSG

SEQ ID NO: 139 - protein, 25 aa, Artificial Sequence
  synthetic polypeptide linker
AEAAAKEAAAKAHELICALLINKER

SEQ ID NO: 140 - protein, 22 aa, Artificial Sequence
  synthetic polypeptide linker
AEAAAKEAAAKEAAAKEAAAKA

SEQ ID NO: 141 - protein, 14 aa, Artificial Sequence
  synthetic polypeptide linker
GGEAAAKEAAAKGG

SEQ ID NO: 142 - protein, 19 aa, Artificial Sequence
  synthetic polypeptide linker
GGGGSGGGGSGAGSGGGGS

SEQ ID NO: 143 - protein, 11 aa, Artificial Sequence
  synthetic polypeptide linker
GGGGGSGGGGS

SEQ ID NO: 144 - protein, 6 aa, Artificial Sequence
  synthetic polypeptide linker
GGGGSG

SEQ ID NO: 145 - protein, 4 aa, Artificial Sequence
  synthetic polypeptide linker
GSGG

SEQ ID NO: 146 - protein, 5 aa, Artificial Sequence
  synthetic polypeptide linker
GSGGS

SEQ ID NO: 147 - protein, 352 aa, Artificial Sequence
  Fusion protein mouse VSTM5-ECD fused to mouse IgG2a Fc N297A
LQSQGVSLYIPQSAINATVQQDILLSVDYICHGVPTIEWKYTPNWGVQRIVEWKPGTPANVSQSHRDRVCTFDNGSIQLFNVSVKDSGYYIVTVTEHPGSSQSGTILLRVSEIRYEDLHEPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 148 - protein, 233 aa, Artificial Sequence
  Fusion protein mouse VSTM5-ECD fused to mouse IgG2a Fc N297A
EPRGPTIKPCPPCKCPAPNLLGGPSVFIFPPKIKDVLMISLSPIVTCVVVDVSEDDPDVQISWFVNNVEVHTAQTQTHREDYASTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 149 - protein, 351 aa, Artificial Sequence
  Fusion protein human VSTM5-ECD fused to human IgG1 Fc C220S N297A
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTIVLHVSEILYEDLHEPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 232 aa, Artificial Sequence
  human IgG1 Fc C220S N297A
EPKSSDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 105 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 152 - protein, 104 aa, Homo Sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 153 - protein, 103 aa, Homo Sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGT

SEQ ID NO: 154 - protein, 106 aa, Homo Sapiens
LQSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

SEQ ID NO: 155 - protein, 105 aa, Homo Sapiens
QSQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

SEQ ID NO: 156 - protein, 104 aa, Homo Sapiens
SQGVSLYIPQATINATVKEDILLSVEYSCHGVPTIEWTYSSNWGTQKIVEWKPGTQANISQSHKDRVCTFDNGSIQLFSVGVRDSGYYVITVTERLGSSQFGTI

(19) United States
(12) Patent Application Publication
(10) Pub. No.: US 2016/0347814 A1
(43) Pub. Date: Dec. 1, 2016

(54) VSTM5 POLYPEPTIDES AND USES THEREOF AS A DRUG FOR TREATMENT OF CANCER, INFECTIOUS DISEASES AND IMMUNE RELATED DISEASES

(71) Applicant: Compugen Ltd., Holon (IL)

(72) Inventors: Zurit LEVINE, Herzliyya (IL); Galit ROTMAN, Herzliyya (IL); Liat DASSA, Yehud (IL); Ofer LEVY, Moshav Mesisraelat Zion (IL); Gad S. COJOCARU, Ramat HaSharon (IL); Amir TOPORIK, Pardes Hannah Karkur (IL); Yossef KLIGER, Rishon LeZiyyon (IL); Ilan VAKNIN, Modiin (IL); Iris HECHT, Tel Aviv-Yafo (IL)

(21) Appl. No.: 15/066,905

(22) Filed: Mar. 10, 2016

Related U.S. Application Data
(63) Continuation of application No. PCT/IL2014/050809, filed on Sep. 11, 2014.
(60) Provisional application No. 61/876,328, filed on Sep. 11, 2013.

(51) Int. Cl. C07K 14/705; A61K 45/06; G01N 33/574; A61K 39/39

(52) U.S. Cl. CPC C07K 14/705; A61K 39/39; A61K 45/06; G01N 33/57492; C07K 2319/31; C07K 2319/30; A61K 38/00

(57) ABSTRACT

This invention relates to VSTM5 proteins, soluble molecules and fusions thereof which are suitable targets for drug development and for treatment of immune related disorders, immunotherapy, treatment of cancer, infectious disorders and/or sepsis.

### FIELD OF THE INVENTION

The present invention, in at least some aspects, relates to VSTM5 proteins, soluble molecules and fusions thereof, and cells which express same and the use thereof as targets for drug development and for treatment of immune related disorders, immunotherapy, treatment of cancer, infectious disorders and/or sepsis.

### BACKGROUND OF THE INVENTION

Naïve T cells must receive two independent signals from antigen-presenting cells (APC) in order to become productively activated. The first, Signal 1, is antigen-specific and occurs when T cell antigen receptors encounter the appropriate antigen-MHC complex on the APC. The fate of the immune response is determined by a second, antigen-independent signal (Signal 2) which is delivered through a T cell costimulatory molecule that engages its APC-expressed ligand. This second signal could be either stimulatory (positive costimulation) or inhibitory (negative costimulation or coinhibition). In the absence of a costimulatory signal, or in the presence of a coinhibitory signal, T-cell activation is impaired or aborted, which may lead to a state of antigen-specific unresponsiveness (known as T-cell anergy), or may result in T-cell apoptotic death.

Costimulatory molecule pairs usually consist of ligands expressed on APCs and their cognate receptors expressed on T cells. The prototype ligand/receptor pairs of costimulatory molecules are B7/CD28 and CD40/CD40L. The B7 family consists of structurally related, cell-surface protein ligands, which may provide stimulatory or inhibitory input to an immune response. Members of the B7 family are structurally related, with the extracellular domain containing at least one variable or constant immunoglobulin domain.

Both positive and negative costimulatory signals play critical roles in the regulation of cell-mediated immune responses, and molecules that mediate these signals have proven to be effective targets for immunomodulation. Based on this knowledge, several therapeutic approaches that involve targeting of costimulatory molecules have been developed, and were shown to be useful for prevention and treatment of cancer by turning on, or preventing the turning off, of immune responses in cancer patients and for prevention and treatment of autoimmune diseases and inflammatory diseases, as well as rejection of allogenic transplantation, each by turning off uncontrolled immune responses, or by induction of “off signal” by negative costimulation (or coinhibition) in subjects with these pathological conditions.

Manipulation of the signals delivered by B7 ligands has shown potential in the treatment of autoimmunity, inflammatory diseases, and transplant rejection.

Therapeutic strategies include blocking of costimulation using monoclonal antibodies to the ligand or to the receptor of a costimulatory pair, or using soluble fusion proteins composed of the costimulatory receptor that may bind and block its appropriate ligand. Another approach is induction of co-inhibition using soluble fusion protein of an inhibitory ligand. These approaches rely, at least partially, on the eventual deletion of auto- or allo-reactive T cells (which are responsible for the pathogenic processes in autoimmune diseases or transplantation, respectively), presumably because in the absence of costimulation (which induces cell survival genes) T cells become highly susceptible to induction of apoptosis. Thus, novel agents that are capable of modulating costimulatory signals, without compromising the immune system's ability to defend against pathogens, are highly advantageous for treatment and prevention of such pathological conditions.

Costimulatory pathways play an important role in tumor development. Interestingly, tumors have been shown to evade immune destruction by impeding T cell activation through inhibition of co-stimulatory factors in the B7-CD28 and TNF families, as well as by affecting regulatory T cells, which inhibit anti-tumor T cell responses (see Wang (2006), “Immune Suppression by Tumor Specific CD4⁺ Regulatory T cells” in Cancer. Semin. Cancer. Biol. 16:73-79; Greenwald, et al. (2005), “The B7 Family Revisited”, Ann. Rev. Immunol. 23:515-48; Watts (2005) “TNF/TNFR Family Members in Co-stimulation of T Cell Responses”, Ann Rev. Immunol. 23:23-68; Sadum, et al. (2007), “Immune Signatures of Murine and Human Cancers Reveal Unique Mechanisms of Tumor Escape and New Targets for Cancer Immunotherapy”, Clin. Canc. Res. 13(13): 4016-4025). Such tumor expressed co-stimulatory molecules have become attractive cancer biomarkers and may serve as tumor-associated antigens (TAAs). Furthermore, costimulatory pathways have been identified as immunologic checkpoints that attenuate T cell dependent immune responses, both at the level of initiation and effector function within tumor metastases. As engineered cancer vaccines continue to improve, it is becoming clear that such immunologic checkpoints are a major barrier to the vaccines' ability to induce therapeutic anti-tumor responses. In that regard, costimulatory molecules can serve as adjuvants for active (vaccination) and passive (antibody-mediated) cancer immunotherapy, providing strategies to thwart immune tolerance and stimulate the immune system.

In addition, such agents could be of use in other types of cancer immunotherapy, such as adoptive immunotherapy, in which tumor-specific T cell populations are expanded and directed to attack and kill tumor cells. Agents capable of augmenting such anti-tumor response have great therapeutic potential and may be of value in the attempt to overcome the obstacles to tumor immunotherapy. Recently, novel agents that modulate several costimulatory pathways were indeed introduced to the clinic as cancer immunotherapy.

Regulating costimulation using agonists and/or antagonists to various costimulatory proteins has been extensively studied as a strategy for treating autoimmune diseases, graft rejection, allergy and cancer. This field has been clinically pioneered by CTLA4-Ig (Abatacept, Orencia®) which is approved for treatment of RA, mutated CTLA4-Ig (Belatacept, Nulojix®) for prevention of acute kidney transplant rejection and by the anti-CTLA4 antibody (Ipilimumab, Yervoy®), recently approved for the treatment of melanoma. Other costimulation regulators are currently in advanced stages of clinical development including anti-PD-1 antibody (BMS-936558) which is in development for treatment of Non-Small Cell Lung cancer and other cancers. Furthermore, such agents are also in clinical development for viral infections, for example the anti PD-1 Ab, MDX-1106, which is being tested for treatment of hepatitis C, and the anti-CTLA-4 Ab CP-675,206 (tremelimumab) which is in a clinical trial in hepatitis C virus-infected patients with hepatocellular carcinoma.

In addition recently researchers have developed compounds which target regulatory T cells (iTregs) for use in immunotherapy. With respect thereto it is known that inducible regulatory T cells (iTregs) are commonly seen in many tumors, and form the major subset of immune suppressor cells in the tumor tissue and moreover represent a major tumor resistance mechanism from immune surveillance. Accordingly, iTregs are therefore viewed as important cellular targets for cancer therapy.

Multiple immune-checkpoint receptors, such as CTLA4, PD-1, TIM3 and LAGS, are expressed at high levels on the surface of iTregs and directly promote Treg cell-mediated suppression of effector immune responses. Therefore, some immune-checkpoint antibodies may, in addition to increasing CTL immunity, further block the immunosuppressive activity of iTregs and thereby enhance anti-tumor immunity. For example, CTLA4 blockade by ipilimumab both enhances effector T cell activity, and inhibits Treg immunosuppressive activity.

B cells play a critical role in recognition of foreign antigens and they produce the antibodies necessary to provide protection against various types of infectious agents. T cell help to B cells is a pivotal process of adaptive immune responses. Follicular helper T (Tfh) cells are a subset of CD4⁺ T cells specialized in B cell help (reviewed by Crotty, Annu. Rev. Immunol. 29: 621-663, 2011). Tfh cells express the B cell homing chemokine receptor, CXCR5, which drives Tfh cell migration into B cell follicles within lymph nodes in a CXCL13-dependent manner. The requirement of Tfh cells for B cell help and T cell-dependent antibody responses indicates that this cell type is of great importance for protective immunity against various types of infectious agents, as well as for rational vaccine design. In addition, regulatory B cells (Bregs) have a role in impairing effective clearance of tumors. The mechanisms for Bregs effects in cancer are not well understood, however one proposed mechanism is through inhibition of cytotoxic CD8⁺ T cells.

NK cells are effector lymphocytes of the innate immune system that are known to be involved in killing of pathological or diseased cells such as cancer and infected cells and pathogens. Natural killer cells have the capacity to kill cellular targets and produce cytokines without prior specific sensitization. NK cells are unique, as they have the ability to recognize stressed cells in the absence of antibodies and MHC, allowing for a much faster immune reaction. This role is especially important because harmful cells that are missing MHC 1 markers cannot be detected and destroyed by other immune cells, such as T cells.

Induction of immune tolerance has long been considered the “holy grail” for autoimmune disease therapy. The immune system has the reciprocal tasks to protect the host against invading pathogens, but simultaneously to prevent damage resulting from unwanted reactions to self-antigens. The latter part is known as immune tolerance and performed by a complex set of interactive and complementary pathways, which regulate immune responses. T cells have the ability to react to a variety of antigens, both self and nonself. Therefore, there are many mechanisms that exist naturally to eliminate potentially self-reactive responses—this is known as natural tolerance. The main mechanism for eliminating potential auto-reactive T cells occurs in the thymus and is known as central tolerance. Some potentially autoreactive T cells escape central tolerance and, therefore, peripheral tolerance mechanisms also exist. Despite these mechanisms, some self-reactive T cells may ‘escape’ and be present in the repertoire; it is believed that their activation may lead to autoimmune disease.

Studies on therapeutic tolerance have attempted to induce and amplify potent physiological mechanisms of tolerance in order to eliminate or neutralize self-reactive T cells and prevent or treat autoimmune diseases. One way to induce tolerance is by manipulation of the interaction between costimulatory ligands and receptors on antigen presenting cells (APCs) and lymphocytes.

CTLA-4 is the most extensively studied costimulatory molecule which down-regulates immune responses. The attributes of immunosuppressive qualities and capacity to induce tolerance have made its recognition as a potential immunotherapeutic agent for autoimmune mediated inflammatory disorders. Abatacept (commercial name. Orencia) is a fusion protein composed of the ECD (extracellular domain) of CTLA-4 fused to the Fc fragment of hIgG1. Abatacept is believed to induce costimulation blockade, which has been approved for treating patients with rheumatoid arthritis, by effectively interfering with the inflammatory cascade.

Induction of disease control with the current therapies, followed by progressive withdrawal in parallel with re-establishing immune tolerance, may be an attractive approach in the future of autoimmune therapies. Furthermore, due to their immune specificity, in the absence of global immunosuppression, such therapies should be safe for chronic use.

Certain mechanisms are known to be widespread in various autoimmune diseases. T helper type 1 (Th1) cells are induced by IL-12 and produce IFN-γ; while T helper type 2 (Th2) cells secrete IL-4, IL-5 and IL-13. Th1 cells can mediate proinflammatory or cell-mediated immune responses, whereas Th2 cells mainly promote certain types of humoral immunity. Some immune related diseases, such as autoimmune reactions, inflammation, chronic infection and sepsis, are characterized by a dysregulation of the pro- versus anti-inflammatory tendencies of the immune system, as well as an imbalance in the Th1 versus Th2 cytokine balance. During inflammation, induction of a shift in the balance from Th1 to Th2 protects the organism from systemic ‘overshooting’ with Th1/pro-inflammatory cytokines, by reducing the inflammatory tendencies of the immune system Immunomodulatory therapies that are associated with a Th1 to Th2 immune shift have protective effects in Th1-mediated autoimmune diseases, such as multiple sclerosis and rheumatoid arthritis. For example, Laquinimod, which has demonstrated efficacy in animal models of several autoimmune diseases including MS, shows immunomodulatory effects through Th1/Th2 shift, and does not lead to immunosuppression. Glatiramer acetate (Copaxone®) also induces Th1/Th2 shift with decreased secretion of proinflammatory cytokines, and increased secretion of antiinflammatory cytokines. Furthermore, glatiramer acetate-specific Th2 cells are able to migrate across the blood-brain barrier and cause in situ bystander suppression of autoaggressive Th1 T cells.

Certain immune cells and immune cell signal transduction pathways are promising targets for new agents for treating immune disorders. For example Th1, Th17, Th2 and regulatory T cells (Tregs) play important roles in modulating autoimmunity and inflammation. Mounting evidence from numerous studies shows the importance of these immune cells in disorders such as rheumatoid arthritis, inflammatory bowel disease, multiple sclerosis, psoriasis, lupus erythematosus, type 1 diabetes and uveitis. Most existing therapies target only one pathway at a time.

### BRIEF SUMMARY OF THE INVENTION

According to at least some embodiments the present invention provides VSTM5 soluble proteins, variants, fragments and fusion proteins thereof, cells or vectors which express same, and compositions comprising same, and methods of use thereof for drug development, for treatment of cancer, infectious diseases, immune related diseases such as autoimmune, inflammatory and allergic diseases, and/or for reducing the undesirable immune activation that follows gene therapy, or for reducing undesirable immune responses during transplant, e.g., cell, tissue and organ transplants, GVHD, adverse immune responses that may occur during pregnancy, and methods and compositions for treatment of same. According to at least some embodiments, the present invention provides soluble VSTM5 proteins, variants, fragments and fusion proteins thereof, and cells which express same that may be used to modulate (enhance or inhibit) the effects of VSTM5 on immunity. As VSTM5 is disclosed herein to elicit immunosuppressive effects on immunity, soluble VSTM5 polypeptides and fusion proteins which antagonize VSTM5 activity may optionally be used as immunostimulatory agents for use in treating diseases wherein immunostimulation is therapeutically beneficial such as cancer, infection and sepsis. Conversely, according to at least some embodiments, soluble VSTM5 polypeptides and fusion proteins which agonize or mimic VSTM5 activity may optionally be used as immuninhibitors and used in treating diseases wherein immunoinhibition is therapeutically beneficial such as immune disorders including autoimmunity, allergy and inflammation, or to suppress undesired immune reactions such as in cell or gene therapy or in recipients of transplanted cells, tissues or organs.

According to at least some embodiments, there is provided VSTM5 proteins, e.g., soluble and non-soluble VSTM5 ECD polypeptides and fragments or conjugates, multimers (heteromultimers and homomultimers), and/or fusions thereof, which modulate at least one effect of VSTM5 on immunity. Further, in at least some embodiments, the invention relates to diagnostic and therapeutic compositions comprising same, and the use thereof for modulating (antagonizing or agonizing) one or more of the effects of VSTM5 on immunity in e.g., cancer, infectious diseases, sepsis and immune related diseases such as autoimmune, allergic and inflammatory conditions, e.g., conditions associated with VSTM5 expression by diseased, stromal or antigen-presenting cells, optionally wherein the endogenous disease pathology is enhanced or inhibited by VSTM5-mediated effects on immunity. Further, in at least some embodiments, the invention relates to diagnostic use thereof, e.g., for detecting or aiding in the diagnosis of cancer, infectious diseases, sepsis and immune related diseases such as autoimmune, allergic and inflammatory conditions, e.g., conditions associated with VSTM5 expression by diseased, stromal or antigen-presenting cells, optionally wherein the endogenous disease pathology is enhanced or inhibited by VSTM5-mediated effects on immunity.

Thus, in various embodiments, VSTM5 soluble proteins, variants, fragments and fusion proteins thereof and compositions comprising same, which have VSTM5-like inhibitory activity on immune responses and/or enhance VSTM5 immune inhibitory activity, are understood by the present inventors to be useful for treatment of immune related diseases and/or for reducing the undesirable immune activation that follows gene transfer or cell therapy or after transplantation of allogeneic or xenogeneic cells, tissues or organs. Furthermore, in various embodiments, VSTM5 soluble proteins, variants, fragments and fusion proteins thereof and compositions comprising same, which inhibit pathogenic T cells and/or NK cells, should attenuate autoimmunity and autoreactive inflammation, allergic responses and inflammation, and thus to be useful for treatment of immune related diseases such as autoimmune, inflammatory and allergic conditions, and/or for reducing the undesirable immune activation that follows gene or cell therapy or after transplantation of heterologous, allogeneic or xenogeneic cells, tissues or organs such as GVHD.

Accordingly, in at least some embodiments, the present invention in some embodiments provides immunostimulatory VSTM5 soluble proteins, variants, fragments and fusion proteins thereof and compositions comprising same, and methods of use thereof for drug development, for immunotherapy and for treatment of cancer, infectious disorders and/or sepsis; and for methods of treatment same. Without wishing to be limited by a single hypothesis, immunostimulatory VSTM5 soluble proteins, variants, fragments and fusion proteins thereof and compositions comprising same which are useful for immunotherapy and for treatment of cancer, infectious disorders and/or sepsis, reduce VSTM5 inhibitory activity on immune responses of cells, such as NK cells, T cells such as CD4⁺ T cells, CD8⁺ T cells or CTLs and Th17 cells. Also, VSTM5 soluble proteins may elicit an inhibitory effect on certain cells that suppress immunity, e.g., antitumor immunity, such as T regulatory cells (Tregs) and myeloid derived suppressor cells (MDSCs) and thereby further promote immunity by inhibiting the inhibitory effects of these immunosuppressive cells on immunity.

Again without wishing to be limited by a single hypothesis, as VSTM5 has a suppressive effect on immune cells such as CD4⁺ T cells, CD8⁺ or CTLs and NK cells, which cells are known to be involved in killing of pathological or diseased cells such as cancer and infected cells and pathogens, VSTM5 soluble proteins variants, fragments and fusion proteins thereof and compositions comprising same which antagonize the inhibitory effects of VSTM5 on T cell or NK cell-mediated immunity may optionally be used for the treatment of cancer, infectious diseases and sepsis and other indications wherein enhanced immune responses and/or the depletion of target cells is therapeutically desired.

Moreover, although again without wishing to be limited by a single hypothesis, as VSTM5 has an inhibitory effect on specific immune cells such as CD4⁺ T cells, CD8⁺ T cells or CTLs, and NK cells, which cells are known to be involved in the pathology of certain immune conditions such as autoimmune and inflammatory disorders, as well as eliciting a potentiating effect on Tregs or MDSCs, VSTM5 soluble proteins variants, fragments and fusion proteins thereof and compositions comprising same which potentiate or agonize the effects of VSTM5 on immunity may optionally be used for treating conditions wherein the suppression of T cell or NK mediated immunity and/or the induction of immune tolerance or prolonged suppression of antigen-specific immunity is therapeutically desirable, e.g., the treatment of autoimmune, inflammatory or allergic conditions, and/or the suppression of undesired immune responses such as to cell or gene therapy, adverse immune responses during pregnancy, and adverse immune responses to transplanted heterologous, allogeneic or xenogeneic cells, organs and tissues and for inhibiting or preventing the onset of graft versus host disease (GVHD) after transplant.

Therefore, in one embodiment the present invention broadly relates to the development of novel “immunomodulatory VSTM5 polypeptides” wherein this includes VSTM5 polypeptides that antagonize or block the effects of VSTM5 on immunity and particularly the effects of VSTM5 on specific types of immune cells and cytokine production (i.e., “immunostimulatory VSTM5 polypeptides or fusion proteins”) and VSTM5 polypeptides that agonize or mimic the effects of VSTM5 on immunity and particularly the effects of VSTM5 on specific types of immune cells and cytokine production (i.e., “immunoinhibitory VSTM5 polypeptides or fusion proteins”).

By “immunostimulatory VSTM5 soluble protein” or “immunostimulatory VSTM5 ECD” or “immunostimulatory VSTM5 fusion protein”, or “immunostimulatory VSTM5 protein” or “immunostimulatory VSTM5 therapeutic and/or diagnostic agent” used herein interchangeably, it is meant any VSTM5 soluble proteins, variants, fragments and fusion proteins thereof and/or compositions comprising same, which stimulate the immune response upon administration to a subject, in order to enhance immunity against cancer cells, infectious diseases, particularly chronic infections or sepsis.

Immunostimulatory VSTM5 soluble proteins comprise an expanding class of agents, which are either antagonists of immune-repressor molecules or agonists of immune-activating receptors. This new class of therapeutic agents has the ability to enhance anti-tumour immunity, comprising a new and promising strategy in cancer therapy.

Reduction of the immunoinhibitory activity of VSTM5 is believed to be desirable in situations in which VSTM5 is abnormally upregulated, and/or situations in which decreased activity of VSTM5 leading to stimulation of immune responses which should have a beneficial effect, such as for example, for immunotherapy and for treatment of cancer, infectious disorders and/or sepsis, although without wishing to be limited by a single hypothesis. Thus, as used herein, “immunostimulatory VSTM5 soluble protein” according to at least some embodiments of the present invention, is a therapeutic agent which reduces or eliminates the inhibitory effect of VSTM5 on immune responses, leading to stimulation of immune responses and/or reversal of antigen-specific immune tolerance or immunosuppression, e.g. by promoting T cell and NK cell-mediated cytotoxic immunity, and in particular by potentiating the cytotoxic activity of a subjects endogenous immune cells, e.g., CD4⁺ T cells, CD8⁺T cells and TH17 cells and NK cells. The subject immunostimulatory VSTM5 therapeutic agents according to at least some embodiments of the invention should have a beneficial effect in treating disorders and/or conditions and/or situations in which decreased activity of VSTM5 is desirable, and thus can be used for treatment of such disorders and/or conditions and/or situations.

By “immunoinhibitory VSTM5 soluble protein” or “immunoinhibitory VSTM5 ECD” or “immunoinhibitory VSTM5 fusion protein” or “immunoinhibitory VSTM5 therapeutic and/or diagnostic agent”, used herein interchangeably, it is meant any VSTM5 soluble protein, variant, fragment and fusion protein thereof and/or composition comprising same, for which its administration to a patient leads to reduction of undesirable immune responses. In particular, VSTM5 soluble protein, variant, fragment and fusion protein thereof and/or composition comprising same, should be suitable for the treatment or prophylaxis of immune related diseases such as autoimmune, inflammatory and allergic conditions and/or for reducing the undesirable immune activation which often occurs after gene or cell therapy or after cell, tissue or organ transplant. Proteins that agonize or mimic VSTM5-like inhibitory activity on immune responses and/or which enhance VSTM5 immune inhibitory activity, are expected to inhibit pathogenic T cells e.g., CD4⁺ T cells, and CD8⁺T cells and inhibit TH17 cells and/or NK cells which are involved in the pathology of numerous immune diseases or conditions wherein the immune system is abnormally upregulated such as during chronic autoimmune, inflammatory and allergic conditions wherein such cells may give rise to tissue destruction or other undesired autoimmune, allergic or inflammatory effects, again without wishing to be limited by a single hypothesis.

Enhancement of the immunoinhibitory activity of VSTM5, without wishing to be limited by a single hypothesis, is believed to be desirable in situations in which VSTM5 is abnormally downregulated, and/or situations in which increased activity of VSTM5 is likely to have a beneficial effect, such as for example, and without wishing to be limited by a single theory, for treatment of immune related conditions and/or for reducing the undesirable immune activation that follows gene or cell therapy and after transplant. As used herein, “immunoinhibitory VSTM5 soluble protein” according to at least some embodiments of the present invention, is a therapeutic agent which mimics or increases the inhibitory activity of VSTM5. Such immunoinhibitory VSTM5 soluble protein is likely to have a beneficial effect in disorders and/or conditions and/or situations in which inhibitory activity of VSTM5 is desirable, and thus can be used for treatment and prophylaxis of such disorders and/or conditions and/or situations. Thus, according to at least some embodiments of the invention, VSTM5 soluble proteins, variants, fragments and fusion proteins thereof and/or compositions comprising same, that function as immunoinhibitory agents can be used as therapeutic agents in situations where the inhibitory effects of VSTM5 on immunity is desirable, and/or situations in which increased inhibitory activity of VSTM5 is likely to have a beneficial effect, such as for example the treatment of immune related conditions such as chronic autoimmune, allergic and inflammatory conditions and/or for reducing the undesirable immune activation that often follows gene therapy, and transplant and which in some individuals may occur during pregnancy.

According to at least some embodiments there is provided an isolated or recombinant VSTM5 polypeptide comprising a fragment of VSTM5 ECD, consisting essentially of an amino acid sequence as set forth in any one of SEQ ID NOs: 1, 12-110, 151-156, or variant thereof that possesses at least 90 or 95% sequence identity therewith.

According to at least some embodiments there is provided an isolated or recombinant VSTM5 polypeptide comprising a multimer or a fusion polypeptide comprising a fragment of VSTM5 ECD, which fragment consists essentially of an amino acid sequence as set forth in any one of SEQ ID NOs: 1, 12-110, 151-156, or variant thereof that possesses at least 90 or 95% sequence identity therewith, wherein such multimer or fusion polypeptide may comprise one or more of such VSTM5 fragments, e.g., 1-10 fragments, directly linked or attached to one another or fused via a linker or multimerization domain.

According to at least some embodiments there is provided a fusion protein comprising the polypeptide comprising a fragment of VSTM5 ECD, consisting essentially of an amino acid sequence as set forth in any one of SEQ ID NOs: 1, 12-110, 151-156, or variant thereof that possesses at least 95% sequence identity therewith, joined to a second fusion partner composed of a heterologous sequence (i.e., a non-VSTM5 polypeptide), fused together directly or indirectly via a peptide linker sequence or a chemical linker. Optionally, the heterologous sequence comprises at least a portion of an immunoglobulin molecule. Optionally and preferably, the immunoglobulin molecule portion is an immunoglobulin heavy chain constant region F_{c} fragment. Optionally and more preferably, the immunoglobulin heavy chain constant region is derived from an immunoglobulin isotype selected from the group consisting of an IgG1, IgG2, IgG3, IgG4, IgM, IgE, IgA and IgD. Optionally, the fusion protein has the amino acid sequence set forth in any one of SEQ ID NOs: 111, 8, 10, 130, 131, 134, 135, 136, 137, 147, 149 and also optionally modulates immune cell response in vitro or in vivo.

According to at least some embodiments, the subject invention provides isolated nucleic acid sequences encoding any one of the foregoing VSTM5 polypeptides comprising a fragment of VSTM5 ECD, consisting essentially of an amino acid sequence as set forth in any one of SEQ ID NOs: 1, 12-110, 151-156 or a variant thereof that possesses at least 90 or 95% sequence identity therewith, or multimers or fusion proteins thereof.

According to at least some embodiments, there is provided an expression vector or a virus, containing at least one isolated nucleic acid sequence as described herein. According to at least some embodiments, there is provided a recombinant cell comprising an expression vector or a virus containing an isolated nucleic acid sequence as described herein, wherein the cell constitutively or inducibly expresses the polypeptide encoded by the DNA segment. According to at least some embodiments, there is provided a method of producing any one of the foregoing VSTM5 polypeptides comprising a fragment of VSTM5 ECD, consisting essentially of an amino acid sequence as set forth in any one of SEQ ID NOs: 1, 12-110, 151-156 or variant thereof that possesses at least 90 or 95% sequence identity therewith, or fusion proteins thereof, comprising culturing the recombinant cell as described herein, under conditions whereby the cell expresses the polypeptide encoded by the DNA segment or nucleic acid and recovering said polypeptide.

According to at least some embodiments of the present invention, there is provided a pharmaceutical composition comprising an isolated or recombinant VSTM5 polypeptide as described herein, or a fusion protein or multimeric VSTM5 polypeptide as described herein; a nucleotide sequence as described herein; an expression vector as described herein; a host cell as described herein, and further comprising a pharmaceutically acceptable diluent or carrier.

According to at least some embodiments of the present invention, there is also provided a pharmaceutical composition comprising a VSTM5 ECD protein set forth in any of SEQ ID NOs: 1, 2, 3, 12-110, 151-156, or variant thereof that possesses at least 90 or 95% sequence identity therewith, and/or fusion protein thereof, for use in cancer immunotherapy treatment in a subject suffering from cancer, wherein the cancer does not express VSTM5 protein or does not express sufficient levels of VSTM5 protein at diagnosis or prior to the treatment, wherein said pharmaceutical composition is administered to the subject in need thereof optionally in combination with another therapeutic agent or agents useful for the treatment of cancer.

According to at least some embodiments of the present invention, there is also provided a pharmaceutical composition comprising a VSTM5 ECD protein set forth in any of SEQ ID NOs: 1, 2, 3, 12-110, 151-156, or variant thereof that possesses at least 90 or 95% sequence identity therewith, and/or fusion protein thereof, for use in treating immune diseases such as any of the autoimmune, inflammatory or allergic condition, e.g., those disclosed herein, in a subject suffering from same wherein the diseased cells do not express VSTM5 protein or does not express sufficient levels of VSTM5 protein at diagnosis or prior to the treatment, wherein said pharmaceutical composition is administered to the subject in need thereof optionally in combination with another therapeutic agent or agents useful for the treatment of the immune condition.

Optionally, the combined use of a VSTM5 antagonist polypeptide according to at least some embodiments of the invention (comprising at least one of a VSTM5 ECD protein set forth in any of SEQ ID NOs: 1, 2, 3, 12-110, 151-156, or variant thereof that possesses at least 95% sequence identity therewith, and/or fusion protein thereof, or a pharmaceutical composition containing same), with another therapeutic agent or therapy converts non-responsive cancers to cancers that respond to immunotherapy or chemotherapy. This may occur for example, in cancers that do not express any VSTM5 or which do not express a sufficient level of VSTM5 upon initial diagnosis and/or prior to the initiation of the therapy, however VSTM5 expression may be induced by the tumor therapy, preferably a combination therapy, or may increase as the cancer progresses, thus making the treated subject resistant to cancer therapy because of the immunosuppressive effects of VSTM5 expression. In such cases the use of an immunostimulatory VSTM5 polypeptide according to the invention may alleviate such immunosuppression and thereby render the cancer more responsive to cancer therapy, e.g., immunotherapy, chemotherapy or drug therapy or other cancer therapy or regimen such as radiation because of the immunostimulatory effects of VSTM5 proteins according to at least some embodiments of the invention.

According to at least some embodiments of the present invention, there is provided any of the foregoing proteins, nucleic sequences, expression vectors or viruses, recombinant cells, or pharmaceutical compositions, wherein said protein, said nucleic sequence, said expression vector or virus, said recombinant cell or said pharmaceutical composition is used for treatment of cancer and/or infectious disease, and is capable of at least one of: increasing immune response, increasing T cell activation, increasing cytotoxic T cell activity, increasing NK cell activity, alleviating T-cell suppression, increasing pro-inflammatory cytokine secretion, increasing IL-2 secretion; increasing interferon-γ production by T-cells, increasing Th1 response, decreasing Th2 response, increasing Th17 immune activity, decreasing or eliminating regulatory T cells or other immune suppressive cells such as T regs or myeloid derived suppressor cells (MDSCs) or the infiltration of such cells to disease sites, decreasing or eliminating regulatory B cells or educing regulatory cell activity, decreasing or eliminating M2 macrophages, reducing M2 macrophage pro-tumorigenic activity, reducing inhibition of T cell activation, reducing inhibition of CTL activation such as the inhibition CD4⁺ and/or CD8⁺ T cell activation, reducing inhibition of NK cell activation, reversing T cell exhaustion, increasing T cell response, increasing Th17 immune responses, increasing activity of cytotoxic cells, stimulating antigen-specific memory responses, eliciting apoptosis or lysis of cancer cells, stimulating cytotoxic or cytostatic effect on cancer cells, inducing direct killing of cancer cells, reversing or alleviating immune tolerance or prolonged immunosuppression, and/or inducing complement dependent cytotoxicity and/or antibody dependent cell-mediated cytotoxicity.

According to at least some embodiments of the present invention, there is provided use of any of the foregoing proteins, nucleic sequences, expression vectors or viruses, recombinant cells, or pharmaceutical compositions, in cancer immunotherapy treatment in a subject suffering from cancer, wherein the cancer does not express VSTM5 or express sufficient levels of VSTM5 protein at diagnosis or prior to the treatment, wherein said treatment comprises administration of said VSTM5 protein, said nucleic sequence, said expression vector or virus, said recombinant cell or said pharmaceutical composition, in combination with any of the therapeutic agents useful for treatment of cancer.

According to at least some embodiments of the present invention, there is also provided a use of any of the foregoing proteins, nucleic sequences, expression vectors or viruses, recombinant cells, or pharmaceutical compositions, for treating a cancer which expresses sufficient levels of VSTM5 protein, in a subject in need thereof, e.g., an amount of VSTM5 that results in immunosuppression, which immunosuppression may result in the inhibition of antitumor immunity

Optionally whether VSTM5 protein levels are sufficient at diagnosis or before, during or after therapy may be determined by applying an IHC assay, comprising determining if a level of VSTM5 protein expression is at least 1 on a scale of 0 to 3.

Optionally whether VSTM5 protein levels are sufficient at diagnosis or before, during or after therapy may be determined by detected in a tissue comprising cancer cells and/or immune infiltrate and/or on immune and/or on stromal cells.

Optionally, said cancer, said immune infiltrate, and/or stromal cells express VSTM5 at a sufficient level, wherein VSTM5 expression on any of the cells listed above may be present prior to cancer treatment and/or induced post treatment. In the latter the treatment or the disease as it progresses may result in increased levels or VSTM5 protein on cells and/or an increased number of VSTM5 expressing cells. By “immune infiltrate” is meant immune cells infiltrating to the tumor or to the area of the cancerous cells. By “expressing VSTM5 at a sufficient level” it is meant that such cells express VSTM5 protein at a high enough level according to an assay. For example, if the assay is IHC (immunohistochemistry), and expression is measured on a scale of 0 to 3 (0-no expression, 1—faint staining, 2—moderate and 3—strong expression), then a sufficient level of VSTM5 expression would optionally be at least 1, preferably be at least 2 and more preferably be at least 3. Optionally VSTM5 specific antibodies or immune molecules as described herein may be used for such an assay.

Alternatively, “expressing VSTM5 at a sufficient level” may refer to cells, e.g., immune other cells such as cancer or infected cells that express VSTM5 at levels wherein VSTM5 polypeptides according to the invention may act directly on these cells and/or wherein the subject immunomodulatory VSTM5 polypeptides are expected to be particularly therapeutically beneficial as the endogenously expressed VSTM5 polypeptides may be exerting immunosuppressive effects on host immunity such as reduced antitumor immunity.

According to at least some embodiments of the present invention, there is also provided a use of any of the foregoing proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156 or variant thereof that possesses at least 90 or 95% sequence identity therewith and/or fusion protein thereof, for treating infectious disease, or for reducing the undesirable immune activation that follows gene therapy, in a subject in need thereof.

According to at least some embodiments of the present invention, there is also provided a use of any of the foregoing proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156 or variant thereof that possesses at least 90 or 95% sequence identity therewith and/or fusion protein thereof, wherein said protein, said nucleic sequence, said expression vector or virus, said recombinant cell, said pharmaceutical composition is used for the treatment of immune related diseases such as autoimmunity, allergy or inflammation, particularly chronic conditions associated therewith, and/or for reducing the undesirable immune activation that follows gene or cell therapy or cell, tissue or organ transplantation and which is capable of at least one of inhibiting, alleviating or preventing undesired immune responses, reducing T cell activity such as Th17, CD4⁺, and/or CD8⁺ T cell activity, reducing NK cell activity, enhancing regulatory cell activity or immunosuppressor cells such as Tregs and/or MDSCs, enhancing T-cell or B-cell suppression, enhancing immune regulatory cell activity, inducing the establishment of immune tolerance or prolonged antigen specific immunosuppression, reducing pro-inflammatory cytokine secretion, re-establishing Th1-Th2 immune balance, reducing immune memory responses to self-antigens, decreasing or eliminating pro-inflammatory immune cells and/or proinflammatory cytokine levels, and/or decreasing or eliminating auto-reactive immune cells.

According to at least some embodiments, the present invention provides immunostimulatory VSTM5 therapeutic agents, wherein said agents are used for treatment of cancer and/or infectious disease, and wherein said agents mediate at least one of the following immune effects: increase immune response, increase T cell activation, increase cytotoxic T cell activity, increase Th17 activity, increase NK cell activity, alleviate T-cell suppression, increase pro-inflammatory cytokine secretion, increase IL-2 secretion; increase interferon-γ production by T-cells, increase Th1 response, decrease Th2 response, decrease or eliminate regulatory T cells such as s Tregs, MDSCs, iMCs, mesenchymal stromal cells, TIE2-expressing and other immunosuppressive monocytes, inflammatory monocytes, neutrophils, macrophages and the like, reduce regulatory cell activity or the activity of MDSCs, iMCs, mesenchymal stromal cells, TIE2-expressing and other immunosuppressive monocytes, inflammatory monocytes, neutrophils, macrophages and the like, decrease or eliminate M2 macrophages, reduce M2 macrophage pro-tumorigenic activity, reduce inhibition of T cell activation, reduce inhibition of CTL (CD4⁺ or CD8⁺) activation, reduce inhibition of NK cell activation, reverse T cell exhaustion, increase T cell response, increase activity of cytotoxic cells, stimulate antigen-specific memory responses, elicit apoptosis or lysis of cancer cells, stimulate cytotoxic or cytostatic effect on cancer cells, induce direct killing of cancer cells, induce complement dependent cytotoxicity and/or induce antibody dependent cell-mediated cytotoxicity in a mammal.

According to at least some embodiments, for any of the above described cancers, optionally each of the above described cancer type or subtype may optionally form a separate embodiment and/or may optionally be combined as embodiments or subembodiments.

According to at least some embodiments, there is provided a method of performing one or more of the following in a subject by administering a VSTM5 protein as described herein or a pharmaceutical composition as described herein to a subject, (i) increases immune response, (ii) increases T cell activation, (iii) increases cytotoxic T cell activity, (iv) increases NK cell activity, (v) increases Th17 activity, (vi) alleviates T-cell suppression, (vii) increases pro-inflammatory cytokine secretion, (viii) increases IL-2 secretion; (ix) increases interferon-γ production by T-cells, (x) increases Th1 response, (xi) decrease Th2 response, (xii) decreases or eliminates cell number and/or activity of at least one of regulatory T cells (Tregs), myeloid derived suppressor cells (MDSCs), iMCs, mesenchymal stromal cells, TIE2-expressing monocytes, (xiii) reduces regulatory cell activity and/or the activity of one or more of myeloid derived suppressor cells (MDSCs), iMCs, mesenchymal stromal cells, TIE2-expressing monocytes, (xiv) decreases or eliminates M2 macrophages, (xv) reduces M2 macrophage pro-tumorigenic activity, (xvi) decreases or eliminates N2 neutrophils, (xvii) reduces N2 neutrophils pro-tumorigenic activity, (xviii) reduces inhibition of T cell activation, (xix) reduces inhibition of CTL activation, (xx) reduces inhibition of NK cell activation, (xxi) reverses T cell exhaustion, (xxii) increases T cell response, (xxiii) increases activity of cytotoxic cells, (xxiv) stimulates antigen-specific memory responses, (xxv) elicits apoptosis or lysis of cancer cells, (xxvi) stimulates cytotoxic or cytostatic effect on cancer cells, (xxvii) induces direct killing of cancer cells, and/or (xxviii) induces complement dependent cytotoxicity and/or (xxix) induces antibody dependent cell-mediated cytotoxicity, with the proviso that said isolated or recombinant VSTM5 polypeptide or fusion protein may elicit an opposite effect to one or more of (i)-(xxix).

As described herein, optionally increasing immune response (which, without being limited by a single hypothesis, would be expected to antagonize the effect of VSTM5) could be used for treating diseases where increased immune response is desirable, including but not limited to cancer and infectious disorders.

According to at least some embodiments of the invention there is provided a diagnostic method for diagnosing a disease in a subject, or aiding in the diagnosis of a disease, e.g. one associated with immunosuppression and/or a disease is selected from the group consisting of cancer, autoimmune disease, inflammatory disease, allergic disease, or infectious disease, wherein the diagnostic method is performed ex vivo, comprising contacting a tissue sample from the subject with any of the foregoing polypeptides and/or with VSTM5 ECD protein set forth in any of SEQ ID NOs: 1, 2, 3, 12-110, 151-156 or variant thereof that possesses at least 90 or 95% sequence identity therewith, and/or with a multimeric or fusion protein thereof, and detecting specific binding to the tissue sample.

According to at least some embodiments of the invention there is provided a diagnostic method for diagnosing or aiding in the diagnosis of a disease in a subject, wherein the disease is selected from the group consisting of cancer, autoimmune disease, inflammatory disease, allergic disease, especially chronic disease conditions, or an infectious disease wherein the diagnostic method is performed in vivo, comprising administering any of the foregoing polypeptides, and/or with VSTM5 ECD protein set forth in any of SEQ ID NOs: 1, 2, 3, 12-110, 151-156 or variant thereof that possesses at least 90 or 95% sequence identity therewith, and/or with a fusion protein thereof, to a subject and detecting specific binding to tissues.

According to at least some embodiments of the invention the invention relates to any of the foregoing uses or methods which further includes a diagnostic method performed before administering any of the foregoing polypeptides, and/or with VSTM5 ECD protein set forth in any of SEQ ID NOs: 1, 2, 3, 12-110, 151-156 or variant thereof that possesses at least 90 or 95% sequence identity therewith, and/or with a multimeric or fusion protein thereof to the subject.

According to at least some embodiments there is provided a diagnostic use as described herein for screening for a disease, aiding in the identification of a disease condition, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. As described herein, the disease is optionally selected from an autoimmune disease and cancer.

According to at least some embodiments, there is provided a use of any of the foregoing proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156 or variant thereof that possesses at least 90 or 95% sequence identity therewith, and/or multimeric or fusion protein thereof for the treatment or diagnosis of cancer, or a combination thereof, wherein said cancer is selected from the group consisting of breast cancer, cervical cancer, ovary cancer, endometrial cancer, melanoma, uveal melanoma, bladder cancer, lung cancer, pancreatic cancer, colorectal cancer, prostate cancer, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, teratocarcinoma, neuroblastoma, glioma, glioblastoma, benign tumor of the skin, keratoacanthomas, renal cancer, anaplastic large-cell lymphoma, esophageal cancer, follicular dendritic cell carcinoma, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, myelodysplasia, Waldenström's macroglobinaemia, nasopharyngeal, neuroendocrine cancer, mesothelioma, angiosarcoma, Kaposi's sarcoma, carcinoid, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL), cancer of unknown origin.

In some embodiments the cancer is selected from the group consisting of B-cell lymphoma, Burkitt's lymphoma, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma cancer, keratoacanthomas, anaplastic large-cell lymphoma, esophageal squamous cells carcinoma, hepatocellular carcinoma cancer, follicular dendritic cell carcinoma, muscle-invasive cancer, seminal vesicle tumor, epidermal carcinoma, cancer of the retina, biliary cancer, small bowel cancer, salivary gland cancer, cancer of connective tissue, myelodysplasia, Waldenström's macroglobinaemia, nasopharyngeal, neuroendocrine cancer, myelodysplastic syndrome, mesothelioma, angiosarcoma, Kaposi's sarcoma, carcinoid, esophagogastric, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL); endometrial cancer, Breast carcinoma, preferably any of ductal-carcinoma, infiltrating ductal carcinoma, lobular carcinoma, mucinous adenocarcinoma, intra duct and invasive ductal carcinoma, and Scirrhous adenocarcinoma, Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum; Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma; Prostate adenocarcinoma, preferably any of Adenocarcinoma Gleason Grade 6 to 9, Infiltrating adenocarcinoma, High grade prostatic intraepithelial neoplasia, undifferentiated carcinoma; Stomach adenocarcinoma, preferably moderately differentiated gastric adenocarcinoma; Ovary carcinoma, preferably any of cystadenocarcinoma, serous papillary cystic carcinoma, Serous papillary cystic carcinoma, Invasive serous papillary carcinoma; Brain cancer, preferably any of Astrocytoma, with the proviso that it is not a grade 2 astrocytoma, preferably grade 4 Astrocytoma, Glioblastoma multiforme; Kidney carcinoma, preferably Clear cell renal cell carcinoma; Liver cancer, preferably any of Hepatocellular carcinoma, preferably Low Grade hepatocellular carcinoma, Fibrolamellar Hepatocellular Carcinoma; Lymphoma, preferably any of, Hodgkin's Lymphoma and High to low grade Non-Hodgkin's Lymphoma, and with the proviso that if the cancer is brain cancer, it is not Astrocytoma grade 2, and if the cancer is Non-Hodgkin's Lymphoma, it is not a large cell Non-Hodgkin's Lymphoma.

With respect to the foregoing cancers, optionally the cancer comprises one or more of non-metastatic, invasive and metastatic forms thereof.

Moreover, as disclosed infra, it has been found that improved outcome can be achieved using the above immunostimulatory VSTM5 therapeutic agents according to at least some embodiments of the invention, for treatment of specific cancers, which highly express VSTM5 and are particularly susceptible to treatment and/or diagnosis with the immunostimulatory VSTM5 therapeutic agents according to various embodiments of the present invention. These specific cancers may optionally include any one or more of:
Breast carcinoma, preferably any of ductal-carcinoma, infiltrating ductal carcinoma, lobular carcinoma, mucinous adenocarcinoma, intra duct and invasive ductal carcinoma, and Scirrhous adenocarcinoma;
Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum;
Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma;
Prostate adenocarcinoma, preferably any of Adenocarcinoma Gleason Grade 6 to 9, Infiltrating adenocarcinoma, High grade prostatic intraepithelial neoplasia, undifferentiated carcinoma;
Stomach adenocarcinoma, preferably moderately differentiated gastric adenocarcinoma;
Ovary carcinoma, preferably any of cystadenocarcinoma, serous papillary cystic carcinoma, Serous papillary cystic carcinoma, Invasive serous papillary carcinoma;
Brain cancer, preferably any of Astrocytoma, with the proviso that it is not a grade 2 astrocytoma, preferably grade 4 Astrocytoma, Glioblastoma multiforme;
Kidney carcinoma, preferably Clear cell renal cell carcinoma;
Liver cancer, preferably any of Hepatocellular carcinoma, preferably Low Grade hepatocellular carcinoma, Fibrolamellar Hepatocellular Carcinoma;
Lymphoma, preferably any of, Hodgkin's Lymphoma and High to low grade Non-Hodgkin's Lymphoma.

Optionally the treatment is combined with another therapeutic agent or therapy useful for treating cancer, in a subject in need thereof.

Optionally, the therapy comprises one or more of radiotherapy, cryotherapy, antibody therapy, chemotherapy, photodynamic therapy, surgery, hormonal deprivation or combination therapy with conventional drugs.

Optionally, the therapeutic agent is selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

Optionally, the immunostimulatory VSTM5 therapeutic agents according to at least some embodiments are administered to a subject simultaneously or sequentially in combination (in either order), in the same or different compositions, with one or more immune potentiating agents, wherein said one or more potentiating agents is other than a VSTM5 polypeptide or multimer or fusion protein containing and optionally may be selected from the group consisting of radiotherapy, conventional/classical anti-cancer agent, therapy potentiating anti-tumor immune responses, targeted therapy potentiating anti-tumor immune responses, therapeutic agents targeting immunosuppressive cells such as Tregs, MDSCs, iMCs, mesenchymal stromal cells, TIE2-expressing monocytes, inflammatory monocytes, neutrophils, macrophages and the like, immunostimulatory antibodies, cytokine therapy, and adoptive cell transfer.

Optionally, the conventional/classical anti-cancer agent is selected from the group consisting of Platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antioestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; inhibitors of 5α-reductase such as finasteride.

Optionally, the targeted therapy agent is selected from the group consisting of histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfiram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib;

Metalloproteinase inhibitors such as marimastat; Inhibitors of urokinase plasminogen activator receptor function; Inhibitors of cathepsin activity and combinations of any of the foregoing.

Optionally, the antibody is selected from the group consisting of cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, and aflibercept.

Optionally, the therapeutic agent targeting immunosuppressive cells Tregs and/or MDSCs or other immunosuppressive cells such as iMCs, mesenchymal stromal cells, TIE2-expressing monocytes, inflammatory monocytes, neutrophils, macrophages and the like is selected from the group consisting of antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Optionally, the Immunostimulatory antibody is selected from the group consisting of antagonistic antibodies targeting immune checkpoints including anti-CTLA4 mAbs, such as ipilimumab, tremelimumab; Anti-PD-1 such as nivolumab BMS-936558/MDX-1106/ONO-4538, AMP224, CT-011, MK-3475; anti-PDL-1 antagonists such as BMS-936559/MDX-1105, MEDI4736, RG-7446/MPDL3280A; anti-LAG-3 such as IMP-321), anti-TIM-3, anti-BTLA, anti-B7-H4, anti-B7-H3, anti-VISTA; agonistic antibodies targeting immunostimulatory proteins, including anti-CD40 mAbs such as CP-870,893, lucatumumab, dacetuzumab; Anti-CD137 mAbs such as BMS-663513 urelumab, PF-05082566; Anti-OX40 mAbs, such as anti-OX40; anti-GITR mAbs such as TRX518; Anti-CD27 mAbs, such as CDX-1127; and Anti-ICOS mAbs.

Optionally, the therapeutic cancer vaccine is selected from from the group consisting of exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, and oncolytic virus vaccines.

Optionally, the cytokine therapy is selected from from the group consisting of one or more of the following cytokines and combinations thereof such as IL-2, IL-6, IL-7, IL-12, IL-15, IL-17, IL-18 and/or IL-21, IL-23, IL-27, GM-CSF, IFNα, IFNα-2b, IFNβ, IFNγ, TNF-α, and TNF-β, which may be delivered by known methods.

Optionally, the adoptive cell transfer therapy is carried out following ex vivo treatment selected from expansion of the patient autologous naturally occurring tumor specific T cells or genetic modification of T cells to confer specificity for tumor antigens.

According to at least some embodiments of the present invention, there is also provided any of the foregoing proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156, or variant thereof that possesses at least 90 or 95% sequence identity therewith, and/or multimers or fusion proteins thereof, any of the foregoing therapeutic and/or diagnostic uses, methods or assays, for the treatment, prophylaxis and/or diagnosis of immune conditions, wherein said immune conditions may be selected from the group consisting of autoimmune disease, allergic diseases, inflammatory diseases, both chronic and non-chronic diseases, transplant rejection, and graft versus host disease. In the case of transplant and cell or gene therapy indications an VSTM5 ECD immunomodulatory protein may be administered prior, concurrent or after transplant or cell or gene therapy. Also, the VSTM5 ECD immunomodulatory protein may be used to contact immune cells or other cells, tissue or organ which is to be infused or transplanted in a recipient ex vivo in order to modulate the expression and/or VSTM5 mediated effects on immune cells prior to transplant or cell or gene therapy. These cells may be autologous or heterologous to the treated subject. For example, and without limitation, bone marrow and/or a host's immune cells may be treated with a VSTM5 ECD immunoinhibitory protein prior to infusion or reinfusion of these cells in order to suppress T cell immunity and potentially avert or alleviate a GVHD or HVG immune reaction.

The term “autoimmune disease” as used herein should be understood to encompass any autoimmune disease and further includes chronic inflammatory conditions. According to at least some embodiments of the invention, the autoimmune diseases should be understood to encompass any disease disorder or condition selected from the group including but not limited to multiple sclerosis, including relapsing-remitting multiple sclerosis, primary progressive multiple sclerosis, and secondary progressive multiple sclerosis; psoriasis; rheumatoid arthritis; psoriatic arthritis, systemic lupus erythematosus (SLE); discoid lupus erythematosus, inflammatory bowel disease, ulcerative colitis; Crohn's disease; benign lymphocytic angiitis, thrombocytopenic purpura, idiopathic thrombocytopenia, idiopathic autoimmune hemolytic anemia, pure red cell aplasia, Sjögren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune hemolytic anaemia, Guillain-Barré syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anaemia, pemphigus, pemphigus vulgaris, cirrhosis, primary biliary cirrhosis, dermatomyositis, polymyositis, fibromyositis, myogelosis, celiac disease, immunoglobulin A nephropathy, Henoch-Schönlein purpura, Evans syndrome, Dermatitis, atopic dermatitis, psoriasis, psoriasis arthropathica, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urtic aria, bullous skin disorders, pemphigoid, bullous pemphigoid, cicatricial pemphigoid, vitiligo, atopic eczema, eczema, chronic urticaria, autoimmune urticaria, normocomplementemic urticarial vasculitis, hypocomplementemic urticarial vasculitis, autoimmune lymphoproliferative syndrome, Devic's disease, sarcoidosis, pernicious anemia, childhood autoimmune hemolytic anemia, idiopathic autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne, normocomplementemic urticarial vasculitis, pericarditis, idiopathic pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, any rheumatic disease, polymyalgia rheumatica, mixed connective tissue disease, inflammatory rheumatism, degenerative rheumatism, extra-articular rheumatism, juvenile arthritis, juvenile rheumatoid arthritis, systemic juvenile idiopathic arthritis, arthritis uratica, muscular rheumatism, chronic polyarthritis, reactive arthritis, Reiter's syndrome, rheumatic fever, relapsing polychondritis, Raynaud's phenomenon, vasculitis, cryoglobulinemic vasculitis, temporal arteritis, giant cell arteritis, Takayasu arteritis, Behcet's disease, chronic inflammatory demyelinating polyneuropathy, autoimmune thyroiditis, insulin dependent diabetes mellitus, type I diabetes, Addison's disease, membranous glomerulonephropathy, polyglandular autoimmune syndromes, Goodpasture's disease, autoimmune gastritis, autoimmune atrophic gastritis, pernicious anaemia, pemphigus, pemphigus vulgaris, cirrhosis, primary biliary cirrhosis, idiopathic pulmonary fibrosis, myositis, dermatomyositis, juvenile dermatomyositis, polymyositis, fibromyositis, myogelosis, celiac disease, celiac sprue dermatitis, immunoglobulin A nephropathy, Henoch-Schönlein purpura, Evans syndrome, atopic dermatitis, psoriasis, psoriasis vulgaris, psoriasis arthropathica, Graves' disease, Graves' ophthalmopathy, scleroderma, systemic scleroderma, progressive systemic scleroderma, diffuse scleroderma, localized scleroderma, Crest syndrome, asthma, allergic asthma, allergy, primary biliary cirrhosis, fibromyalgia, chronic fatigue and immune dysfunction syndrome (CFIDS), autoimmune inner ear disease, Hyper IgD syndrome, Schnitzler's syndrome, autoimmune retinopathy, age-related macular degeneration, atherosclerosis, chronic prostatitis, alopecia, alopecia areata, alopecia universalis, alopecia totalis, autoimmune thrombocytopenic purpura, idiopathic thrombocytopenic purpura, pure red cell aplasia, and TNF receptor-associated periodic syndrome (TRAPS).

In some embodiments the autoimmune disease is one of relapsing-remitting multiple sclerosis, primary progressive multiple sclerosis, secondary progressive multiple sclerosis; progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, acute multiple sclerosis, conditions relating to multiple sclerosis, psoriatic arthritis, gout and pseudo-gout, juvenile idiopathic arthritis, Still's disease, rheumatoid vasculitis, conditions relating to rheumatoid arthritis, discoid lupus erythematosus, lupus arthritis, lupus pneumonitis, lupus nephritis, conditions relating to systemic lupus erythematosus include osteoarticular tuberculosis, antiphospholipid antibody syndrome, inflammation of various parts of the heart, such as pericarditis, myocarditis, and endocarditis, lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myelopathy, cranial neuropathy, polyneuropathy, vasculitis, collagenous colitis, lymphocytic colitis, ischaemia colitis, diversion colitis, Behçet's disease, indeterminate colitis, thrombocytopenic purpura, idiopathic autoimmune hemolytic anemia, pure red cell aplasia, cryoglobulinemic vasculitis, ANCA-associated vasculitis, antiphospholipid syndrome, autoimmune hemolytic anaemia, Guillain-Barré syndrome, chronic immune polyneuropathy, autoimmune thyroiditis, idiopathic diabetes, juvenile type ldiabetes, maturity onset diabetes of the young, latent autoimmune diabetes in adults, gestational diabetes, conditions relating to type 1 diabetes, membranous glomerulonephropathy, autoimmune gastritis, pemphigus vulgaris, cirrhosis, fibromyositis, celiac disease, immunoglobulin A nephropathy, Henoch-Schönlein purpura, Evans syndrome, atopic dermatitis, psoriasis, Graves' ophthalmopathy, systemic scleroderma, asthma, allergy, anterior uveitis (or iridocyclitis), intermediate uveitis (pars planitis), posterior uveitis (or chorioretinitis), panuveitic form, hepatitis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, prevention of development of autoimmune anti-factor VIII Antibodies in acquired hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, selected from the group consisting of psoriasis, Non pustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis, drug-induced psoriasis, Inverse psoriasis, Napkin psoriasis, Seborrheic-like psoriasis, Guttate psoriasis, Nail psoriasis, Psoriatic arthritis, atopic dermatitis, eczema, rosacea, urticaria, and acne, normocomplementemic urticarial vasculitis, pericarditis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis, Dermatitis, bullous pemphigoid, cicatricial pemphigoid, vitiligo, atopic eczema, eczema, chronic urticaria, autoimmune urticaria, normocomplementemic urticarial vasculitis, hypocomplementemic urticarial vasculitis, autoimmune lymphoproliferative syndrome, sarcoidosis, pernicious anemia, idiopathic autoimmune hemolytic anemia, idiopathic pericarditis, a rheumatic disease, polymyalgia rheumatica, mixed connective tissue disease, inflammatory rheumatism, degenerative rheumatism, extra-articular rheumatism, juvenile arthritis, juvenile rheumatoid arthritis, systemic juvenile idiopathic arthritis, arthritis uratica, muscular rheumatism, chronic polyarthritis, reactive arthritis, Reiter's syndrome, rheumatic fever, relapsing polychondritis, Raynaud's phenomenon, vasculitis, cryoglobulinemic vasculitis, temporal arteritis, giant cell arteritis, Takayasu arteritis, Behcet's disease, chronic inflammatory demyelinating polyneuropathy, autoimmune thyroiditis, insulin dependent diabetes mellitus, type I diabetes, Addison's disease, membranous glomerulonephropathy, polyglandular autoimmune syndromes, Goodpasture's disease, autoimmune gastritis, autoimmune atrophic gastritis, pernicious anaemia, pemphigus, pemphigus vulgaris, cirrhosis, primary biliary cirrhosis, idiopathic pulmonary fibrosis, myositis, dermatomyositis, juvenile dermatomyositis, polymyositis, fibromyositis, myogelosis, celiac disease, celiac sprue dermatitis, immunoglobulin A nephropathy, Henoch-Schönlein purpura, Evans syndrome, atopic dermatitis, psoriasis, psoriasis vulgaris, psoriasis arthropathica, Graves' disease, Graves' ophthalmopathy, scleroderma, systemic scleroderma, progressive systemic scleroderma, diffuse scleroderma, localized scleroderma, Crest syndrome, asthma, allergic asthma, allergy, primary biliary cirrhosis, fibromyalgia, chronic fatigue and immune dysfunction syndrome (CFIDS), autoimmune inner ear disease, alopecia, alopecia areata, alopecia universalis, alopecia totalis, autoimmune thrombocytopenic purpura, idiopathic thrombocytopenic purpura, pure red cell aplasia, and TNF receptor-associated periodic syndrome (TRAPS).

In some preferred embodiments, the autoimmune disease includes but is not limited to any of the types and subtypes of any of multiple sclerosis, rheumatoid arthritis, type I diabetes, psoriasis, systemic lupus erythematosus, inflammatory bowel disease, uveitis, or Sjögren's syndrome or related conditions thereof.

As described herein, VSTM5 polypeptides which modulate immunity may optionally be used to treat or detect “immune related diseases (or disorders or conditions)”. These phrases or terms are used interchangeably and encompass any disease, disorder or condition selected from the group including but not limited to autoimmune diseases, inflammatory disorders, allergic disorders, e.g., chronic allergic disorders such as asthma, and immune disorders associated with graft transplantation rejection, such as acute and chronic rejection of organ or tissue transplantation, allogenic stem cell transplantation, autologous stem cell transplantation, bone marrow transplantation, and graft versus host disease.

As further noted, the present VSTM5 polypeptides which modulate immunity may be used to treat “inflammatory disorders” and/or “inflammation”. These phrases or terms are used interchangeably herein and include e.g., inflammatory abnormalities characterized by dysregulated immune response to harmful stimuli, such as pathogens, damaged cells, or irritants. Inflammatory disorders underlie a vast variety of human diseases. Non-immune diseases with etiological origins in inflammatory processes include but are not limited to cancer, atherosclerosis, and ischemic heart disease. Examples of disorders associated with inflammation include but are not limited to: Chronic prostatitis, Glomerulonephritis, Hypersensitivities, Pelvic inflammatory disease, Reperfusion injury, Sarcoidosis, Vasculitis, Interstitial cystitis, normocomplementemic urticarial vasculitis, pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, TNF receptor-associated periodic syndrome (TRAPSP), gingivitis, periodontitis, hepatitis, cirrhosis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne.

In some embodiments the treatment may be optionally combined with another moiety useful for treating immune related condition, e.g., a moiety useful for treating immune related condition is selected from immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-α blockers or antagonists, or any other biological agent targeting any inflammatory cytokine, nonsteroidal antiinflammatory drugs/Cox-2 inhibitors, hydroxychloroquine, sulphasalazopryine, gold salts, etanercept, infliximab, mycophenolate mofetil, basiliximab, atacicept, rituximab, cytoxan, interferon β-1a, interferon β-1b, glatiramer acetate, mitoxantrone hydrochloride, anakinra and/or other biologics and/or intravenous immunoglobulin (IVIG), interferons such as IFN-β-1a (REBIF®, AVONEX® and CINNOVEX®) and IFN-β-1b (BETASERON®); EXTAVIA®, BETAFERON®, ZIFERON®); glatiramer acetate (COPAXONE®), a polypeptide; natalizumab (TYSABRI®), mitoxantrone (NOVANTRONE®), a cytotoxic agent, a calcineurin inhibitor, e.g., cyclosporin A or FK506; an immunosuppressive macrolide, e.g. rapamycin or a derivative thereof; e.g. 40-O-(2-hydroxy) ethyl-rapamycin, a lymphocyte homing agent, e.g. FTY720 or an analog thereof, corticosteroids; cyclophosphamide; azathioprene; methotrexate; leflunomide or an analog thereof; mizoribine; mycophenolic acid; mycophenolate mofetil; 15-deoxyspergualine or an analog thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, e.g. CTLA4-Ig (abatacept, ORENCIA®, belatacept), CD28-Ig, B7-H4-Ig, or other costimulatory agents, or adhesion molecule inhibitors, e.g. mAbs or low molecular weight inhibitors including LFA-1 antagonists, Selectin antagonists and VLA-4 antagonists, or another immunomodulatory agent.

According to at least some embodiments of the present invention, there is also provided any of the foregoing proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156 or variant thereof that possesses at least 90 or 95% sequence identity therewith, and/or fusion protein thereof, any of the foregoing therapeutic and/or diagnostic use, method or assay, for treatment and/or diagnosis of infectious disease, wherein said infectious disease is chronic infectious disease and is selected from the disease caused by bacterial infection, viral infection, fungal infection and/or other parasite infection.

Optionally said infectious disease is selected from hepatitis B, hepatitis C, infectious mononucleosis, AIDS, tuberculosis, malaria and schistosomiasis.

Optionally said infectious disease is one that may result in sepsis, e.g., severe sepsis, septic shock, systemic inflammatory response syndrome (SIRS), Bacteremia, Septicemia, Toxemia, or Septic syndrome.

Optionally the treatment is combined with another moiety useful for treating an infectious disease, in a subject in need thereof.

Optionally, said other moiety is another therapeutic agent useful for treating bacterial infection, viral infection, fungal infection, parasitic infection or sepsis.

According to at least some embodiments of the present invention, there is also provided any of the foregoing VSTM5 proteins, nucleic sequences, expression vectors or viruses, recombinant cells, or pharmaceutical compositions, or the pharmaceutical composition comprising a VSTM5 ECD protein set forth in any of SEQ ID NOs: 1, 2, 3, 12-110, 151-156 or variant thereof that possesses at least 90 or 95% sequence identity therewith, and/or fusion protein thereof, any of the foregoing therapeutic and/or diagnostic use, method or assay, for reducing the undesirable immune activation that follows gene therapy.

Optionally the treatment is combined with another moiety useful for reducing the undesirable immune activation that follows gene therapy, in a subject in need thereof.

According to at least some embodiments, there is provided a method for potentiating a secondary immune response to an antigen in a patient, which method comprises administering effective amount of any of the foregoing polypeptides, fusion proteins, nucleotide sequences, expression vectors or viruses, host cells, or pharmaceutical compositions.

Optionally the antigen is a cancer antigen, a viral antigen or a bacterial antigen, and the patient has received treatment with an anticancer vaccine or a viral vaccine.

According to at least some embodiments of the invention, the method of immunotherapy in a patient optionally, comprises one of the following:
in vivo or ex vivo tolerance induction, comprising administering an effective amount of any of an isolated or recombinant VSTM5 polypeptide as described herein, or a multimer or fusion protein as described herein; a nucleotide sequence as described herein; an expression vector as described herein; a host cell as described herein, or a pharmaceutical composition, to a patient or to leukocytes isolated from the patient, in order to induce differentiation of tolerogenic regulatory cells;
ex vivo enrichment and expansion of said cells; and/or
reinfusion of the tolerogenic regulatory cells to said patient.

According to at least some embodiments there is provided a method of using at least one of: any of an isolated or recombinant VSTM5 polypeptide as described herein, or a fusion protein as described herein; a nucleotide sequence as described herein; an expression vector as described herein; a host cell as described herein, or a pharmaceutical composition; as a cancer vaccine adjuvant, comprising administration to a patient an immunogenic amount of a tumor associated antigen preparation of interest; and a cancer vaccine adjuvant in a formulation suitable for immunization, wherein the immune response against the tumor associated antigen in the presence of the cancer vaccine adjuvant is stronger than in the absence of the cancer vaccine adjuvant.

According to at least some embodiments there is provided a method for combining therapeutic vaccination with an antigen along with the administration of any of the isolated or recombinant VSTM5 polypeptides described herein, or a multimer or fusion protein as described herein; a nucleotide sequence as described herein; an expression vector as described herein; a host cell as described herein, or a pharmaceutical composition, for treatment of infection.

According to at least some embodiments, there is provided a method for combining any of an isolated or recombinant polypeptide as described herein, or a fusion protein as described herein; a nucleotide sequence as described herein; an expression vector as described herein; a host cell as described herein, or a pharmaceutical composition, an adjuvant, and an antigen in a vaccine, in order to increase the immune response.

Optionally the antigen is a viral antigen, bacterial antigen, fungal antigen, parasite antigen, and/or other pathogen's antigen.

According to at least some embodiments, any one of the foregoing therapeutic agents according to the present invention can be used for adoptive immunotherapy Immune tolerance or immunological tolerance or prolonged immunosuppression is the process by which the immune system does not attack an antigen. It can be either ‘natural’ or ‘self-tolerance’, where the body does not mount an immune response to self-antigens, or ‘induced tolerance’, where tolerance to external antigens can be created by manipulating the immune system. It occurs in three forms: central tolerance, peripheral tolerance and acquired tolerance. Without wishing to be bound by a single theory, tolerance employs regulatory immune cells-including Tregs—or potentially other immunosuppressive cells such as MDSCs, iMSCs, monocytes, neutrophils, macrophages, that directly suppress autoreactive cells, as well as several other immune cell subsets with immunoregulatory properties—including CD8⁺ T cells and other types of CD4⁺ T cells (Tr1, Th3), Th17 cells, in addition to natural killer (NK), NKT cells, dendritic cells (DC) and B cells.

Tolerance or prolonged immunosuppression can be induced by blocking costimulation or upon engagement of a co-inhibitory B7 with its counter receptor. Transfer of tolerance involves isolation of the cells that have been induced for tolerance either in vivo (i.e. prior to cell isolation) or ex-vivo, enrichment and expansion of these cells ex vivo, followed by reinfusion of the expanded cells to the patient. This method can be used for treatment of autoimmune diseases as recited herein, immune related diseases as recited herein, transplantation and graft rejection. Thus, according to at least some embodiments, the invention optionally provides methods for tolerance induction, comprising in vivo or ex vivo treatment administration of effective amount of any one of isolated soluble VSTM5 polypeptide, or a polypeptide comprising the extracellular domain of VSTM5, or fragment thereof, or a fusion thereof to a heterologous sequence, to a patient or to leukocytes isolated from the patient, in order to induce differentiation of tolerogenic regulatory cells, followed by ex-vivo enrichment and expansion of said cells and reinfusion of the tolerogenic regulatory cells to said patient.

According to at least some embodiments, there is provided an isolated polypeptide comprising a fragment of a VSTM5 ECD, wherein said fragment consists essentially of or consists of an amino acid sequence as set forth in any one of SEQ ID NOs: 1, 12-110, 151-156, or a variant thereof that possesses at least 95% sequence identity therewith.

According to at least some embodiments, there is provided an isolated polypeptide comprising at least two VSTM5 ECD polypeptide fragments, wherein said fragments are the same or different and are selected from the amino acid sequences set forth in any one of SEQ ID NOs: 1, 12-110, 151-156, or a variant thereof that possesses at least 95% sequence identity therewith, but wherein a sequence of said isolated polypeptide has less than 95% sequence identity with an amino acid sequence as set forth in any one of SEQ ID NOs: 1, 12-110, 151-156. Optionally the isolated polypeptide comprises 2-10 of said VSTM5 ECD polypeptide fragments. Optionally said fragments are intervened by a heterologous linker, wherein said linker is not a fragment of a VSTM5 polypeptide. Optionally said linker is directly or indirectly conjugated to said fragments. Optionally said linker is an amino acid spacer. Optionally said amino acid spacer is of sufficient length of amino acid residues so that the different fragments can successfully bind to their individual targets. Optionally said linker is a peptide comprising 5-50 amino acid residues, more preferably 5-25 amino acid residues. Optionally said linker is a peptide comprising 5-15 amino acid residues. Optionally said linker comprises or consists essentially of glycine, serine, and/or alanine residues or predominantly (at least 50, 60, 70 or 80% of the residues) consists of glycine, serine, and/or alanine residues. Optionally said linker comprises at least 4-40, 4-30, 4-20, or 4-12 glycine, serine, and/or alanine residues.

According to at least some embodiments, there is provided a fusion protein comprising the isolated polypeptide of any of the preceding claims, or SEQ ID NOs 2 or 3, joined to a heterologous polypeptide and/or half-life extending moiety, with the proviso that said heterologous polypeptide or said half-life extending moiety is not a fragment of a VSTM5 polypeptide. Optionally said isolated polypeptide and said heterologous molecule are intervened by a heterologous linker, with the proviso that said linker does not comprise a polypeptide that is a fragment of a VSTM5 polypeptide. Optionally said linker is directly or indirectly conjugated to said fragments. Optionally said linker is an amino acid spacer. Optionally said amino acid spacer is of sufficient length of amino acid residues so that the different fragments can successfully bind to their individual targets. Optionally said linker is a peptide comprising 5-50 amino acid residues, more preferably 5-25 amino acid residues. Optionally said linker is a peptide comprising 5-15 amino acid residues. Optionally said linker comprises or consists essentially of glycine, serine, and/or alanine residues or predominantly (at least 50, 60, 70 or 80% of the residues) consists of glycine, serine, and/or alanine residues. Optionally said linker comprises at least 4-40, 4-30, 4-20, or 4-12 glycine, serine, and/or alanine residues.

Optionally the fusion protein of any of the foregoing or as described herein, comprises or further comprises a half-life extending moiety. Optionally the half-life extending moiety comprises polyethylene glycol (PEG), monomethoxy PEG (mPEG), an XTEN molecule, an rPEG molecule, an adnectin, a serum albumin, human serum albumin, immunoglobulin constant region or fragment thereof, or acyl group. Optionally the addition of said heterologous polypeptide, half-life extending moiety, or other heterologous molecule increases the in vivo half-life of said fusion protein by at least about 2-fold, at least about 3-fold, at least about 4-fold, at least about 5-fold, at least about 10-fold, or more, as compared to the identical molecule without such said heterologous polypeptide, half-life extending moiety, or other heterologous molecule.

Optionally the fusion protein of any of the foregoing or as described herein, comprises an immunoglobulin molecule or a fragment thereof. Optionally at least one of the heterologous polypeptides is a human or non-human immunoglobulin Fc polypeptide or fragment that comprises heavy and/or light chain CH2 and CH3 domains. Optionally at least one of the heterologous polypeptides is a human or non-human immunoglobulin Fc polypeptide or fragment that comprises heavy chain CH2 and CH3 domains.

Optionally the fusion protein of any of the foregoing or as described herein, comprises heavy and/or light chain CH1 domains.

Optionally the fusion protein of any of the foregoing or as described herein, lacks heavy and/or light chain CH1 domains.

Optionally the fusion protein of any of the foregoing or as described herein, lacks heavy chain CH1 domains.

Optionally said immunoglobulin molecule or a fragment thereof comprises a hinge region. Optionally said hinge region is an intact hinge region. Optionally said immunoglobulin molecule or a fragment thereof does not feature a hinge region.

Optionally the fusion protein of any of the foregoing or as described herein comprises a human immunoglobulin molecule or a fragment thereof. Optionally said heterologous polypeptide comprises or consists of an Fc fragment of the immunoglobulin heavy chain constant region. Optionally said heterologous polypeptide comprises or consists of an Fc fragment and hinge region of a human immunoglobulin heavy chain constant region.

Optionally the fusion protein of any of the foregoing or as described herein comprises an immunoglobulin heavy chain constant region derived from an immunoglobulin isotype selected from the group consisting of an IgG1, IgG2, IgG3, IgG4, IgM, IgE, IgA and IgD.

Optionally the fusion protein of any of the foregoing or as described herein comprises a human immunoglobulin heavy chain constant region selected from the group consisting of a human IgG1, IgG2, IgG3, and IgG4.

Optionally the fusion protein of any of the foregoing or as described herein comprises a mouse IgG1, IgG2a or IgG2b immunoglobulin heavy chain constant region or fragment thereof.

Optionally the fusion protein of any of the foregoing or as described herein comprises an immunoglobulin Fc region that contains at least one mutation that alters effector function and/or glycosylation. Optionally said effector function is selected from FcR binding, complement binding, ADCC activity, CDC activity, degranulation, phagocytosis, and/or cytokine release.

Optionally the heterologous sequence comprises at least a portion of an immunoglobulin molecule that specifically binds to a target cell or comprises another moiety that specifically binds to a target cell. Optionally the target cell is a cancerous, immune, infectious agent cell, an infected cell, an immune cell, an inflammatory cell, a disease site or a cell which is to be transplanted into a human recipient. Optionally said infectious agent cell is selected from the group consisting of a virus, bacterium, mycoplasm, fungus, yeast or parasite. Optionally said infected cell is infected with an infectious agent selected from the group consisting of a virus, bacterium, mycoplasm, fungus, yeast or parasite. Optionally at least one of the heterologous polypeptides is a receptor, hormone, cytokine, antigen, B-cell target, NK cell target, T cell target, TNF receptor superfamily member, Hedgehog family member, a receptor tyrosine kinase, a proteoglycan-related molecule, a TGF-β superfamily member, a Wnt-related molecule, a receptor ligand, a Dendritic cell target, a myeloid cell target, a monocyte/macrophage cell target or an angiogenesis target. Optionally the antigen is a tumor antigen, autoantigen, allergen, or an infectious agent antigen. Optionally the at least one heterologous polypeptide includes an immunomodulatory polypeptide. Optionally the T cell target is selected from the group consisting of 2B4/SLAMF4, IL-2 Rα, 4-1BB/TNFRSF9, IL-2Rβ, ALCAM, B7-1/CD80, IL-4R, B7-H3, BLAME/SLAMF8, BTLA, IL-6R, CCR3, IL-7 Rα, CCR4, CXCR1/IL-8 RA, CCR5, CCR6, IL-10 R α, CCR7, IL-10 CCR8, IL-12 Rβ1, CCR9, IL-12 Rβ 2, CD2, IL-13Rα1, IL-13, CD3, CD4, ILT2/CD85j, ILT3/CD85k, ILT4/CD85d, ILT5/CD85a, Integrin α4/CD49d, CD5, IntegrinαE/CD103, CD6, Integrin α M/CD11b, CD8, Integrin α X/CD11c, Integrin β2/CD18, KIR/CD158, CD27/TNFRSF7, KIR2DL1, CD28, KIR2DL3, CD30/TNFRSF8, KIR2DL4/CD158d, CD31/PECAM-1, KIR2DS4, CD40 Ligand/TNFSF5, LAG-3, CD43, LAIR1, CD45, LAIR2, CD83, Leukotriene B4 R1, CD84/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 Rγ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP β1, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, GITR/TNFRSF18, TNF R1/TNFRSF1A, Granulysin, TNF R11/TNFRSF1B, HVEM/TNFRSF14, TRAIL R1/TNFRSF10A, ICAM-1/CD54, TRAIL R2/TNFRSF10B, ICAM-2/CD102, TRAIL R3/TNFRSF10C, IFN-γR1, TRAIL R4/TNFRSF10D, IFN-γR2, TSLP, IL-1 RI and TSLP R. Optionally the monocyte/macrophage cell target is selected from the group consisting of B7-1/CD80, ILT4/CD85d, B7-H1, ILT5/CD85a, Common β Chain, Integrin α4/CD49d, BLAME/SLAMF8, Integrin α X/CD11c, CCL6/C10, Integrin β2/CD18, CD155/PVR, Integrin β3/CD61, CD31/PECAM-1, Latexin, CD36/SR-B3, Leukotriene B4 R1, CD40/TNFRSF5, LIMPII/SR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc γR1/CD64, Osteopontin, Fc γ RIIB/CD32b, PD-L2, Fc γRIIC/CD32c, Siglec-3/CD33, Fcγ RIIA/CD32a, SIGNR1/CD209, Fcγ RIII/CD16, SLAM, GM-CSF R α, TCCR/WSX-1, ICAM-2/CD102, TLR3, IFN-γ R1, TLR4, IFN-γ R2, TREM-1, IL-1 RII, TREM-2, ILT2/CD85j, TREM-3, ILT3/CD85k, TREML1/TLT-1, 2B4/SLAMF4, IL-10 R α, ALCAM, IL-10 R β, Aminopeptidase N/ANPEP, ILT2/CD85j, Common β Chain, ILT3/CD85k, C1q R1/CD93, ILT4/CD85d, CCR1, ILT5/CD85a, CCR2, Integrin α4/CD49d, CCR5, Integrin α M/CD11b, CCR8, Integrin α X/CD11c, CD155/PVR, Integrin β2/CD18, CD14, Integrin β3/CD61, CD36/SR-B3, LAIR1, CD43, LAIR2, CD45, Leukotriene B4 R1, CD68, LIMPII/SR-B2, CD84/SLAMF5, LMIR1/CD300A, CD97, LMIR2/CD300c, CD163, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc γ R1/CD64, PSGL-1, Fc γ RIII/CD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-1, IL-6 R, TREM-2, CXCR1/IL-8 RA, TREM-3 and TREML1/TLT-1. Optionally the Dendritic cell target is selected from the group consisting of CD36/SR-B3, LOX-1/SR-E1, CD68, MARCO, CD163, SR-AI/MSR, CD5L, SREC-I, CL-P1/COLEC12, SREC-II, LIMPII/SR-B2, RP105, TLR4, TLR1, TLR5, TLR2, TLR6, TLR3, TLR9, 4-1BB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, Integrin α4/CD49d, Aag, Integrin β2/CD18, AMICA, Langerin, B7-2/CD86, Leukotriene B4 R1, B7-H3, LMIR1/CD300A, BLAME/SLAMF8, LMIR2/CD300c, C1q R1/CD93, LMIR3/CD300LF, CCR6, LMIR5/CD300LB, CCR7, LMIR6/CD300LE, CD40/TNFRSF5, MAG/Siglec-4a, CD43, MCAM, CD45, MD-1, CD68, MD-2, CD83, MDL-1/CLEC5A, CD84/SLAMF5, MMR, CD97, NCAM-L1, CD2F-10/SLAMF9, Osteoactivin/GPNMB, Chem 23, PD-L2, CLEC-1, RP105, CLEC-2, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc γ R1/CD64, TLR3, Fc γ RIIB/CD32b, TREM-1, Fc γ RIIC/CD32c, TREM-2, Fc γ RIIA/CD32a, TREM-3, Fc γ RIII/CD16, TREML1/TLT-1, ICAM-2/CD102 and Vanilloid R1. Optionally the TNF receptor superfamily member is selected from the group consisting of 4-1BB/TNFRSF9, NGF R/TNFRSF16, BAFF R/TNFRSF13C, Osteoprotegerin/TNFRSF11B, BCMA/TNFRSF17, OX40/TNFRSF4, CD27/TNFRSF7, RANK/TNFRSF11A, CD30/TNFRSF8, RELT/TNFRSF19L, CD40/TNFRSF5, TACI/TNFRSF13B, DcR3/TNFRSF6B, TNF RI/TNFRSF1A, DcTRAIL R1/TNFRSF23, TNF RII/TNFRSF1B, DcTRAIL R2/TNFRSF22, TRAIL R1/TNFRSF10A, DR3/TNFRSF25, TRAIL R2/TNFRSF10B, DR6/TNFRSF21, TRAIL R3/TNFRSF10C, EDAR, TRAIL R4/TNFRSF10D, Fas/TNFRSF6, TROY/TNFRSF19, GITR/TNFRSF18, TWEAK R/TNFRSF12, HVEM/TNFRSF14, XEDAR, Lymphotoxin β R/TNFRSF3, 4-1BB Ligand/TNFSF9, Lymphotoxin, APRIUTNFSF13, Lymphotoxin β R/TNFSF3, BAFF/TNFSF13C, OX40 Ligand/TNFSF4, CD27 Ligand/TNFSF7, TL1A/TNFSF15, CD30 Ligand/TNFSF8, TNF-α/TNFSF1A, CD40 Ligand/TNFSF5, TNF-β/TNFSF1B, EDA-A2, TRAIL/TNFSF10, Fas Ligand/TNFSF6, TRANCE/TNFSF11, GITR Ligand/TNFSF18, TWEAK/TNFSF12 and LIGHT/TNFSF14. Optionally the Hedgehog family member is selected from the group consisting of Patched and Smoothened. Optionally the receptor tyrosine kinase is selected from the group consisting of Ax1, FGF R4, C1q R1/CD93, FGF R5, DDR1, Flt-3, DDR2, HGF R, Dtk, IGF-I R, EGF R, IGF-II R, Eph, INSRR, EphA1, Insulin R/CD220, EphA2, M-CSF R, EphA3, Mer, EphA4, MSP R/Ron, EphA5, MuSK, EphA6, PDGF R α, EphA7, PDGF R β, EphA8, Ret, EphB1, ROR1, EphB2, ROR2, EphB3, SCF R/c-kit, EphB4, Tie-1, EphB6, Tie-2, ErbB2, TrkA, ErbB3, TrkB, ErbB4, TrkC, FGF R1, VEGF R1/Flt-1, FGF R2, VEGF R2/Flk-1, FGF R3 and VEGF R3/Flt-4. Optionally the Transforming Growth Factor (TGF)-β superfamily member is selected from the group consisting of Activin RIA/ALK-2, GFR α-1, Activin RIB/ALK-4, GFR α2, Activin RHA, GFR α-3, Activin RIIB, GFR α-4, ALK-1, MIS RII, ALK-7, Ret, BMPR-IA/ALK-3, TGF-betβa R1/ALK-5, BMPR-IB/ALK-6, TGF-β RII, BMPR-II, TGF-β RIIb, Endoglin/CD 105 and TGF-β RIII. Optionally the Wnt-related molecule selected from the group consisting of Frizzled-1, Frizzled-8, Frizzled-2, Frizzled-9, Frizzled-3, sFRP-1, Frizzled-4, sFRP-2, Frizzled-5, s1-RP-3, Frizzled-6, sFRP-4, Frizzled-7, MFRP, LRP 5, LRP 6, Wnt-1, Wnt-8a, Wnt-3a, Wnt-10b, Wnt-4, Wnt-11, Wnt-5a, Wnt-9a and Wnt-7a. Optionally the receptor ligand is selected from the group consisting of 4-1BB Ligand/TNFSF9, Lymphotoxin, APRIL/TNFSF13, Lymphotoxin β/TNFSF3, BAFF/TNFSF13C, OX40 Ligand/TNFSF4, CD27 Ligand/TNFSF7, TL1A/TNFSF15, CD30 Ligand/TNFSF8, TNF-α/TNFSF1A, CD40 Ligand/TNFSF5, TNF-β/TNFSF1B, EDA-A2, TRAIL/TNFSF10, Fas Ligand/TNFSF6, TRANCE/TNFSF11, GITR Ligand/TNFSF18, TWEAK/TNFSF12, LIGHT/TNFSF14, Amphiregulin, NRG1 isoform GGF2, Betacellulin, NRG1 Isoform SMDF, EGF, NRG1-α/HRG1-α, Epigen, NRG1-β1/HRG1-β1, Epiregulin, TGF-α, HB-EGF, TMEFF1/Tomoregulin-1, Neuregulin-3, TMEFF2, IGF-I, IGF-II, Insulin, Activin A, Activin B, Activin AB, Activin C, BMP-2, BMP-7, BMP-3, BMP-8, BMP-3b/GDF-10, BMP-9, BMP-4, BMP-15, BMP-5, Decapentaplegic, BMP-6, GDF-1, GDF-8, GDF-3, GDF-9, GDF-5, GDF-11, GDF-6, GDF-15, GDF-7, Artemin, Neurturin, GDNF, Persephin, TGF-θ, TGF-β2, TGF-β1, TGF-β3, LAP (TGF-β1), TGF-β5, Latent TGF-β1, Latent TGF-β bp1, TGF-β1.2, Lefty, Nodal, MIS/AMH, FGF acidic, FGF-12, FGF basic, FGF-13, FGF-3, FGF-16, FGF-4, FGF-17, FGF-5, FGF-19, FGF-6, FGF-20, FGF-8, FGF-21, FGF-9, FGF-23, FGF-10, KGF/FGF-7, FGF-11, Neuropilin-1, P1GF, Neuropilin-2, P1GF-2, PDGF, PDGF-A, VEGF, PDGF-B, VEGF-B, PDGF-C, VEGF-C, PDGF-D, VEGF-D and PDGF-AB. Optionally the tumor antigen is selected from the group consisting of Squamous Cell Carcinoma Antigen 1 (SCCA-1), (PROTEIN T4-A), Squamous Cell Carcinoma Antigen 2 (SCCA-2), Ovarian carcinoma antigen CA125 (1A1-3B; KIAA0049), MUCIN 1 (TUMOR-ASSOCIATED MUCIN; Carcinoma-Associated Mucin; Polymorphic Epithelial Mucin; PEM; PEMT; EPISIALIN; Tumor-Associated Epithelial Membrane Antigen; EMA; H23AG; Peanut-Reactive Urinary Mucin; PUM; and Breast Carcinoma-Associated Antigen DF3), CTCL tumor antigen sel-1, CTCL tumor antigen se14-3, CTCL tumor antigen se20-4, CTCL tumor antigen se20-9, CTCL tumor antigen se33-1, CTCL tumor antigen se37-2, CTCL tumor antigen se57-1, CTCL tumor antigen se89-1, Prostate-specific membrane antigen, 5T4 oncofetal trophoblast glycoprotein, Orf73 Kaposi's sarcoma-associated herpesvirus, MAGE-C1 (cancer/testis antigen CT7), MAGE-B1 ANTIGEN (MAGE-XP Antigen; DAM10), MAGE-B2 Antigen (DAM6), MAGE-2 ANTIGEN, MAGE-4a antigen, MAGE-4b antigen, Colon cancer antigen NY-CO-45, Lung cancer antigen NY-LU-12 variant A, Cancer associated surface antigen, Adenocarcinoma antigen ART1, Paraneoplastic associated brain-testis-cancer antigen (onconeuronal antigen MA2; paraneoplastic neuronal antigen), Neuro-oncological ventral antigen 2 (NOVA2), Hepatocellular carcinoma antigen gene 520, Tumor-Associated Antigen CO-029, Tumor-associated antigen MAGE-X2, Synovial sarcoma, X breakpoint 2, Squamous cell carcinoma antigen recognized by T cell, Serologically defined colon cancer antigen 1, Serologically defined breast cancer antigen NY-BR-15, Serologically defined breast cancer antigen NY-BR-16, Chromogranin A, parathyroid secretory protein 1, DUPAN-2, CA 19-9, CA 72-4, CA 195 and L6. Optionally the B cell target is selected from the group consisting of CD10, CD19, CD20, CD21, CD22, CD23, CD24, CD37, CD38, CD39, CD40, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138 and CDw150. Optionally the angiogenesis target is selected from the group consisting of Angiopoietin-1, Angiopoietin-like 2, Angiopoietin-2, Angiopoietin-like 3, Angiopoietin-3, Angiopoietin-like 7/CDT6, Angiopoietin-4, Tie-1, Angiopoietin-like 1, Tie-2, Angiogenin, iNOS, Coagulation Factor III/Tissue Factor, nNOS, CTGF/CCN2, NOV/CCN3, DANCE, OSM, EDG-1, Plfr, EG-VEGF/PK1, Proliferin, Endostatin, ROBO4, Erythropoietin, Thrombospondin-1, Kininostatin, Thrombospondin-2, MFG-E8, Thrombospondin-4, Nitric Oxide, VGSQ, eNOS, EphA1, EphA5, EphA2, EphA6, EphA3, EphA7, EphA4, EphA8, EphB1, EphB4, EphB2, EphB6, EphB3, Ephrin-A1, Ephrin-A4, Ephrin-A2, Ephrin-A5, Ephrin-A3, Ephrin-B1, Ephrin-B3, Ephrin-B2, FGF acidic, FGF-12, FGF basic, FGF-13, FGF-3, FGF-16, FGF-4, FGF-17, FGF-5, FGF-19, FGF-6, FGF-20, FGF-8, FGF-21, FGF-9, FGF-23, FGF-10, KGF/FGF-7, FGF-11, FGF R1, FGF R4, FGF R2, FGF R5, FGF R3, Neuropilin-1, Neuropilin-2, Semaphorin 3A, Semaphorin 6B, Semaphorin 3C, Semaphorin 6C, Semaphorin 3E, Semaphorin 6D, Semaphorin 6A, Semaphorin 7A, MMP, MMP-11, MMP-1, MMP-12, MMP-2, MMP-13, MMP-3, MMP-14, MMP-7, MMP-15, MMP-8, MMP-16/MT3-MMP, MMP-9, MMP-24/MT5-MMP, MMP-10, MMP-25/MT6-MMP, TIMP-1, TIMP-3, TIMP-2, TIMP-4, ACE, IL-13 R α1, IL-13, C1q R1/CD93, Integrin α4/CD49d, VE-Cadherin, Integrin β2/CD18, CD31/PECAM-1, KLF4, CD36/SR-B3, LYVE-1, CD151, MCAM, CL-P1/COLEC12, Nectin-2/CD112, Coagulation Factor III/Tissue Factor, E-Selectin, D6, P-Selectin, DC-SIGNR/CD299, SLAM, EMMPRIN/CD147, Tie-2, Endoglin/CD105, TNF RI/TNFRSF1A, EPCR, TNF RII/TNFRSF1B, Erythropoietin R, TRAIL R1/TNFRSF10A, ESAM, TRAIL R2/TNFRSF10B, FABP5, VCAM-1, ICAM-1/CD54, VEGF R2/Flk-1, ICAM-2/CD102, VEGF R3/Flt-4, IL-1 RI and VGSQ.

According to at least some embodiments, there is provided an isolated polypeptide or the fusion protein according to any of the foregoing or as described herein which agonizes at least one immune inhibitory effect of VSTM5; optionally which mediates at least one of the following effects: (i) decreases immune response, (ii) decreases T cell activation, (iii) decreases cytotoxic T cell activity, (iv) decreases natural killer (NK) cell activity, (v) decreases T-cell activity, (vi) decreases pro-inflammatory cytokine secretion, (vii) decreases IL-2 secretion; (viii) decreases interferon-γ production by T-cells, (ix) decreases Th1 response, (x) decreases Th2 response, (xi) increases cell number and/or activity of regulatory T cells, (xii) increases regulatory cell activity and/or one or more of myeloid derived suppressor cells (MDSCs), iMCs, mesenchymal stromal cells, TIE2-expressing monocytes, (xiii) increases regulatory cell activity and/or the activity of one or more of myeloid derived suppressor cells (MDSCs), iMCs, mesenchymal stromal cells, TIE2-expressing monocytes, (xiii) increases M2 macrophages, (xiv) increases M2 macrophage activity, (xv) increases N2 neutrophils, (xvi) increases N2 neutrophils activity, (xvii) increases inhibition of T cell activation, (xviii) increases inhibition of CTL activation, (xix) increases inhibition of NK cell activation, (xx) increases T cell exhaustion, (xxi) decreases T cell response, (xxii) decreases activity of cytotoxic cells, (xxiii) reduces antigen-specific memory responses, (xxiv) inhibits apoptosis or lysis of cells, (xxv) decreases cytotoxic or cytostatic effect on cells, (xxvi) reduces direct killing of cells, (xxvii) decreases Th17 activity, and/or (xxviii) reduces complement dependent cytotoxicity and/or antibody dependent cell-mediated cytotoxicity, with the proviso that said isolated or recombinant VSTM5 polypeptide or fusion protein may elicit an opposite effect to one or more of (i)-(xxviii).

According to at least some embodiments, there is provided an isolated polypeptide or fusion protein according to any of the foregoing or as described herein which antagonizes at least one immune inhibitory effect of VSTM5; optionally which mediates at least one of the following effects (i) increases immune response, (ii) increases T cell activation, (iii) increases cytotoxic T cell activity, (iv) increases NK cell activity, (v) alleviates T-cell suppression, (vi) increases pro-inflammatory cytokine secretion, (vii) increases IL-2 secretion; (viii) increases interferon-γ production by T-cells, (ix) increases Th1 response, (x) decrease Th2 response, (xi) decreases or eliminates cell number and/or activity of at least one of regulatory T cells (Tregs), myeloid derived suppressor cells (MDSCs), iMCs, mesenchymal stromal cells, TIE2-expressing monocytes, (xii) reduces regulatory cell activity, and/or the activity of one or more of myeloid derived suppressor cells (MDSCs), iMCs, mesenchymal stromal cells, TIE2-expressing monocytes, (xiii) decreases or eliminates M2 macrophages, (xiv) reduces M2 macrophage pro-tumorigenic activity, (xv) decreases or eliminates N2 neutrophils, (xvi) reduces N2 neutrophils pro-tumorigenic activity, (xvii) reduces inhibition of T cell activation, (xviii) reduces inhibition of CTL activation, (xix) reduces inhibition of NK cell activation, (xx) reverses T cell exhaustion, (xxi) increases T cell response, (xxii) increases activity of cytotoxic cells, (xxiii) stimulates antigen-specific memory responses, (xxiv) elicits apoptosis or lysis of cancer cells, (xxv) stimulates cytotoxic or cytostatic effect on cancer cells, (xxiv) induces direct killing of cancer cells, (xxvi) increases Th17 activity and/or (xxvii) induces complement dependent cytotoxicity and/or antibody dependent cell-mediated cytotoxicity, with the proviso that said isolated or recombinant VSTM5 polypeptide or fusion protein may elicit an opposite effect to one or more of (i)-(xxvii).

According to at least some embodiments, there is provided an isolated polypeptide or fusion protein according to any of the forgoing or as described herein which agonizes or antagonizes at least one effect of VSTM5 on T cells, natural killer (NK) cells or the production of one or more proinflammatory cytokines.

According to at least some embodiments, there is provided an isolated polypeptide or fusion protein according to any of the forgoing or as described herein which inhibits or promotes one or more of CTL activity, CD4+ T cell activation and/or CD4+ T cell proliferation and/or cell depletion or the secretion of proinflammatory cytokines.

According to at least some embodiments, there is provided an isolated polypeptide or fusion protein according to any of the forgoing or as described herein which inhibits or promotes NK cell activity.

According to at least some embodiments, there is provided an isolated polypeptide or fusion protein according to any of the forgoing or as described herein which inhibits or promotes the differentiation, proliferation and/or activity of Tregs, MDSCs, iMCs, mesenchymal stromal cells, TIE2-expressing monocytes, and/or the infiltration of Tregs (Tregs), MDSCs iMCs, mesenchymal stromal cells, TIE2-expressing monocytes. Optionally said Tregs are inducible Tregs.

According to at least some embodiments, there is provided an isolated polypeptide or fusion protein according to any of the forgoing or as described herein which specifically binds to a receptor expressed by NK cells.

According to at least some embodiments, there is provided an isolated polypeptide or fusion protein according to any of the forgoing or as described herein which specifically binds to a receptor expressed by activated T cells.

According to at least some embodiments, there is provided an isolated polynucleotide encoding an isolated polypeptide or fusion protein according to any of the foregoing or as described herein.

According to at least some embodiments, there is provided an expression vector or a virus, comprising at least one polynucleotide as described herein.

According to at least some embodiments, there is provided a recombinant cell comprising an expression vector as described herein or a virus containing a polynucleotide as described herein, wherein the cell constitutively or inducibly expresses the polypeptide encoded by the DNA segment.

According to at least some embodiments, there is provided a method of producing an isolated polypeptide or fusion protein according to any of the forgoing or as described herein, comprising culturing the recombinant cell as described herein, under conditions whereby the cell expresses the polypeptide encoded by the DNA segment or nucleic acid and recovering said polypeptide.

According to at least some embodiments, there is provided a pharmaceutical composition comprising the isolated protein or fusion protein according to any of the forgoing or as described herein, or comprising a VSTM5 ECD protein set forth in any of SEQ ID NOs: 2, 3, the polynucleotide as described herein, the expression vector or virus as described herein, or the recombinant cell as described herein; optionally provided for use in treatment in a subject suffering from cancer; also optionally provided for use in immunotherapy treatment of cancer; also optionally, wherein the cancer does not express sufficient levels of VSTM5 protein at diagnosis or prior to the treatment, or alternatively and optionally wherein the cancer does express sufficient levels of VSTM5 protein at diagnosis or prior to the treatment.

Optionally said pharmaceutical composition, isolated polypeptide, fusion protein, polynucleotide, expression vector, virus or cell is administered to the subject in need thereof in combination with a therapeutic agent useful for treatment of cancer.

Optionally the pharmaceutical composition, cancer immunotherapy, the isolated polypeptide or fusion protein, the polynucleotide, the expression vector or virus, the recombinant cell or the use according to the foregoing or as described herein is provided for performing at least one of the following: (i) increasing immune response, (ii) increasing T cell activation, (iii) increasing cytotoxic T cell activity, (iv) increasing NK cell activity, (v) increasing Th17 activity, (vi) alleviating T-cell suppression, (vii) increasing pro-inflammatory cytokine secretion, (viii) increasing IL-2 secretion; (ix) increasing interferon-γ production by T-cells, (x) increasing Th1 response, (xi) decreasing Th2 response, (xii) decreasing or eliminating at least one of regulatory T cells (Tregs), myeloid derived suppressor cells (MDSCs), iMCs, mesenchymal stromal cells, TIE2-expressing monocytes, (xiii) reducing regulatory cell activity and/or the activity of one or more of myeloid derived suppressor cells (MDSCs), iMCs, mesenchymal stromal cells, TIE2-expressing monocytes, (xiv) decreasing or eliminating M2 macrophages, (xv) reducing M2 macrophage pro-tumorigenic activity, (xvi) decreases or eliminates N2 neutrophils, (xvii) reduces N2 neutrophils pro-tumorigenic activity, (xviii) reducing inhibition of T cell activation, (xix) reducing inhibition of CTL activation, (xx) reducing inhibition of NK cell activation, (xxi) reversing T cell exhaustion, (xxii) increasing T cell response, (xxiii) increasing activity of cytotoxic cells, (xxiv) stimulating antigen-specific memory responses, (xxv) eliciting apoptosis or lysis of cancer cells, (xxvi) stimulating cytotoxic or cytostatic effect on cancer cells, (xxvii) inducing direct killing of cancer cells, and/or (xxviii) inducing complement dependent cytotoxicity and/or (xxix) inducing antibody dependent cell-mediated cytotoxicity.

Optionally the pharmaceutical composition, cancer immunotherapy, the isolated polypeptide or fusion protein, the polynucleotide, the expression vector or virus, the recombinant cell or the use according to the foregoing or as described herein further comprises administering an additional therapy comprising one or more of radiotherapy, cryotherapy, antibody therapy, chemotherapy, photodynamic therapy, surgery, hormonal deprivation, targeted therapy agent, a cancer vaccine or combination therapy with conventional drugs.

Optionally the therapeutic agent or additional therapy is selected from the group consisting of cytotoxic drugs, tumor vaccines, antibodies, peptides, pepti-bodies, small molecules, chemotherapeutic agents, cytotoxic and cytostatic agents, immunological modifiers, interferons, interleukins, immunostimulatory growth hormones, cytokines, vitamins, minerals, aromatase inhibitors, RNAi, Histone Deacetylase Inhibitors, and proteasome inhibitors.

Optionally the pharmaceutical composition, cancer immunotherapy, the isolated polypeptide or fusion protein, the polynucleotide, the expression vector or virus, the recombinant cell or the use according to the foregoing or as described herein, administered to a subject simultaneously or sequentially in combination with one or more therapeutic agents, additional therapy or potentiating agents to obtain a therapeutic effect, wherein said one or more potentiating agents is selected from the group consisting of radiotherapy, conventional/classical anti-cancer therapy potentiating anti-tumor immune responses, Targeted therapy potentiating anti-tumor immune responses, Therapeutic agents targeting immunosuppressive cells Tregs and/or MDSCs, Immunostimulatory antibodies, Cytokine therapy, and Adoptive cell transfer.

Optionally the conventional/classical anti-cancer agent is selected from the group consisting of platinum based compounds, antibiotics with anti-cancer activity, Anthracyclines, Anthracenediones, alkylating agents, antimetabolites, Antimitotic agents, Taxanes, Taxoids, microtubule inhibitors, Vinca alkaloids, Folate antagonists, Topoisomerase inhibitors, Antiestrogens, Antiandrogens, Aromatase inhibitors, GnRh analogs, inhibitors of 5α-reductase, bisphosphonates and antibodies.

Optionally the Targeted therapy agent is selected from the group consisting of histone deacetylase (HDAC) inhibitors, proteasome inhibitors, mTOR pathway inhibitors, JAK2 inhibitors, tyrosine kinase inhibitors (TKIs), PI3K inhibitors, Protein kinase inhibitors, Inhibitors of serine/threonine kinases, inhibitors of intracellular signaling, inhibitors of Ras/Raf signaling, MEK inhibitors, AKT inhibitors, inhibitors of survival signaling proteins, cyclin dependent kinase inhibitors, therapeutic monoclonal antibodies, TRAIL pathway agonists, anti-angiogenic agents, metalloproteinase inhibitors, cathepsin inhibitors, inhibitors of urokinase plasminogen activator receptor function, immunoconjugates, antibody drug conjugates, antibody fragments, bispecfic antibodies, bispecific T cell engagers (BiTEs).

Optionally the antibody is selected from cetuximab, panitumumab, nimotuzumab, trastuzumab, pertuzumab, rituximab, ofatumumab, veltuzumab, alemtuzumab, labetuzumab, adecatumumab, oregovomab, onartuzumab; apomab, mapatumumab, lexatumumab, conatumumab, tigatuzumab, catumaxomab, blinatumomab, ibritumomab triuxetan, tositumomab, brentuximab vedotin, gemtuzumab ozogamicin, clivatuzumab tetraxetan, pemtumomab, trastuzumab emtansine, bevacizumab, etaracizumab, volociximab, ramucirumab, aflibercept.

Optionally the Therapeutic agent targeting immunosuppressive cells Tregs and/or MDSCs is selected from antimitotic drugs, cyclophosphamide, gemcitabine, mitoxantrone, fludarabine, thalidomide, thalidomide derivatives, COX-2 inhibitors, depleting or killing antibodies that directly target Tregs through recognition of Treg cell surface receptors, anti-CD25 daclizumab, basiliximab, ligand-directed toxins, denileukin diftitox (Ontak)—a fusion protein of human IL-2 and diphtheria toxin, or LMB-2—a fusion between an scFv against CD25 and the pseudomonas exotoxin, antibodies targeting Treg cell surface receptors, TLR modulators, agents that interfere with the adenosinergic pathway, ectonucleotidase inhibitors, or inhibitors of the A2A adenosine receptor, TGF-β inhibitors, chemokine receptor inhibitors, retinoic acid, all-trans retinoic acid (ATRA), Vitamin D3, phosphodiesterase 5 inhibitors, sildenafil, ROS inhibitors and nitroaspirin.

Optionally the Immunostimulatory antibody is selected from antagonistic antibodies targeting one or more of CTLA4, PD-1, PDL-1, LAG-3, TIM-3, BTLA, B7-H4, B7-H3, VISTA, and/or Agonistic antibodies targeting one or more of CD40, CD137, OX40, GITR, CD27, CD28 or ICOS.

Optionally the therapeutic cancer vaccine is selected from exogenous cancer vaccines including proteins or peptides used to mount an immunogenic response to a tumor antigen, recombinant virus and bacteria vectors encoding tumor antigens, DNA-based vaccines encoding tumor antigens, proteins targeted to dendritic cell-based vaccines, whole tumor cell vaccines, gene modified tumor cells expressing GM-CSF, ICOS and/or Flt3-ligand, oncolytic virus vaccines.

Optionally the cytokine therapy is selected from one or more of the cytokines IL-2, IL-7, IL-12, IL-15, IL-17, IL-18 and IL-21, IL23, IL-27, GM-CSF, IFNα (interferon alpha), IFNα-2b, IFNβ, IFNγ, and their different strategies for delivery.

Optionally the adoptive cell transfer therapy is carried out following ex vivo treatment selected from expansion of the patient autologous naturally occurring tumor specific T cells or genetic modification of T cells to confer specificity for tumor antigens.

According to at least some embodiments, there is provided an assay for diagnosing or aiding in the diagnosis of a disease in a subject, wherein the disease is selected from the group consisting of cancer, autoimmune disease, or an infectious disease, comprising the isolated polypeptide or fusion protein of any of the foregoing or as described herein, and/or with VSTM5 ECD protein set forth in any of SEQ ID NOs: 2, 3, and a detector for detecting specific binding of the isolated protein or fusion protein to a tissue sample taken from the subject.

According to at least some embodiments, there is provided a diagnostic method for diagnosing or aiding in the diagnosis of a disease in a subject, wherein the disease is selected from the group consisting of cancer, autoimmune disease, or infectious disease, comprising using the assay of any of the foregoing or as described herein for performing the method.

According to at least some embodiments, there is provided a diagnostic method for diagnosing or aiding in the diagnosis of a disease in a subject, wherein the disease is selected from the group consisting of cancer, autoimmune disease, or infectious disease, wherein the diagnostic method is performed ex vivo, and comprises contacting a tissue sample from the subject with the isolated polypeptide or fusion protein of any of the foregoing or as described herein and/or with VSTM5 ECD protein set forth in any of SEQ ID NOs: 2, 3, and detecting specific binding to the tissue sample.

According to at least some embodiments, there is provided a diagnostic method for diagnosing or aiding in the diagnosis of a disease in a subject, wherein the disease is selected from the group consisting of cancer, autoimmune disease, or an infectious disease, wherein the diagnostic method is performed in vivo, comprising administering the isolated polypeptide or fusion protein of any of the foregoing or as described herein, and/or with VSTM5 ECD protein set forth in any of SEQ ID NOs: 2, 3, to a subject and detecting specific binding to tissues.

Optionally the method according to any of the foregoing or as described herein, or use of the assay according to any of the foregoing or as described herein, wherein the diagnostic method is performed before therapy or treatment comprising administering the isolated polypeptide or fusion protein according to any of the foregoing or as described herein, the polynucleotide according to any of the foregoing or as described herein, the expression vector or virus according to any of the foregoing or as described herein, the recombinant cell according to any of the foregoing or as described herein, the pharmaceutical composition according to any of the foregoing or as described herein, the use according to any of the foregoing or as described herein, or the protein, the polynucleotide, the expression vector or virus, the recombinant cell, the pharmaceutical composition, or the use according to any of the foregoing or as described herein, to the subject.

Optionally the method according to any of the foregoing or as described herein, I provided for screening for a disease, screening for VSTM5-mediated immunosuppression, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations.

Optionally the isolated polypeptide or fusion protein according to any of the foregoing or as described herein, the polynucleotide according to any of the foregoing or as described herein, the expression vector or virus according to any of the foregoing or as described herein, the recombinant cell according to any of the foregoing or as described herein, the pharmaceutical composition according to any of the foregoing or as described herein, the use according to any of the foregoing or as described herein, or the protein, the polynucleotide, the expression vector or virus, the recombinant cell, the pharmaceutical composition, the use according to any of the foregoing or as described herein, the assay according to any of the foregoing or as described herein or the method according to any of the foregoing or as described herein, wherein said cancer is selected from the group consisting of breast cancer, cervical cancer, ovary cancer, endometrial cancer, melanoma, uveal melanoma, bladder cancer, lung cancer, pancreatic cancer, colorectal cancer, prostate cancer, leukemia, acute lymphocytic leukemia, chronic lymphocytic leukemia, B-cell lymphoma, Burkitt's lymphoma, multiple myeloma, Non-Hodgkin's lymphoma, myeloid leukemia, acute myelogenous leukemia (AML), chronic myelogenous leukemia, thyroid cancer, thyroid follicular cancer, myelodysplastic syndrome (MDS), fibrosarcomas and rhabdomyosarcomas, teratocarcinoma, neuroblastoma, glioma, glioblastoma, benign tumor of the skin, keratoacanthomas, renal cancer, anaplastic large-cell lymphoma, esophageal cancer, follicular dendritic cell carcinoma, seminal vesicle tumor, epidermal carcinoma, spleen cancer, bladder cancer, head and neck cancer, stomach cancer, liver cancer, bone cancer, brain cancer, cancer of the retina, biliary cancer, small bowel cancer, salivary gland cancer, cancer of uterus, cancer of testicles, cancer of connective tissue, myelodysplasia, Waldenström's macroglobinaemia, nasopharyngeal, neuroendocrine cancer, mesothelioma, angiosarcoma, Kaposi's sarcoma, carcinoid, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL), cancer of unknown origin either primary or metastatic, wherein the cancer is non-metastatic, invasive or metastatic. Optionally said breast cancer is breast carcinoma, and is selected from the group consisting of ductal-carcinoma, infiltrating ductal carcinoma, lobular carcinoma, mucinous adenocarcinoma, intra duct and invasive ductal carcinoma, and Scirrhous adenocarcinoma. Optionally said colon cancer is selected from the group consisting of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum. Optionally said lung cancer is selected from the group consisting of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma. Optionally said prostate cancer is prostate adenocarcinoma and is selected from the group consisting of Adenocarcinoma Gleason Grade 6 to 9, Infiltrating adenocarcinoma, High grade prostatic intraepithelial neoplasia, undifferentiated carcinoma. Optionally said stomach cancer is moderately differentiated gastric adenocarcinoma. Optionally said ovarian cancer is selected from the group consisting of cystadenocarcinoma, serous papillary cystic carcinoma, Serous papillary cystic carcinoma, Invasive serous papillary carcinoma. Optionally said brain cancer is selected from the group consisting of Astrocytoma, with the proviso that it is not a grade 2 astrocytoma, preferably grade 4 Astrocytoma, or Glioblastoma multiforme. Optionally said brain cancer is astrocytoma. Optionally said kidney cancer is clear cell renal cell carcinoma. Optionally the liver cancer is Hepatocellular carcinoma. Optionally said Hepatocellular carcinoma is Low Grade hepatocellular carcinoma or Fibrolamellar Hepatocellular Carcinoma. Optionally said lymphoma is selected from the group consisting of Hodgkin's Lymphoma and High to low grade Non-Hodgkin's Lymphoma.

Optionally the isolated protein, the fusion protein, the polynucleotide, the expression vector or virus, the recombinant cell, the pharmaceutical composition, the assay or the method according to any of the foregoing or as described herein, is provided for treating a subject suffering from a disease selected from the group consisting of B-cell lymphoma, Burkitt's lymphoma, thyroid cancer, thyroid follicular cancer, myelodysplastic syndrome (MDS), fibrosarcomas and rhabdomyosarcomas, melanoma, uveal melanoma, teratocarcinoma, neuroblastoma, glioma, glioblastoma cancer, keratoacanthomas, anaplastic large-cell lymphoma, esophageal squamous cells carcinoma, hepatocellular carcinoma cancer, follicular dendritic cell carcinoma, muscle-invasive cancer, seminal vesicle tumor, epidermal carcinoma, cancer of the retina, biliary cancer, small bowel cancer, salivary gland cancer, cancer of connective tissue, myelodysplasia, Waldenström's macroglobinaemia, nasopharyngeal, neuroendocrine cancer, myelodysplastic syndrome, mesothelioma, angiosarcoma, Kaposi's sarcoma, carcinoid, esophagogastric, fallopian tube cancer, peritoneal cancer, papillary serous müllerian cancer, malignant ascites, gastrointestinal stromal tumor (GIST), Li-Fraumeni syndrome and Von Hippel-Lindau syndrome (VHL); endometrial cancer, Breast carcinoma, preferably any of ductal-carcinoma, infiltrating ductal carcinoma, lobular carcinoma, mucinous adenocarcinoma, intra duct and invasive ductal carcinoma, and Scirrhous adenocarcinoma, Colorectal adenocarcinoma, preferably any of Poorly to Well Differentiated invasive and noninvasive Adenocarcinoma, Poorly to Well Differentiated Adenocarcinoma of the cecum, Well to Poorly Differentiated Adenocarcinoma of the colon, Tubular adenocarcinoma, preferably Grade 2 Tubular adenocarcinoma of the ascending colon, colon adenocarcinoma Duke's stage C1, invasive adenocarcinoma, Adenocarcinoma of the rectum, preferably Grade 3 Adenocarcinoma of the rectum, Moderately Differentiated Adenocarcinoma of the rectum, Moderately Differentiated Mucinous adenocarcinoma of the rectum; Lung cancer, preferably any of Well to Poorly differentiated Non-small cell carcinoma, Squamous Cell Carcinoma, preferably well to poorly Differentiated Squamous Cell Carcinoma, keratinizing squamous cell carcinoma, adenocarcinoma, preferably poorly to well differentiated adenocarcinoma, large cell adenocarcinoma, Small cell lung cancer, preferably Small cell lung carcinoma, more preferably undifferentiated Small cell lung carcinoma; Prostate adenocarcinoma, preferably any of Adenocarcinoma Gleason Grade 6 to 9, Infiltrating adenocarcinoma, High grade prostatic intraepithelial neoplasia, undifferentiated carcinoma; Stomach adenocarcinoma, preferably moderately differentiated gastric adenocarcinoma; Ovary carcinoma, preferably any of cystadenocarcinoma, serous papillary cystic carcinoma, Serous papillary cystic carcinoma, Invasive serous papillary carcinoma; Brain cancer, preferably any of Astrocytoma, with the proviso that it is not a grade 2 astrocytoma, preferably grade 4 Astrocytoma, Glioblastoma multiforme; Kidney carcinoma, preferably Clear cell renal cell carcinoma; Liver cancer, preferably any of Hepatocellular carcinoma, preferably Low Grade hepatocellular carcinoma, Fibrolamellar Hepatocellular Carcinoma; Lymphoma, preferably any of, Hodgkin's Lymphoma and High to low grade Non-Hodgkin's Lymphoma, and with the proviso that if the cancer is brain cancer, it is not Astrocytoma grade 2, and if the cancer is Non-Hodgkin's Lymphoma, it is not a large cell Non-Hodgkin's Lymphoma, and wherein the cancer is non-metastatic, invasive or metastatic.

According to at least some embodiments, there is provided a pharmaceutical composition according to any of the forgoing or as described herein, the isolated polypeptide or fusion protein according to any of the forgoing or as described herein, the polynucleotide according to any of the forgoing or as described herein, the expression vector or virus according to any of the forgoing or as described herein, or the recombinant cell according to any of the forgoing or as described herein, for use in the treatment of an immune condition in a subject suffering from same. Optionally said pharmaceutical composition, isolated polypeptide, fusion protein, polynucleotide, expression vector, virus or cell is administered to the subject in need thereof in combination with a therapeutic agent useful for treatment of an immune condition.

Optionally the pharmaceutical composition, the isolated polypeptide or fusion protein, the polynucleotide, the expression vector or virus, the recombinant cell or the use according to any of the forgoing or as described herein, is provided for treating an immune condition, in a subject in need thereof.

Optionally the pharmaceutical composition, the isolated polypeptide or fusion protein, the polynucleotide, the expression vector or virus, the recombinant cell or the use according to any of the forgoing or as described herein, is provided for treatment of treatment of immune related diseases and/or for reducing the undesirable immune activation that follows gene or cell therapy, or transplantation of cells, tissues, and/or organs into a subject, and is capable of at least one of inhibiting immune response, reducing T cell activity, reducing NK cell activity, enhancing regulatory cell activity, enhancing T-cell suppression, enhancing immune regulatory cell activity, inducing establishment of immune tolerance, reducing pro-inflammatory cytokine secretion, re-establishing Th1-Th2 immune balance, reducing immune memory responses to self-antigens, decreasing or eliminating pro-inflammatory immune cells, decreasing or eliminating auto-reactive immune cells.

Optionally the pharmaceutical composition, the isolated polypeptide or fusion protein, the polynucleotide, the expression vector or virus, the recombinant cell or the use according to any of the forgoing or as described herein, is provided for performing at least one of the following: (i) decreasing immune response, (ii) decreasing T cell activation, (iii) decreasing cytotoxic T cell activity, (iv) decreasing natural killer (NK) cell activity, (v) decreasing T-cell activity, (vi) decreasing Th17 activity, (vii) decreasing pro-inflammatory cytokine secretion, (viii) decreasing IL-2 secretion; (ix) decreasing interferon-γ production by T-cells, (x) decreasing Th1 response, (xi) decreasing Th2 response, (xii) increasing regulatory T cells and/or one or more of myeloid derived suppressor cells (MDSCs), iMCs, mesenchymal stromal cells, TIE2-expressing monocytes, (xiii) increasing regulatory cell activity and/or the activity of one or more of myeloid derived suppressor cells (MDSCs), iMCs, mesenchymal stromal cells, TIE2-expressing monocytes, (xiv) increasing M2 macrophages, (xv) increasing M2 macrophage activity, (xvi) increasing N2 neutrophils, (xvii) increasing N2 neutrophils activity, (xviii) increasing inhibition of T cell activation, (xix) increasing inhibition of CTL activation, (xx) increasing inhibition of NK cell activation, (xxi) increasing T cell exhaustion, (xxii) decreasing T cell response, (xxiii) decreasing activity of cytotoxic cells, (xxiv) reducing antigen-specific memory responses, (xxv) inhibiting apoptosis or lysis of cells, (xxvi) decreasing cytotoxic or cytostatic effect on cells, (xxvii) reducing direct killing of cells, and/or (xxviii) reducing complement dependent cytotoxicity and/or (xxix) reducing antibody dependent cell-mediated cytotoxicity.

Optionally said immune condition is selected from the group consisting of autoimmune disease, transplant rejection, and graft versus host disease.

Optionally said autoimmune disease is selected from the group consisting of multiple sclerosis, psoriasis; rheumatoid arthritis; psoriatic arthritis, systemic lupus erythematosus (SLE); discoid lupus erythematosus, inflammatory bowel disease, ulcerative colitis; Crohn's disease; benign lymphocytic angiitis, thrombocytopenic purpura, idiopathic thrombocytopenia, idiopathic autoimmune hemolytic anemia, pure red cell aplasia, Sjögren's syndrome, rheumatic disease, connective tissue disease, inflammatory rheumatism, degenerative rheumatism, extra-articular rheumatism, juvenile rheumatoid arthritis, arthritis uratica, muscular rheumatism, chronic polyarthritis, cryoglobulinemic vasculitis, ANCA-associated vasculitis, antiphospholipid syndrome, myasthenia gravis, autoimmune hemolytic anaemia, Guillain-Barre syndrome, chronic immune polyneuropathy, autoimmune thyroiditis, insulin dependent diabetes mellitus, type I diabetes, Addison's disease, membranous glomerulonephropathy, Goodpasture's disease, autoimmune gastritis, autoimmune atrophic gastritis, pernicious anaemia, pemphigus, pemphigus vulgaris, cirrhosis, primary biliary cirrhosis, dermatomyositis, polymyositis, fibromyositis, myogelosis, celiac disease, immunoglobulin A nephropathy, Henoch-Schönlein purpura, Evans syndrome, Dermatitis, atopic dermatitis, psoriasis, psoriasis arthropathica, Graves' disease, Graves' ophthalmopathy, scleroderma, systemic scleroderma, progressive systemic scleroderma, asthma, allergy, primary biliary cirrhosis, Hashimoto's thyroiditis, primary myxedema, sympathetic ophthalmia, autoimmune uveitis, hepatitis, chronic action hepatitis, collagen diseases, ankylosing spondylitis, periarthritis humeroscapularis, panarteritis nodosa, chondrocalcinosis, Wegener's granulomatosis, microscopic polyangiitis, chronic urticaria, bullous skin disorders, pemphigoid, atopic eczema, bullous pemphigoid, cicatricial pemphigoid, vitiligo, atopic eczema, eczema, chronic urticaria, autoimmune urticaria, normocomplementemic urticarial vasculitis, hypocomplementemic urticarial vasculitis, autoimmune lymphoproliferative syndrome, Devic's disease, sarcoidosis, pernicious anemia, childhood autoimmune hemolytic anemia, idiopathic autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, normocomplementemic urticarial vasculitis, pericarditis, idiopathic pericarditis, myositis, anti-synthetase syndrome, scleritis, macrophage activation syndrome, Behçet's Syndrome, PAPA Syndrome, Blau's Syndrome, gout, adult and juvenile Still's disease, cryopyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, a rheumatic disease, polymyalgia rheumatica, mixed connective tissue disease, inflammatory rheumatism, degenerative rheumatism, extra-articular rheumatism, juvenile arthritis, juvenile rheumatoid arthritis, systemic juvenile idiopathic arthritis, arthritis uratica, muscular rheumatism, chronic polyarthritis, reactive arthritis, Reiter's syndrome, rheumatic fever, relapsing polychondritis, Raynaud's phenomenon, vasculitis, cryoglobulinemic vasculitis, temporal arteritis, giant cell arteritis, Takayasu arteritis, Behcet's disease, chronic inflammatory demyelinating polyneuropathy, autoimmune thyroiditis, insulin dependent diabetes mellitus, type I diabetes, Addison's disease, membranous glomerulonephropathy, polyglandular autoimmune syndromes, Goodpasture's disease, autoimmune gastritis, autoimmune atrophic gastritis, pernicious anaemia, pemphigus, pemphigus vulgaris, cirrhosis, primary biliary cirrhosis, idiopathic pulmonary fibrosis, myositis, dermatomyositis, juvenile dermatomyositis, polymyositis, fibromyositis, myogelosis, celiac disease, celiac sprue dermatitis, immunoglobulin A nephropathy, Henoch-Schonlein purpura, Evans syndrome, atopic dermatitis, psoriasis, psoriasis vulgaris, psoriasis arthropathica, Graves' disease, Graves' ophthalmopathy, scleroderma, systemic scleroderma, progressive systemic scleroderma, diffuse scleroderma, localized scleroderma, Crest syndrome, asthma, allergic asthma, allergy, primary biliary cirrhosis, fibromyalgia, chronic fatigue and immune dysfunction syndrome (CFIDS), autoimmune inner ear disease, Hyper IgD syndrome, Schnitzler's syndrome, autoimmune retinopathy, age-related macular degeneration, atherosclerosis, chronic prostatitis, alopecia, alopecia areata, alopecia universalis, alopecia totalis, utoimmune thrombocytopenic purpura, idiopathic thrombocytopenic purpura, pure red cell aplasia, and TNF receptor-associated periodic syndrome (TRAPS).

Optionally the pharmaceutical composition, the isolated polypeptide or fusion protein, the polynucleotide, the expression vector or virus, the recombinant cell or the use according to any of the forgoing or as described herein, is provided for treating an autoimmune disease selected from relapsing-remitting multiple sclerosis, primary progressive multiple sclerosis, secondary progressive multiple sclerosis; progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, acute multiple sclerosis, conditions relating to multiple sclerosis, psoriatic arthritis, gout and pseudo-gout, juvenile idiopathic arthritis, Still's disease, rheumatoid vasculitis, conditions relating to rheumatoid arthritis, discoid lupus, lupus arthritis, lupus pneumonitis, lupus nephritis. Conditions relating to systemic lupus erythematosus include osteoarticular tuberculosis, antiphospholipid antibody syndrome, inflammation of various parts of the heart, such as pericarditis, myocarditis, and endocarditis, Lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myelopathy, cranial neuropathy, polyneuropathy, vasculitis, Collagenous colitis, Lymphocytic colitis, Ischaemic colitis, Diversion colitis, Behçet's disease, Indeterminate colitis, thrombocytopenic purpura, idiopathic autoimmune hemolytic anemia, pure red cell aplasia, cryoglobulinemic vasculitis, ANCA-associated vasculitis, antiphospholipid syndrome, autoimmune hemolytic anaemia, Guillain-Barré syndrome, chronic immune polyneuropathy, autoimmune thyroiditis, idiopathic diabetes, juvenile type ldiabetes, maturity onset diabetes of the young, latent autoimmune diabetes in adults, gestational diabetes, conditions relating to type 1 diabetes, membranous glomerulonephropathy, autoimmune gastritis, pemphigus vulgaris, cirrhosis, fibromyositis, celiac disease, immunoglobulin A nephropathy, Henoch-Schönlein purpura, Evans syndrome, atopic dermatitis, psoriasis, Graves' ophthalmopathy, systemic scleroderma, asthma, allergy, anterior uveitis (or iridocyclitis), intermediate uveitis (pars planitis), posterior uveitis (or chorioretinitis), panuveitic form, hepatitis, Wegener's granulomatosis, microscopic polyangiitis, chronic urticaria, bullous skin disorders, pemphigoid, atopic eczema, Devic's disease, childhood autoimmune hemolytic anemia, Refractory or chronic Autoimmune Cytopenias, Prevention of development of Autoimmune Anti-Factor VIII Antibodies in Acquired Hemophilia A, Cold Agglutinin Disease, Neuromyelitis Optica, Stiff Person Syndrome, gingivitis, periodontitis, pancreatitis, myocarditis, vasculitis, gastritis, gout, gouty arthritis, and inflammatory skin disorders, selected from the group consisting of psoriasis, Nonpustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis, drug-induced psoriasis, Inverse psoriasis, Napkin psoriasis, Seborrheic-like psoriasis, Guttate psoriasis, Nail psoriasis, Psoriatic arthritis, atopic dermatitis, eczema, rosacea, urticaria, and acne, normocomplementemic urticarial vasculitis, pericarditis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, autoimmune retinopathy, age-related macular degeneration, atherosclerosis, chronic prostatitis and TNF receptor-associated periodic syndrome (TRAPS).

Optionally the treatment is combined with another moiety useful for treating said condition.

Optionally said other moiety useful for treating immune related condition is selected from immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-alpha blockers or antagonists, or any other biological agent targeting any inflammatory cytokine, nonsteroidal antiinflammatory drugs/Cox-2 inhibitors, hydroxychloroquine, sulfasalazine, gold salts, etanercept, infliximab, mycophenolate mofetil, basiliximab, atacicept, rituximab, Cytoxan® (cyclophosphamide), interferon beta-1a, interferon beta-1b, glatiramer acetate, mitoxantrone hydrochloride, anakinra and/or other biologics and/or intravenous immunoglobulin (IVIG), interferons such as IFN-beta-1a (REBIF®. AVONEX® and CINNOVEX®) and IFN-beta-1b (BETASERON®); EXTAVIA®, BETASERON®, ZIFERON®); glatiramer acetate (COPAXONE®), a polypeptide; natalizumab (TYSABRI®), mitoxantrone (NOVANTRONE®), a cytotoxic agent, a calcineurin inhibitor; cyclosporin A; FK506; an immunosuppressive macrolide; rapamycin; a rapamycin derivative; 40-O-(2-hydroxy)ethyl-rapamycin, a lymphocyte homing agent, FTY720; an analog of FTY720; corticosteroids; cyclophosphamide; azathioprine; methotrexate; leflunomide or an analog thereof; mizoribine; mycophenolic acid; mycophenolate mofetil; 15-deoxyspergualine or an analog thereof; immunosuppressive monoclonal antibodies, monoclonal antibodies to leukocyte receptors, monoclonal antibodies to MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, CTLA4-Ig (abatacept, ORENCIA®, belatacept), CD28-Ig, B7-H4-Ig, or other costimulatory agents, or adhesion molecule inhibitors, mAbs or low molecular weight inhibitors, LFA-1 antagonists, Selectin antagonists and VLA-4 antagonists, or another immunomodulatory agent.

Optionally the pharmaceutical composition according to any of the foregoing or as described herein, the isolated polypeptide or fusion protein according to any of the foregoing or as described herein, the polynucleotide according to any of the foregoing or as described herein, the expression vector or virus according to any of the foregoing or as described herein, or the recombinant cell according to any of the foregoing or as described herein, is provided for use in treatment of infectious disease in a subject suffering from same.

Optionally said protein, said polynucleotide, said expression vector or virus, said recombinant cell, said pharmaceutical composition or said use is used for treatment of infectious disease and is capable of at least one of the following: (i) increasing immune response, (ii) increasing T cell activation, (iii) increasing cytotoxic T cell activity, (iv) increasing NK cell activity, (v) increasing Th17 activity, (vi) alleviating T-cell suppression, (vii) increasing pro-inflammatory cytokine secretion, (viii) increasing IL-2 secretion; (ix) increasing interferon-γ production by T-cells, (x) increasing Th1 response, (xi) decreasing Th2 response, (xii) decreasing or eliminating at least one of regulatory T cells (Tregs), myeloid derived suppressor cells (MDSCs), iMCs, mesenchymal stromal cells, TIE2-expressing monocytes, (xiii) reducing regulatory cell activity and/or the activity of one or more of myeloid derived suppressor cells (MDSCs), iMCs, mesenchymal stromal cells, TIE2-expressing monocytes, (xiv) decreasing or eliminating M2 macrophages, (xv) reducing M2 macrophage pro-tumorigenic activity, (xvi) decreasing N2 neutrophils, (xvii) decreasing N2 neutrophils activity, (xviii) reducing inhibition of T cell activation, (xix) reducing inhibition of CTL activation, (xx) reducing inhibition of NK cell activation, (xxi) reversing T cell exhaustion, (xxii) increasing T cell response, (xxiii) increasing activity of cytotoxic cells, (xxiv) stimulating antigen-specific memory responses, (xxv) eliciting apoptosis or lysis of cancer cells, (xxvi) stimulating cytotoxic or cytostatic effect on cancer cells, (xxvii) inducing direct killing of cancer cells, and/or (xxviii) inducing complement dependent cytotoxicity and/or (xxix) inducing antibody dependent cell-mediated cytotoxicity.

Optionally said infectious disease is chronic infectious disease and is selected from the disease caused by bacterial infection, viral infection, fungal infection and/or other parasite infection.

Optionally said infectious disease results in sepsis.

Optionally the infectious disease is selected from hepatitis B, hepatitis C, infectious mononucleosis, AIDS, tuberculosis, malaria and schistosomiasis.

Optionally the treatment is combined with another moiety useful for treating infectious disease, or with another moiety useful for reducing the undesirable immune activation that follows gene therapy, in a subject in need thereof.

Optionally said other moiety is a therapeutic agent useful for treating bacterial infection, viral infection, fungal infection, parasitic infection or sepsis.

As mentioned above the invention relates to soluble and insoluble VSTM5 proteins, soluble molecules and multimers or fusions preferably those that modulate (agonize or antagonize) at least one effect of VSTM5 on immunity. “VSTM5” or “V-Set And Transmembrane Domain-Containing Protein 5” is described by Taylor et al., “Human chromosome 11 DNA sequence and analysis including novel gene identification”, Nature 440, 497-500 (2006). Taylor discloses a DNA sequence coding a polypeptide 100% identical to the VSTM5 amino acid sequence (SEQ ID NO: 6). The reference does not characterize the activity of this protein or more specifically its immunosuppressive effects on T cell and NK immunity.

US patent application number US20080299042, assigned to Biogen Idec, Inc., discloses sequences of numerous nucleic acid molecules that encode membrane associated proteins, the proteins themselves, and antibodies to the proteins. Also disclosed are methods of treating cancer and autoimmune diseases, specifically referencing colon cancer, lung cancer, pancreatic cancer and ovarian cancer. Included in the application is sequence SEQ ID NO: 1709, which is a sequence identical at 155 of 186 amino acid residues to the VSTM5 amino acid sequence. The reference does not characterize the activity of this protein or more specifically its immunosuppressive effects on T cell or NK cell immunity.

PCT application WO2003025148 assigned to Hyseq, discloses SEQ ID NO 332, which is identical to the wild type VSTM5. The '148 application states that the disclosed polypeptides are useful for raising antibodies, as markers for tissues in which the corresponding polypeptide is expressed, for re-engineering damaged or diseased tissues, for treating myeloid or lymphoid cell disorders, in bone cartilage, tendon, ligament and/or nerve tissue growth or regeneration, in wound healing, in tissue repair and replacement, in healing of burns, incisions and ulcers, and in treating cancer. The reference does not characterize the activity of this protein or more specifically its immunosuppressive effects on T cell or NK immunity.

VSTM5 protein is disclosed in PCT Application No: PCT/US2008/075122, (Sequence 43 therein), owned in common with the present application, which is hereby incorporated by reference, as if fully set forth herein. More particularly this PCT application identified VSTM5 in this application as Sequence 43, which corresponds to residues 29-147 of the sequence referred to in this application as AI216611_P0. This PCT application further teaches that AI216611 and other proteins are differentially expressed by some cancers, and suggests their potential use as targets and potential use in immunotherapy, cancer therapy, and drug development. The application also teaches that these polypeptides possess a B7-like structure and may be costimulatory molecules. This application further discloses methods of treating cancer and autoimmune diseases using the extracellular domain of the VSTM5 protein. However, the above referenced publication, patents and/or patent applications does not teach or suggest the use of specific portions of the VSTM5 ECD, having an amino acid sequence set forth in any of SEQ ID NOs: 1, 12-110, 151-156.

Also, the above referenced publication, patents and/or patent applications do not teach or suggest the use the VSTM5 proteins according to at least some embodiments of the invention, for treatment and/or diagnosis of specific cancer as described herein. Examples of the utility of such proteins for treatment and/or diagnosis of cancer are given below. Without wishing to be limited in any way, it is expected that VSTM5 proteins which reduce VSTM5 immune inhibitory activity induce immunostimulatory activity, and can thereby be used for treating conditions where upregulation of immunity is desired such as cancer immunotherapy or treatment of infection or sepsis.

### BRIEF DESCRIPTION OF THE FIGURES

FIGS. 1-1 to 1-10 contain the complete clinical profiles of multitumor tissue microarray samples analyzed for VSTM5 expression (See Example 1).

FIGS. 2-1 and 2-2 contain the complete clinical profiles of full section lymphoid tissue samples analyzed for VSTM5 expression (See Example 1).

FIGS. 3-1 to 3-10 contain the tissue description of the “TOP4” tissues which comprise triplicate tissue core samples from 120 patients analyzed for VSTM5 expression. (See Example 1)

FIGS. 4-1 to 4-10 contain the immunoreactivity results (IHC values or scores) for individual samples assayed for VSTM5 expression (multitumor tissue arrays of FIGS. 1-1 to 1-10). (See Example 1).

FIGS. 5-1 to 5-10 contain a summary of the IHC values or scores of the TOP4 tissue microarray samples. (See Example 1).

FIG. 6 Schematic presentation of elevation of endogenous expression of the immune checkpoint ligand (PDL-1) by the induction of anti-tumor immunity

FIG. 7 presents the results of the western blot analysis of ectopically expressed human VSTM5 proteins using an anti-VSTM5 antibody, described in details in Example 2 herein. Whole cell extracts (30 ug) of HEK293T cell pools, previously transfected with expression construct encoding human VSTM5 (lane 1), empty vector (lane 2) or with expression construct encoding human VSTM5-EGFP (lane 3), were analyzed by WB using an anti-VSTM5 antibody.

FIGS. 8A to 8B present the results of cell surface expression of human VSTM5 (A) and VSTM5-EGFP (B) proteins by FACS analysis, described in details in Example 2 herein. The anti-VSTM5 pAb (10 ug/ml) was used to analyze HEK-293T cells stably expressing the human VSTM5 proteins. Rabbit IgG was used as Isotype control to the pAb. Cells expressing the empty vector (pRp=pIRESpuro3) were used as negative control. Detection was carried out by donkey anti-rabbit FITC or PE-conjugated secondary Ab and analyzed by FACS.

FIG. 9 presents a schematic illustration of the experimental setting of in-vitro testing of the effect of VSTM5, expressed on HEK 293T cells, on the activation of Jurkat cells by plate bound anti-CD3, as described in details in Example 3 herein.

FIGS. 10A to 10D demonstrate that VSTM5 (SEQ ID NO: 132) expressed on HEK-293T cells inhibits Jurkat cells activation, as described in details in Example 3 herein. HEK-293T cells expressing VSTM5 (SEQ ID NO: 132) or the empty vector (pRp) were seeded at 25,000 (A) or 50,000 (B) cells per well, in wells pre-coated with 2 μg/ml of anti-CD3. Jurkat cells were added 2 hours later at 50,000 cells per well, and the co-cultures were incubated 0. N. Cells were analyzed for the expression of CD69 by flow cytometry. As reference, CD69 values of untreated Jurkat cells (UT), i.e., not treated with anti-CD3, are shown. ΔMFI values of CD69 between untreated and anti-CD3 treated Jurkat cells in the presence of 50,000 HEK-293 transfected cells are presented in (C) The percentage of inhibition of Jurkat cells activation in the presence of 293T_VSTM5 cells is presented in (D). * indicates value significantly different from that of the empty vector (p<0.05, Student's t-test).

FIGS. 11A to 11B demonstrate that VSTM5 (SEQ ID NO: 132) expressed on HEK-293T cells inhibits H9 cells activation as measured by IL-2 secretion. HEK 293T cells expressing VSTM5 or the empty vector (pRp3.1) were seeded at 50,000 or 75,000 cells per well, in wells pre-coated with 0, 0.1 and 0.2 μg/ml of anti-CD3 antibody. H9 cells were added 2 hours later at 50,000 cells per well, and the co-cultures were incubated O.N. Supernatants, depleted from the cells, were analyzed for concentration of hIL-2 in presence of 50,000 transfected cells (A.) or 75,000 HEK 293T transfected cells in (B.) Standard deviation of triplicates are indicated */* * represents significant difference when compared to control group (p<0.05 and p<0.005 respectively, Student's t-test, 2-tail)

FIGS. 12A to 12D present VSTM5-ECD-Ig suppression of CD4 T cell activation, described in details in Example 4 herein. (A-B) CD4⁺CD25⁻CD62L⁺ T cells (1×10⁵ perwell) were stimulated with plate bound anti-CD3 mAb (2 μg/ml) in the presence of 2, 4 or 8 μg/ml of VSTM5-hECD-mIg (SEQ ID NO: 131) or control mIg (i.e. 1:1, 1:2, 1:4 anti-CD3:tested protein ratio, respectively). Culture supernatants were collected at 48 hrs post-stimulation and mouse IL-2 or IFNγ levels were analyzed by ELISA. Results are shown as Mean±Standard errors of triplicate samples. (C) CFSE-labeled CD4⁺CD25⁻ cells were stimulated for 72 h with immobilized anti-CD3 mAb (0.5 μg/ml) in the presence of 0.5 or 1 ug/ml of VSTM5-hECD-mIg (SEQ ID NO: 131) or control mIg (1:1, 1:2 anti-CD3:tested protein ratio, respectively). M1 marker refers to the fraction of dividing cells (CFSE^{low}), presented in the histograms as % CFSElow CD4⁺ T cells. (D) CD4⁺CD25⁻ T cells (1×10⁵ per well) were stimulated with immobilized anti-CD3 mAb (2 μg/ml) in the presence of 10 ug/ml of VSTM5-mECD-mIg (SEQ ID NO: 8) or control Ig, or in the absence of additional proteins (PBS). The expression of CD69 was analyzed by flow cytometry at 48 h post-stimulation.

FIG. 13 demonstrates that VSTM5 hECD-hIg (SEQ ID NO:130) inhibits human T cell proliferation induced by anti-CD3 and anti-CD28 in the presence of irradiated autologous PBMCs, as described in details in Example 5 herein. 1.5×10⁵ naïve CD4⁺ T cells were activated with anti-CD3 (0.5 mg/ml), anti-CD28 (0.5 mg/ml) in the presence of 1.5×10⁵ irradiated autologous PBMCs. VSTM5 hECD-hIg (SEQ ID NO:130) or hIgG1 control Ig (Synagis) was added to the culture at the indicated concentrations. Proliferation was evaluated using H3-tymidine incorporation at 72 hours. Shown are averages of three donors tested.

FIG. 14 demonstrates dose dependent inhibition of T cell activation by VSTM5 ECD-Ig (SEQ ID NO: 130) fusion protein. CD25 expression from CD8 T cells at 1:1 ratio of cell:bead.

FIGS. 15A to 15B demonstrate that VSTM5 hECD-hIg (SEQ ID NOs: 130) and VSTM5 mECD-mIg (SEQ ID NOs: 8) bind H9. (A) H9 cells were incubated with a dose titration of VSTM5-Fc H: H (circles) or control human IgG1 (Synagis, squares). (B) H9 cells were incubated with a dose titration of VSTM5-Fc M: M (triangles) or control mouse IgG2a (Mopc173, inverted triangles). Binding was detected by FACS analysis following the three-step detection protocol, described in Example 6 herein.

FIGS. 16A to 16B demonstrate that binding of biotinylated VSTM5-Fc to H9 cells can be competed off with unlabeled VSTM5-Fc in a dose dependent manner. In (A) H9 cells were incubated with a dose titration of biotinylated VSTM5-Fc H: H. Binding was detected by FACS analysis following the two-step detection protocol (VSTM5—squares; human IgG1 control—circles). In (B) unlabeled VSTM5-Fc H: H (circles) or human IgG1 isotype control (triangles) was incubated with H9 cells prior to binding with 44 nM biotinylated VSTM5-Fc H: H (diamond), as described in the competition assay protocol in Example 7 herein.

FIGS. 17A to 17B show that VSTM5 ECD fused to human IgG1 Fc binds to activated but not to resting human CD4⁺ T cells. (A) FACS histograms of binding of B7H1-Ig or VSTM5 hECD fused to human IgG1 Fc (SEQ ID NO: 130) compared to control Ig to live resting and activated CD4⁺ cells (B) Values of histograms represent the geometric mean fluorescent intensity (gMFI) of resting and activated CD4⁺ cells. Each bar is the mean±SD of duplicate samples.

FIGS. 18A to 18B show that VSTM5 ECD fused to human IgG1 Fc binds to activated but not resting human CD8⁺ T cells. (A) FACS histograms of binding of B7H1-Ig or VSTM5 ECD fused to human IgG1 Fc (SEQ ID NO: 130) compared to control Ig to live resting and activated CD4⁺ cells (B) Values of histograms represent the geometric mean fluorescent intensity (gMFI) of resting and activated CD4⁺ cells. Each bar is the mean±SD of duplicate samples.

FIG. 19 shows that VSTM5 mECD-mIg (SEQ ID NO: 8) enhances iTreg cell differentiation. CD4+CD25− T cells were activated for 4 days in 96 well plates using immobilized anti-CD3 (5 μg/ml) and soluble anti-CD28 (1 μg/ml) in the presence of purified CD11c⁺ dendritic cells (APCs) at a 1:5 cell ratio. Soluble VSTM5-mECD-mIg (SEQ ID NO: 8) was added at 10 μg/ml. Cultures were treated with iTreg driving conditions, i.e., TGFβ (5 ng/ml) and mIL-2 (5 ng/ml). Development of Foxp3+CD4+ iTreg cells was assessed by flow cytometry.

FIGS. 20A to 20B show that VSTM5 mECD-mIg (SEQ ID NO: 8) enhances iTreg cell differentiation in the presence of TGF-β and IL-2. CD4⁺CD25⁻ T cells were cultured for 5 days with immobilized anti-CD3 (2 ug/ml) together with VSTM5 mECD-mIg (SEQ ID NO: 8) at 10 μg/ml in the presence or absence of TGFβ (10 ng/ml), with or without IL-2 (5 ng/ml). Development of Foxp3⁺CD25⁺ iTreg cells was assessed by flow cytometric analysis. FIG. 20A presents representative plots of gated CD4⁺ cells. The values represent the percentage of CD25⁺Foxp3⁺ of total CD4⁺ cells or total Tregs cell count/μl. FIG. 20B shows plots representing average percentage or total cell count of iTregs from triplicate cultures for each condition.

FIGS. 21A to 21B shows that VSTM5-ECD fused to F_{c} of human IgG1 (SEQ ID NO:130) binds to primary activated NK cells Human NK cell clones from one donor were incubated with 5μ of human IgG1 (SEQ ID NO:130) binds to primary activated NK cells H. Examples of high binding clones are shown in (A), and examples of low binding clones in (B).

FIG. 22 presents VSTM5 over expression on cancer cell lines. Human cancer cell lines were transduced with retroviral vector encoding only DSRED (red fluorescent protein) only or also VSTM5 (SEQ ID NO: 132, as described in the examples. Expression levels of VSTM5 were evaluated by FACS analysis using a commercial rabbit polyclonal antibody and rabbit IgG as isotype control, and evaluated with an anti-rabbit secondary antibody.

FIGS. 23A to 23D show that VSTM5 over expression on cancer cell lines reduces their susceptibility to killing by NK cells. Human polyclonal NK cells were co-incubated with human cancer cell lines (Hela—FIG. 23A, RKO—FIG. 23B, 8866—FIG. 23C and BJAB—FIG. 23D) over expressing VSTM5 (SEQ ID NO:132) or transfected with empty vector (dsred) and percentage of killing was assessed as described in the examples The Y axis shows % of killing. The X axis shows effector to target (E:T) ratios (two-fold serial dilutions of effector cells), that range from 40:1 to 5:1 in the experiments with HeLa and RKO, and 30:1 to 15:1 in the experiments with BJAB and 8866. * P value <0.05, ** P value <0.02, *** P value <0.01

FIG. 24 presents schematic illustration of the experimental system used in Example 10 herein.

FIG. 25 presents the results of FACS analysis performed on VSTM5 transduced melanoma cells SK-mel-23, mel-624, and mel-624.38 and mel-888 using a specific polyclonal antibody that recognizes VSTM5, in order to assess the levels of membrane expression of this protein. The percent of cells staining positive for the protein is provided for each cell line.

FIG. 26 presents the results of FACS analysis performed on TCR F4 transduced stimulated CD8⁺ cells (CTLs) using a specific monoclonal antibody that recognizes the extracellular domain of the β-chain from the transduced specific TCR, in order to assess the levels of membrane expression of this specific TCR.

FIG. 27A shows the effect of VSTM5 expressed on melanoma cell lines on activation of F4 TCR expressing CTLs in a co-culture assay, as observed by IFNγ secretion. The graphs show two independent experiments with CTLs from different donors transduced with F4 TCR. *p=0.01.

FIG. 27B presents a summary of several experiments using three melanoma cell lines (SK-mel-23, mel-624 and mel-624.38) overexpressing VSTM5, in a co-culture assay to evaluate the effect on activation of F4 TCR expressing CTLs. The dots represent the level of IFNγ secretion obtained in independent experiments, whereby 100% is defined as the level of secretion using the respective melanoma cell line transduced with empty vector. The left panels show results using cells with relatively low expression of VSTM5, the right panels show results using cells with relatively low expression of VSTM5.

FIG. 27C shows the effect of VSTM5 expressed on melanoma cell lines on activation of F4 TCR expressing CTLs in a co-culture assay, as observed by IL-2 secretion. The graphs show two independent experiments with F4 TCR transduced CTLs from different donors. *p=0.01.

FIG. 27D shows the effect of VSTM5 expressed on melanoma cells on reduction of TNFα secretion from F4 TCR expressing CTLs in a co-culture assay. The graph shows one experiment with F4 TCR transduced CTLs from one donor. *p=0.01.

FIG. 28 demonstrates the susceptibility of mel-624 melanoma cell lines overexpressing VSTM5 or transfected with empty vector, to killing by F4 transduced or non-transduced (‘w/o’) lymphocytes from one donor. The Effector to Target ratio was 1:1 or 1:3. Percentages are of double positive cells stained for CFSE and PI, and indicate level of cell killing.

FIGS. 29A to 29B shows the effect of VSTM5 mECD-mIg, fusion protein on T cell proliferation and IFNγ secretion under Th0, Th1, Th2 or Th17 differentiation-driving conditions. (FIG. 29A) Bars represent the number of CD4+ cells per well (FIG. 29B) IFNγ levels (pg/ml) in culture supernatants as analyzed by ELISA. Results are shown as Mean±Standard errors of triplicate samples. (***; P value<0.001; ANOVA followed by bonferroni posttests).

FIGS. 30A to 30B shows the effect of VSTM5 mECD-mIg fusion protein on Th17 differentiation. (FIG. 30A) Representative density plots show the percentage of CD4⁺IL17⁺ cells out of total CD4 cells, under Th0 and Th17 driving conditions. (FIG. 30B) Bars indicate the number of CD4⁺IL-17⁺ cells per well under Th17 driving conditions. Results are shown as Mean±Standard errors of triplicate samples (* P value<0.01; ANOVA followed by Bonferroni posttests).

### DETAILED DESCRIPTION OF THE INVENTION

The present invention, in at least some embodiments, relates to any one of the proteins referred to as VSTM5, and its corresponding nucleic acid sequence, and portions and variants thereof and fusion proteins containing same, and the use thereof as a therapeutic and/or diagnostic agent, for various diseases, as described herein. Optionally, the present invention, in at least some embodiments, relates to immunostimulatory VSTM5 therapeutic and/or diagnostic agents for the treatment of cancer, infectious diseases, particularly chronic infections or sepsis. Further optionally, the present invention, in at least some embodiments, relates to immunoinhibitory VSTM5 therapeutic and/or diagnostic agents for the treatment of immune related conditions and/or for reducing the undesirable immune activation that follows gene or cell therapy or after transplant.

As used herein, the term VSTM5 refers to any one of the proteins set forth in anyone of SEQ ID NOs: 6, 7, 132, and/or amino acid sequences corresponding to VSTM5 V-set domain set forth in SEQ ID NO: 1, and/or VSTM5 ECD set forth in any one of SEQ ID NOs 2, 3, and/or fragments and/or epitopes of the VSTM5 ECD, as set forth in any of SEQ ID NOs: 12-110, 151-156 and/or variants thereof that possesses at least 90 or 95% sequence identity therewith, and/or nucleic acid sequences encoding for same. Optionally, the VSTM5 polypeptide or protein will modulate (agonize or antagonize) at least one effect of VSTM5 on immunity and/or on specific immune cell types and/or will be useful as an immunogen for generating anti-VSTM5 antibodies which will modulate (agonize or antagonize) at least one effect of VSTM5 on immunity and/or on specific immune cell types. Optionally the term VSTM5 refers to any one of the proteins above that are differentially expressed in cancer, on the cancer cells or on the immune cells infiltrating the tumor, and/or on stromal cells, prior to or following cancer therapy, optionally prior to or following combination immunotherapy of cancer, as detailed herein.

According to at least some embodiments, the present invention provides immunostimulatory VSTM5 therapeutic agents, wherein said agents are used for treatment of cancer and/or infectious disease, and wherein said agents mediate at least one of the following immune effects: increase immune response, increase T cell activation, increase cytotoxic T cell activity, increase Th17 activity, increase NK cell activity, alleviate T-cell suppression, increase pro-inflammatory cytokine secretion, increase IL-2 secretion; increase interferon-γ production by T-cells, increase Th1 response, decrease Th2 response, decrease or eliminate regulatory T cells such as s Tregs, MDSCs, iMCs, mesenchymal stromal cells, TIE2-expressing and other immunosuppressive monocytes, inflammatory monocytes, neutrophils, macrophages and the like, reduce regulatory cell activity or the activity of MDSCs, iMCs, mesenchymal stromal cells, TIE2-expressing and other immunosuppressive monocytes, inflammatory monocytes, neutrophils, macrophages and the like, decrease or eliminate M2 macrophages, reduce M2 macrophage pro-tumorigenic activity, reduce inhibition of T cell activation, reduce inhibition of CTL (CD4⁺ or CD8⁺) activation, reduce inhibition of NK cell activation, reverse T cell exhaustion, increase T cell response, increase activity of cytotoxic cells, stimulate antigen-specific memory responses, elicit apoptosis or lysis of cancer cells, stimulate cytotoxic or cytostatic effect on cancer cells, induce direct killing of cancer cells, induce complement dependent cytotoxicity and/or induce antibody dependent cell-mediated cytotoxicity in a mammal.

According to at least some embodiments, the present invention provides immunoinhibitory VSTM5 therapeutic agents, wherein said agents are used for treatment of immune related diseases and/or for reducing the undesirable immune activation that follows gene therapy, and wherein said agents mediate at least one of the following immune effects: inhibiting immune response, reducing T cell activity, reducing NK cell activity, enhancing regulatory cell activity, enhancing T-cell suppression, enhancing immune regulatory cell activity, inducing establishment of immune tolerance, reducing pro-inflammatory cytokine secretion, re-establishing Th1-Th2 immune balance, reducing immune memory responses to self-antigens, decreasing or eliminating pro-inflammatory immune cells, decreasing or eliminating auto-reactive or pro-inflammatory immune cells.

According to at least some embodiments, for any of the above described cancers, optionally each of the above described cancer type or subtype may optionally form a separate embodiment and/or may optionally be combined as embodiments or subembodiments.

According to at least some embodiments, there is provided a method of performing one or more of the following in a subject by administering a VSTM5 protein as described herein or a pharmaceutical composition as described herein to a subject.
(a) upregulating proinflammatory cytokine secretion,
(b) increasing T-cell proliferation and/or expansion,
(c) increasing interferon-γ production
(d) increasing IL-2 secretion
(e) stimulating anti-tumor immune responses;
(f) inhibiting cancer cell growth, (g) promotes antigenic specific T cell immunity, (g) promoting CD4⁺ and/or CD8⁺ T cell activation,
(i) reducing immunosuppressive cell activity
(j) alleviating T-cell suppression,
(k) promoting cytotoxic cell activity,
(h) alleviating apoptosis or lysis of cancer cells, and/or
(k) promoting cytotoxic or cytostatic effect on cancer cells, comprising

In order that the present invention in various embodiments may be more readily understood, certain terms are first defined. Additional definitions are set forth throughout the specification, especially the detailed description.

### DEFINITIONS

Unless defined otherwise, all technical and scientific terms used herein have the same meaning as those commonly understood by one of ordinary skill in the art to which this invention belongs. Although methods and materials similar or equivalent to those described herein may be used in the invention or testing of the present invention, suitable methods and materials are described herein. The materials, methods and examples are illustrative only, and are not intended to be limiting. The nomenclatures utilized in connection with, and the laboratory procedures and techniques of, analytical chemistry, synthetic organic chemistry, and medicinal and pharmaceutical chemistry described herein are those well-known and commonly used in the art. Standard techniques may be used for chemical syntheses, chemical analyses, pharmaceutical preparation, formulation, and delivery, and treatment of patients.

As used in the description herein and throughout the claims that follow, the meaning of “a,” “an,” and “the” includes plural reference unless the context clearly dictates otherwise.

“Activating receptor,” as used herein, refers broadly to immune cell receptors that bind antigen, complexed antigen (e.g., in the context of MHC molecules), Ig-fusion proteins, ligands, or antibodies. Activating receptors but are not limited to T cell receptors (TCRs), B cell receptors (BCRs), cytokine receptors, LPS receptors, complement receptors, and Fc receptors. For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes. For example, T cell receptors are present on T cells and are associated with CD3 molecules. T cell receptors are stimulated by antigen in the context of MHC molecules (as well as by polyclonal T cell activating reagents). T cell activation via the TCR results in numerous changes, e.g., protein phosphorylation, membrane lipid changes, ion fluxes, cyclic nucleotide alterations, RNA transcription changes, protein synthesis changes, and cell volume changes.

“Adjuvant” as used herein, refers to an agent used to stimulate the immune system and increase the response to a vaccine, without having any specific antigenic effect in itself.

“Aids in the diagnosis” or “aids in the detection” of a disease herein means that the expression level of a particular marker polypeptide or expressed RNA is detected alone or in association with one or more other markers in order to assess whether a subject has cells characteristic of a particular disease condition or the onset of a particular disease condition or comprises immune dysfunction such as immunosuppression characterized by VSTM5 expression or abnormal immune upregulation characterized by cells having reduced VSTM5 levels, such as during autoimmunity, inflammation or allergic responses, e.g., in individuals with chronic and non-chronic diseases.

“Allergic disease,” as used herein, refers broadly to a disease involving allergic reactions. More specifically, an “allergic disease” is defined as a disease for which an allergen is identified, where there is a strong correlation between exposure to that allergen and the onset of pathological change, and where that pathological change has been proven to have an immunological mechanism. Herein, an immunological mechanism means that leukocytes show an immune response to allergen stimulation.

“Amino acid,” as used herein refers broadly to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified (e.g., hydroxyproline, γ-carboxyglutamate, and O-phosphoserine.) Amino acid analogs refers to compounds that have the same basic chemical structure as a naturally occurring amino acid (i.e., a carbon that is bound to a hydrogen, a carboxyl group, an amino group), and an R group (e.g., homoserine, norleucine, methionine sulfoxide, methionine methyl sulfonium.) Analogs may have modified R groups (e.g., norleucine) or modified peptide backbones, but retain the same basic chemical structure as a naturally occurring amino acid Amino acid mimetics refers to chemical compounds that have a structure that is different from the general chemical structure of an amino acid, but that functions in a manner similar to a naturally occurring amino acid.

“Anergy” or “tolerance,” or “prolonged antigen-specific T cell suppression” or “prolonged immunosuppression” as used herein refers broadly to refractivity to activating receptor-mediated stimulation. Refractivity is generally antigen-specific and persists after exposure to the tolerizing antigen has ceased. For example, anergy in T cells (as opposed to unresponsiveness) is characterized by lack of cytokine production, e.g., IL-2. T cell anergy occurs when T cells are exposed to antigen and receive a first signal (a T cell receptor or CD-3 mediated signal) in the absence of a second signal (a costimulatory signal). Under these conditions, reexposure of the cells to the same antigen (even if reexposure occurs in the presence of a costimulatory molecule) results in failure to produce cytokines and, thus, failure to proliferate. Anergic T cells can, however, mount responses to unrelated antigens and can proliferate if cultured with cytokines (e.g., IL-2). For example, T cell anergy can also be observed by the lack of IL-2 production by T lymphocytes as measured by ELISA or by a proliferation assay using an indicator cell line. Alternatively, a reporter gene construct can be used. For example, anergic T cells fail to initiate IL-2 gene transcription induced by a heterologous promoter under the control of the 5′ IL-2 gene enhancer or by a multimer of the AP1 sequence that can be found within the enhancer (Kang et al. (1992) Science 257:1134). Modulation of a costimulatory signal results in modulation of effector function of an immune cell.

“Antibody”, as used herein, refers broadly to an “antigen-binding portion” of an antibody (also used interchangeably with “antibody portion,” “antigen-binding fragment,” “antibody fragment”), as well as whole antibody molecules. The term “antigen-binding portion”, as used herein, refers to one or more fragments of an antibody that retain the ability to specifically bind to an antigen (e.g., VSTM5 or specific portions thereof)). The term “antibody” as referred to herein includes whole polyclonal and monoclonal antibodies and any antigen-binding fragment (i.e., “antigen-binding portion”) or single chains thereof. An “antibody” refers to a glycoprotein comprising at least two heavy (H) chains and two light (L) chains inter-connected by disulfide bonds, or an antigen-binding portion thereof. Each heavy chain is comprised of at least one heavy chain variable region (abbreviated herein as V_{H}) and a heavy chain constant region. The heavy chain constant region is comprised of three domains, C_{H1}, C_{H2} and C_{H3}. Each light chain is comprised of at least one light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. More generally, the term “antibody” is intended to include any polypeptide chain-containing molecular structure with a specific shape that fits to and recognizes an epitope, where one or more non-covalent binding interactions stabilize the complex between the molecular structure and the epitope. The archetypal antibody molecule is the immunoglobulin, and all types of immunoglobulins, IgG, IgM, IgA, IgE, IgD, etc., from all sources, e.g. human, rodent, rabbit, cow, sheep, pig, dog, other mammals, chicken, other avians, etc., are considered to be “antibodies.”

The antigen-binding function of an antibody can be performed by fragments of a full-length antibody. Non-limiting examples of antigen-binding fragments encompassed within the term “antigen-binding portion” of an antibody include (a) a Fab fragment, a monovalent fragment consisting of the V_{L}, V_{H}, C_{L} and Cm domains; (b) a F(ab′)2 fragment, a bivalent fragment comprising two Fab fragments linked by a disulfide bridge at the hinge region; (c) a F_{d} fragment consisting of the V_{H} and C_{H1} domains; (d) a Fᵥ fragment consisting of the V_{L} and V_{H} domains of a single arm of an antibody; (e) a dAb fragment (Ward, et al. (1989) Nature 341: 544-546), which consists of a V_{H} domain; and (f) an isolated complementarily determining region (CDR). Furthermore, although the two domains of the Fᵥ fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv). See e.g., Bird, et al. (1988) Science 242: 423-426; Huston, et al. (1988) Proc Natl. Acad. Sci. USA 85: 5879-5883; and Osbourn, et al. (1998) Nat. Biotechnol. 16: 778. Single chain antibodies are also intended to be encompassed within the term “antigen-binding portion” of an antibody. Any V_{H} and V_{L} sequences of specific scFv can be linked to human immunoglobulin constant region cDNA or genomic sequences, in order to generate expression vectors encoding complete IgG molecules or other isotypes. V_{H} and V_{L} can also be used in the generation of Fab, Fv, or other fragments of immunoglobulins using either protein chemistry or recombinant DNA technology. Other forms of single chain antibodies, such as diabodies are also encompassed. Diabodies are bivalent, bispecific antibodies in which V_{H} and VL domains are expressed on a single polypeptide chain, but using a linker that is too short to allow for pairing between the two domains on the same chain, thereby forcing the domains to pair with complementary domains of another chain and creating two antigen-binding sites. See e.g. Holliger, et al. (1993) Proc Natl. Acad. Sci. USA 90: 6444-6448; Poljak, et al. (1994) Structure 2: 1121-1123. Still further, an antibody or antigen-binding portion thereof (antigen-binding fragment, antibody fragment, antibody portion) may be part of a larger immunoadhesion molecules, formed by covalent or noncovalent association of the antibody or antibody portion with one or more other proteins or peptides. Examples of immunoadhesion molecules include use of the streptavidin core region to make a tetrameric scFv molecule (Kipriyanov, et al. (1995) Hum. Antibodies Hybridomas 6: 93-101) and use of a cysteine residue, a marker peptide and a C-terminal polyhistidine tag to make bivalent and biotinylated scFv molecules. Kipriyanov, et al. (1994) Mol. Immunol. 31: 1047-1058. Antibody portions, such as Fab and F (ab′) 2 fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein. Antibodies may be polyclonal, monoclonal, xenogeneic, allogeneic, syngeneic, or modified forms thereof, e.g., humanized, or chimeric antibodies.

“Antibody recognizing an antigen” and “an antibody specific for an antigen” is used interchangeably herein with the term “an antibody which binds specifically to an antigen” and refers to an immunoglobulin or fragment thereof that specifically binds an antigen.

“Antigen,” as used herein, refers broadly to a molecule or a portion of a molecule capable of being bound by an antibody which is additionally capable of inducing an animal to produce an antibody capable of binding to an epitope of that antigen. An antigen may have one epitope, or have more than one epitope. The specific reaction referred to herein indicates that the antigen will react, in a highly selective manner, with its corresponding antibody and not with the multitude of other antibodies which may be evoked by other antigens. In the case of a desired enhanced immune response to particular antigens of interest, antigens include, but are not limited to; infectious disease antigens for which a protective immune response may be elicited are exemplary.

“Antigen presenting cell,” as used herein, refers broadly to professional antigen presenting cells (e.g., B lymphocytes, monocytes, dendritic cells, and Langerhans cells) as well as other antigen presenting cells (e.g., keratinocytes, endothelial cells, astrocytes, fibroblasts, and oligodendrocytes).

“Antisense nucleic acid molecule,” as used herein, refers broadly to a nucleotide sequence which is complementary to a “sense” nucleic acid encoding a protein (e.g., complementary to the coding strand of a double-stranded cDNA molecule) complementary to an mRNA sequence or complementary to the coding strand of a gene. Accordingly, an antisense nucleic acid molecule can hydrogen bond to a sense nucleic acid molecule.

“Apoptosis,” as used herein, refers broadly to programmed cell death which can be characterized using techniques which are known in the art. Apoptotic cell death can be characterized by cell shrinkage, membrane blebbing, and chromatin condensation culminating in cell fragmentation. Cells undergoing apoptosis also display a characteristic pattern of internucleosomal DNA cleavage.

“Asthma,” as used herein, refers broadly to an allergic disorder of the respiratory system characterized by inflammation, narrowing of the airways and increased reactivity of the airways to inhaled agents. Asthma is frequently, although not exclusively, associated with atopic or allergic symptoms.

“Autoimmunity” or “autoimmune disease or condition,” as used herein, refers broadly to a disease or disorder arising from and directed against an individual's own tissues or a co-segregate or manifestation thereof or resulting condition therefrom, and includes. Herein autoimmune conditions include inflammatory or allergic conditions, e.g., chronic diseases characterized by a host immune reaction against self-antigens potentially associated with tissue destruction such as rheumatoid arthritis.

“B cell receptor” (BCR),” as used herein, refers broadly to the complex between membrane Ig (mIg) and other transmembrane polypeptides (e.g., Igα. and Igβ) found on B cells. The signal transduction function of mIg is triggered by crosslinking of receptor molecules by oligomeric or multimeric antigens. B cells can also be activated by anti-immunoglobulin antibodies. Upon BCR activation, numerous changes occur in B cells, including tyrosine phosphorylation.

“Cancer,” as used herein, refers broadly to any neoplastic disease (whether invasive or metastatic) characterized by abnormal and uncontrolled cell division causing malignant growth or tumor (e.g., unregulated cell growth.) The term “cancer” or “cancerous” as used herein should be understood to encompass any neoplastic disease (whether invasive, non-invasive or metastatic) which is characterized by abnormal and uncontrolled cell division causing malignant growth or tumor, non-limiting examples of which are described herein. This includes any physiological condition in mammals that is typically characterized by unregulated cell growth. Examples of cancer are exemplified in the working examples. Further cancers include but are not limited to, carcinoma, lymphoma, blastoma, sarcoma, and leukemia. More particular examples of such cancers include squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD).

Other cancers amenable for treatment by the present invention include, but are not limited to, carcinoma, lymphoma, blastoma, sarcoma, and leukemia or lymphoid malignancies. More particular examples of such cancers include colorectal, bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. Preferably, the cancer is selected from the group consisting of colorectal cancer, breast cancer, colorectal cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In an exemplary embodiment the cancer is an early or advanced (including metastatic) bladder, ovarian or melanoma. In another embodiment the cancer is colorectal cancer. The cancerous conditions amenable for treatment of the invention include cancers that express or do not express VSTM5 and further include non-metastatic or non-invasive as well as invasive or metastatic cancers wherein VSTM5 expression by immune, stromal or diseased cells suppress antitumor responses and anti-invasive immune responses. The method of the present invention is particularly suitable for the treatment of vascularized tumors.

Cancers herein further include, but are not limited to, carcinoma, lymphoma, blastoma, sarcoma, and leukemia or lymphoid malignancies. More particular examples of such cancers include colorectal, bladder, ovarian, melanoma, squamous cell cancer, lung cancer (including small-cell lung cancer, non-small cell lung cancer, adenocarcinoma of the lung, and squamous carcinoma of the lung), cancer of the peritoneum, hepatocellular cancer, gastric or stomach cancer (including gastrointestinal cancer), pancreatic cancer, glioblastoma, cervical cancer, ovarian cancer, liver cancer, bladder cancer, hepatoma, breast cancer, colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome. In some preferred embodiments the cancer is selected from the group consisting of colorectal cancer, breast cancer, colorectal cancer, rectal cancer, non-small cell lung cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma. In an exemplary embodiment the cancer is an early or advanced (including metastatic) bladder, ovarian or melanoma. In another embodiment the cancer is colorectal cancer. Cancers according to the invention include cancers that express or do not express VSTM5 and further include non-metastatic or non-invasive as well as invasive or metastatic cancers wherein VSTM5 expression by immune, stromal or diseased cells suppress antitumor responses and anti-invasive immune responses, and those characterized by vascularized tumors.

“Cancer therapy” herein refers to any method which prevents or treats cancer or ameliorates one or more of the symptoms of cancer. Typically such therapies will comprise the administration of an immunostimulatory VSTM5 polypeptide or fusion protein, conjugate, multimer (homomultimer or heteromultimer) or composition containing according to the invention either alone or more typically in combination with chemotherapy or radiotherapy or other biologics and for enhancing the activity thereof, i.e., in individuals wherein VSTM5 expression suppress antitumor responses and the efficacy of chemotherapy or radiotherapy or biologic efficacy. Any chemotherapeutic agent exhibiting anticancer activity can be used according to the present invention. Preferably, the chemotherapeutic agent is selected from the group consisting of alkylating agents, antimetabolites, folic acid analogs, pyrimidine analogs, purine analogs and related inhibitors, vinca alkaloids, epipodophyllotoxins, antibiotics, L-Asparaginase, topoisomerase inhibitor, interferons, platinum coordination complexes, anthracenedione substituted urea, methyl hydrazine derivatives, adrenocortical suppressant, adrenocorticosteroids, progestins, estrogens, antiestrogen, androgens, antiandrogen, and gonadotropin-releasing hormone analog. More preferably, the chemotherapeutic agent is selected from the group consisting of 5-fluorouracil (5-FU), leucovorin (LV), irenotecan, oxaliplatin, capecitabine, paclitaxel and doxetaxel. Two or more chemotherapeutic agents can be used in a cocktail to be administered in combination with administration of the anti-VEGF antibody. One preferred combination chemotherapy is fluorouracil-based, comprising 5-FU and one or more other chemotherapeutic agent(s). Suitable dosing regimens of combination chemotherapies are known in the art and described in, for example, Saltz et al. Proc ASCO 18:233 (1999) and Douillard et al. Lancet 355:1041-7(2000). The biologic may be another immune potentiators such as antibodies to PD-L1, PD-L2, CTLA-4, or VISTA as well as PD-L1, PD-L2, CTLA-4 or VISTA fusion proteins as well as cytokines, growth factor antagonists and agonists, hormones and anti-cytokine antibodies.

“Chimeric antibody,” as used herein, refers broadly to an antibody molecule in which the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, the variable region or a portion thereof, is altered, replaced or exchanged with a variable region having a different or altered antigen specificity.

“Coding region,” as used herein, refers broadly to regions of a nucleotide sequence comprising codons which are translated into amino acid residues, whereas the term “noncoding region” refers to regions of a nucleotide sequence that are not translated into amino acids (e.g., 5′ and 3′ untranslated regions).

“Conservatively modified variants,” as used herein, applies to both amino acid and nucleic acid sequences, and with respect to particular nucleic acid sequences, refers broadly to conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. “Silent variations” are one species of conservatively modified nucleic acid variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) may be modified to yield a functionally identical molecule.

“Complementarily determining region,” “hypervariable region,” or “CDR,” as used herein, refers broadly to one or more of the hyper-variable or complementarily determining regions (CDRs) found in the variable regions of light or heavy chains of an antibody. See Kabat, et al. (1987) Sequences of Proteins of Immunological Interest National Institutes of Health, Bethesda, Md. These expressions include the hypervariable regions as defined by Kabat, et al. (1983) Sequences of Proteins of Immunological Interest, U. S. Dept. of Health and Human Services or the hypervariable loops in 3-dimensional structures of antibodies. Chothia and Lesk (1987) J. Mol. Biol. 196: 901-917. The CDRs in each chain are held in close proximity by framework regions and, with the CDRs from the other chain, contribute to the formation of the antigen-binding site. Within the CDRs there are select amino acids that have been described as the selectivity determining regions (SDRs) which represent the critical contact residues used by the CDR in the antibody-antigen interaction. (Kashmiri Methods 36: 25-34(2005)).

“Control amount,” as used herein, refers broadly to a marker can be any amount or a range of amounts to be compared against a test amount of a marker. For example, a control amount of a marker may be the amount of a marker in a patient with a particular disease or condition or a person without such a disease or condition. A control amount can be either in absolute amount (e.g., microgram/ml) or a relative amount (e.g., relative intensity of signals).

“Costimulatory receptor,” as used herein, refers broadly to receptors which transmit a costimulatory signal to an immune cell, e.g., CD28 or ICOS. As used herein, the term “inhibitory receptors” includes receptors which transmit a negative signal to an immune cell, e.g., a T cell or an NK cell.

“Costimulate,” as used herein, refers broadly to the ability of a costimulatory molecule to provide a second, non-activating, receptor-mediated signal (a “costimulatory signal”) that induces proliferation or effector function. For example, a costimulatory signal can result in cytokine secretion (e.g., in a T cell that has received a T cell-receptor-mediated signal) Immune cells that have received a cell receptor-mediated signal (e.g., via an activating receptor) may be referred to herein as “activated immune cells.” With respect to T cells, transmission of a costimulatory signal to a T cell involves a signaling pathway that is not inhibited by cyclosporin A. In addition, a costimulatory signal can induce cytokine secretion (e.g., IL-2 and/or IL-10) in a T cell and/or can prevent the induction of unresponsiveness to antigen, the induction of anergy, or the induction of cell death in the T cell.

“Costimulatory polypeptide” or “costimulatory molecule” herein refers to a polypeptide that, upon interaction with a cell-surface molecule on T cells, modulates T cell responses.

“Costimulatory signaling” as used herein is the signaling activity resulting from the interaction between costimulatory polypeptides on antigen presenting cells and their receptors on T cells during antigen-specific T cell responses. Without wishing to be limited by a single hypothesis, the antigen-specific T cell response is believed to be mediated by two signals: 1) engagement of the T cell Receptor (TCR) with antigenic peptide presented in the context of MHC (signal 1), and 2) a second antigen-independent signal delivered by contact between different costimulatory receptor/ligand pairs (signal 2). Without wishing to be limited by a single hypothesis, this “second signal” is critical in determining the type of T cell response (activation vs inhibition) as well as the strength and duration of that response, and is regulated by both positive and negative signals from costimulatory molecules, such as the B7 family of proteins.

“B7” polypeptide herein means a member of the B7 family of proteins that costimulate T cells including, but not limited to B7-1, B7-2, B7-DC, B7-H5, B7-H1, B7-H2, B7-H3, B7-H4, B7-H6, B7-S3 and biologically active fragments and/or variants thereof. Representative biologically active fragments include the extracellular domain or fragments of the extracellular domain that costimulate T cells.

“Cytoplasmic domain,” as used herein, refers broadly to the portion of a protein which extends into the cytoplasm of a cell.

“Diagnostic,” as used herein, refers broadly to identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Diagnosing,” or “aiding in the diagnosis” as used herein refers broadly to classifying a disease or a symptom, and/or determining the likelihood that an individual has a disease condition (e.g., based on absence or presence of VSTM5 expression, and/or increased or decreased expression by immune, stromal and/or putative diseased cells); determining a severity of the disease, monitoring disease progression, forecasting an outcome of a disease and/or prospects of recovery. The term “detecting” may also optionally encompass any of the foregoing. Diagnosis of a disease according to the present invention may, in some embodiments, be affected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

“Effective amount,” as used herein, refers broadly to the amount of a compound, antibody, antigen, or cells that, when administered to a patient for treating a disease, is sufficient to effect such treatment for the disease. The effective amount may be an amount effective for prophylaxis, and/or an amount effective for prevention. The effective amount may be an amount effective to reduce, an amount effective to prevent the incidence of signs/symptoms, to reduce the severity of the incidence of signs/symptoms, to eliminate the incidence of signs/symptoms, to slow the development of the incidence of signs/symptoms, to prevent the development of the incidence of signs/symptoms, and/or effect prophylaxis of the incidence of signs/symptoms. The “effective amount” may vary depending on the disease and its severity and the age, weight, medical history, susceptibility, and pre-existing conditions, of the patient to be treated. The term “effective amount” is synonymous with “therapeutically effective amount” for purposes of this invention.

“Extracellular domain” or “ECD” as used herein refers broadly to the portion of a protein that extends from the surface of a cell.

“Expression vector,” as used herein, refers broadly to any recombinant expression system for the purpose of expressing a nucleic acid sequence of the invention in vitro or in vivo, constitutively or inducibly, in any cell, including prokaryotic, yeast, fungal, plant, insect or mammalian cell. The term includes linear or circular expression systems. The term includes expression systems that remain episomal or integrate into the host cell genome. The expression systems can have the ability to self-replicate or not, i.e., drive only transient expression in a cell. The term includes recombinant expression cassettes which contain only the minimum elements needed for transcription of the recombinant nucleic acid.

“Family,” as used herein, refers broadly to the polypeptide and nucleic acid molecules of the invention is intended to mean two or more polypeptide or nucleic acid molecules having a common structural domain or motif and having sufficient amino acid or nucleotide sequence homology as defined herein. Family members can be naturally or non-naturally occurring and can be from either the same or different species. For example, a family can contain a first polypeptide of human origin, as well as other, distinct polypeptides of human origin or alternatively, can contain homologues of non-human origin (e.g., monkey polypeptides.) Members of a family may also have common functional characteristics.

“F_{c} receptor” (FcRs) as used herein, refers broadly to cell surface receptors for the Fc portion of immunoglobulin molecules (Igs). Fc receptors are found on many cells which participate in immune responses. Among the human FcRs that have been identified so far are those which recognize IgG (designated FcγR), IgE (FcεR1), IgA (FcαR), and polymerized IgM/A (FcεμR). FcRs are found in the following cell types: FcεRI (mast cells), FcεRII (many leukocytes), FcαR (neutrophils), and FcμαR (glandular epithelium, hepatocytes). (Hogg Immunol. Today 9: 185-86 (1988)). The widely studied FcγRs are central in cellular immune defenses, and are responsible for stimulating the release of mediators of inflammation and hydrolytic enzymes involved in the pathogenesis of autoimmune disease. (Unkeless Annu. Rev. Immunol. 6: 251-87 (1988)). The FcγRs provide a crucial link between effector cells and the lymphocytes that secrete Ig, since the macrophage/monocyte, polymorphonuclear leukocyte, and natural killer (NK) cell FcγRs confer an element of specific recognition mediated by IgG. Human leukocytes have at least three different receptors for IgG: hFcμRI (found on monocytes/macrophages), hFcγRII (on monocytes, neutrophils, eosinophils, platelets, possibly B cells, and the K562 cell line), and FcγIII (on NK cells, neutrophils, eosinophils, and macrophages).

“Framework region” or “FR,” as used herein refers broadly to one or more of the framework regions within the variable regions of the light and heavy chains of an antibody. See Kabat, et al. “Sequences of Proteins of Immunological Interest” National Institutes of Health, Bethesda, Md. (1987). These expressions include those amino acid sequence regions interposed between the CDRs within the variable regions of the light and heavy chains of an antibody.

“Heterologous,” as used herein, refers broadly to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid (e.g., a promoter from one source and a coding region from another source.) Similarly, a heterologous protein indicates that the protein comprises two or more subsequences that are not found in the same relationship to each other in nature (e.g., a fusion protein).

“High affinity,” as used herein, refers broadly to an antibody or fusion protein having a K_{D} of at least 10⁻⁶ M, more preferably 10⁻⁷ M, even more preferably at least 10⁻⁸ M and even more preferably at least 10⁻⁹ M, 10⁻¹⁰ M, 10⁻¹¹ M, or 10⁻¹² M for a target antigen or receptor.

“High affinity” for an IgG antibody or fusion protein herein refers to an antibody having a K_{D} of 10⁻⁶ M or less, more preferably 10⁻⁷ M or less, preferably 10⁻⁸ M or less, more preferably 10⁻⁹ M or less and even more preferably 10⁻¹⁰ M, 10⁻¹¹ M, or 10⁻¹² M or less for a target antigen or receptor. With particular respect to antibodies, “high affinity” binding can vary for different antibody isotypes. For example, “high affinity” binding for an IgM isotype refers to an antibody having a K_{D} of 10⁻⁷M or less, more preferably 10⁻⁸ M or less.

“Homology,” as used herein, refers broadly to a degree of similarity between a nucleic acid sequence and a reference nucleic acid sequence or between a polypeptide sequence and a reference polypeptide sequence. Homology may be partial or complete. Complete homology indicates that the nucleic acid or amino acid sequences are identical. A partially homologous nucleic acid or amino acid sequence is one that is not identical to the reference nucleic acid or amino acid sequence. The degree of homology can be determined by sequence comparison, for example using BlastP software of the National Center of Biotechnology Information (NCBI) using default parameters. The term “sequence identity” may be used interchangeably with “homology.”

“Host cell,” as used herein, refers broadly to refer to a cell into which a nucleic acid molecule of the invention, such as a recombinant expression vector of the invention, has been introduced. Host cells may be prokaryotic cells (e.g., E. coli), or eukaryotic cells such as yeast, insect (e.g., SF9), amphibian, or mammalian cells such as CHO, HeLa, HEK-293, e.g., cultured cells, explants, and cells in vivo. The terms “host cell” and “recombinant host cell” are used interchangeably herein. It should be understood that such terms refer not only to the particular subject cell but to the progeny or potential progeny of such a cell. Because certain modifications may occur in succeeding generations due to either mutation or environmental influences, progeny may not, in fact, be identical to the parent cell, but are still included within the scope of the term as used herein.

“Human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell. This includes fully human monoclonal antibodies and conjugates and variants thereof, e.g., which are bound to effector agents such as therapeutics or diagnostic agents.

“Humanized antibody,” as used herein, refers broadly to include antibodies made by a non-human cell having variable and constant regions which have been altered to more closely resemble antibodies that would be made by a human cell. For example, by altering the non-human antibody amino acid sequence to incorporate amino acids found in human germline immunoglobulin sequences. The humanized antibodies of the invention may include amino acid residues not encoded by human germline immunoglobulin sequences (e.g., mutations introduced by random or site-specific mutagenesis in vitro or by somatic mutation in vivo), for example in the CDRs. The term “humanized antibody”, as used herein, also includes antibodies in which CDR sequences derived from the germline of another mammalian species, such as a mouse, have been grafted onto human framework sequences.

“Hybridization,” as used herein, refers broadly to the physical interaction of complementary (including partially complementary) polynucleotide strands by the formation of hydrogen bonds between complementary nucleotides when the strands are arranged antiparallel to each other.

“IgV domain” and “IgC domain” as used herein, refer broadly to Ig superfamily member domains. These domains correspond to structural units that have distinct folding patterns called Ig folds. Ig folds are comprised of a sandwich of two β sheets, each consisting of antiparallel β strands of 5-10 amino acids with a conserved disulfide bond between the two sheets in most, but not all, domains. IgC domains of Ig, TCR, and MHC molecules share the same types of sequence patterns and are called the C1 set within the Ig superfamily Other IgC domains fall within other sets. IgV domains also share sequence patterns and are called V set domains. IgV domains are longer than C-domains and form an additional pair of β strands.

“Immune cell,” as used herein, refers broadly to cells that are of hematopoietic origin and that play a role in the immune response Immune cells include but are not limited to lymphocytes, such as B cells and T cells; natural killer cells; dendritic cells, and myeloid cells, such as monocytes, macrophages, eosinophils, mast cells, basophils, and granulocytes.

“Immunoassay,” as used herein, refers broadly to an assay that uses an antibody to specifically bind an antigen. The immunoassay may be characterized by the use of specific binding properties of a particular antibody to isolate, target, and/or quantify the antigen.

“Immune related disease (or disorder or condition)” as used herein should be understood to encompass any disease disorder or condition selected from the group including but not limited to autoimmune diseases, inflammatory disorders and immune disorders associated with graft transplantation rejection, such as acute and chronic rejection of organ transplantation, allogenic stem cell transplantation, autologous stem cell transplantation, bone marrow transplantation, and graft versus host disease.

“Immune response,” as used herein, refers broadly to T cell-mediated and/or B cell-mediated immune responses that are influenced by modulation of T cell costimulation. Exemplary immune responses include B cell responses (e.g., antibody production) T cell responses (e.g., cytokine production, and cellular cytotoxicity) and activation of cytokine responsive cells, e.g., macrophages. As used herein, the term “downmodulation” with reference to the immune response includes a diminution in any one or more immune responses, while the term “upmodulation” with reference to the immune response includes an increase in any one or more immune responses. It will be understood that upmodulation of one type of immune response may lead to a corresponding downmodulation in another type of immune response. For example, upmodulation of the production of certain cytokines (e.g., IL-10) can lead to downmodulation of cellular immune responses.

“Immunologic”, “immunological” or “immune” response herein refer to the development of a humoral (antibody mediated) and/or a cellular (mediated by antigen-specific T cells or their secretion products) response directed against a peptide in a recipient patient. Such a response can be an active response induced by administration of immunogen or a passive response induced by administration of antibody or primed T-cells. Without wishing to be limited by a single hypothesis, a cellular immune response is elicited by the presentation of polypeptide epitopes in association with Class II or Class I MHC molecules to activate antigen-specific CD4⁺ T helper cells and/or CD8⁺ cytotoxic T cells, respectively. The response may also involve activation of monocytes, macrophages, NK cells, basophils, dendritic cells, astrocytes, microglia cells, eosinophils, activation or recruitment of neutrophils or other components of innate immunity. The presence of a cell-mediated immunological response can be determined by proliferation assays (CD4⁺ T cells) or CTL (cytotoxic T lymphocyte) assays. The relative contributions of humoral and cellular responses to the protective or therapeutic effect of an immunogen can be distinguished by separately isolating antibodies and T cells from an immunized syngeneic animal and measuring protective or therapeutic effect in a second subject.

“Immunogenic agent” or “immunogen” is a moiety capable of inducing an immunological response against itself on administration to a mammal, optionally in conjunction with an adjuvant.

“Infectious agent” herein refers to any pathogen or agent that infects mammalian cells, preferably human cells and causes a disease condition. Examples thereof include bacteria, yeast, fungi, protozoans, mycoplasma, viruses, prions, and parasites. Examples of such infectious agents include by way of example those involved in (a) viral diseases such as, for example, diseases resulting from infection by an adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenza virus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV); (b) bacterial diseases such as, for example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella; (c) other infectious diseases, such chlamydia, fungal diseases including but not limited to candidiasis, aspergillosis, histoplasmosis, cryptococcal meningitis, parasitic diseases including but not limited to malaria, pneumocystis carnii pneumonia, leishmaniasis, cryptosporidiosis, toxoplasmosis, and trypanosome infection and prions that cause human disease such as Creutzfeldt-Jakob Disease (CJD), variant Creutzfeldt-Jakob Disease (vCJD), Gerstmann-Stra ussler-Scheinker syndrome, Fatal Familial Insomnia and kuru.

“Infectious agent antigen” herein means a compound, e.g., peptide, polypeptide, glycopeptide, glycoprotein, and the like, or a conjugate, fragment or variant thereof, which compound is expressed by a specific infectious agent and which antigen may be used to elicit a specific immune response, e.g., antibody or cell-mediated immune response against the infectious agent such as a virus. Typically the antigen will comprise a moiety, e.g., polypeptide or glycoprotein expressed on the surface of the virus or other infectious agent, such as a capsid protein or other membrane protein.

“Inflammatory bowel disease” herein comprises any inflammatory bowel condition and especially includes inflammatory bowel disease, Crohn's disease, ulcerative colitis (UC), collagenous colitis, lymphocytic colitis, ischemic colitis, diversion colitis, Behçet's disease, and indeterminate colitis.

“Inflammatory disorders”, “inflammatory conditions” and/or “inflammation”, used interchangeably herein, refers broadly to chronic or acute inflammatory diseases, and expressly includes inflammatory autoimmune diseases and inflammatory allergic conditions. These conditions include by way of example inflammatory abnormalities characterized by dysregulated immune response to harmful stimuli, such as pathogens, damaged cells, or irritants. Inflammatory disorders underlie a vast variety of human diseases. Non-immune diseases with etiological origins in inflammatory processes include cancer, atherosclerosis, and ischemic heart disease. Examples of disorders associated with inflammation include: Chronic prostatitis, Glomerulonephritis, Hypersensitivities, Pelvic inflammatory disease, Reperfusion injury, Sarcoidosis, Vasculitis, Interstitial cystitis, normocomplementemic urticarial vasculitis, pericarditis, myositis, anti-synthetase syndrome, scleritis, macrophage activation syndrome, Behçet's Syndrome, PAPA Syndrome, Blau's Syndrome, gout, adult and juvenile Still's disease, cryropyrinopathy, Muckle-Wells syndrome, familial cold-induced auto-inflammatory syndrome, neonatal onset multisystemic inflammatory disease, familial Mediterranean fever, chronic infantile neurologic, cutaneous and articular syndrome, systemic juvenile idiopathic arthritis, Hyper IgD syndrome, Schnitzler's syndrome, TNF receptor-associated periodic syndrome (TRAPSP), gingivitis, periodontitis, hepatitis, cirrhosis, pancreatitis, myocarditis, vasculitis, gastritis, gout, gouty arthritis, and inflammatory skin disorders, selected from the group consisting of psoriasis, atopic dermatitis, eczema, rosacea, urticaria, and acne.

“Inhibitory signal,” as used herein, refers broadly to a signal transmitted via an inhibitory receptor molecule on an immune cell. A signal antagonizes a signal via an activating receptor (e.g., via a TCR, CD3, BCR, or Fc molecule) and can result, e.g., in inhibition of: second messenger generation; proliferation; or effector function in the immune cell, e.g., reduced phagocytosis, antibody production, or cellular cytotoxicity, or the failure of the immune cell to produce mediators (e.g., cytokines (e.g., IL-2) and/or mediators of allergic responses); or the development of anergy.

“Isolated,” as used herein, refers broadly to material removed from its original environment in which it naturally occurs, and thus is altered by the hand of man from its natural environment and includes “recombinant” polypeptides. Isolated material may be, for example, exogenous nucleic acid included in a vector system, exogenous nucleic acid contained within a host cell, or any material which has been removed from its original environment and thus altered by the hand of man (e.g., “isolated antibody”). For example, “isolated” or “purified,” as used herein, refers broadly to a protein, DNA, antibody, RNA, or biologically active portion thereof, that is substantially free of cellular material or other contaminating proteins from the cell or tissue source from which the biological substance is derived, or substantially free from chemical precursors or other chemicals when chemically synthesized. As used herein the term “isolated” refers to a compound of interest (for example a polynucleotide or a polypeptide) that is in an environment different from that in which the compound naturally occurs e.g., separated from its natural milieu such as by concentrating a peptide to a concentration at which it is not found in nature. “Isolated” includes compounds that are within samples that are substantially enriched for the compound of interest and/or in which the compound of interest is partially or substantially purified.

“Isolated antibody”, as used herein, is intended to refer to an antibody that is substantially free of other antibodies having different antigenic specificities (e.g., an isolated antibody that specifically binds VSTM5 is substantially free of antibodies that specifically bind antigens other than VSTM5). Moreover, an isolated antibody may be substantially free of other cellular material and/or chemicals.

“Isotype” herein refers to the antibody class (e.g., IgM or IgG1) that is encoded by the heavy chain constant region genes.

“K-assoc” or “Kₐ”, as used herein, refers broadly to the association rate of a particular antibody-antigen interaction, whereas the term “K_{diss}” or “K_{d},” as used herein, refers to the dissociation rate of a particular antibody-antigen interaction. The term “K_{D}”, as used herein, is intended to refer to the dissociation constant, which is obtained from the ratio of K_{d} to Kₐ (i.e., K_{d}/Kₐ) and is expressed as a molar concentration (M). K_{D} values for antibodies can be determined using methods well established in the art such as plasmon resonance (BIAcore®), ELISA and KINEXA. A preferred method for determining the K_{D} of an antibody is by using surface Plasmon resonance, preferably using a biosensor system such as a BIAcore® system or by ELISA.

“Label” or a “detectable moiety” as used herein, refers broadly to a composition detectable by spectroscopic, photochemical, biochemical, immunochemical, chemical, or other physical means.

“Low stringency,” “medium stringency,” “high stringency,” or “very high stringency conditions,” as used herein, refers broadly to conditions for nucleic acid hybridization and washing. Guidance for performing hybridization reactions can be found in Ausubel, et al., Short Protocols in Molecular Biology (5th Ed.) John Wiley & Sons, NY (2002). Exemplary specific hybridization conditions include but are not limited to: (1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); (2) medium stringency hybridization conditions in 6×SSC at about 45° C., followed by one or more washes in 0.2×SSC, 0.1% SDS at 60° C.; (3) high stringency hybridization conditions in 6×SSC at about 45° C. followed by one or more washes in 0.2×. SSC, 0.1% SDS at 65° C.; and (4) very high stringency hybridization conditions are 0.5M sodium phosphate, 7% SDS at 65° C., followed by one or more washes at 0.2×SSC, and 1% SDS at 65° C.

“Mammal,” as used herein, refers broadly to any and all warm-blooded vertebrate animals of the class Mammalia, including humans, characterized by a covering of hair on the skin and, in the female, milk-producing mammary glands for nourishing the young. Examples of mammals include but are not limited to alpacas, armadillos, capybaras, cats, camels, chimpanzees, chinchillas, cattle, dogs, goats, gorillas, hamsters, horses, humans, lemurs, llamas, mice, non-human primates, pigs, rats, sheep, shrews, squirrels, tapirs, and voles. Mammals include but are not limited to bovine, canine, equine, feline, murine, ovine, porcine, primate, and rodent species. Mammal also includes any and all those listed on the Mammal Species of the World maintained by the National Museum of Natural History, Smithsonian Institution in Washington D. C.

“Multiple sclerosis” includes by way of example multiple sclerosis, benign multiple sclerosis, relapsing remitting multiple sclerosis, secondary progressive multiple sclerosis, primary progressive multiple sclerosis, progressive relapsing multiple sclerosis, chronic progressive multiple sclerosis, transitional/progressive multiple sclerosis, rapidly worsening multiple sclerosis, clinically-definite multiple sclerosis, malignant multiple sclerosis, also known as Marburg's Variant, and acute multiple sclerosis. Optionally, “conditions relating to multiple sclerosis” include, e.g., Devic's disease, also known as Neuromyelitis Optica; acute disseminated encephalomyelitis, acute demyelinating optic neuritis, demyelinative transverse myelitis, Miller-Fisher syndrome, encephalomyeloradiculoneuropathy, acute demyelinative polyneuropathy, tumefactive multiple sclerosis and Balo's concentric sclerosis.

“Naturally-occurring nucleic acid molecule,” as used herein, refers broadly refers to an RNA or DNA molecule having a nucleotide sequence that occurs in nature (e.g., encodes a natural protein).

“Nucleic acid” or “nucleic acid sequence,” as used herein, refers broadly to a deoxy-ribonucleotide or ribonucleotide oligonucleotide in either single- or double-stranded form. The term encompasses nucleic acids, i.e., oligonucleotides, containing known analogs of natural nucleotides. The term also encompasses nucleic-acid-like structures with synthetic backbones. Unless otherwise indicated, a particular nucleic acid sequence also implicitly encompasses conservatively modified variants thereof (e.g., degenerate codon substitutions) and complementary sequences, as well as the sequence explicitly indicated. The term nucleic acid is used interchangeably with gene, cDNA, mRNA, oligonucleotide, and polynucleotide.

“Oligomerization domain”, or “multimerization domain” or “dimerization domain” are used interchangeably herein, and refer broadly to a domain that when attached to a VSTM5 extracellular domain or fragment thereof, facilitates oligomerization. Said oligomerization domains comprise self-associating. α.-helices, for example, leucine zippers, that can be further stabilized by additional disulfide bonds. The domains are designed to be compatible with vectorial folding across a membrane, a process thought to facilitate in vivo folding of the polypeptide into a functional binding protein. Examples thereof are known in the art and include by way of example coiled GCN4, and COMP. The α-helical coiled coil is probably the most widespread subunit oligomerization motif found in proteins. Accordingly, coiled coils fulfill a variety of different functions. In several families of transcriptional activators, for example, short leucine zippers play an important role in positioning the DNA-binding regions on the DNA. Ellenberger, et al. (1992) Cell 71: 1223-1237. Coiled coils are also used to form oligomers of intermediate filament proteins. Coiled-coil proteins furthermore appear to play an important role in both vesicle and viral membrane fusion. (Skehel and Wiley Cell 95: 871-874(1998)). In both cases hydrophobic sequences, embedded in the membranes to be fused, are located at the same end of the rod-shaped complex composed of a bundle of long α-helices. This molecular arrangement is believed to cause close membrane apposition as the complexes are assembled for membrane fusion. The coiled coil is often used to control oligomerization. It is found in many types of proteins, including transcription factors include, but not limited to GCN4, viral fusion peptides, SNARE complexes and certain tRNA synthetases, among others. Very long coiled coils are found in proteins such as tropomyosin, intermediate filaments and spindle-pole-body components. Coiled coils involve a number of α-helices that are supercoiled around each other in a highly organized manner that associate in a parallel or an antiparallel orientation. Although dimers and trimers are the most common. The helices may be from the same or from different proteins. The coiled-coil is formed by component helices coming together to bury their hydrophobic seams. As the hydrophobic seams twist around each helix, so the helices also twist to coil around each other, burying the hydrophobic seams and forming a supercoil. It is the characteristic interdigitation of side chains between neighboring helices, known as knobs-into-holes packing, that defines the structure as a coiled coil. The helices do not have to run in the same direction for this type of interaction to occur, although parallel conformation is more common Antiparallel conformation is very rare in trimers and unknown in pentamers, but more common in intramolecular dimers, where the two helices are often connected by a short loop. In the extracellular space, the heterotrimeric coiled-coil protein laminin plays an important role in the formation of basement membranes. Other examples are the thrombospondins and cartilage oligomeric matrix protein (COMP) in which three (thrombospondins 1 and 2) or five (thrombospondins 3, 4 and COMP) chains are connected. The molecules have a flower bouquet-like appearance, and the reason for their oligomeric structure is probably the multivalent interaction of the C-terminal domains with cellular receptors. The yeast transcriptional activator GCN4 is 1 of over 30 identified eukaryotic proteins containing the basic region leucine zipper (bZIP) DNA-binding motif. Ellenberger, et al. Cell 71: 1223-1237 (1992). The bZIP dimer is a pair of continuous a helices that form a parallel coiled-coil over their carboxy-terminal 34 residues and gradually diverge toward their amino termini to pass through the major groove of the DNA binding site. The coiled-coil dimerization interface is oriented almost perpendicular to the DNA axis, giving the complex the appearance of the letter T. bZIP contains a 4-3 heptad repeat of hydrophobic and nonpolar residues that pack together in a parallel α-helical coiled-coil. (Ellenberger, et al. Cell 71: 1223-1237 (1992)). The stability of the dimer results from the side-by-side packing of leucines and nonpolar residues in positions a and d of the heptad repeat, as well as a limited number of intra- and interhelical salt bridges, shown in a crystal structure of the GCN4 leucine zipper peptide. (Ellenberger, et al. Cell 71: 1223-1237 (1992)). Another example is CMP (matrilin-1) isolated from bovine tracheal cartilage as a homotrimer of subunits of Mr 52,000 (Paulsson & Heinegard (1981), Biochem. J. 197: 367-375), where each subunit consists of a vWFA1 module, a single EGF domain, a vWFA2 module and a coiled coil domain spanning five heptads. (Kiss, et al. J. Biol. Chem. 264:8126-8134 (1989); Hauser and Paulsson J. Biol. Chem. 269: 25747-25753 (1994)). Electron microscopy of purified CMP showed a bouquet-like trimer structure in which each subunit forms an ellipsoid emerging from a common point corresponding to the coiled coil. (Hauser and Paulsson J. Biol. Chem. 269: 25747-25753 (1994)). The coiled coil domain in matrilin-1 has been extensively studied. The trimeric structure is retained after complete reduction of interchain disulfide bonds under non-denaturing conditions. (Hauser and Paulsson J. Biol. Chem. 269: 25747-25753 (1994)). Yet another example is Cartilage Oligomeric Matrix Protein (COMP). A non-collagenous glycoprotein, COMP, was first identified in cartilage. (Hedbom, et al. J. Biol. Chem. 267:6132-6136(1992)). The protein is a 524 kDa homopentamer of five subunits which consists of an N-terminal heptad repeat region (cc) followed by four epidermal growth factor (EGF)-like domains (EF), seven calcium-binding domains (T3) and a C-terminal globular domain (TC). According to this domain organization, COMP belongs to the family of thrombospondins. Heptad repeats (abcdefg)ₙ with preferentially hydrophobic residues at positions a and d form-helical coiled-coil domains. (Cohen and Parry Science 263: 488-489 (1994)). Recently, the recombinant five-stranded coiled-coil domain of COMP (COMPcc) was crystallized and its structure was solved at 0.2 nm resolution. (Malashkevich, et al. Science 274: 761-765 (1996)).

“Operatively linked”, as used herein, refers broadly to when two DNA fragments are joined such that the amino acid sequences encoded by the two DNA fragments remain in-frame.

“Paratope,” as used herein, refers broadly to the part of an antibody which recognizes an antigen (e.g., the antigen-binding site of an antibody.) Paratopes may be a small region (e.g., 15-22 amino acids) of the antibody's Fv region and may contain parts of the antibody's heavy and light chains. See Goldsby, et al. Antigens (Chapter 3) Immunology (5^{th} Ed.) New York: W. H. Freeman and Company, pages 57-75.

“Patient,” or “subject” or “recipient”, “individual”, or “treated individual” are used interchangeably herein, and refers broadly to any animal that is in need of treatment either to alleviate a disease state or to prevent the occurrence or reoccurrence of a disease state. Also, “Patient” as used herein, refers broadly to any animal that has risk factors, a history of disease, susceptibility, symptoms, and signs, was previously diagnosed, is at risk for, or is a member of a patient population for a disease. The patient may be a clinical patient such as a human or a veterinary patient such as a companion, domesticated, livestock, exotic, or zoo animal.

“Polypeptide,” “peptide” and “protein,” are used interchangeably and refer broadly to a polymer of amino acid residues s of any length, regardless of modification (e.g., phosphorylation or glycosylation). The terms apply to amino acid polymers in which one or more amino acid residue is an analog or mimetic of a corresponding naturally occurring amino acid, as well as to naturally occurring amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. Polypeptides can be modified, e.g., by the addition of carbohydrate residues to form glycoproteins. The terms “polypeptide,” “peptide” and “protein” expressly include glycoproteins, as well as non-glycoproteins.

“Promoter,” as used herein, refers broadly to an array of nucleic acid sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation.

“Prophylactically effective amount,” as used herein, refers broadly to the amount of a compound that, when administered to a patient for prophylaxis of a disease or prevention of the reoccurrence of a disease, is sufficient to effect such prophylaxis for the disease or reoccurrence. The prophylactically effective amount may be an amount effective to prevent the incidence of signs and/or symptoms. The “prophylactically effective amount” may vary depending on the disease and its severity and the age, weight, medical history, predisposition to conditions, preexisting conditions, of the patient to be treated.

“Prophylactic vaccine” and/or “Prophylactic vaccination” refers to a vaccine used to prevent a disease or symptoms associated with a disease such as cancer or an infectious condition.

“Prophylaxis,” as used herein, refers broadly to a course of therapy where signs and/or symptoms are not present in the patient, are in remission, or were previously present in a patient. Prophylaxis includes preventing disease occurring subsequent to treatment of a disease in a patient. Further, prevention includes treating patients who may potentially develop the disease, especially patients who are susceptible to the disease (e.g., members of a patent population, those with risk factors, or at risk for developing the disease).

“Psoriasis” herein includes one or more of psoriasis, Nonpustular Psoriasis including Psoriasis vulgaris and Psoriatic erythroderma (erythrodermic psoriasis), Pustular psoriasis including Generalized pustular psoriasis (pustular psoriasis of von Zumbusch), Pustulosis palmaris et plantaris (persistent palmoplantar pustulosis, pustular psoriasis of the Barber type, pustular psoriasis of the extremities), Annular pustular psoriasis, Acrodermatitis continua, Impetigo herpetiformis. Optionally, conditions relating to psoriasis include e.g., drug-induced psoriasis, Inverse psoriasis, Napkin psoriasis, Seborrheic-like psoriasis, Guttate psoriasis, Nail psoriasis, and Psoriatic arthritis.

“Recombinant” as used herein, refers broadly with reference to a product, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The term “recombinant human antibody”, as used herein, includes all human antibodies that are prepared, expressed, created or isolated by recombinant means, such as (a) antibodies isolated from an animal (e.g., a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

“Rheumatoid arthritis” includes by way of example rheumatoid arthritis, gout and pseudo-gout, juvenile idiopathic arthritis, juvenile rheumatoid arthritis, Still's disease, ankylosing spondylitis, rheumatoid vasculitis, as well as other conditions relating to rheumatoid arthritis such as e.g., osteoarthritis, sarcoidosis, Henoch-Schönlein purpura, Psoriatic arthritis, Reactive arthritis, Spondyloarthropathy, septic arthritis, Hemochromatosis, Hepatitis, vasculitis, Wegener's granulomatosis, Lyme disease, Familial Mediterranean fever, Hyperimmunoglobulinemia D with recurrent fever, TNF receptor associated periodic syndrome, and Enteropathic arthritis associated with inflammatory bowel disease.

“Signal sequence” or “signal peptide,” as used herein, refers broadly to a peptide containing about 15 or more amino acids which occurs at the N-terminus of secretory and membrane bound polypeptides and which contains a large number of hydrophobic amino acid residues. For example, a signal sequence contains at least about 10-30 amino acid residues, preferably about 15-25 amino acid residues, more preferably about 18-20 amino acid residues, and even more preferably about 19 amino acid residues, and has at least about 35-65%, preferably about 38-50%, and more preferably about 40-45% hydrophobic amino acid residues (e.g., Valine, Leucine, Isoleucine or Phenylalanine). A “signal sequence,” also referred to in the art as a “signal peptide,” serves to direct a polypeptide containing such a sequence to a lipid bilayer, and is cleaved in secreted.

“Sjögren's syndrome” herein includes one or more of Sjögren's syndrome, Primary Sjögren's syndrome and Secondary Sjögren's syndrome, as well as conditions relating to Sjögren's syndrome including connective tissue disease, such as rheumatoid arthritis, systemic lupus erythematosus, or scleroderma. Other complications include pneumonia, pulmonary fibrosis, interstitial nephritis, inflammation of the tissue around the kidney's filters, glomerulonephritis, renal tubular acidosis, carpal tunnel syndrome, peripheral neuropathy, cranial neuropathy, primary biliary cirrhosis (PBC), cirrhosis, Inflammation in the esophagus, stomach, pancreas, and liver (including hepatitis), Polymyositis, Raynaud's phenomenon, Vasculitis, Autoimmune thyroid problems, and lymphoma.

“Specifically (or selectively) binds” to an antibody or “specifically (or selectively) immunoreactive with,” or “specifically interacts or binds,” as used herein, refers broadly to a protein or peptide (or other epitope), refers, in some embodiments, to a binding reaction that is determinative of the presence of the protein in a heterogeneous population of proteins and other biologics. For example, under designated immunoassay conditions, the specified antibodies bind to a particular protein at least two times greater than the background (non-specific signal) and do not substantially bind in a significant amount to other proteins present in the sample. Typically a specific or selective reaction will be at least twice background signal or noise and more typically more than about 10 to 100 times background.

“Specifically hybridizable” and “complementary” as used herein, refer broadly to a nucleic acid can form hydrogen bond(s) with another nucleic acid sequence by either traditional Watson-Crick or other non-traditional types. The binding free energy for a nucleic acid molecule with its complementary sequence is sufficient to allow the relevant function of the nucleic acid to proceed, e.g., RNAi activity. Determination of binding free energies for nucleic acid molecules is well known in the art. (See, e.g., Turner, et al. CSH Symp. Quant. Biol. LII: 123-33 (1987); Frier, et al. PNAS 83: 9373-77 1986); Turner, et al. J. Am. Chem. Soc. 109: 3783-85 (1987)). A percent complementarity indicates the percentage of contiguous residues in a nucleic acid molecule that can form hydrogen bonds (e.g., Watson-Crick base pairing) with a second nucleic acid sequence (e.g., about at least 5, 6, 7, 8, 9, 10 out of 10 being about at least 50%, 60%, 70%, 80%, 90%, and 100% complementary, inclusive). “Perfectly complementary” or 100% complementarity refers broadly all of the contiguous residues of a nucleic acid sequence hydrogen bonding with the same number of contiguous residues in a second nucleic acid sequence.

“Substantial complementarity” refers to polynucleotide strands exhibiting about at least 90% complementarity, excluding regions of the polynucleotide strands, such as overhangs, that are selected so as to be noncomplementary. Specific binding requires a sufficient degree of complementarity to avoid non-specific binding of the oligomeric compound to non-target sequences under conditions in which specific binding is desired, i.e., under physiological conditions in the case of in vivo assays or therapeutic treatment, or in the case of in vitro assays, under conditions in which the assays are performed. The non-target sequences typically may differ by at least 5 nucleotides.

“Signs” of disease, as used herein, refers broadly to any abnormality indicative of disease, discoverable on examination of the patient; an objective indication of disease, in contrast to a symptom, which is a subjective indication of disease.

“Solid support,” “support,” and “substrate,” as used herein, refers broadly to any material that provides a solid or semi-solid structure with which another material can be attached including but not limited to smooth supports (e.g., metal, glass, plastic, silicon, and ceramic surfaces) as well as textured and porous materials.

“Soluble ectodomain (ECD)” or “ectodomain” or “soluble VSTM5 protein(s)/molecule(s)” of VSTM5 as used herein means non-cell-surface-bound VSTM5 molecules or any portion thereof, including, but not limited to: VSTM5 fusion proteins or VSTM5 ECD-Ig fusion proteins, wherein the extracellular domain of VSTM5 or fragment thereof is fused to an immunoglobulin (Ig) moiety rendering the fusion molecule soluble, or fragments and derivatives thereof, proteins with the extracellular domain of VSTM5 fused or joined with a portion of a biologically active or chemically active protein such as the papillomavirus E7 gene product, melanoma-associated antigen p97 or HIV env protein, or fragments and derivatives thereof; hybrid (chimeric) fusion proteins such as VSTM5-Ig, or fragments and derivatives thereof. Such fusion proteins are described in greater detail below.

“Soluble VSTM5 protein(s)/molecule(s)” herein also include VSTM5 molecules with the transmembrane domain removed to render the protein soluble, or fragments and derivatives thereof; fragments, portions or derivatives thereof, and soluble VSTM5 mutant molecules. The soluble VSTM5 molecules used in the methods according to at least some embodiments of the invention may or may not include a signal (leader) peptide sequence.

“Subject” or “patient” or “individual” in the context of therapy or diagnosis herein includes any human or nonhuman animal. The term “nonhuman animal” includes all vertebrates, e.g., mammals and non-mammals, such as nonhuman primates, sheep, dogs, cats, horses, cows, chickens, amphibians, reptiles, etc., i.e., anyone suitable to be treated according to the present invention include, but are not limited to, avian and mammalian subjects, and are preferably mammalian. Any mammalian subject in need of being treated according to the present invention is suitable. Human subjects of both genders and at any stage of development (i.e., neonate, infant, juvenile, adolescent, and adult) can be treated according to the present invention. The present invention may also be carried out on animal subjects, particularly mammalian subjects such as mice, rats, dogs, cats, cattle, goats, sheep, and horses for veterinary purposes, and for drug screening and drug development purposes. “Subjects” is used interchangeably with “individuals” and “patients.”

“Substantially free of chemical precursors or other chemicals,” as used herein, refers broadly to preparations of VSTM5 protein in which the protein is separated from chemical precursors or other chemicals which are involved in the synthesis of the protein. In one embodiment, the language “substantially free of chemical precursors or other chemicals” includes preparations of VSTM5 protein having less than about 30% (by dry weight) of chemical precursors or non-VSTM5 chemicals, more preferably less than about 20% chemical precursors or non-VSTM5 chemicals, still more preferably less than about 10% chemical precursors or non-VSTM5 chemicals, and most preferably less than about 5% chemical precursors or non-VSTM5 chemicals.

“Symptoms” of disease as used herein, refers broadly to any morbid phenomenon or departure from the normal in structure, function, or sensation, experienced by the patient and indicative of disease.

“Systemic lupus erythematosus”, as used herein comprises one or more of systemic lupus erythematosus, discoid lupus, lupus arthritis, lupus pneumonitis, lupus nephritis. Conditions relating to systemic lupus erythematosus include osteoarticular tuberculosis, antiphospholipid antibody syndrome, inflammation of various parts of the heart, such as pericarditis, myocarditis, and endocarditis, Lung and pleura inflammation, pleuritis, pleural effusion, chronic diffuse interstitial lung disease, pulmonary hypertension, pulmonary emboli, pulmonary hemorrhage, and shrinking lung syndrome, lupus headache, Guillain-Barré syndrome, aseptic meningitis, demyelinating syndrome, mononeuropathy, mononeuritis multiplex, myasthenia gravis, myelopathy, cranial neuropathy, polyneuropathy, vasculitis.

“T cell,” as used herein, refers broadly to CD4⁺ T cells and CD8⁺ T cells. The term T cell also includes both T helper 1 type T cells and T helper 2 type T cells.

“Therapy,” “therapeutic,” “treating,” or “treatment”, as used herein, refers broadly to treating a disease, arresting, or reducing the development of the disease or its clinical symptoms, and/or relieving the disease, causing regression of the disease or its clinical symptoms. Therapy encompasses prophylaxis, treatment, remedy, reduction, alleviation, and/or providing relief from a disease, signs, and/or symptoms of a disease. Therapy encompasses an alleviation of signs and/or symptoms in patients with ongoing disease signs and/or symptoms (e.g., inflammation, pain). Therapy also encompasses “prophylaxis”. The term “reduced”, for purpose of therapy, refers broadly to the clinical significant reduction in signs and/or symptoms. Therapy includes treating relapses or recurrent signs and/or symptoms (e.g., inflammation, pain). Therapy encompasses but is not limited to precluding the appearance of signs and/or symptoms anytime as well as reducing existing signs and/or symptoms and eliminating existing signs and/or symptoms. Therapy includes treating chronic disease (“maintenance”) and acute disease. For example, treatment includes treating or preventing relapses or the recurrence of signs and/or symptoms (e.g., inflammation, pain).

“Therapeutic vaccine” and/or “therapeutic vaccination” refers to a vaccine used to treat a disease such as cancer or an infectious condition.

“Treg cell” (sometimes also referred to as suppressor T cells or inducible Treg cells or iTregs) as used herein refers to a subpopulation of T cells which modulate the immune system and maintain tolerance to self-antigens and can abrogate autoimmune diseases. Foxp3⁺ CD4⁺CD25⁺ regulatory T cells (Tregs) are critical in maintaining peripheral tolerance under normal

“Transmembrane domain,” as used herein, refers broadly to an amino acid sequence of about 15 amino acid residues in length which spans the plasma membrane. More preferably, a transmembrane domain includes about at least 20, 25, 30, 35, 40, or 45 amino acid residues and spans the plasma membrane. Transmembrane domains are rich in hydrophobic residues, and typically have an α-helical structure. In an embodiment, at least 50%, 60%, 70%, 80%, 90%, 95% or more of the amino acids of a transmembrane domain are hydrophobic, e.g., leucines, isoleucines, tyrosines, or tryptophans. Transmembrane domains are described in, for example, Zagotta, et al. Annu. Rev. Neurosci. 19:235-263 (1996).

“Transgenic animal,” as used herein, refers broadly to a non-human animal, preferably a mammal, more preferably a mouse, in which one or more of the cells of the animal includes a “transgene”. The term “transgene” refers to exogenous DNA which is integrated into the genome of a cell from which a transgenic animal develops and which remains in the genome of the mature animal, for example directing the expression of an encoded gene product in one or more cell types or tissues of the transgenic animal.

“Tumor,” as used herein, refers broadly to at least one cell or cell mass in the form of a tissue neoformation, in particular in the form of a spontaneous, autonomous and irreversible excess growth, which is more or less disinhibited, of endogenous tissue, which growth is as a rule associated with the more or less pronounced loss of specific cell and tissue functions. This cell or cell mass is not effectively inhibited, in regard to its growth, by itself or by the regulatory mechanisms of the host organism, e.g., colorectal cancer, melanoma or carcinoma. Tumor antigens not only include antigens present in or on the malignant cells themselves, but also include antigens present on the stromal supporting tissue of tumors including endothelial cells and other blood vessel components.

“Type 1 diabetes” herein includes one or more of type 1 diabetes, insulin-dependent diabetes mellitus, idiopathic diabetes, juvenile type ldiabetes, maturity onset diabetes of the young, latent autoimmune diabetes in adults, gestational diabetes. Conditions relating to type 1 diabetes include, neuropathy including polyneuropathy, mononeuropathy, peripheral neuropathy and autonomicneuropathy; eye complications: glaucoma, cataracts, and retinopathy.

“Unresponsiveness,” as used herein, refers broadly to refractivity of immune cells to stimulation, e.g., and stimulation via an activating receptor or a cytokine. Unresponsiveness can occur, e.g., because of exposure to immunosuppressants or high doses of antigen.

“Uveitis” as used herein comprises one or more of uveitis, anterior uveitis (or iridocyclitis), intermediate uveitis (pars planitis), posterior uveitis (or chorioretinitis) and the panuveitic form.

“Vaccine” as used herein, refers to a biological preparation that as improves immunity to a particular disease, e.g., cancer or an infectious disease, wherein the vaccine includes a disease specific antigen, e.g., a cancer antigen or infectious agent antigen, against which immune responses are elicited. A vaccine typically includes an adjuvant as immune potentiator to stimulate the immune system. This includes prophylactic (which prevent disease) and therapeutic vaccines (which treat the disease or its symptoms).

“Variable region” or “VR,” as used herein, refers broadly to the domains within each pair of light and heavy chains in an antibody that are involved directly in binding the antibody to the antigen. Each heavy chain has at one end a variable domain (V_{H}) followed by a number of constant domains. Each light chain has a variable domain (V_{L}) at one end and a constant domain at its other end; the constant domain of the light chain is aligned with the first constant domain of the heavy chain, and the light chain variable domain is aligned with the variable domain of the heavy chain.

“Vector,” as used herein, refers broadly to a nucleic acid molecule capable of transporting another nucleic acid molecule to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively linked. Vectors are referred to herein as “recombinant expression vectors” or simply “expression vectors”. In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions. The techniques and procedures are generally performed according to conventional methods well known in the art and as described in various general and more specific references that are cited and discussed throughout the present specification. See, e.g., Sambrook, et al. Molec. Cloning: Lab. Manual [3rd Ed] Cold Spring Harbor Laboratory Press (2001). Standard techniques may be used for recombinant DNA, oligonucleotide synthesis, and tissue culture, and transformation (e.g., electroporation, lipofection). Enzymatic reactions and purification techniques may be performed according to manufacturer's specifications or as commonly accomplished in the art or as described herein.

Having defined certain terms and phrases used in the present application, specific types VSTM5 polypeptides, fusion proteins, and methods for the production and use thereof which are embraced by the invention are further described below.

Fragments of VSTM5 Polypeptides

The term the “soluble ectodomain (ECD)” or “ectodomain” or “soluble” form of VSTM5 refers also to the nucleic acid sequences encoding the corresponding proteins. Optionally, the VSTM5 ECD proteins and fragments thereof refer to any one of the polypeptide sequences listed in any of SEQ ID NOs: 1-3, 22-110, 151-156, and/or variants thereof possessing at least 80% sequence identity, more preferably at least 90% sequence identity therewith and even more preferably at least 95, 96, 97, 98 or 99% sequence identity therewith, and/or fusions and or conjugates thereof, and/or polynucleotides encoding same.

Optionally, the fragment is of at least about 96 and so forth amino acids of the extracellular domain of VSTM5 protein, set forth in SEQ ID NO: 2, up to 120 amino acids of the VSTM5 protein extracellular domain, optionally including any integral value between 96 and 120 amino acids in length. Preferably, the fragment is of at least about 115 and up to 120 amino acids of the VSTM5 protein extracellular domain, optionally including any integral value between 115 and 120 amino acids in length. Also preferably the fragment is of at least about 117 up to 119 amino acids of the VSTM5 protein extracellular domain, optionally including any integral value between 117 and 119 amino acids in length. More preferably, the fragment is about 119 amino acids. The VSTM5 fragment protein according to at least some embodiments of the invention may or may not include a signal peptide sequence, and may or may not include 1, 2, 3, 4, or 5 contiguous amino acids from the VSTM5 transmembrane domain.

In particular, the fragments of the extracellular domain of VSTM5 can include any sequence corresponding to any portion of or comprising the IgV domain of the extracellular domain of VSTM5, having any sequence corresponding to residues of VSTM5 (SEQ ID NO:1) starting from any position between 40 and 44 and ending at any position between 135 and 139.

The VSTM5 proteins contain an immunoglobulin domain within the extracellular domain, the IgV domain (or V domain), which is related to the variable domain of antibodies. The IgV domain may be responsible for receptor binding, by analogy to the other B7 family members. The Ig domain of the extracellular domain includes one disulfide bond formed between intra domain cysteine residues, as is typical for this fold and may be important for structure-function. In SEQ ID NO: 1 these cysteines are located at residues 18 and 57.

In one embodiment, there is provided a soluble fragment of VSTM5; as described in greater detail below with regard to the section on fusion proteins, such a soluble fragment may optionally be described as a first fusion partner. Useful fragments are those that alone or when comprised in fusion proteins or multimerized retain the ability to bind to their natural receptor or receptors, e.g., expressed on T and NK cells, and/or which modulate (inhibit or promote) T cell and/or NK cell activation. A VSTM5 polypeptide that is a fragment of full-length VSTM5 typically has at least 20 percent, 30 percent, 40 percent, 50 percent, 60 percent, 70 percent, 80 percent, 90 percent, 95 percent, 98 percent, 99 percent, 100 percent, or even more than 100 percent of the ability to bind its natural receptor(s) and/or the modulation (agonism or antagonism) of one or more of the functional effects of VSTM5 on immunity and on specific immune cells as compared to full-length VSTM5. Soluble VSTM5 polypeptide fragments are fragments of VSTM5 polypeptides that may be shed, secreted or otherwise extracted from the producing cells. In other embodiments, the soluble fragments of VSTM5 polypeptides include fragments of the VSTM5 extracellular domain that retain VSTM5 biological activity, such as fragments that retain the ability to bind to their natural receptor or receptors and/or which modulate (inhibit or promote) T or NK cell activation. The extracellular domain can include 1, 2, 3, 4, or 5 contiguous amino acids from the transmembrane domain, and/or 1, 2, 3, 4, or 5 contiguous amino acids from the signal sequence. Alternatively, the extracellular domain can have 1, 2, 3, 4, 5 or more amino acids removed from the C-terminus, N-terminus, or both.

In some embodiments the VSTM5 extracellular domain polypeptide comprises the amino acid sequence of the IgV domain as set forth in any one of SEQ ID NO: 1, 62-85, or fragments or variants thereof. In other embodiments the VSTM5 extracellular domain polypeptide consists essentially of the amino acid sequence of the IgV domain as set forth in any one of SEQ ID NOs: 1, 62-85.

In some embodiments the VSTM5 extracellular domain polypeptide comprises the amino acid sequence of the IgC2 domain as set forth in any one of SEQ ID NO: 86-110, or fragments or variants thereof. In other embodiments the VSTM5 extracellular domain polypeptide consists essentially of the amino acid sequence of the IgV domain as set forth in any one of SEQ ID NOs: 86-110.

Generally, the VSTM5 polypeptide fragments are expressed from nucleic acids that include sequences that encode a signal sequence. The signal sequence is generally cleaved from the immature polypeptide to produce the mature polypeptide lacking the signal sequence. The signal sequence of VSTM5 can be replaced by the signal sequence of another polypeptide using standard molecule biology techniques to affect the expression levels, secretion, solubility, or other property of the polypeptide. The signal peptide sequence that is used to replace the VSTM5 signal peptide sequence can be any known in the art.

Preferably such “soluble ectodomain (ECD)” or “ectodomain” or “soluble” form of VSTM5 will modulate (agonize or antagonize) one or more of VSTM5's effects on immunity and specific types of immune cells such as cytotoxic or effector T cells, Tregs and NK cells.

Optionally, the VSTM5 ECD fragments refer also to any one of the polypeptide sequences listed in any of SEQ ID NOs 12-21, which are reasonably expected to comprise functional regions of the VSTM5 protein. This expectation is based on a systematic analysis of a set of protein database sequences (PDBs) which contained complexes of Ig proteins (for example PDB li85 which describe the complex of CTLA4 and CD86). The intermolecular contact residues from each PDB were collected and projected on the sequence of VSTM5. Several regions with clusters of interacting residues supported by several contact maps were identified and synthesized as a series of peptides and are reasonably expected to mimic the structure of the intact full length protein and thereby modulate one or more of the effects of VSTM5 on immunity and on specific immune cell types.

### Variants of VSTM5 Polypeptides

In at least some embodiments, the present invention encompasses useful variants of VSTM5 polypeptides including those that increase biological activity, as indicated by any of the assays described herein, or that increase half-life or stability of the protein. Soluble VSTM5 proteins or fragments, or fusions thereof having VSTM5 proteins activity, respectively, can be engineered to increase biological activity. In a further embodiment, the VSTM5 proteins or fusion protein is modified with at least one amino acid substitution, deletion, or insertion that increases the binding of the molecule to an immune cell, for example a T cell, and transmits an inhibitory signal into the T cell. An isolated or recombinant VSTM5 polypeptide or fusion protein according to any of the foregoing claims which comprises at least one half-life extending moiety.

For example such half-life extending moieties may include by way of example polyethylene glycol (PEG), monomethoxy PEG (mPEG), an XTEN molecule, an rPEG molecule, an adnectin, a serum albumin, human serum albumin, immunoglobulin constant region or fragment thereof, or acyl group. In some embodiments the half-life modified isolated or recombinant VSTM5 polypeptide or fusion protein according to the invention which comprises a heterologous polypeptide, or half-life extending moiety, or other heterologous molecule may increase the in vivo half-life of the VSTM5 polypeptide or fusion protein by at least about 2-fold, at least about 3-fold, at least about 4-fold, at least about 5-fold, at least about 10-fold, or more compared to an otherwise identical molecule that lacks said heterologous polypeptide, half-life extending moiety, or other heterologous molecule.

Other optional variants are those VSTM5 proteins that are engineered to selectively bind to one type of T cell versus other immune cells or to NK cells. For example, the VSTM5 polypeptide can be engineered to bind optionally to Tregs, Th0, Th1, Th17, Th2 or Th22 cells or to NK cells. Preferential binding refers to binding that is at least 10%, 20%, 30%, 40%, 50%, 60% f 70%, 80%, 90%, 95%, or greater for one type of cell over another type of cell. Still other variants of VSTM5 protein can be engineered to have reduced binding to immune cells relative to wild-type VSTM5 protein, respectively. These variants can be used in combination with variants having stronger binding properties to modulate the immune response with a moderate impact.

Also optionally, variant VSTM5 protein can be engineered to have an increased half-life relative to wild-type. These variants typically are modified to resist enzymatic degradation. Exemplary modifications include modified amino acid residues and modified peptide bonds that resist enzymatic degradation. Various modifications to achieve this are known in the art.

Preferably such variant form of VSTM5 will modulate (agonize or antagonize) one or more of VSTM5's effects on immunity and on specific types of immune cells such as cytotoxic or effector T cells, Tregs, MDSCs, and other suppressor cell types; and NK cells.

Nucleic Acids

A “nucleic acid fragment” or an “oligonucleotide” or a “polynucleotide” are used herein interchangeably to refer to a polymer of nucleic acid residues. A polynucleotide sequence of the present invention refers to a single or double stranded nucleic acid sequences which is isolated and provided in the form of an RNA sequence, a complementary polynucleotide sequence (cDNA), a genomic polynucleotide sequence and/or a composite polynucleotide sequences (e.g., a combination of the above).

Thus, the present invention encompasses nucleic acid sequences described hereinabove; fragments thereof, sequences hybridizable therewith, sequences homologous thereto [e.g., at least 90%, at least 95, 96, 97, 98 or 99% or more identical to the nucleic acid sequences set forth herein], sequences encoding similar polypeptides with different codon usage, altered sequences characterized by mutations, such as deletion, insertion or substitution of one or more nucleotides, either naturally occurring or man induced, either randomly or in a targeted fashion. The present invention also encompasses homologous nucleic acid sequences (i.e., which form a part of a polynucleotide sequence of the present invention), which include sequence regions unique to the polynucleotides of the present invention.

Thus, the present invention also encompasses polypeptides encoded by the polynucleotide sequences of the present invention. The present invention also encompasses homologues of these polypeptides, such homologues can be at least 90%, at least 95, 96, 97, 98 or 99% or more homologous to the amino acid sequences set forth below, as can be determined using BlastP software of the National Center of Biotechnology Information (NCBI) using default parameters. As mentioned hereinabove, biomolecular sequences of the present invention can be efficiently utilized as tissue or pathological markers and as putative drugs or drug targets for treating or preventing a disease. Oligonucleotides designed for carrying out the methods of the present invention for any of the sequences provided herein (designed as described above) can be generated according to any oligonucleotide synthesis method known in the art such as enzymatic synthesis or solid phase synthesis. Oligonucleotides used according to this aspect of the present invention are those having a length selected from a range of about 10 to about 200 bases preferably about 15 to about 150 bases, more preferably about 20 to about 100 bases, most preferably about 20 to about 50 bases.

Preferably such nucleic acids, comprising a region encoding for a “soluble ectodomain (ECD)” or “ectodomain” or “soluble” form of VSTM5 will modulate (agonize or antagonize) one or more of VSTM5's effects on immunity and specific types of immune cells such as cytotoxic or effector T cells, Tregs and NK cells.

Peptides

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

Polypeptide products can be biochemically synthesized such as by employing standard solid phase techniques. Such methods include exclusive solid phase synthesis, partial solid phase synthesis methods, fragment condensation, classical solution synthesis. These methods are preferably used when the peptide is relatively short (i.e., 10 kDa) and/or when it cannot be produced by recombinant techniques (i.e., not encoded by a nucleic acid sequence) and therefore involves different chemistry.

Solid phase polypeptide synthesis procedures are well known in the art and further described by John Morrow Stewart and Janis Dillaha Young, Solid Phase Peptide Syntheses (2nd Ed., Pierce Chemical Company, 1984).

Synthetic polypeptides can be purified by preparative high performance liquid chromatography (Creighton T. Proteins, Structures and Molecular Principles. WH Freeman and Co. N.Y. (1983)) and the composition of which can be confirmed via amino acid sequencing.

In cases where large amounts of a polypeptide are desired, it can be generated using recombinant techniques such as described by Bitter et al., Methods in Enzymol. 153:516-544(1987), Studier et al. Methods in Enzymol. 185:60-89 (1990), Brisson et al. Nature 310:511-514 (1984), Takamatsu et al. EMBO J. 6:307-311 (1987), Coruzzi et al. EMBO J. 3:1671-1680 (1984) and Brogli et al., Science 224:838-843 (1984), and Gurley et al. Mol. Cell. Biol. 6:559-565 (1986).

It will be appreciated that peptides identified according to the teachings of the present invention may be degradation products, synthetic peptides or recombinant peptides as well as peptidomimetics, typically, synthetic peptides and peptoids and semipeptoids which are peptide analogs, which may have, for example, modifications rendering the peptides more stable while in a body or more capable of penetrating into cells. Such modifications include, but are not limited to N terminus modification, C terminus modification, peptide bond modification, including, but not limited to, CH₂—NH, CH₂—S, CH₂—S═O, O═C—NH, CH₂—O, CH₂—CH₂, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH₃)—CO—), ester bonds (—C(R)H—C—O—O—C(R)—N—), ketomethylen bonds (—CO—CH₂—), α-aza bonds (—NH—N(R)—CO—), wherein R is any alkyl, e.g., methyl, carba bonds (—CH2-NH—), hydroxyethylene bonds (—CH(OH)—CH₂—), thioamide bonds (—CS—NH—), olefinic double bonds (—CH═CH—), retro amide bonds (—NH—CO—), peptide derivatives (—N(R)—CH₂—CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted by synthetic non-natural acids such as Phenylglycine, TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

Since the peptides of the present invention are preferably utilized in therapeutics which requires the peptides to be in soluble form, the peptides of the present invention preferably include one or more non-natural or natural polar amino acids, including but not limited to serine and threonine which are capable of increasing peptide solubility due to their hydroxyl-containing side chain.

Expression Systems

To enable cellular expression of the polynucleotides of the present invention, a nucleic acid construct according to the present invention may be used, which includes at least a coding region of one of the above nucleic acid sequences, and further includes at least one cis acting regulatory element. As used herein, the phrase “cis acting regulatory element” refers to a polynucleotide sequence, preferably a promoter, which binds a trans acting regulator and regulates the transcription of a coding sequence located downstream thereto.

Any suitable promoter sequence can be used by the nucleic acid construct of the present invention.

Preferably, the promoter utilized by the nucleic acid construct of the present invention is active in the specific cell population transformed. Examples of cell type-specific and/or tissue-specific promoters include promoters such as albumin that is liver specific (Pinkert et al., Genes Dev. 1:268-277 (1987)), lymphoid specific promoters (Calame et al., Adv. Immunol. 43:235-275 (1988)), in particular promoters of T-cell receptors ([Winoto et al., EMBO J. 8:729-733 (1989)) and immunoglobulins; [Banerji et al. Cell 33729-740 (1983)], neuron-specific promoters such as the neurofilament promoter (Byrne et al. Proc. Natl. Acad. Sci. USA 86:5473-5477 (1989)), pancreas-specific promoters (Edlunch et al. Science 230:912-916 (1985)) or mammary gland-specific promoters such as the milk whey promoter (U.S. Pat. No. 4,873,316 and European Application Publication No. 264,166). The nucleic acid construct of the present invention can further include an enhancer, which can be adjacent or distant to the promoter sequence and can function in up regulating the transcription therefrom.

The nucleic acid construct of the present invention preferably further includes an appropriate selectable marker and/or an origin of replication. Preferably, the nucleic acid construct utilized is a shuttle vector, which can propagate both in E. coli (wherein the construct comprises an appropriate selectable marker and origin of replication) and be compatible for propagation in cells, or integration in a gene and a tissue of choice. The construct according to the present invention can be, for example, a plasmid, a bacmid, a phagemid, a cosmid, a phage, a virus or an artificial chromosome.

Examples of suitable constructs include, but are not limited to, pcDNA3, pcDNA3.1 (+/−), pGL3, PzeoSV2 (+/−), pDisplay, pEF/myc/cyto, pCMV/myc/cyto each of which is commercially available from Invitrogen Co. (www.invitrogen.com). Examples of retroviral vector and packaging systems are those sold by Clontech, San Diego, Calif., including Retro-X vectors pLNCX and pLXSN, which permit cloning into multiple cloning sites and the transgene is transcribed from CMV promoter. Vectors derived from Mo-MuLV are also included such as pBabe, where the transgene will be transcribed from the 5′LTR promoter.

Currently preferred in vivo nucleic acid transfer techniques include transfection with viral or non-viral constructs, such as adenovirus, lentivirus, herpes simplex I virus, or adeno-associated virus (AAV) and lipid-based systems. Useful lipids for lipid-mediated transfer of the gene are, for example, DOTMA, DOPE, and DC-Chol (Tonkinson et al., Cancer Invest. 14(1): 54-65 (1996))) The most common constructs used in gene therapy are viruses, most preferably adenoviruses, AAV, lentiviruses, or retroviruses. A viral construct such as a retroviral construct includes at least one transcriptional promoter/enhancer or locus-defining elements, or other elements that control gene expression by other means such as alternate splicing, nuclear RNA export, or post-translational modification of messenger. Such vector constructs also include a packaging signal, long terminal repeats (LTRs) or portions thereof, and positive and negative strand primer binding sites appropriate to the virus used, unless it is already present in the viral construct. In addition, such a construct typically includes a signal sequence for secretion of the peptide from a host cell in which it is placed. Preferably the signal sequence for this purpose is a mammalian signal sequence or the signal sequence of the polypeptides of the present invention. Optionally, the construct may also include a signal that directs polyadenylation, as well as one or more restriction sites and a translation termination sequence. By way of example, such constructs will typically include a 5′ LTR, a tRNA binding site, a packaging signal, an origin of second-strand DNA synthesis, and a 3′ LTR or a portion thereof. Other vectors can be used that are non-viral, such as cationic lipids, polylysine, and dendrimers.

Recombinant Expression Vectors and Host Cells

Another aspect of the invention pertains to vectors, preferably expression vectors, containing a nucleic acid encoding a protein according to at least some embodiments of the invention, or derivatives, fragments, analogs or homologs thereof. As used herein, the term “vector” refers to a nucleic acid molecule capable of transporting another nucleic acid to which it has been linked. Examples of vector types are plasmids and viral vectors. Certain vectors are capable of autonomous replication in a host cell into which they are introduced (e.g., bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. The invention is intended to include such forms of expression vectors, such as plasmids, viral vectors (e.g., replication defective retroviruses, adenoviruses and adeno-associated viruses), which serve equivalent functions.

The recombinant expression vectors according to at least some embodiments of the invention comprise a nucleic acid according to at least some embodiments of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequences in a manner that allows for expression of the nucleotide sequence (e.g., in an in vitro transcription/translation system or in a host cell when the vector is introduced into the host cell).

The term “regulatory sequence” is intended to include promoters, enhancers and other expression control elements (e.g., polyadenylation signals). Such regulatory sequences are described, for example, in Goeddel, Gene Expression Technology: Methods in Enzymology 185, Academic Press, San Diego, Calif. (1990). Regulatory sequences include those that direct constitutive expression of a nucleotide sequence in many types of host cell and those that direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, etc. The expression vectors according to at least some embodiments of the invention can be introduced into host cells to thereby produce proteins or peptides, including fusion proteins or peptides, encoded by nucleic acids as described herein.

The recombinant expression vectors according to at least some embodiments of the invention can be designed for production of variant proteins in prokaryotic or eukaryotic cells. For example, proteins according to at least some embodiments of the invention can be expressed in bacterial cells such as Escherichia coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, to the amino or C terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin, PreScission, TEV and enterokinase. Typical fusion expression vectors include pGEX (Pharmacia Biotech Inc; Smith and Johnson, Gene 67: 31-40 (1988)), pMAL (New England Biolabs, Beverly, Mass.) and pRITS (Pharmacia, Piscataway, N.J.) that fuse glutathione S-transferase (GST), maltose E binding protein, or protein A, respectively, to the target recombinant protein.

In another embodiment, the expression vector encoding for the protein of the invention is a yeast expression vector. Examples of vectors for expression in yeast Saccharomyces cerevisiae include pYepSec1 (Baldari, et al., EMBO J. 6: 229-234(1987)), pMFa (Kurjan and Herskowitz, Cell 30: 933-943(1982)), pJRY88 (Schultz et al., Gene 54: 113-123 (1987)), pYES2 (Invitrogen Corporation, San Diego, Calif.), and picZ (InVitrogen Corp, San Diego, Calif.).

Alternatively, polypeptides of the present invention can be produced in insect cells using baculovirus expression vectors. Baculovirus vectors available for expression of proteins in cultured insect cells (e.g., SF9 cells) include the pAc series (Smith, et al., Mol. Cell. Biol. 3: 2156-2165 (1983)) and the pVL series (Lucklow and Summers, Virology 170: 31-39(1989)).

In yet another embodiment, a nucleic acid of the invention is expressed in mammalian cells using a mammalian expression vector. Examples of mammalian expression vectors include pCDM8 (Seed, Nature 329: 840(1987)) and pMT2PC (Kaufman, et al., EMBO J. 6: 187-195(1987)), pIRESpuro (Clontech), pUB6 (Invitrogen), pCEP4 (Invitrogen) pREP4 (Invitrogen), pcDNA3 (Invitrogen). When used in mammalian cells, the expression vector's control functions are often provided by viral regulatory elements. For example, commonly used promoters are derived from polyoma, adenovirus 2, cytomegalovirus, Rous Sarcoma Virus, and simian virus 40. For other suitable expression systems for both prokaryotic and eukaryotic cells see, e.g., Chapters 16 and 17 of Sambrook, et al., Molecular Cloning: A Laboratory Manual, 2^{nd} ed., Cold Spring Harbor Laboratory, Cold Spring Harbor Laboratory Press, Cold Spring Harbor, N.Y., (1989).

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., Genes Dev. 1: 268-277(1987)), lymphoid-specific promoters (Calame and Eaton, Adv. Immunol. 43: 235-275(1988)), in particular promoters of T cell receptors (Winoto and Baltimore, EMBO J. 8: 729-733 (1989)) and immunoglobulins (Banerji, et al., Cell 33: 729-740 (1983); Queen and Baltimore, Cell 33: 741-748 (1983)), neuron-specific promoters (e.g., the neurofilament promoter; Byrne and Ruddle, Proc. Natl. Acad. Sci. USA 86: 5473-5477 (1989)), pancreas-specific promoters (Edlund, et al., Science 230: 912-916 (1985)), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, Science 249: 374-379(1990)) and the α-fetoprotein promoter (Campes and Tilghman, Genes Dev. 3: 537-546 (1989)).

According to at least some embodiments the invention further provides a recombinant expression vector comprising a DNA molecule according to at least some embodiments of the invention cloned into the expression vector in an antisense orientation. That is, the DNA molecule is operatively-linked to a regulatory sequence in a manner that allows for expression (by transcription of the DNA molecule) of an RNA molecule that is antisense to mRNA encoding for protein according to at least some embodiments of the invention. Regulatory sequences operatively linked to a nucleic acid cloned in the antisense orientation can be chosen that direct the continuous expression of the antisense RNA molecule in a variety of cell types, for instance viral promoters and/or enhancers, or regulatory sequences can be chosen that direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e.g., Weintraub, et al., “Antisense RNA as a molecular tool for genetic analysis,” Reviews-Trends in Genetics, Vol. 1(1) (1986).

According to at least some embodiments the present invention pertains to host cells into which a recombinant expression vector according to at least some embodiments of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, protein according to at least some embodiments of the invention can be produced in bacterial cells such as E. coli, insect cells, yeast, plant or mammalian cells (such as Chinese hamster ovary cells (CHO) or COS or 293 cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e.g., DNA) into a host cell, including calcium phosphate or calcium chloride co-precipitation, DEAE-dextran-mediated transfection, lipofection, or electroporation. Suitable methods for transforming or transfecting host cells can be found in Sambrook, et al. (Molecular Cloning: A Laboratory Manual, 2^{nd} ed., Cold Spring Harbor Laboratory, Cold Spring Harbor Laboratory Press, Cold Spring Harbor, N.Y., (1989)), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e.g., resistance to antibiotics) is generally introduced into the host cells along with the gene of interest. Various selectable markers include those that confer resistance to drugs, such as G418, hygromycin, puromycin, blasticidin and methotrexate. Nucleic acids encoding a selectable marker can be introduced into a host cell on the same vector as that encoding protein according to at least some embodiments of the invention or can be introduced on a separate vector. Cells stably transfected with the introduced nucleic acid can be identified by drug selection (e.g., cells that have incorporated the selectable marker gene will survive, while the other cells die).

A host cell according to at least some embodiments of the invention, such as a prokaryotic or eukaryotic host cell in culture, can be used to produce (i.e., express) protein according to at least some embodiments of the invention. Accordingly, the invention further provides methods for producing proteins according to at least some embodiments of the invention using the host cells according to at least some embodiments of the invention. In one embodiment, the method comprises culturing the host cell of the present invention (into which a recombinant expression vector encoding protein according to at least some embodiments of the invention has been introduced) in a suitable medium such that the protein according to at least some embodiments of the invention is produced. In another embodiment, the method further comprises isolating protein according to at least some embodiments of the invention from the medium or the host cell. For efficient production of the protein, it is preferable to place the nucleotide sequences encoding the protein according to at least some embodiments of the invention under the control of expression control sequences optimized for expression in a desired host. For example, the sequences may include optimized transcriptional and/or translational regulatory sequences (such as altered Kozak sequences).

It should be noted, that according to at least some embodiments of the present invention the VSTM5 proteins according to at least some embodiments of the invention may be isolated as naturally-occurring polypeptides, or from any source whether natural, synthetic, semi-synthetic or recombinant. Accordingly, the VSTM5 proteins may be isolated as naturally-occurring proteins from any species, particularly mammalian, including bovine, ovine, porcine, murine, equine, and preferably human. Alternatively, the VSTM5 proteins may be isolated as recombinant polypeptides that are expressed in prokaryote or eukaryote host cells, or isolated as a chemically synthesized polypeptide.

A skilled artisan can readily employ standard isolation methods to obtain isolated VSTM5 proteins. The nature and degree of isolation will depend on the source and the intended use of the isolated molecules.

Transgenic Animals and Plants

According to at least some embodiments the invention also provides transgenic non-human animals and transgenic plants comprising one or more nucleic acid molecules according to at least some embodiments of the invention that may be used to produce the polypeptides according to at least some embodiments of the invention. The polypeptides can be produced in and recovered from tissue or bodily fluids, such as milk, blood or urine, of goats, cows, horses, pigs, rats, mice, rabbits, hamsters or other mammals. See, e.g., U.S. Pat. Nos. 5,827,690, 5,756,687, 5,750,172, and 5,741,957.

Non-human transgenic animals and transgenic plants are produced by introducing one or more nucleic acid molecules according to at least some embodiments of the invention into the animal or plant by standard transgenic techniques. The transgenic cells used for making the transgenic animal can be embryonic stem cells, somatic cells or fertilized egg cells. The transgenic non-human organisms can be chimeric, nonchimeric heterozygotes, and nonchimeric homozygotes. (See, e.g., Hogan et al. Manipulating the Mouse Embryo: A Laboratory Manual 2^{nd} ed. Cold Spring Harbor Press (1999); Jackson et al., Mouse Genetics and Transgenics: A Practical Approach, Oxford University Press (2000); and Pinkert, Transgenic Animal Technology: A Laboratory Handbook, Academic Press (1999)).

Protein Modifications

Fusion Proteins

According to at least some embodiments, VSTM5 fusion polypeptides have a first fusion partner comprising all or a part of a VSTM5 protein fused to a second polypeptide directly or via a linker peptide sequence or a chemical linker useful to connect the two proteins. The VSTM5 polypeptide may optionally be fused to a second polypeptide to form a fusion protein as described herein. The presence of the second polypeptide can alter the solubility, stability, affinity and/or valency of the VSTM5 fusion polypeptide. As used herein, “valency” refers to the number of binding sites available per molecule. In one embodiment the second polypeptide is a polypeptide from a different source or different protein.

According to at least some embodiments, the VSTM5 protein or fragment is selected for its activity for the treatment of immune related disorder, infectious disorder, sepsis, cancer, and/or for blocking the undesirable immune activation that follows gene transfer, as described herein.

In one embodiment, the second polypeptide contains one or more domains of an immunoglobulin heavy chain constant region, preferably having an amino acid sequence corresponding to the hinge, C_{H2} and C_{H3} regions of a human immunoglobulin Cγ1, Cγ2, Cγ3 or Cγ4 chain or to the hinge, C_{H2} and C_{H3} regions of a murine immunoglobulin Cγ2a chain. SEQ ID NO: 113 provides an exemplary sequence for the hinge, C_{H2} and C_{H3} regions of a human immunoglobulin Cy1. An additional exemplary VSTM5-IgG1 fusion protein according to the invention is contained in SEQ ID NO:115 (described infra).

According to at least some embodiments, the fusion protein is a dimeric fusion protein which optionally is capable of cross-linking two or more targets. In an optional dimeric fusion protein, the dimer results from the covalent bonding of Cys residue in the hinge region of two of the Ig heavy chains that are the same Cys residues that are disulfide linked in dimerized normal Ig heavy chains. Such proteins are referred to as VSTM5 polypeptides, fragments or fusion proteins thereof.

In one embodiment, the immunoglobulin constant domain may contain one or more amino acid insertions, deletions or substitutions that enhance or decrease binding to specific cell types, increase the bioavailablity, or increase the stability of the VSTM5 polypeptides, fusion proteins, or fragments thereof. Suitable amino acid substitutions include conservative and non-conservative substitutions, as described above.

The fusion proteins optionally contain a domain that functions to dimerize or multimerize two or more fusion proteins. The peptide/polypeptide linker domain can either be a separate domain, or alternatively can be contained within one of the other domains (VSTM5 polypeptide or second polypeptide) of the fusion protein. Similarly, the domain that functions to dimerize or multimerize the fusion proteins can either be a separate domain, or alternatively can be contained within one of the other domains (VSTM5 polypeptide, second polypeptide or peptide/polypeptide linker domain) of the fusion protein. In one embodiment, the dimerization/multimerization domain and the peptide/polypeptide linker domain are the same. Further specific, illustrative and non-limiting examples of dimerization/multimerization domains and linkers are given below.

Fusion proteins disclosed herein according to at least some embodiments of the present invention are of formula I: N—R1-R2-R3-C wherein “N” represents the N-terminus of the fusion protein, “C” represents the C-terminus of the fusion protein. In the further embodiment, “R1” is a VSTM5 polypeptide, “R2” is an optional peptide/polypeptide or chemical linker domain, and “R3” is a second polypeptide. Alternatively, R3 may be a VSTM5 polypeptide and R1 may be a second polypeptide. Various non-limiting examples of linkers are described in greater detail below.

Optionally, the fusion protein comprises the VSTM5 polypeptide fragments as described herein, fused, optionally by a linker peptide of one or more amino acids (e.g. GS) to one or more “half-life extending moieties”. A “half-life extending moiety” is any moiety, for example, a polypeptide, small molecule or polymer, that, when appended to protein, extends the in vivo half-life of that protein in the body of a subject (e.g., in the plasma of the subject). For example, a half-life extending moiety is, in an embodiment of the invention, polyethylene glycol (PEG), monomethoxy PEG (mPEG), XTEN molecule, an rPEG molecule, an adnectin, a serum albumin, human serum albumin, immunoglobulin constant region or fragment thereof, or acyl group. In an embodiment of the invention, PEG is a 5, 10, 12, 20, 30, 40 or 50 kDa moiety or larger or comprises about 12000 ethylene glycol units (PEG12000). An isolated or recombinant VSTM5 polypeptide or fusion protein according to the invention may optionally comprise at least one half-life extending moiety. Half-life extending moieties include PEG's, an XTEN molecule, an rPEG molecule, an adnectin, a serum albumin, human serum albumin, immunoglobulin constant region or fragment thereof, or acyl group. In some embodiments the heterologous polypeptide, half-life extending moiety, or other heterologous molecule contained in a VSTM5 polypeptide or fusion protein according to the invention may increase the in vivo half-life of said isolated or recombinant VSTM5 polypeptide or fusion protein by at least about 2-fold, at least about 3-fold, at least about 4-fold, at least about 5-fold, at least about 10-fold, or more compared to an otherwise identical molecule that lacks said heterologous polypeptide, half-life extending moiety, or other heterologous molecule.

The fusion protein may also optionally be prepared by chemical synthetic methods and the “join” effected chemically, either during synthesis or post-synthesis. Cross-linking and other such methods may optionally be used (optionally also with the above described genetic level fusion methods), as described for example in U.S. Pat. No. 5,547,853 to Wallner et al, which is hereby incorporated by reference as if fully set forth herein as a non-limiting example only.

According to the present invention, a fusion protein may be prepared from a protein of the invention by fusion with a portion of an immunoglobulin comprising a constant region of an immunoglobulin. More preferably, the portion of the immunoglobulin comprises a heavy chain constant region which is optionally and more preferably a human heavy chain constant region. The heavy chain constant region is most preferably an IgG heavy chain constant region, and optionally and most preferably is an Fc chain, most preferably an IgG F_{c} fragment that comprises the hinge, C_{H2} and C_{H3} domains. The Fc chain may optionally be a known or “wild type” Fc chain, or alternatively may be mutated or truncated. The F_{c} portion of the fusion protein may optionally be varied by isotype or subclass, may be a chimeric or hybrid, and/or may be modified, for example to improve effector functions, control of half-life, tissue accessibility, augment biophysical characteristics such as stability, and improve efficiency of production (and less costly). Many modifications useful in construction of disclosed fusion proteins and methods for making them are known in the art, see for example Mueller, et al, Mol. Immun., 34(6):441-452 (1997), Swann, et al., Cur. Opin. Immun., 20:493-499 (2008), and Presta, Cur. Opin. Immun. 20:460-470 (2008). In some embodiments the Fc region is the native IgG1, IgG2, IgG3 or IgG4 Fc region. In some embodiments the F_{c} region is a hybrid, for example a chimeric consisting of IgG2/IgG4 Fc constant regions.

Modifications to the Fc region include, but are not limited to, IgG4 modified to prevent binding to Fcγ receptors and complement, IgG1 modified to improve binding to one or more Fc γ receptors, IgG1 modified to minimize effector function (amino acid changes), IgG1 with altered/no glycan (typically by changing expression host or substituting the Asn at position 297), and IgG1 with altered pH-dependent binding to FcRn. The Fc region may include the entire hinge region, or less than the entire hinge region.

In another embodiment, the Fc domain may contain one or more amino acid insertions, deletions or substitutions that reduce binding to the low affinity inhibitory Fc receptor CD32B (FcγRIIB) and retain wild-type levels of binding to or enhance binding to the low affinity activating Fc receptor CD16A (FcγRIIIA) Another embodiment includes IgG2-4 hybrids and IgG4 mutants that have reduced binding to FcR (F_{c} receptor) which increase their half-life. Representative IgG2-4 hybrids and IgG4 mutants are described in Angal, S. et al., Molecular Immunology, 30(1):105-108 (1993); Mueller, J. et al., Molecular Immunology, 34(6): 441-452 (1997); and U.S. Pat. No. 6,982,323 to Wang et al. In some embodiments the IgG1 and/or IgG2 domain is deleted; for example, Angal et al. Molecular Immunology, 30(1):105-108 (1993) describe IgG1 and IgG2 having serine 241 replaced with a proline.

In a further embodiment, the Fc domain contains amino acid insertions, deletions or substitutions that enhance binding to CD16A. A large number of substitutions in the Fc domain of human IgG1 that increase binding to CD16A and reduce binding to CD32B are known in the art and are described in Stavenhagen, et al., Cancer Res., 57(18):8882-90 (2007). Exemplary variants of human IgG1 Fc domains with reduced binding to CD32B and/or increased binding to CD16A contain F243L, R929P, Y300L, V3051 or P296L substitutions. These amino acid substitutions may be present in a human IgG1 Fc domain in any combination.

In one embodiment, the human IgG1 Fc domain variant contains a F243L, R929P and Y300L substitution. In another embodiment, the human IgG1 Fc domain variant contains a F243L, R929P, Y300L, V3051 and P296L substitution. In another embodiment, the human IgG1 Fc domain variant contains an N297A/Q substitution, as these mutations abolish FcγR binding. Non-limiting, illustrative, exemplary types of mutations are described in US Patent Application No. 20060034852, published on Feb. 16, 2006, hereby incorporated by reference as if fully set forth herein. The term “Fc chain” also optionally comprises any type of Fc fragment.

Several of the specific amino acid residues that are important for antibody constant region-mediated activity in the IgG subclass have been identified. Inclusion, substitution or exclusion of these specific amino acids therefore allows for inclusion or exclusion of specific immunoglobulin constant region-mediated activity. Furthermore, specific changes may result in aglycosylation for example and/or other desired changes to the Fc chain. At least some changes may optionally be made to block a function of Fc which is considered to be undesirable, such as an undesirable immune system effect, as described in greater detail below.

Non-limiting, illustrative examples of mutations to Fc which may be made to modulate the activity of the fusion protein include the following changes (given with regard to the Fc sequence nomenclature as given by Kabat, from Kabat E A et al: Sequences of Proteins of Immunological Interest, US Department of Health and Human Services, NIH, (1991)): 220C->S; 233-238 ELLGGP->EAEGAP; 265D->A, preferably in combination with 434N->A; 297N->A (for example to block N-glycosylation); 318-322 EYKCK->AYACA; 330-331AP->SS; or a combination thereof (see for example M. Clark, Chemical Immunol and Antibody Engineering, pp 1-31 for a description of these mutations and their effect). The construct for the Fc chain which features the above changes optionally and preferably comprises a combination of the hinge region with the C_{H2} and C_{H3} domains.

The above mutations may optionally be implemented to enhance desired properties or alternatively to block non-desired properties. For example, aglycosylation of antibodies was shown to maintain the desired binding functionality while blocking depletion of T-cells or triggering cytokine release, which may optionally be undesired functions (see M. Clark, Chemical Immunol and Antibody Engineering, pp 1-31). Substitution of 331proline for serine may block the ability to activate complement, which may optionally be considered an undesired function (see M. Clark, Chemical Immunol and Antibody Engineering, pp 1-31). Changing the alanine to serine at position 330 in combination with this change may also enhance the desired effect of blocking the ability to activate complement. Residues 235 and 237 were shown to be involved in antibody-dependent cell-mediated cytotoxicity (ADCC), such that changing the block of residues from 233-238 as described may also block such activity if ADCC is considered to be an undesirable function.

Residue 220 is normally a cysteine for F_{c} from IgG1, which is the site at which the heavy chain forms a covalent linkage with the light chain. Optionally, this residue may be changed to another amino acid residue (e.g., serine), to avoid any type of covalent linkage (see M. Clark, Chemical Immunol and Antibody Engineering, pp 1-31) or by deletion or truncation.

The above changes to residues 265 and 434 may optionally be implemented to reduce or block binding to the Fc receptor, which may optionally block undesired functionality of Fc related to its immune system functions (see “Binding site on Human IgG1 for Fc Receptors”, Shields et al, Vol 276, pp 6591-6604, 2001).

The above changes are intended as illustrations only of optional changes and are not meant to be limiting in any way. Furthermore, the above explanation is provided for descriptive purposes only, without wishing to be bound by a single hypothesis.

In a further embodiment, the fusion protein includes the extracellular domain of VSTM5, or a fragment thereof fused to an Ig F_{c} region. Recombinant IgVSTM5 polypeptides, fragments or fusion proteins thereof fusion proteins can be prepared by fusing the coding region of the extracellular domain of VSTM5 or a fragment thereof to the Fc region of human IgG1 or mouse IgG2a, as described previously (Chapoval, et al., Methods Mol. Med, 45:247-255 (2000)).

Optionally, VSTM5 ECD refers also to fusion protein, comprising an amino acid sequence of human VSTM5 ECD fused to human immunoglobulin F_{c}. Optionally, said fusion protein comprises the amino acid sequence of the human VSTM5 ECD set forth in SEQ ID NOs: 2, 3 or fragment thereof set forth in any of SEQ ID NOs: 1, 12-110, 151-156, fused to human IgG1 F_{c} set forth in any one of SEQ ID NOs:113, 114, 115, 150. Optionally, the amino acid sequence of said fusion protein is set forth in SEQ ID NO:130, 134, 135.

The aforementioned exemplary fusion proteins can incorporate any combination of the variants described herein. In another embodiment the terminal lysine of the aforementioned exemplary fusion proteins is deleted.

The disclosed fusion proteins can be isolated using standard molecular biology techniques. For example, an expression vector containing a DNA sequence encoding a VSTM5 polypeptides, fragments or fusion proteins thereof fusion protein is transfected into 293 cells by calcium phosphate precipitation and cultured in serum-free DMEM. The supernatant is collected at 72 h and the fusion protein is purified by Protein G, or preferably Protein A SEPHAROSE® columns (Pharmacia, Uppsala, Sweden). Optionally, a DNA sequence encoding a VSTM5 polypeptides, fragments or fusion proteins thereof fusion protein is transfected into GPEx® retrovectors and expressed in CHO—S cells following four rounds of retrovector transduction. The protein is clarified from supernatants using protein A chromatography.

In another embodiment the second polypeptide may have a conjugation domain through which additional molecules can be bound to the VSTM5 fusion proteins. In one such embodiment, the conjugated molecule is capable of targeting the fusion protein to a particular organ or tissue; further specific, illustrative, non-limiting examples of such targeting domains and/or molecules are given below.

In another such embodiment the conjugated molecule is another immunomodulatory agent that can enhance or augment the effects of the VSTM5 fusion protein. In another embodiment the conjugated molecule is Polyethylene Glycol (PEG).

In some embodiments VSTM5 polypeptides or fusion proteins according to the invention will comprise a binding domain, wherein the binding protein is capable of cross-linking two or more targets.

In some embodiments VSTM5 polypeptides or fusion proteins according to the invention will comprise another binding moiety, wherein the binding moiety targets a tumor cell, infectious agent, e.g., a virus, bacterium, mycoplasm, fungus, yeast or parasite, or cell infected thereby, an immune cell, or a disease site.

In some embodiments VSTM5 polypeptides or fusion proteins according to the invention will comprise at least one heterologous polypeptide which may be a receptor, hormone, cytokine, antigen, B-cell target, NK cell target, T cell target, TNF receptor superfamily member, Hedgehog family member, a receptor tyrosine kinase, a proteoglycan-related molecule, a TGF-β superfamily member, a Wnt-related molecule, a receptor ligand, a Dendritic cell target, a myeloid cell target, a monocyte/macrophage cell target or an angiogenesis target.

In some embodiments VSTM5 polypeptides or fusion proteins according to the invention will comprise at least one antigen, e.g., a tumor antigen, autoantigen, allergen, or an infectious agent antigen.

In some embodiments VSTM5 polypeptides or fusion proteins according to the invention will comprise a T cell target selected from the group consisting of 2B4/SLAMF4, IL-2 Rα, 4-1BB/TNFRSF9, IL-2Rβ, ALCAM, B7-1/CD80, IL-4R, B7-H3, BLAME/SLAMF8, BTLA, IL-6R, CCR3, IL-7 Rα, CCR4, CXCR1/IL-8 RA, CCR5, CCR6, IL-10 R α, CCR7, IL-10 RP, CCR8, IL-12 Rβ1, CCR9, IL-12 RP 2, CD2, IL-13Rα1, IL-13, CD3, CD4, ILT2/CD85j, ILT3/CD85k, ILT4/CD85d, ILT5/CD85a, Integrin α4/CD49d, CD5, IntegrinαE/CD103, CD6, Integrin α M/CD11b, CD8, Integrin α X/CD11c, Integrin β2/CD18, KIR/CD158, CD27/TNFRSF7, KIR2DL1, CD28, KIR2DL3, CD30/TNFRSF8, KIR2DL4/CD158d, CD31/PECAM-1, KIR2DS4, CD40 Ligand/TNFSF5, LAG-3, CD43, LAIR1, CD45, LAIR2, CD83, Leukotriene B4 R1, CD84/SLAMF5, NCAM-L1, CD94, NKG2A, CD97, NKG2C, CD229/SLAMF3, NKG2D, CD2F-10/SLAMF9, NT-4, CD69, NTB-A/SLAMF6, Common γ Chain/IL-2 Rγ, Osteopontin, CRACC/SLAMF7, PD-1, CRTAM, PSGL-1, CTLA-4, RANK/TNFRSF11A, CX3CR1, CX3CL1, L-Selectin, CXCR3, SIRP (31, CXCR4, SLAM, CXCR6, TCCR/WSX-1, DNAM-1, Thymopoietin, EMMPRIN/CD147, TIM-1, EphB6, TIM-2, Fas/TNFRSF6, TIM-3, Fas Ligand/TNFSF6, TIM-4, Fcγ RIII/CD16, TIM-6, GITR/TNFRSF18, TNF R1/TNFRSF1A, Granulysin, TNF R11/TNFRSF1B, HVEM/TNFRSF14, TRAIL R1/TNFRSF10A, ICAM-1/CD54, TRAIL R2/TNFRSF10B, ICAM-2/CD102, TRAIL R3/TNFRSF10C, IFN-γR1, TRAIL R4/TNFRSF10D, IFN-γR2, TSLP, IL-1 RI and TSLP R.

In some embodiments VSTM5 polypeptides or fusion proteins according to the invention will comprise at least one monocyte/macrophage cell target selected from the group consisting of B7-1/CD80, ILT4/CD85d, B7-H1, ILT5/CD85a, Common β Chain, Integrin α4/CD49d, BLAME/SLAMF8, Integrin α X/CD11c, CCL6/C10, Integrin β2/CD18, CD155/PVR, Integrin β3/CD61, CD31/PECAM-1, Latexin, CD36/SR-B3, Leukotriene B4 R1, CD40/TNFRSF5, LIMPII/SR-B2, CD43, LMIR1/CD300A, CD45, LMIR2/CD300c, CD68, LMIR3/CD300LF, CD84/SLAMF5, LMIR5/CD300LB, CD97, LMIR6/CD300LE, CD163, LRP-1, CD2F-10/SLAMF9, MARCO, CRACC/SLAMF7, MD-1, ECF-L, MD-2, EMMPRIN/CD147, MGL2, Endoglin/CD105, Osteoactivin/GPNMB, Fc γR1/CD64, Osteopontin, Fc γ RIIB/CD32b, PD-L2, Fc γRIIC/CD32c, Siglec-3/CD33, Fcγ RIIA/CD32a, SIGNR1/CD209, Fcγ RIII/CD16, SLAM, GM-CSF R α, TCCR/WSX-1, ICAM-2/CD102, TLR3, IFN-γ R1, TLR4, IFN-γ R2, TREM-1, IL-1 RII, TREM-2, ILT2/CD85j, TREM-3, ILT3/CD85k, TREML1/TLT-1, 2B4/SLAMF4, IL-10 R α, ALCAM, IL-10 Aminopeptidase N/ANPEP, ILT2/CD85j, Common β Chain, ILT3/CD85k, C1q R1/CD93, ILT4/CD85d, CCR1, ILT5/CD85a, CCR2, Integrin α4/CD49d, CCR5, Integrin α M/CD11b, CCR8, Integrin α X/CD11c, CD155/PVR, Integrin β2/CD18, CD14, Integrin β3/CD61, CD36/SR-B3, LAIR1, CD43, LAIR2, CD45, Leukotriene B4 R1, CD68, LIMPII/SR-B2, CD84/SLAMF5, LMIR1/CD300A, CD97, LMIR2/CD300c, CD163, LMIR3/CD300LF, Coagulation Factor III/Tissue Factor, LMIR5/CD300LB, CX3CR1, CX3CL1, LMIR6/CD300LE, CXCR4, LRP-1, CXCR6, M-CSF R, DEP-1/CD148, MD-1, DNAM-1, MD-2, EMMPRIN/CD147, MMR, Endoglin/CD105, NCAM-L1, Fc γ R1/CD64, PSGL-1, Fc γ RIII/CD16, RP105, G-CSF R, L-Selectin, GM-CSF R α, Siglec-3/CD33, HVEM/TNFRSF14, SLAM, ICAM-1/CD54, TCCR/WSX-1, ICAM-2/CD102, TREM-1, IL-6 R, TREM-2, CXCR1/IL-8 RA, TREM-3 and TREML1/TLT-1.

In some embodiments VSTM5 polypeptides or fusion proteins according to the invention will comprise at least one Dendritic cell target is selected from the group consisting of CD36/SR-B3, LOX-1/SR-E1, CD68, MARCO, CD163, SR-AI/MSR, CD5L, SREC-I, CL-P1/COLEC12, SREC-II, LIMPII/SR-B2, RP105, TLR4, TLR1, TLR5, TLR2, TLR6, TLR3, TLR9, 4-1BB Ligand/TNFSF9, IL-12/IL-23 p40, 4-Amino-1,8-naphthalimide, ILT2/CD85j, CCL21/6Ckine, ILT3/CD85k, 8-oxo-dG, ILT4/CD85d, 8D6A, ILT5/CD85a, A2B5, Integrin α4/CD49d, Aag, Integrin β2/CD18, AMICA, Langerin, B7-2/CD86, Leukotriene B4 R1, B7-H3, LMIR1/CD300A, BLAME/SLAMF8, LMIR2/CD300c, C1q R1/CD93, LMIR3/CD300LF, CCR6, LMIR5/CD300LB, CCR7, LMIR6/CD300LE, CD40/TNFRSF5, MAG/Siglec-4a, CD43, MCAM, CD45, MD-1, CD68, MD-2, CD83, MDL-1/CLEC5A, CD84/SLAMF5, MMR, CD97, NCAM-L1, CD2F-10/SLAMF9, Osteoactivin/GPNMB, Chem 23, PD-L2, CLEC-1, RP105, CLEC-2, Siglec-2/CD22, CRACC/SLAMF7, Siglec-3/CD33, DC-SIGN, Siglec-5, DC-SIGNR/CD299, Siglec-6, DCAR, Siglec-7, DCIR/CLEC4A, Siglec-9, DEC-205, Siglec-10, Dectin-1/CLEC7A, Siglec-F, Dectin-2/CLEC6A, SIGNR1/CD209, DEP-1/CD148, SIGNR4, DLEC, SLAM, EMMPRIN/CD147, TCCR/WSX-1, Fc γ R1/CD64, TLR3, Fc γ RIIB/CD32b, TREM-1, Fc γ RIIC/CD32c, TREM-2, Fc γ RIIA/CD32a, TREM-3, Fc γ RIII/CD16, TREML1/TLT-1, ICAM-2/CD102 and Vanilloid R1.

In some embodiments VSTM5 polypeptides or fusion proteins according to the invention will comprise at least one TNF receptor superfamily member is selected from the group consisting of 4-1BB/TNFRSF9, NGF R/TNFRSF16, BAFF R/TNFRSF13C, Osteoprotegerin/TNFRSF11B, BCMA/TNFRSF17, OX40/TNFRSF4, CD27/TNFRSF7, RANK/TNFRSF11A, CD30/TNFRSF8, RELT/TNFRSF19L, CD40/TNFRSF5, TACI/TNFRSF13B, DcR3/TNFRSF6B, TNF RI/TNFRSF1A, DcTRAIL R1/TNFRSF23, TNF RII/TNFRSF1B, DcTRAIL R2/TNFRSF22, TRAIL R1/TNFRSF10A, DR3/TNFRSF25, TRAIL R2/TNFRSF10B, DR6/TNFRSF21, TRAIL R3/TNFRSF10C, EDAR, TRAIL R4/TNFRSF10D, Fas/TNFRSF6, TROY/TNFRSF19, GITR/TNFRSF18, TWEAK R/TNFRSF12, HVEM/TNFRSF14, XEDAR, Lymphotoxin β R/TNFRSF3, 4-1BB Ligand/TNFSF9, Lymphotoxin, APRIL/TNFSF13, Lymphotoxin β/TNFSF3, BAFF/TNFSF13C, OX40 Ligand/TNFSF4, CD27 Ligand/TNFSF7, TL1A/TNFSF15, CD30 Ligand/TNFSF8, TNF-α/TNFSF1A, CD40 Ligand/TNFSF5, TNF-β/TNFSF1B, EDA-A2, TRAIL/TNFSF10, Fas Ligand/TNFSF6, TRANCE/TNFSF11, GITR Ligand/TNFSF18, TWEAK/TNFSF12 and LIGHT/TNFSF14.

In some embodiments VSTM5 polypeptides or fusion proteins according to the invention will comprise at least one Hedgehog family member selected from the group consisting of Patched and Smoothened.

In some embodiments VSTM5 polypeptides or fusion proteins according to the invention will comprise at least one receptor tyrosine kinase selected from the group consisting of Ax1, FGF R4, C1q R1/CD93, FGF R5, DDR1, Flt-3, DDR2, HGF R, Dtk, IGF-I R, EGF R, IGF-II R, Eph, INSRR, EphA1, Insulin R/CD220, EphA2, M-CSF R, EphA3, Mer, EphA4, MSP R/Ron, EphA5, MuSK, EphA6, PDGF R α, EphA7, PDGF R β, EphA8, Ret, EphB1, ROR1, EphB2, ROR2, EphB3, SCF R/c-kit, EphB4, Tie-1, EphB6, Tie-2, ErbB2, TrkA, ErbB3, TrkB, ErbB4, TrkC, FGF R1, VEGF R1/Flt-1, FGF R2, VEGF R2/Flk-1, FGF R3 and VEGF R3/Flt-4.

In some embodiments VSTM5 polypeptides or fusion proteins according to the invention will comprise at least one Transforming Growth Factor (TGF)-β superfamily member selected from the group consisting of Activin RIA/ALK-2, GFR α-1, Activin RIB/ALK-4, GFR α2, Activin RHA, GFR α-3, Activin RIIB, GFR α-4, ALK-1, MIS RII, ALK-7, Ret, BMPR-IA/ALK-3, TGF-betβa R1/ALK-5, BMPR-IB/ALK-6, TGF-β RII, BMPR-II, TGF-β RIIb, Endoglin/CD 105 and TGF-β RIII.

In some embodiments VSTM5 polypeptides or fusion proteins according to the invention will comprise at least one Wnt-related molecule selected from the group consisting of Frizzled-1, Frizzled-8, Frizzled-2, Frizzled-9, Frizzled-3, sFRP-1, Frizzled-4, sFRP-2, Frizzled-5, sFRP-3, Frizzled-6, sFRP-4, Frizzled-7, MFRP, LRP 5, LRP 6, Wnt-1, Wnt-8a, Wnt-3a, Wnt-10b, Wnt-4, Wnt-11, Wnt-5a, Wnt-9a and Wnt-7a.

In some embodiments VSTM5 polypeptides or fusion proteins according to the invention will comprise at least one receptor ligand selected from the group consisting of 4-1BB Ligand/TNFSF9, Lymphotoxin, APRIL/TNFSF13, Lymphotoxin β/TNFSF3, BAFF/TNFSF13C, OX40 Ligand/TNFSF4, CD27 Ligand/TNFSF7, TL1A/TNFSF15, CD30 Ligand/TNFSF8, TNF-α/TNFSF1A, CD40 Ligand/TNFSF5, TNF-β/TNFSF1B, EDA-A2, TRAIL/TNFSF10, Fas Ligand/TNFSF6, TRANCE/TNFSF11, GITR Ligand/TNFSF18, TWEAK/TNFSF12, LIGHT/TNFSF14, Amphiregulin, NRG1 isoform GGF2, Betacellulin, NRG1 Isoform SMDF, EGF, NRG1-α/HRG1-α, Epigen, NRG1-β1/HRG1-β1, Epiregulin, TGF-α, HB-EGF, TMEFF1/Tomoregulin-1, Neuregulin-3, TMEFF2, IGF-I, IGF-II, Insulin, Activin A, Activin B, Activin AB, Activin C, BMP-2, BMP-7, BMP-3, BMP-8, BMP-3b/GDF-10, BMP-9, BMP-4, BMP-15, BMP-5, Decapentaplegic, BMP-6, GDF-1, GDF-8, GDF-3, GDF-9, GDF-5, GDF-11, GDF-6, GDF-15, GDF-7, Artemin, Neurturin, GDNF, Persephin, TGF-β, TGF-β2, TGF-β1, TGF-β3, LAP (TGF-β1), TGF-β5, Latent TGF-β1, Latent TGF-β bp1, TGF-β1.2, Lefty, Nodal, MIS/AMH, FGF acidic, FGF-12, FGF basic, FGF-13, FGF-3, FGF-16, FGF-4, FGF-17, FGF-5, FGF-19, FGF-6, FGF-20, FGF-8, FGF-21, FGF-9, FGF-23, FGF-10, KGF/FGF-7, FGF-11, Neuropilin-1, P1GF, Neuropilin-2, P1GF-2, PDGF, PDGF-A, VEGF, PDGF-B, VEGF-B, PDGF-C, VEGF-C, PDGF-D, VEGF-D and PDGF-AB.

In some embodiments VSTM5 polypeptides or fusion proteins according to the invention will comprise at least one tumor antigen selected from the group consisting of Squamous Cell Carcinoma Antigen 1 (SCCA-1), (PROTEIN T4-A), Squamous Cell Carcinoma Antigen 2 (SCCA-2), Ovarian carcinoma antigen CA125 (1A1-3B; KIAA0049), MUCIN 1 (TUMOR-ASSOCIATED MUCIN; Carcinoma-Associated Mucin; Polymorphic Epithelial Mucin; PEM; PEMT; EPISIALIN; Tumor-Associated Epithelial Membrane Antigen; EMA; H23AG; Peanut-Reactive Urinary Mucin; PUM; and Breast Carcinoma-Associated Antigen DF3), CTCL tumor antigen sel-1, CTCL tumor antigen se14-3, CTCL tumor antigen se20-4, CTCL tumor antigen se20-9, CTCL tumor antigen se33-1, CTCL tumor antigen se37-2, CTCL tumor antigen se57-1, CTCL tumor antigen se89-1, Prostate-specific membrane antigen, 5T4 oncofetal trophoblast glycoprotein, Orf73 Kaposi's sarcoma-associated herpesvirus, MAGE-C1 (cancer/testis antigen CT7), MAGE-B1 ANTIGEN (MAGE-XP Antigen; DAM10), MAGE-B2 Antigen (DAM6), MAGE-2 ANTIGEN, MAGE-4a antigen, MAGE-4b antigen, Colon cancer antigen NY-CO-45, Lung cancer antigen NY-LU-12 variant A, Cancer associated surface antigen, Adenocarcinoma antigen ART1, Paraneoplastic associated brain-testis-cancer antigen (onconeuronal antigen MA2; paraneoplastic neuronal antigen), Neuro-oncological ventral antigen 2 (NOVA2), Hepatocellular carcinoma antigen gene 520, Tumor-Associated Antigen CO-029, Tumor-associated antigen MAGE-X2, Synovial sarcoma, X breakpoint 2, Squamous cell carcinoma antigen recognized by T cell, Serologically defined colon cancer antigen 1, Serologically defined breast cancer antigen NY-BR-15, Serologically defined breast cancer antigen NY-BR-16, Chromogranin A, parathyroid secretory protein 1, DUPAN-2, CA 19-9, CA 72-4, CA 195 and L6.

In some embodiments VSTM5 polypeptides or fusion proteins according to the invention will comprise at least one B cell target selected from the group consisting of CD10, CD19, CD20, CD21, CD22, CD23, CD24, CD37, CD38, CD39, CD40, CD72, CD73, CD74, CDw75, CDw76, CD77, CD78, CD79a/b, CD80, CD81, CD82, CD83, CD84, CD85, CD86, CD89, CD98, CD126, CD127, CDw130, CD138 and CDw150.

In some embodiment VSTM5 polypeptides or fusion proteins according to the invention will comprise at least one angiogenesis target is selected from the group consisting of Angiopoietin-1, Angiopoietin-like 2, Angiopoietin-2, Angiopoietin-like 3, Angiopoietin-3, Angiopoietin-like 7/CDT6, Angiopoietin-4, Tie-1, Angiopoietin-like 1, Tie-2, Angiogenin, iNOS, Coagulation Factor III/Tissue Factor, nNOS, CTGF/CCN2, NOV/CCN3, DANCE, OSM, EDG-1, Plfr, EG-VEGF/PK1, Proliferin, Endostatin, ROBO4, Erythropoietin, Thrombospondin-1, Kininostatin, Thrombospondin-2, MFG-E8, Thrombospondin-4, Nitric Oxide, VGSQ, eNOS, EphA1, EphA5, EphA2, EphA6, EphA3, EphA7, EphA4, EphA8, EphB1, EphB4, EphB2, EphB6, EphB3, Ephrin-AL Ephrin-A4, Ephrin-A2, Ephrin-A5, Ephrin-A3, Ephrin-B1, Ephrin-B3, Ephrin-B2, FGF acidic, FGF-12, FGF basic, FGF-13, FGF-3, FGF-16, FGF-4, FGF-17, FGF-5, FGF-19, FGF-6, FGF-20, FGF-8, FGF-21, FGF-9, FGF-23, FGF-10, KGF/FGF-7, FGF-11, FGF R1, FGF R4, FGF R2, FGF R5, FGF R3, Neuropilin-1, Neuropilin-2, Semaphorin 3A, Semaphorin 6B, Semaphorin 3C, Semaphorin 6C, Semaphorin 3E, Semaphorin 6D, Semaphorin 6A, Semaphorin 7A, MMP, MMP-11, MMP-1, MMP-12, MMP-2, MMP-13, MMP-3, MMP-14, MMP-7, MMP-15, MMP-8, MMP-16/MT3-MMP, MMP-9, MMP-24/MT5-MMP, MMP-10, MMP-25/MT6-MMP, TIMP-1, TIMP-3, TIMP-2, TIMP-4, ACE, IL-13 R α1, IL-13, C1q R1/CD93, Integrin α 4/CD49d, VE-Cadherin, Integrin β2/CD18, CD31/PECAM-1, KLF4, CD36/SR-B3, LYVE-1, CD151, MCAM, CL-P1/COLEC12, Nectin-2/CD112, Coagulation Factor III/Tissue Factor, E-Selectin, D6, P-Selectin, DC-SIGNR/CD299, SLAM, EMMPRIN/CD147, Tie-2, Endoglin/CD105, TNF RI/TNFRSF1A, EPCR, TNF RII/TNFRSF1B, Erythropoietin R, TRAIL R1/TNFRSF10A, ESAM, TRAIL R2/TNFRSF10B, FABP5, VCAM-1, ICAM-1/CD54, VEGF R2/Flk-1, ICAM-2/CD102, VEGF R3/Flt-4, IL-1 RI and VGSQ.

In some embodiments VSTM5 polypeptides or fusion proteins according to the invention will comprise a VSTM5 polypeptide according to the invention and at least one heterologous polypeptide and/or or binding moiety or VSTM5 polypeptides are linked to one another by an amino acid spacer.

In some embodiments VSTM5 polypeptides or fusion proteins according to the invention will a VSTM5 polypeptide and at least one heterologous polypeptide and/or or binding moiety or VSTM5 polypeptides are linked to one another by an amino acid spacer of sufficient length of amino acid residues so that the different moieties can successfully bind to their individual targets.

In some embodiments VSTM5 polypeptides or fusion proteins according to the invention will comprise 2-10 of any of the VSTM5 ECD polypeptide fragments disclosed herein.

In some embodiments VSTM5 polypeptides or fusion proteins according to the invention will comprise one or more VSTM5 polypeptide(s) and at least one heterologous polypeptide optionally intervened by a heterologous linker which optionally comprises a polypeptide that is not a fragment of a VSTM5 polypeptide.

In some embodiments VSTM5 polypeptides or fusion proteins according to the invention will comprise a linker which is a peptide comprising 5-50 amino acid residues, more preferably 5-25 amino acid residues.

In some embodiments VSTM5 polypeptides or fusion proteins according to the invention will comprise a linker which comprises, consists essentially of, glycine, serine, and/or alanine residues.

In some embodiments VSTM5 polypeptides or fusion proteins according to the invention will comprise a linker which comprises 5-50, 5-25, 5-15, 4-14, 4-12, or more amino acid residues, e.g., which may include or consist of glycine, serine, and/or alanine residues.

Peptide or Polypeptide Linker Domain

The disclosed VSTM5 fusion proteins optionally contain a peptide or polypeptide linker domain that separates the VSTM5 polypeptide from the second polypeptide. Various non-limiting examples of such linker domains are described herein. In one embodiment, the linker domain contains the hinge region of an immunoglobulin. In a further embodiment, the hinge region is derived from a human immunoglobulin. Suitable human immunoglobulins that the hinge can be derived from include IgG, IgD and IgA. In a further embodiment, the hinge region is derived from human IgG Amino acid sequences of immunoglobulin hinge regions and other domains are well known in the art. In one embodiment, VSTM5 fusion polypeptides contain the hinge, C_{H2} and C_{H3} regions of a human immunoglobulin Cγ1 chain, optionally with the Cys at position 220 (according to full length human IgG1, position 5 in SEQ ID NO:113) replaced with a Ser (SEQ ID NO: 114) having at least 85%, 90%, 95%, 99% or 100% sequence homology to amino acid sequence set forth in SEQ ID NO:113:

EPKSCDKTHTCPPCPAPELLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK

The hinge can be further shortened to remove amino acids 1, 2, 3, 4, 5, or combinations thereof of any one of SEQ ID NOs: 113, 114. In one embodiment, amino acids 1-5 of any one of SEQ ID NOs: 113 or 114 are deleted. Exemplary VSTM5 fusion polypeptides comprised of the hinge, CH2 and CH3 regions of a human immunoglobulin Cγ1 chain with the Cys at position 220 replaced with a Ser are set forth in SEQ ID NOs:-130.

In another embodiment, VSTM5 fusion polypeptides contain the C_{H2} and C_{H3} regions of a human immunoglobulin Cγ1 chain having at least 85%, 90%, 95%, 99% or 100% sequence homology to amino acid sequence set forth in SEQ ID NO:115:

APELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPA
PIEKTISKAKGQPREPQVYTLPPSRDELTKNQVSLTCLVKGFYPSDIAVE
WESNGQPENNYKTTPPVLDSDGSFFLYSKLTVDKSRWQQGNVFSCSVMHE
ALHNHYTQKSLSLSPGK

In another embodiment, the VSTM5 fusion polypeptides contain the CH2 and CH3 regions of a murine immunoglobulin Cγ2a chain at least 85%, 90%, 95%, 99% or 100% sequence homology to amino acid sequence set forth in SEQ ID NO: 112:

EPRGPTIKPCPPCKCPAPNLLGGPSVFIFPPKIKDVLMISLSPIVTCVVV
DVSEDDPDVQISWFVNNVEVHTAQTQTHREDYNSTLRVVSALPIQHQDWM
SGKEFKCKVNNKDLPAPIERTISKPKGSVRAPQVYVLPPPEEEMTKKQVT
LTCMVTDFMPEDIYVEWTNNGKTELNYKNTEPVLDSDGSYFMYSKLRVEK
KNWVERNSYSCSVVHEGLHNHHTTKSFSRTPGK.

In another embodiment, the linker domain optionally contains a hinge region of an immunoglobulin as described above, and further includes one or more additional immunoglobulin domains.

Other suitable peptide/polypeptide linker domains optionally include naturally occurring or non-naturally occurring peptides or polypeptides. Peptide linker sequences are at least 2 amino acids in length. Optionally the peptide or polypeptide domains are flexible peptides or polypeptides. A “flexible linker” herein refers to a peptide or polypeptide containing two or more amino acid residues joined by peptide bond(s) that provides increased rotational freedom for two polypeptides linked thereby than the two linked polypeptides would have in the absence of the flexible linker. Such rotational freedom allows two or more antigen binding sites joined by the flexible linker to each access target antigen(s) more efficiently. Exemplary flexible peptides/polypeptides include, but are not limited to, the amino acid sequences Gly-Ser (SEQ ID NO:117), Gly-Ser-Gly-Ser (SEQ ID NO:118), Ala-Ser (SEQ ID NO:119), Gly-Gly-Gly-Ser (SEQ ID NO:120), Gly4-Ser (SEQ ID NO:121), (Gly4-Ser)2 (SEQ ID NO:122), (Gly4-Ser)3 (SEQ ID NO:123), (Gly4-Ser)4 (SEQ ID NO: 124), [Gly4-Set]2 Gly-Ala-Gly-Ser-Gly4-Ser (SEQ ID NO: 142), Gly-(Gly4-Ser)2 (SEQ ID NO:143), Gly4-Ser-Gly (SEQ ID NO:144), Gly-Ser-Gly2 (SEQ ID NO:145) and Gly-Ser-Gly2-Ser (SEQ ID NO:146). Additional flexible peptide/polypeptide sequences are well known in the art. Other suitable peptide linker domains optionally include the TEV linker ENLYFQG, a linear epitope recognized by the Tobacco Etch Virus protease. Exemplary peptides/polypeptides include, but are not limited to, GSENLYFQGSG (SEQ ID NO:138). Other suitable peptide linker domains include helix forming linkers such as Ala-(Glu-Ala-Ala-Ala-Lys)n-Ala (n=1-5). Additional helix forming peptide/polypeptide sequences are well known in the art. Non-limiting examples of such linkers are depicted in SEQ ID NOs:125-129, 139-141.

In some optionally embodiments VSTM5 fragments, e.g., ECD fragments, are linked to each other (multimers) and/or one or more VSTM5 fragments, e.g., ECD fragments, are linked to a heterologous polypeptide such as an immunoglobulin or fragment thereof, especially an immunoglobulin heavy chain or fragment thereof by a peptide linker, preferably a “flexible linker” sequence. The linker sequence should allow effective positioning of the VSTM5 fragments and the heterologous polypeptide such as an immunoglobulin polypeptide or domains thereof to allow functional activity of both moieties and the domains thereof. Successful presentation of the polypeptide fusion can modulate the activity of a cell either to induce or to inhibit T-cell proliferation, or to initiate or inhibit an immune response to a particular site. This can be determined in appropriate assays such as disclosed herein below, including the in vitro assays that includes sequential steps of culturing T cells to proliferate same, and contacting the T cells with a fusion polypeptide according to the invention or a cell expressing same and then evaluating whether the fusion polypeptide promotes or inhibits T cell proliferation.

As used herein, the phrase “effective positioning of the heterologous polypeptide and the VSTM5 polypeptide”, or other similar phrase, is intended to mean that the domains of these moieties are positioned so that VSTM5 domains and heterologous polypeptide domains are capable of interacting with immune or other target cells, e.g., cancer or other VSTM5 expressing cells to initiate or inhibit an immune reaction, or to inhibit or stimulate cell development.

With respect to VSTM5-Ig fusion proteins the linker sequence also preferably permits effective positioning of the F_{c} domain and VSTM5 domains to allow functional activity of each domain. In certain embodiments, the Fc domains are effectively positioned to allow proper fusion protein complex formation and/or interactions with F_{c} receptors on immune cells or proteins of the complement system to stimulate F_{c}-mediated effects including opsonization, cell lysis, degranulation of mast cells, basophils, and eosinophils, and other Fc receptor-dependent processes; activation of the complement pathway; and enhanced in vivo half-life of the fusion protein complex.

Linker sequences are discussed supra in connection with fusion proteins according to the invention. Linker sequences can optionally be used to link two or more VSTM5 polypeptides of the biologically active polypeptide to generate a single-chain molecule with the desired functional activity. In some preferred embodiments the linker sequence comprises from about 5 to 20 amino acids, more preferably from about 7 or 8 to about 16 amino acids. The linker sequence is preferably flexible so as not hold the VSTM5 polypeptide and moiety linked thereto, e.g., an effector molecule in a single undesired conformation. The linker sequence can be used, e.g., to space the recognition site from the fused molecule. Specifically, the peptide linker sequence can be positioned between the biologically active VSTM5 polypeptide and the effector molecule, e.g., to chemically cross-link same and to provide molecular flexibility. The linker in some embodiments will predominantly comprise amino acids with small side chains, such as glycine, alanine and serine, to provide for flexibility. Preferably about 80 or 90 percent or greater of the linker sequence comprise glycine, alanine or serine residues, particularly glycine and serine residues. Other suitable linker sequences include flexible linker designs that have been used successfully to join antibody variable regions together, see Whitlow, M. et al., (1991) Methods: A Companion to Methods in Enzymology 2:97-105. In some examples, for covalently linking an effector molecule to a VSTM5 molecule, the amino sequence of the linker should be capable of spanning a suitable distance from the C-terminal residue of the VSTM5 polypeptide to the N-terminal residue of the effector molecule. Suitable linker sequences can be readily identified empirically. Additionally, suitable size and sequences of linker sequences also can be determined by known computer modeling techniques based on the predicted size and shape of the fusion polypeptide. Other linker sequences are discussed supra in connection with fusion proteins according to the invention.

Optionally a polypeptide as described herein comprises 2-20 VSTM5 ECD polypeptide fragments linked together, such that the polypeptide has less than 95% homology to a VSTM5 sequence as described herein.

Optionally the fragments are intervened by a heterologous linker which optionally comprises a polypeptide that is not a fragment of a VSTM5 polypeptide.

Optionally the linker is a peptide comprising 5-50 amino acid residues, more preferably 5-25 amino acid residues.

Optionally the linker comprises, consists essentially of, or consists of 4-12 glycine, serine, and/or alanine residues.

Dimerization, Multimerization and Targeting Domains

The fusion proteins disclosed herein optionally contain a dimerization or multimerization or oligomerization domain that functions to dimerize, oligomerize or multimerize two or more fusion proteins, which may be the same or different (heteromultimers or homomultimers). For example a VSTM5 fusion protein may be attached to another VSTM5 fusion protein or another moiety, e.g. another costimulatory fusion protein. The domain that functions to dimerize or multimerize the fusion proteins can either be a separate domain, or alternatively can be contained within one of the other domains (VSTM5 polypeptide, second polypeptide, or peptide/polypeptide linker domain) of the fusion protein.

Dimerization or multimerization can occur between or among two or more fusion proteins through dimerization or multimerization domains. Alternatively, dimerization or multimerization of fusion proteins can occur by chemical crosslinking. The dimers or multimers that are formed can be homodimeric/homomultimeric or heterodimeric/heteromultimeric. The second polypeptide “partner” in the VSTM5 fusion polypeptides may be comprised of one or more other proteins, protein fragments or peptides as described herein, including but not limited to any immunoglobulin (Ig) protein or portion thereof, preferably the Fc region, or a portion of a biologically or chemically active protein such as the papillomavirus E7 gene product, melanoma-associated antigen p97), and HIV env protein (gp120). The “partner” is optionally selected to provide a soluble dimer/multimer and/or for one or more other biological activities as described herein.

A “dimerization domain” is formed by the association of at least two amino acid residues or of at least two peptides or polypeptides (which may have the same, or different, amino acid sequences). The peptides or polypeptides may interact with each other through covalent and/or non-covalent associations). Optional dimerization domains contain at least one cysteine that is capable of forming an intermolecular disulfide bond with a cysteine on the partner fusion protein. The dimerization domain can contain one or more cysteine residues such that disulfide bond(s) can form between the partner fusion proteins. In one embodiment, dimerization domains contain one, two or three to about ten cysteine residues. In a further embodiment, the dimerization domain is the hinge region of an immunoglobulin.

Additional exemplary dimerization domains can be any known in the art and include, but not limited to, coiled coils, acid patches, zinc fingers, calcium hands, a CH1-CL pair, an “interface” with an engineered “knob” and/or “protuberance” as described in U.S. Pat. No. 5,821,333, leucine zippers (e.g., from jun and/or fos) (U.S. Pat. No. 5,932,448), and/or the yeast transcriptional activator GCN4, SH2 (src homology 2), SH3 (src Homology 3) (Vidal, et al, Biochemistry, 43, 7336-44 ((2004)), phosphotyrosine binding (PTB) (Zhou, et al., Nature, 378:584-592 (1995)), WW (Sudol, Prog. Biochys. Mol Biol., 65:113-132 (1996)), PDZ (Kim, et al., Nature, 378: 85-88 (1995); Komau, et al, Science, 269. 1737-1740 (1995)) 14-3-3, WD40 (Hu5 et al., J Biol Chem., 273: 33489-33494 (1998)) EH, Lim, “An isoleucine zipper, a receptor dimer pair (e.g., interleukin-8 receptor (IL-8R); and integrin heterodimers such as LFA-I and GPIIIb/IIIa), or the dimerization region(s) thereof, dimeric ligand polypeptides (e.g., nerve growth factor (NGF), neurotrophin-3 (NT-3), interleukin-8 (IL-8), vascular endothelial growth factor (VEGF), VEGF-C, VEGF-D, PDGF members, and brain-derived neurotrophic factor (BDNF) (Arakawa, et al., J Biol. Chem., 269(45): 27833-27839 (1994) and Radziejewski, et al., Biochem., 32(48): 1350 (1993)) and can also be variants of these domains in which the affinity is altered. The polypeptide pairs can be identified by methods known in the art, including yeast two hybrid screens. Yeast two hybrid screens are described in U.S. Pat. Nos. 5,283,173 and 6,562,576. Affinities between a pair of interacting domains can be determined using methods known in the art, including as described in Katahira, et al., J. Biol Chem, 277, 9242-9246 (2002)). Alternatively, a library of peptide sequences can be screened for heterodimerization, for example, using the methods described in WO 01/00814. Useful methods for protein-protein interactions are also described in U.S. Pat. No. 6,790,624.

A “multimerization domain” or “oligomerization domain” referred to herein is a domain that causes three or more peptides or polypeptides to interact with each other through covalent and/or non-covalent association(s). Suitable multimerization or oligomerization domains include, but are not limited to, coiled-coil domains. A coiled-coil is a peptide sequence with a contiguous pattern of mainly hydrophobic residues spaced 3 and 4 residues apart, usually in a sequence of seven amino acids (heptad repeat) or eleven amino acids (undecad repeat), which assembles (folds) to form a multimeric bundle of helices. Coiled-coils with sequences including some irregular distribution of the 3 and 4 residues spacing are also contemplated. Hydrophobic residues are in particular the hydrophobic amino acids Val, Ile, Leu, Met, Tyr, Phe and Trp. “Mainly hydrophobic” means that at least 50% of the residues must be selected from the mentioned hydrophobic amino acids.

The coiled coil domain may be derived from laminin. In the extracellular space, the heterotrimeric coiled coil protein laminin plays an important role in the formation of basement membranes. Apparently, the multifunctional oligomeric structure is required for laminin function. Coiled coil domains may also be derived from the thrombospondins in which three (TSP-I and TSP-2) or five (TSP-3, TSP-4 and TSP-5) chains are connected, or from COMP (COMPcc) (Guo, et at., EMBO J, 1998, 17: 5265-5272) which folds into a parallel five-stranded coiled coil (Malashkevich, et al., Science, 274: 761-765 (1996)). Additional non-limiting examples of coiled-coil domains derived from other proteins, and other domains that mediate polypeptide multimerization are known in the art such as the vasodilator-stimulated phosphoprotein (VASP) domain, matrilin-1 (CMP), viral fusion peptides, soluble NSF (N-ethylmaleimide-sensitive factor) Attachment Protein receptor (SNARE) complexes, leucine-rich repeats, certain tRNA synthetases, are suitable for use in the disclosed fusion proteins.

In another embodiment, VSTM5 polypeptides, fusion proteins, or fragments thereof can be induced to form multimers by binding to a second multivalent polypeptide, such as an antibody. Antibodies suitable for use to multimerize VSTM5 polypeptides, fusion proteins, or fragments thereof include, but are not limited to, IgM antibodies and cross-linked, multivalent IgG, IgA, IgD, or IgE complexes.

Dimerization or multimerization can occur between or among two or more fusion proteins through dimerization or multimerization domains, including those described above. Alternatively, dimerization or multimerization of fusion proteins can occur by chemical crosslinking. Fusion protein dimers can be homodimers or heterodimers. Fusion protein multimers can be homomultimers or heteromultimers. Fusion protein dimers as disclosed herein are of formula II:

N—R1-R2-R3-C

N—R4-R5-R6-C or, alternatively, are of formula III:

N—R1-R2-R3-C

C—R4-R5-R6-N

wherein the fusion proteins of the dimer provided by formula II are defined as being in a parallel orientation and the fusion proteins of the dimer provided by formula III are defined as being in an antiparallel orientation. Parallel and antiparallel dimers are also referred to as cis and trans dimers, respectively. “N” and “C” represent the N- and C-termini of the fusion protein, respectively. The fusion protein constituents “R1”, “R2” and “R3” are as defined above with respect to formula I. With respect to both formula II and formula III, “R4” is a VSTM5 polypeptide or a second polypeptide, “R5” is an optional peptide/polypeptide linker domain, and “R6” is a VSTM5 polypeptide or a second polypeptide, wherein “R6” is a VSTM5 polypeptide when “R4” is a second polypeptide, and “R6′” is a second polypeptide when “R4” is a VSTM5 polypeptide. In one embodiment, “R1” is a VSTM5 polypeptide, “R4” is also a VSTM5 polypeptide, and “R3” and “R6” are both second polypeptides.

Fusion protein dimers of formula II are defined as homodimers when “R1”=“R4”, “R2”=“R5” and “R3”=“R6”. Similarly, fusion protein dimers of formula III are defined as homodimers when “R1”=“R6”, “R2”=“R5” and “R3”=“R4”. Fusion protein dimers are defined as heterodimers when these conditions are not met for any reason. For example, heterodimers may contain domain orientations that meet these conditions (i.e., for a dimer according to formula II, “R1” and “R4” are both VSTM5 polypeptides, “R2” and “R5” are both peptide/polypeptide linker domains and “R3” and “R6” are both second polypeptides), however the species of one or more of these domains is not identical. For example, although “R3” and “R6” may both be VSTM5 polypeptides, one polypeptide may contain a wild-type VSTM5 amino acid sequence while the other polypeptide may be a variant VSTM5 polypeptide. An exemplary variant VSTM5 polypeptide is VSTM5, polypeptide that has been modified to have increased or decreased binding to a target cell, increased activity on immune cells, increased or decreased half-life or stability. Dimers of fusion proteins that contain either a C_{H1} or C_{L} region of an immunoglobulin as part of the polypeptide linker domain preferably form heterodimers wherein one fusion protein of the dimer contains a C_{H1} region and the other fusion protein of the dimer contains a C_{L} region.

Fusion proteins can also be used to form multimers. As with dimers, multimers may be parallel multimers, in which all fusion proteins of the multimer are aligned in the same orientation with respect to their N- and C-termini. Multimers may be antiparallel multimers, in which the fusion proteins of the multimer are alternatively aligned in opposite orientations with respect to their N- and C-termini. Multimers (parallel or antiparallel) can be either homomultimers or heteromultimers. The fusion protein is optionally produced in dimeric form; more preferably, the fusion is performed at the genetic level as described below, by joining polynucleotide sequences corresponding to the two (or more) proteins, portions of proteins and/or peptides, such that a joined or fused protein is produced by a cell according to the joined polynucleotide sequence. A description of preparation for such fusion proteins is described with regard to U.S. Pat. No. 5,851,795 to Linsley et al, which is hereby incorporated by reference as if fully set forth herein as a non-limiting example only.

Targeting Domains

The VSTM5 polypeptides and fusion proteins can contain a targeting domain to target the molecule to specific sites in the body. Optional targeting domains target the molecule to areas of inflammation. Exemplary targeting domains are antibodies, or antigen binding fragments thereof that are specific for inflamed tissue or to a proinflammatory cytokine including but not limited to IL17, IL-4, IL-6, IL-12, IL-21, IL-22, IL-23, MIF, TNF-α, and TNF-β and combinations thereof. In the case of neurological disorders such as Multiple Sclerosis, the targeting domain may target the molecule to the CNS or may bind to VCAM-I on the vascular epithelium. Additional targeting domains can be peptide aptamers specific for a proinflammatory molecule. In other embodiments, the VSTM5 fusion protein can include a binding partner specific for a polypeptide displayed on the surface of an immune cell, for example a T cell. In still other embodiments, the targeting domain specifically targets activated immune cells. Optional immune cells that are targeted include Th0, Th1, Th17, Th2 and Th22 T cells, other cells that secrete, or cause other cells to secrete inflammatory molecules including, but not limited to, IL-1β, TNF-α, TGF-β, IFN-γ, IL-17, IL-6, IL-23, IL-22, IL-21, and MMPs, and Tregs. For example, a targeting domain for Tregs may bind specifically to CD25.

Other targeting moieties or heterologous polypeptides that optionally may be attached or contained within VSTM5 polypeptides or fusion proteins according to the invention are discussed supra in connection with the syntheseis of exemplary fusion proteins according to the invention.

The above changes are intended as illustrations only of optional changes and are not meant to be limiting in any way. Furthermore, the above explanation is provided for descriptive purposes only, without wishing to be bound by a single hypothesis.

Addition of Groups

If a protein according to the present invention is a linear molecule, it is possible to place various functional groups at various points on the linear molecule which are susceptible to or suitable for chemical modification. Functional groups can be added to the termini of linear forms of the protein according to at least some embodiments of the invention. In some embodiments, the functional groups improve the activity of the protein with regard to one or more characteristics, including but not limited to, improvement in stability, penetration (through cellular membranes and/or tissue barriers), tissue localization, efficacy, decreased clearance, decreased toxicity, improved selectivity, improved resistance to expulsion by cellular pumps, and the like. For convenience sake and without wishing to be limiting, the free N-terminus of one of the sequences contained in the compositions according to at least some embodiments of the invention will be termed as the N-terminus of the composition, and the free C-terminal of the sequence will be considered as the C-terminus of the composition. Either the C-terminus or the N-terminus of the sequences, or both, can be linked to a carboxylic acid functional groups or an amine functional group, respectively.

Non-limiting examples of suitable functional groups are described in Green and Wuts, “Protecting Groups in Organic Synthesis”, John Wiley and Sons, Chapters 5 and 7, (1991), the teachings of which are incorporated herein by reference. Preferred protecting groups are those that facilitate transport of the active ingredient attached thereto into a cell, for example, by reducing the hydrophilicity and increasing the lipophilicity of the active ingredient, these being an example for “a moiety for transport across cellular membranes”.

These moieties can optionally and preferably be cleaved in vivo, either by hydrolysis or enzymatically, inside the cell. (Ditter et al., J. Pharm Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al., Arch. Biochem. Biophys. 239:538 (1985) and Singhal et al., FASEB J. 1:220 (1987)). Hydroxyl protecting groups include esters, carbonates and carbamate protecting groups Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a composition of the present invention is protected, preferably with a methyl, ethyl, benzyl or substituted benzyl ester, more preferably as a benzyl ester.

Non-limiting, illustrative examples of N-terminal protecting groups include acyl groups (—CO—R1) and alkoxy carbonyl or aryloxy carbonyl groups (—CO—O—R1), wherein R1 is an aliphatic, substituted aliphatic, benzyl, substituted benzyl, aromatic or a substituted aromatic group. Specific examples of acyl groups include but are not limited to acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3-O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)-O—CO—, Adamantan, naphtalen, myristoleyl, toluen, biphenyl, cinnamoyl, nitrobenzoy, toluoyl, furoyl, benzoyl, cyclohexane, norbornane, or Z-caproic. In order to facilitate the N-acylation, one to four glycine residues can be present in the N-terminus of the molecule.

The carboxyl group at the C-terminus of the compound can be protected, for example, by a group including but not limited to an amide (i.e., the hydroxyl group at the C-terminus is replaced with —NH₂, —NHR₂ and —NR₂R₃) or ester (i.e., the hydroxyl group at the C-terminus is replaced with —OR₂). R₂ and R₃ are optionally independently an aliphatic, substituted aliphatic, benzyl, substituted benzyl, aryl or a substituted aryl group. In addition, taken together with the nitrogen atom, R₂ and R₃ can optionally form a C4 to C8 heterocyclic ring with from about 0-2 additional heteroatoms such as nitrogen, oxygen or sulfur. Non-limiting suitable examples of suitable heterocyclic rings include piperidinyl, pyrrolidinyl, morpholino, thiomorpholino or piperazinyl. Examples of C-terminal protecting groups include but are not limited to —NH₂, —NHCH₃, —N(CH₃)₂, —NH(ethyl), —N(ethyl)₂, —N(methyl) (ethyl), —NH(benzyl), —N(C1-C4 alkyl)(benzyl), —NH(phenyl), —N(C1-C4 alkyl) (phenyl), —OCH₃, —O-(ethyl), —O-(n-propyl), —O-(n-butyl), —O-(iso-propyl), —O-(sec-butyl), —O-(t-butyl), —O-benzyl and —O-phenyl.

Substitution by Peptidomimetic Moieties

A “peptidomimetic organic moiety” can optionally be substituted for amino acid residues in the composition of this invention both as conservative and as non-conservative substitutions. These moieties are also termed “non-natural amino acids” and may optionally replace amino acid residues, amino acids or act as spacer groups within the peptides in lieu of deleted amino acids. The peptidomimetic organic moieties optionally and preferably have steric, electronic or configurational properties similar to the replaced amino acid and such peptidomimetics are used to replace amino acids in the essential positions, and are considered conservative substitutions. However such similarities are not necessarily required. According to preferred embodiments of the present invention, one or more peptidomimetics are selected such that the composition at least substantially retains its physiological activity as compared to the native protein according to the present invention.

Peptidomimetics may optionally be used to inhibit degradation of the peptides by enzymatic or other degradative processes. The peptidomimetics can optionally and preferably be produced by organic synthetic techniques. Non-limiting examples of suitable peptidomimetics include D amino acids of the corresponding L amino acids, tetrazol (Zabrocki et al., J. Am. Chem. Soc. 110:5875-5880 (1988)); isosteres of amide bonds (Jones et al., Tetrahedron Lett. 29: 3853-3856 (1988)); LL-3-amino-2-propenidone-6-carboxylic acid (LL-Acp) (Kemp et al., J. Org. Chem. 50:5834-5838 (1985)). Similar analogs are shown in Kemp et al., Tetrahedron Lett. 29:5081-5082 (1988) as well as Kemp et al., Tetrahedron Lett. 29:5057-5060 (1988), Kemp et al., Tetrahedron Lett. 29:4935-4938 (1988) and Kemp et al., J. Org. Chem. 54:109-115 (1987). Other suitable but exemplary peptidomimetics are shown in Nagal and Sato, Tetrahedron Lett. 26:647-650 (1985); Di Maio et al., J. Chem. Soc. Perkin Trans., 1687 (1985); Kahn et al., Tetrahedron Lett. 30:2317 (1989); Olson et al., J. Am. Chem. Soc. 112:323-333 (1990); and Garvey et al., J. Org. Chem. 56:436 (1990). Further suitable exemplary peptidomimetics include hydroxy-1,2,3,4-tetrahydroisoquinoline-3-carboxylate (Miyake et al., J. Takeda Res. Labs 43:53-76 (1989)); 1, 2,3,4-tetrahydro-isoquinoline-3-carboxylate (Kazmierski et al., J. Am. Chem. Soc. 133:2275-2283 (1991)); histidine isoquinolone carboxylic acid (HIC) (Zechel et al., Int. J. Pep. Protein Res. 43 (1991)); (2S,S)-methyl-phenylalanine, (2S,3R)-methyl-phenylalanine, (2R,3S)-methyl-phenylalanine and (2R,3R)-methyl-phenylalanine (Kazmierski and Hruby, Tetrahedron Lett. (1991)).

Exemplary, illustrative but non-limiting non-natural amino acids include β-amino acids (β3 and β2), homo-amino acids, cyclic amino acids, aromatic amino acids, Pro and Pyr derivatives, 3-substituted Alanine derivatives, Glycine derivatives, ring-substituted Phe and Tyr Derivatives, linear core amino acids or diamino acids. They are available from a variety of suppliers, such as Sigma-Aldrich (USA).

Protein Chemical Modifications

In the present invention, according to at least some embodiments, any part of a protein according to at least some embodiments of the invention may optionally be chemically modified, i.e. changed by addition of functional groups. For example the side amino acid residues appearing in the native sequence may optionally be modified, although as described below alternatively other parts of the protein may optionally be modified, in addition to or in place of the side amino acid residues. The modification may optionally be performed during synthesis of the molecule if a chemical synthetic process is followed, for example by adding a chemically modified amino acid. However, chemical modification of an amino acid when it is already present in the molecule (“in situ” modification) is also possible.

The amino acid of any of the sequence regions of the molecule can optionally be modified according to any one of the following exemplary types of modification (in the peptide conceptually viewed as “chemically modified”). Non-limiting exemplary types of modification include carboxymethylation, acylation, phosphorylation, glycosylation or fatty acylation. Ether bonds can optionally be used to join the serine or threonine hydroxyl to the hydroxyl of a sugar. Amide bonds can optionally be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also optionally be formed between amino acids and carbohydrates. Fatty acid acyl derivatives can optionally be made, for example, by acylation of a free amino group (e.g., lysine) (Toth et al., Peptides: Chemistry, Structure and Biology, Rivier and Marshal, eds., ESCOM Publ., Leiden, 1078-1079 (1990)).

As used herein the term “chemical modification”, when referring to a protein or peptide according to the present invention, refers to a protein or peptide where at least one of its amino acid residues is modified either by natural processes, such as processing or other post-translational modifications, or by chemical modification techniques which are well known in the art. Examples of the numerous known modifications typically include, but are not limited to: acetylation, acylation, amidation, ADP-ribosylation, glycosylation, GPI anchor formation, covalent attachment of a lipid or lipid derivative, methylation, myristoylation, pegylation, prenylation, phosphorylation, ubiquitination, or any similar process.

Other types of modifications optionally include the addition of a cycloalkane moiety to a biological molecule, such as a protein, as described in PCT Application No. WO 2006/050262, hereby incorporated by reference as if fully set forth herein. These moieties are designed for use with biomolecules and may optionally be used to impart various properties to proteins.

Furthermore, optionally any point on a protein may be modified. For example, pegylation of a glycosylation moiety on a protein may optionally be performed, as described in PCT Application No. WO 2006/050247, hereby incorporated by reference as if fully set forth herein. One or more polyethylene glycol (PEG) groups may optionally be added to O-linked and/or N-linked glycosylation. The PEG group may optionally be branched or linear. Optionally any type of water-soluble polymer may be attached to a glycosylation site on a protein through a glycosyl linker.

Altered Glycosylation

Proteins according to at least some embodiments of the invention may be modified to have an altered glycosylation pattern (i.e., altered from the original or native glycosylation pattern). As used herein, “altered” means having one or more carbohydrate moieties deleted, and/or having at least one glycosylation site added to the original protein.

Glycosylation of proteins is typically either N-linked or O-linked. N-linked refers to the attachment of the carbohydrate moiety to the side chain of an asparagine residue. The tripeptide sequences, asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-acetylgalactosamine, galactose, or xylose to a hydroxyamino acid, most commonly serine or threonine, although 5-hydroxyproline or 5-hydroxylysine may also be used.

Addition of glycosylation sites to proteins according to at least some embodiments of the invention is conveniently accomplished by altering the amino acid sequence of the protein such that it contains one or more of the above-described tripeptide sequences (for N-linked glycosylation sites). The alteration may also be made by the addition of, or substitution by, one or more serine or threonine residues in the sequence of the original protein (for O-linked glycosylation sites). The protein's amino acid sequence may also be altered by introducing changes at the DNA level.

Another means of increasing the number of carbohydrate moieties on proteins is by chemical or enzymatic coupling of glycosides to the amino acid residues of the protein. Depending on the coupling mode used, the sugars may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine. These methods are described in WO 87/05330, and in Aplin and Wriston, CRC Crit. Rev. Biochem., 22: 259-306 (1981).

Removal of any carbohydrate moieties present on proteins according to at least some embodiments of the invention may be accomplished chemically or enzymatically. Chemical deglycosylation requires exposure of the protein to trifluoromethanesulfonic acid, or an equivalent compound. This treatment results in the cleavage of most or all sugars except the linking sugar (N-acetylglucosamine or N-acetylgalactosamine), leaving the amino acid sequence intact.

Chemical deglycosylation is described by Hakimuddin et al., Arch. Biochem. Biophys., 259: 52 (1987); and Edge et al., Anal. Biochem., 118: 131 (1981). Enzymatic cleavage of carbohydrate moieties on proteins can be achieved by the use of a variety of endo- and exo-glycosidases as described by Thotakura et al., Meth. Enzymol., 138: 350 (1987).

Uses of Proteins and Pharmaceutical Compositions

The term “treatment” as used herein, refers to both therapeutic treatment and prophylactic or preventative measures, which in this Example relates to treatment of cancer; however, also as described below, uses of VSTM5 therapeutic agents and pharmaceutical compositions are also provided for treatment of infectious disease, sepsis, and/or autoimmune conditions, and/or for inhibiting an undesirable immune activation that follows gene therapy. Those in need of treatment include those already with cancer as well as those in which the cancer is to be prevented. Hence, the mammal to be treated herein may have been diagnosed as having the cancer or may be predisposed or susceptible to the cancer. As used herein the term “treating” refers to preventing, delaying the onset of, curing, reversing, attenuating, alleviating, minimizing, suppressing, halting the deleterious effects or stabilizing of discernible symptoms of the above-described cancerous diseases, disorders or conditions. It also includes managing the cancer as described above. By “manage” it is meant reducing the severity of the disease, reducing the frequency of episodes of the disease, reducing the duration of such episodes, reducing the severity of such episodes, slowing/reducing cancer cell growth or proliferation, slowing progression of at least one symptom, ameliorization of at least one measurable physical parameter and the like.

“Mammal” for purposes of treatment refers to any animal classified as a mammal, including humans, domestic and farm animals, and zoo, sports, or pet animals, such as dogs, horses, cats, cows, etc. Preferably, the mammal is human. Preferably the mammal is a human which is diagnosed with one of the disease, disorder or conditions described hereinabove, or alternatively are predisposed to at least one type of cancer.

The term “therapeutically effective amount” refers to an amount of agent according to the present invention that is effective to treat a disease or disorder in a mammal. The therapeutic agents of the present invention can be provided to the subject alone or as part of a pharmaceutical composition where they are mixed with a pharmaceutically acceptable carrier.

As used herein “VSTM5 therapeutic agent” is any one of the VSTM5 ECD polypeptides or fragments thereof, comprising a fragment of VSTM5 ECD, consisting essentially of an amino acid sequence as set forth in any one of SEQ ID NOs: 1-3, 12-110, or variant thereof that possesses at least 95% sequence identity therewith, a fusion proteins comprising same, nucleic acid sequences encoding same, expression vector, a host cell, and/or a pharmaceutical composition comprising same, further comprising a pharmaceutically acceptable diluent or carrier, adapted for treatment of any of the diseases as described herein.

A “therapeutically effective dosage” of VSTM5 therapeutic agent according to at least some embodiments of the present invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of VSTM5 positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

Cancer Immunotherapy

Unlike tumor-targeted therapies, which are aimed at inhibiting molecular pathways that are crucial for tumor growth and development, and/or depleting tumor cells, cancer immunotherapy is aimed to stimulate the patient's own immune system to eliminate cancer cells, providing long-lived tumor destruction. Various approaches can be used in cancer immunotherapy; among them are therapeutic cancer vaccines to induce tumor-specific T cell responses, and immunostimulatory antibodies (i.e. antagonists of inhibitory receptors=immune checkpoints) to remove immunosuppressive pathways.

Clinical responses with targeted therapy or conventional anti-cancer therapies tend to be transient as cancer cells develop resistance, and tumor recurrence takes place. However, the clinical use of cancer immunotherapy in the past few years has shown that this type of therapy can have durable clinical responses, showing dramatic impact on long term survival. However, although responses are long term, only a small number of patients respond (as opposed to conventional or targeted therapy, where a large number of patients respond, but responses are transient).

By the time a tumor is detected clinically, it has already evaded the immune-defense system by acquiring immunoresistant and immunosuppressive properties and creating an immunosuppressive tumor microenvironment through various mechanisms and a variety of immune cells. Thus, in cancer immunotherapy it is becoming increasingly clear that a combination of therapies is be required for clinical efficacy.

Combination approaches are needed and expected to increase the number of patients benefiting from immunotherapy and expand the number and types of cancers that are responsive, expanding the potential cancer indications for checkpoint agents well beyond the initial indications currently showing efficacy of immune checkpoint blockade as monotherapy. The combination of immunomodulatory approaches is meant to maximize the outcomes and overcome the resistance mechanisms of most tumors to a single approach. Thus, tumors traditionally thought of as non-immunogenic can likely become immunogenic and respond to immunotherapy though co-administration of pro-immunogenic therapies designed to increase the patient's anti-tumor immune responses. Potential priming agents are detailed herein below.

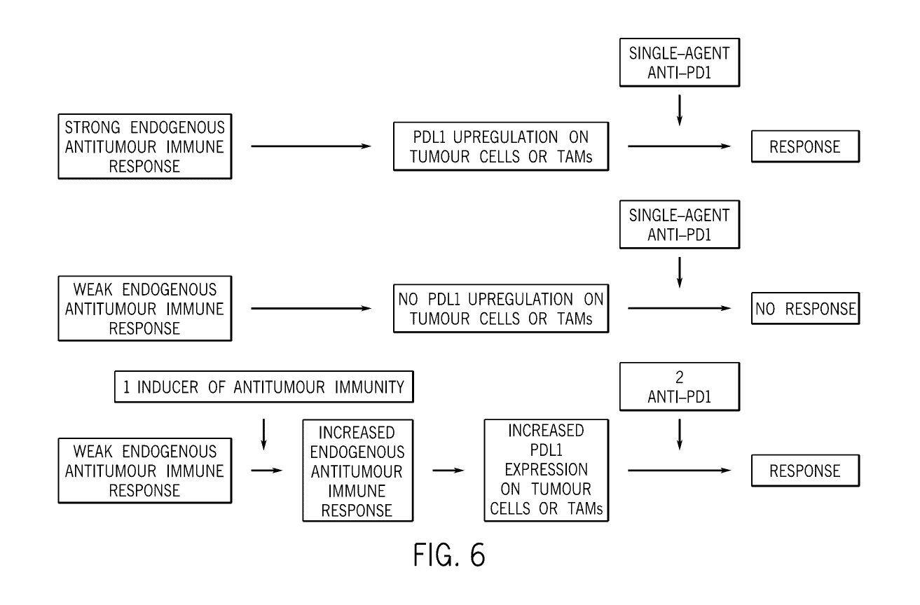

Without wishing to be limited by a single hypothesis, the underlying scientific rationale for the dramatic increased efficacy of combination therapy considers that immune checkpoint blockade as a monotherapy will induce tumor regressions only when there is pre-existing strong anti-tumor immune response to be ‘unleashed’ when the pathway is blocked. However, in most patients and tumor types the endogenous anti-tumor immune responses are weak, and thus the induction of anti-tumor immunity is required for the immune checkpoint blockade to be effective, as shown in the FIG. 6 (which depicts the case of the PDL-1/PD-1 immune checkpoint). As can be appreciated from FIG. 6, the endogenous expression of the immune checkpoint ligand (PDL-1 in this case) is elevated by the induction of anti-tumor immunity, and thus expression in the patient's original tumor is not a prerequisite for the combination therapy to be effective.

According to at least some embodiments of the present invention, immunostimulatory VSTM5 therapeutic agents and/or a pharmaceutical composition comprising same, are used for treatment of all types of cancer in cancer immunotherapy in combination therapy.

Immunostimulatory VSTM5 therapeutic agent and/or a pharmaceutical composition comprising same, according to at least some embodiments of the present invention can be administered in combination with other potentiating agents and/or other therapies. According to at least some embodiments, the immunostimulatory VSTM5 therapeutic agent could be used in combination with any of the known in the art standard of care cancer treatment (as can be found, for example, in http://www.cancer.gov/cancer topics).

For example, the combination therapy can include an immunostimulatory VSTM5 therapeutic agent and/or a pharmaceutical composition comprising same, combined with at least one other therapeutic or immune modulatory agent, other compounds or immunotherapies, or immunostimulatory strategy, including, but not limited to, tumor vaccines, adoptive T cell therapy, Treg depletion, antibodies (e.g. bevacizumab, Erbitux, Ipilimumab), peptides, pepti-bodies, small molecules, chemotherapeutic agents such as cytotoxic and cytostatic agents (e.g. paclitaxel, cisplatin, vinorelbine, docetaxel, gemcitabine, temozolomide, irinotecan, SFU, carboplatin), immunological modifiers such as interferons and interleukins, immunostimulatory antibodies, growth hormones or other cytokines, folic acid, vitamins, minerals, aromatase inhibitors, RNAi, Histone Deacetylase Inhibitors, proteasome inhibitors, and so forth. In another example, the combination therapy can include an anti-VSTM5 antibody or VSTM5 modulating agent, such as a small molecule such as a peptide, ribozyme, aptamer, siRNA, or other drug that binds VSTM5, combined with at least one other therapeutic or immune modulatory agent.

According to at least some embodiments of the present invention, therapeutic agents that can be used in combination with immunostimulatory VSTM5 therapeutic agents, are potentiating agents that enhance anti-tumor responses.

Various strategies are available for combining an immunostimulatory VSTM5 therapeutic agent and/or a pharmaceutical composition comprising same with potentiating agents for cancer immunotherapy. According to at least some embodiments of the present invention, immunostimulatory VSTM5 therapeutic agent and/or a pharmaceutical composition comprising same for cancer immunotherapy is used in combination with potentiating agents that are primarily geared to increase endogenous anti-tumor responses, such as radiotherapy, cryotherapy, conventional/classical chemotherapy potentiating anti-tumor immune responses, targeted therapy potentiating anti-tumor immune responses, anti-angiogenic therapy, therapeutic agents targeting immunosuppressive cells such as Tregs and MDSCs, immunostimulatory antibodies, cytokine therapy, therapeutic cancer vaccines, and/or adoptive cell transfer.

Without wishing to be limited by a single hypothesis, the scientific rationale behind the combined use with some chemotherapy or anti-cancer conventional drugs is that cancer cell death, a consequence of the cytotoxic action of most chemotherapeutic compounds, may result in increased levels of tumor antigen leading to enhanced antigen presentation and stimulation of anti-tumor immune responses (i.e., immunogenic cell death), resulting in potentiating effects with the immunostimulatory VSTM5 therapeutic agent (Zitvogel et al., J. Clin. Invest. 118:1991-2001 (2008), Galluzzi et al., Nat Rev Drug Discov. February 3; 11(3):215-33(2012)) Other combination therapies that may potentiate anti-tumor responses through tumor cell death are radiotherapy, cryotherapy, surgery, and hormone deprivation. Each of these cancer therapies creates a source of tumor antigen in the host.

According to at least some embodiments of the invention, classical chemotherapies and conventional anti-cancer therapies as agents potentiating anti-tumor immune responses for combination with immunostimulatory VSTM5 therapeutic agent are selected from the group consisting of but not limited to: Platinum based compounds such as oxaliplatin, cisplatin, carboplatin; Antibiotics with anti-cancer activity, such as dactinomycin, bleomycin, mitomycin-C, mithramycin and Anthracyclines, such as doxorubicin, daunorubicin, epirubicin, idarubicin; Anthracenediones, such as mitoxantrone; Alkylating agents, such as dacarbazine, melphalan, cyclophosphamide, temozolomide, chlorambucil, busulphan, nitrogen mustard, nitrosoureas; Antimetabolites, such as fluorouracil, raltitrexed, gemcitabine, cytosine arabinoside, hydroxyurea and Folate antagonists, such as methotrexate, trimethoprim, pyrimethamine, pemetrexed; Antimitotic agents such as polokinase inhibitors and Microtubule inhibitors, such as Taxanes and Taxoids, such as paclitaxel, docetaxel; Vinca alkaloids such as vincristine, vinblastine, vindesine, vinorelbine; Topoisomerase inhibitors, such as etoposide, teniposide, amsacrine, topotecan, irinotecan, camptothecin; Cytostatic agents including Antioestrogens such as tamoxifen, fulvestrant, toremifene, raloxifene, droloxifene, iodoxyfene, Antiandrogens such as bicalutamide, flutamide, nilutamide and cyproterone acetate, Progestogens such as megestrol acetate, Aromatase inhibitors such as anastrozole, letrozole, vorozole, exemestane; GnRH analogs, such as leuprorelin, goserelin, buserelin, degarelix; and inhibitors of 5α-reductase such as finasteride.

According to at least some embodiments of the present invention, immunostimulatory VSTM5 therapeutic agent and/or a pharmaceutical composition comprising same for cancer immunotherapy is used in combination with Bisphosphonates, especially amino-bisphosphonates (ABP), which have shown to have anti-cancer activity. Some of the activities associated with ABPs are on human γδT cells that straddle the interface of innate and adaptive immunity and have potent anti-tumour activity.

Targeted therapies can also stimulate tumor-specific immune response by inducing the immunogenic death of tumor cells or by engaging immune effector mechanisms (Galluzzi et al, Nat Rev Drug Discov. 1(3):215-33(2012)), Vanneman and Dranoff, Nat Rev Cancer 12(4):237-51(2012)). In addition, according to at least some embodiments of the present invention, immunostimulatory VSTM5 therapeutic agent and/or a pharmaceutical composition comprising same for cancer immunotherapy is used in combination with any of the following: certain therapeutic monoclonal antibodies, trastuzumab, that favor the generation of tumor-specific cytotoxic CD8⁺ T cells, and NK cells infiltration to the tumor and NK cell mediated cytotoxicity; certain tyrosine kinase inhibitors (TKIs) that promote cancer-directed immune responses by increasing MHC class II expression, decreased levels of tumor infiltrating immunosuppressive cells—Tregs and MDScs, reducing the expression of the immunosuppressive enzyme IDO by tumor cells, and/or inhibition of DC functions; Histone deacetylase (HDAC) inhibitors which were found increase the expression of NK-activating receptor ligands on the surface of cancer cells, thereby facilitating tumor cell recognition by NK cells, while proteasome inhibitors were found to sensitize tumor cells to CTL-mediated or NK-mediated cell lysis.

According to at least some embodiments of the invention, Targeted therapies used as agents for combination with immunostimulatory VSTM5 therapeutic agent and/or a pharmaceutical composition comprising same for treatment of cancer are selected from the group consisting of but not limited to: histone deacetylase (HDAC) inhibitors, such as vorinostat, romidepsin, panobinostat, belinostat, mocetinostat, abexinostat, entinostat, resminostat, givinostat, quisinostat, sodium butyrate; Proteasome inhibitors, such as bortezomib, carfilzomib, disulfiram; mTOR pathway inhibitors, such as temsirolimus, rapamycin, everolimus; PI3K inhibitors, such as perifosine, CAL101, PX-866, IPI-145, BAY 80-6946; B-raf inhibitors such as vemurafenib, sorafenib; JAK2 inhibitors, such as lestaurtinib, pacritinib; Tyrosine kinase inhibitors (TKIs), such as erlotinib, imatinib, sunitinib, lapatinib, gefitinib, sorafenib, nilotinib, toceranib, bosutinib, neratinib, vatalanib, regorafenib, cabozantinib; other Protein kinase inhibitors, such as crizotinib; Inhibitors of serine/threonine kinases for example Ras/Raf signalling inhibitors such as farnesyl transferase inhibitors; Inhibitors of serine proteases for example matriptase, hepsin, urokinase; Inhibitors of intracellular signaling such as tipifarnib, perifosine; Inhibitors of cell signalling through MEK and/or AKT kinases; aurora kinase inhibitors such as AZD1152, PH739358, VX-680, MLN8054, R763, MP235, MP529, VX-528, AX39459; Cyclin dependent kinase inhibitors such as CDK2 and/or CDK4 inhibitors; Inhibitors of survival signaling proteins including Bcl-2, Bcl-XL, such as ABT-737; HSP90 inhibitors; Therapeutic monoclonal antibodies, such as anti-EGFR mAbs cetuximab, panitumumab, nimotuzumab, anti-ERBB2 mAbs trastuzumab, pertuzumab, anti-CD20 mAbs such as rituximab, ofatumumab, veltuzumab and mAbs targeting other tumor antigens such as alemtuzumab, labetuzumab, adecatumumab, oregovomab, onartuzumab; TRAIL pathway agonists, such as dulanermin (soluble rhTRAIL), apomab, mapatumumab, lexatumumab, conatumumab, tigatuzumab; Antibody fragments, bi-specific antibodies and bi-specific T-cell engagers (BiTEs), such as catumaxomab, blinatumomab; Antibody drug conjugates (ADC) and other immunoconjugates, such as ibritumomab triuxetan, tositumomab, brentuximab vedotin, gemtuzumab ozogamicin, clivatuzumab tetraxetan, pemtumomab, trastuzumab emtansine; Anti-angiogenic therapy such as bevacizumab, etaracizumab, volociximab, ramucirumab, aflibercept, sorafenib, sunitinib, regorafenib, axitinib, nintedanib, motesanib, pazopanib, cediranib; Metalloproteinase inhibitors such as marimastat; Inhibitors of urokinase plasminogen activator receptor function; Inhibitors of cathepsin activity and combinations of any of the foregoing.

Other cancer immunotherapies that also increase endogenous anti-tumor responses could also potentiate the effect of the immunostimulatory VSTM5 therapeutic agent by enhancing immune effector mechanisms, such as Adoptive T cell therapy, Therapeutic cancer vaccines, reduced immune suppressive cells and their function, Cytokine therapy, Immunostimulatory antibodies and combinations of any of the foregoing.

According to at least some embodiments of the present invention, immunostimulatory VSTM5 therapeutic agent and/or a pharmaceutical composition comprising same for cancer immunotherapy is used in combination with Therapeutic agents targeting regulatory immunosuppressive cells such as regulatory T cells (Tregs) and myeloid derived suppressor cells (MDSCs). A number of commonly used chemotherapeutics exert non-specific targeting of Tregs and reduce the number or the immunosuppressive capacity of Tregs or MDSCs (Facciabene et al Cancer Res; 72:2162-2171 (2012); Byrne et al, Cancer Res; 71(22); 1-6 (2011); Gabrilovich and Nagaraj Nat Rev. Immunol. 4:941-52 (2009)). In this regard, metronomic therapy with some chemotherapy drugs results in immunostimulatory rather than immunosuppressive effects, via modulation of regulatory cells (e.g., Tregs) or MDSCs. Thus, according to at least some embodiments of the present invention, immunostimulatory VSTM5 therapeutic agent and/or a pharmaceutical composition comprising same for cancer immunotherapy is used in combination with drugs selected from but not limited to cyclophosphamide, gemcitabine, mitoxantrone, fludarabine, fludarabine, docetaxel, paclitaxel, thalidomide, thalidomide derivatives and combinations of any of the foregoing.

In addition, according to at least some embodiments of the present invention, immunostimulatory VSTM5 therapeutic agent and/or a pharmaceutical composition comprising same for cancer immunotherapy is used in combination with novel Treg-specific targeting agents including: 1) depleting or killing antibodies that directly target Tregs through recognition of Treg cell surface receptors such as anti-CD25 mAbs daclizumab, basiliximab; 2) ligand-directed toxins such as denileukin diftitox (Ontak)—a fusion protein of human IL-2 and diphtheria toxin, or LMB-2—a fusion between an scFv against CD25 and the Pseudomonas exotoxin; and 3) antibodies targeting Treg cell surface receptors such as CTLA4, PD-1, OX40 and GITR.

According to at least some embodiments of the present invention, immunostimulatory VSTM5 therapeutic agent and/or a pharmaceutical composition comprising same for cancer immunotherapy is used in combination with any of the options described below for disrupting Treg induction and/or function, including TLR (toll like receptors) agonists; agents that interfere with the adenosinergic pathway, such as ectonucleotidase inhibitors, or inhibitors of the A2A adenosine receptor; TGF-β inhibitors, such as fresolimumab, lerdelimumab, metelimumab, trabedersen, LY2157299, and LY210976; or blockade of Tregs recruitment to tumor tissues including chemokine receptor inhibitors, such as the CCR4/CCL2/CCL22 pathway.

According to at least some embodiments of the present invention, immunostimulatory VSTM5 therapeutic agent and/or a pharmaceutical composition comprising same for cancer immunotherapy is used in combination with any of the options described below for inhibiting the immunosuppressive tumor microenvironment, including inhibitors of cytokines and enzymes which exert immunosuppressive activities, such as IDO (indoleamine-2,3-dioxygenase) inhibitors; inhibitors of anti-inflammatory cytokines which promote an immunosuppressive microenvironment, such as IL-10, IL-35, IL-4 and IL-13; and Bevacizumab which reduces Tregs and favors the differentiation of DCs.

According to at least some embodiments of the present invention, immunostimulatory VSTM5 therapeutic agent and/or a pharmaceutical composition comprising same for cancer immunotherapy is used in combination with any of the options described below for targeting MDSCs (myeloid-derived suppressive cells), including promoting their differentiation into mature myeloid cells that do not have suppressive functions by Vitamin D3, or Vitamin A metabolites, such as retinoic acid, all-trans retinoic acid (ATRA); inhibition of MDSCs suppressive activity by COX2 inhibitors, and phosphodiesterase 5 inhibitors like sildenafil, ROS inhibitors such as nitroaspirin.

According to at least some embodiments of the present invention, immunostimulatory VSTM5 therapeutic agent and/or a pharmaceutical composition comprising same for cancer immunotherapy is used in combination with Immunostimulatory antibodies as agents potentiating anti-tumor immune responses (Pardoll Nature Reviews Cancer, 12:252-64 (2012)).

Immunostimulatory antibodies promote anti-tumor immunity by directly modulating immune functions, i.e., by blocking other inhibitory targets or enhancing immunostimulatory proteins. According to at least some embodiments of the present invention, immunostimulatory VSTM5 therapeutic agent and/or a pharmaceutical composition comprising same for cancer immunotherapy is used in combination with antagonistic antibodies targeting immune checkpoints including anti-CTLA4 mAbs, such as ipilimumab, tremelimumab; Anti-PD-1 such as nivolumab BMS-936558/MDX-1106/ONO-4538, AMP224, CT-011, MK-3475; anti-PDL-1 antagonists such as BMS-936559/MDX-1105, MEDI4736, RG-7446/MPDL3280A; anti-LAG-3 such as IMP-321), anti-TIM-3, anti-BTLA, anti-B7-H4, anti-B7-H3, anti-VISTA; agonistic antibodies targeting immunostimulatory proteins, including anti-CD40 mAbs such as CP-870,893, lucatumumab, dacetuzumab; Anti-CD137 mAbs such as BMS-663513 urelumab, PF-05082566; Anti-OX40 mAbs, such as anti-OX40; anti-GITR mAbs such as TRX518; Anti-CD27 mAbs, such as CDX-1127; Anti-ICOS mAbs and combinations of any of the foregoing.

Cytokines are molecular messengers that allow the cells of the immune system to communicate with one another to generate a coordinated, robust, but self-limited response to a target antigen. Cytokine-based therapies embody a direct attempt to stimulate the patient's own immune system to reject cancer. The growing interest over the past two decades in harnessing the immune system to eradicate cancer has been accompanied by heightened efforts to characterize cytokines and exploit their vast signaling networks to develop cancer treatments. Cytokines directly stimulate immune effector cells and stromal cells at the tumor site and enhance tumor cell recognition by cytotoxic effector cells. Numerous animal tumor model studies have demonstrated that cytokines have broad anti-tumor activity and this has been translated into a number of cytokine-based approaches for cancer therapy (Lee and Margolin Cancers 3: 3856-3893 (2011)). A number of cytokines are in preclinical or clinical development as agents potentiating anti-tumor immune responses for cancer immunotherapy, including among others: IL-2, IL-7, IL-12, IL-15, IL-17, IL-18 and/or IL-21, IL-23, IL-27, GM-CSF, IFNα (interferon α), IFNβ, IFNγ and combinations of any of the foregoing.

Several cytokines have been approved for therapy of cancer and many more are under development. However, therapeutic efficacy is often hampered by severe side effects and poor pharmacokinetic properties. Thus, in addition to systemic administration of cytokines, a variety of strategies can be employed for the delivery of therapeutic cytokines and their localization to the tumor site, in order to improve their pharmacokinetics, as well as their efficacy and/or toxicity, including antibody-cytokine fusion molecules (immunocytokines), chemical conjugation to polyethylene glycol (PEGylation), transgenic expression of cytokines in autologous whole tumor cells, incorporation of cytokine genes into DNA vaccines, recombinant viral vectors to deliver cytokine genes, etc. In the case of immunocytokines, fusion of cytokines to tumor-specific antibodies or antibody fragments allows for targeted delivery and therefore improved efficacy and pharmacokinetics, and reduced side effects.

According to at least some embodiments of the present invention, immunostimulatory VSTM5 therapeutic agent and/or a pharmaceutical composition comprising same for cancer immunotherapy is used in combination with Cytokine therapy, involving the use of cytokines as agents potentiating anti-tumor immune responses, including cytokines such as IL-2, IL-7, IL-12, IL-15, IL-17, IL-18 and IL-21, IL-23, IL-27, GM-CSF, IFNα, IFNα-2b, IFNβ, IFNγ, and their different strategies for delivery, as described above.

Cancer vaccines are used to treat existing cancer (therapeutic) or prevent the development of cancer in certain high-risk individuals (prophylactic). Therapeutic cancer vaccines allow for improved priming of T cells and improved antigen presentation, and can be used as therapeutic agents for potentiating anti-tumor immune responses (Mellman et al., Nature. 2011 Dec. 21; 480(7378):480-9.; Schlom J Natl Cancer Inst. 104(8):599-613 (2012)).

Several types of therapeutic cancer vaccines are in preclinical and clinical development. These include for example:
1) Whole tumor cell vaccines, in which cancer cells removed during surgery are treated to enhance their immunogenicity, and injected into the patient to induce immune responses against antigens in the tumor cells. The tumor cell vaccine can be autologous, i.e. a patient's own tumor, or allogeneic which typically contain two or three established and characterized human tumor cell lines of a given tumor type, such as the GVAX vaccine platforms.
2) Tumor antigen vaccines, in which a tumor antigen (or a combination of a few tumor antigens), usually proteins or peptides, are administered to boost the immune system (possibly with an adjuvant and/or with immune modulators or attractants of dendritic cells such as GM-CSF). The tumor antigens may be specific for a certain type of cancer, but they are not made for a specific patient.
3) Vector-based tumor antigen vaccines and DNA vaccines can be used as a way to provide a steady supply of antigens to stimulate an anti-tumor immune response. Vectors encoding for tumor antigens are injected into the patient (possibly with proinflammatory or other attractants such as GM-CSF), taken up by cells in vivo to make the specific antigens, which would then provoke the desired immune response. Vectors may be used to deliver more than one tumor antigen at a time, to increase the immune response. In addition, recombinant virus, bacteria or yeast vectors should trigger their own immune responses, which may also enhance the overall immune response.
4) Oncolytic virus vaccines, such as OncoVex/T-VEC, which involves the intratumoral injection of replication-conditional herpes simplex virus which preferentially infects cancer cells. The virus, which is also engineered to express GM-CSF, is able to replicate inside a cancer cell causing its lysis, releasing new viruses and an array of tumor antigens, and secreting GM-CSF in the process. Thus, such oncolytic virus vaccines enhance DCs function in the tumor microenvironment to stimulate anti-tumor immune responses.
5) Dendritic cell vaccines (Palucka and Banchereau Nat. Rev. Cancer 12(4):265-77 (2012)): Dendritic cells (DCs) phagocytose tumor cells and present tumor antigens to tumor specific T cells. In this approach, DCs are isolated from the cancer patient and primed for presenting tumor-specific T cells. To this end several methods can be used: DCs are loaded with tumor cells or lysates; DCs are loaded with fusion proteins or peptides of tumor antigens; coupling of tumor antigens to DC-targeting mAbs. The DCs are treated in the presence of a stimulating factor (such as GM-CSF), activated and matured ex vivo, and then re-infused back into the patient in order provoke an immune response to the cancer cells. Dendritic cells can also be primed in vivo by injection of patients with irradiated whole tumor cells engineered to secrete stimulating cytokines (such as GM-CSF). Similar approaches can be carried out with monocytes. Sipuleucel-T (Provenge), a therapeutic cancer vaccine which has been approved for treatment of advanced prostate cancer, is an example of a dendritic cell vaccine.

Thus, according to at least some embodiments of the present invention, immunostimulatory VSTM5 therapeutic agent and/or a pharmaceutical composition comprising same for cancer immunotherapy is used in combination with Therapeutic cancer vaccines. Non limiting examples of such therapeutic cancer vaccines include Whole tumor cell vaccines, Tumor antigen vaccines, Vector-based vaccines, Oncolytic virus vaccines, Dendritic-cell vaccines, as described above.

One approach to cancer immunotherapy is based on adoptive T cell therapy or adoptive cell transfer (ACT), which involves the ex vivo identification and expansion of autologous naturally occurring tumor specific T cells, which are then adoptively transferred back into the cancer patient (Restifo et al., Nat Rev Immunol., 12(4):269-81 (2012)). Cells that are infused back into a patient after ex vivo expansion can traffic to the tumor and mediate its destruction. Prior to this adoptive transfer, hosts can be immunodepleted by irradiation and/or chemotherapy. The combination of lymphodepletion, adoptive cell transfer, and a T cell growth factor (such as IL-2); can lead to prolonged tumor eradication in tumor patients. A more novel approach involves the ex vivo genetic modification of normal peripheral blood T cells to confer specificity for tumor-associated antigens. For example, clones of TCRs of T cells with particularly good anti-tumor responses can be inserted into viral expression vectors and used to infect autologous T cells from the patient to be treated. Another option is the use of chimeric antigen receptors (CARs) which are essentially a chimeric immunoglobulin-TCR molecule, also known as a T-body. CARs have antibody-like specificities and recognize MHC-nonrestricted structures on the surface of target cells (the extracellular target-binding module), grafted onto the TCR intracellular domains capable of activating T cells (Restifo et al., Nat Rev Immunol.12(4):269-81 (2012), and Shi et al., Cancer Lett. 328(2):191-7) (2013).

According to at least some embodiments of the present invention, immunostimulatory VSTM5 therapeutic agent and/or a pharmaceutical composition comprising same for cancer immunotherapy is used in combination with Adoptive cell transfer to potentiate anti-tumor immune responses, including genetically modified T cells, as described above.

The immunostimulatory VSTM5 therapeutic agent and/or a pharmaceutical composition comprising same can be co-administered together with one or more other therapeutic agents, which acts in conjunction with or synergistically with the composition according to at least some embodiments of the present invention to treat or prevent the cancer. The immunostimulatory VSTM5 therapeutic agent and/or a pharmaceutical composition comprising same and the one or more other therapeutic agents can be administered in either order or simultaneously. The other therapeutic agents are for example, a cytotoxic agent, a radiotoxic agent or an immunosuppressive agent. The composition can be linked to the agent (as an immunocomplex) or can be administered separately from the agent. In the latter case (separate administration), the composition can be administered before, after or concurrently with the agent or can be co-administered with other known therapies, e.g., an anti-cancer therapy, e.g., radiation. Such therapeutic agents include, among others, anti-neoplastic agents such as doxorubicin (Adriamycin), cisplatin bleomycin sulfate, carmustine, chlorambucil, and cyclophosphamide hydroxyurea which, by themselves, are only effective at levels which are toxic or subtoxic to a patient. Cisplatin is intravenously administered as a 100 mg/dose once every four weeks and Adriamycin is intravenously administered as a 60-75 mg/ml dose once every 21 days. Co-administration of the human anti-VSTM5 antibodies, or antigen binding fragments and/or alternative scaffolds thereof, according to at least some embodiments of the present invention with chemotherapeutic agents provides two anti-cancer agents which operate via different mechanisms which yield a cytotoxic effect to human tumor cells. Such co-administration can solve problems due to development of resistance to drugs or a change in the antigenicity of the tumor cells which would render them unreactive with the antibody. In other embodiments, the subject can be additionally treated with an agent that modulates, e.g., enhances or inhibits, the expression or activity of Fcy or Fcy receptors by, for example, treating the subject with a cytokine. Preferred cytokines for administration during treatment with the multispecific molecule include of granulocyte colony-stimulating factor (G-CSF), granulocyte-macrophage colony-stimulating factor (GM-CSF), interferon-γ (IFN-γ), and tumor necrosis factor (TNF).

Target-specific effector cells, e.g., effector cells linked to compositions (e.g., human antibodies, multispecific and bispecific molecules) according to at least some embodiments of the present invention can also be used as therapeutic agents. Effector cells for targeting can be human leukocytes such as macrophages, neutrophils or monocytes. Other cells include eosinophils, natural killer cells and other IgG- or IgA-receptor bearing cells. If desired, effector cells can be obtained from the subject to be treated. The target-specific effector cells can be administered as a suspension of cells in a physiologically acceptable solution. The number of cells administered can be in the order of 10⁻⁸ to 10⁻⁹ but will vary depending on the therapeutic purpose. In general, the amount will be sufficient to obtain localization at the target cell, e.g., a tumor cell expressing VSTM5 proteins, and to effect cell killing by, e.g., phagocytosis. Routes of administration can also vary.

Therapy with target-specific effector cells can be performed in conjunction with other techniques for removal of targeted cells. For example, anti-tumor therapy using the immunostimulatory VSTM5 therapeutic agent and/or a pharmaceutical composition comprising same according to at least some embodiments of the present invention and/or effector cells armed with these compositions can be used in conjunction with chemotherapy. Additionally, combination immunotherapy may be used to direct two distinct cytotoxic effector populations toward tumor cell rejection. For example, immunostimulatory VSTM5 therapeutic agent linked to anti-Fc-γ RI or anti-CD3 may be used in conjunction with IgG- or IgA-receptor specific binding agents.

Bispecific and multispecific molecules according to at least some embodiments of the present invention can also be used to modulate FcγR or FcγR levels on effector cells, such as by capping and elimination of receptors on the cell surface. Mixtures of anti-F_{c} receptors can also be used for this purpose.

The immunostimulatory VSTM5 therapeutic agent and/or a pharmaceutical composition comprising same according to at least some embodiments of the present invention which have complement binding sites, such as portions from IgG1, IgG2, or IgG3 or IgM which bind complement, can also be used in the presence of complement. In one embodiment, ex vivo treatment of a population of cells comprising target cells with a binding agent according to at least some embodiments of the present invention and appropriate effector cells can be supplemented by the addition of complement or serum containing complement. Phagocytosis of target cells coated with a binding agent according to at least some embodiments of the present invention can be improved by binding of complement proteins. In another embodiment target cells coated with the compositions (e.g., human antibodies, multispecific and bispecific molecules) according to at least some embodiments of the present invention can also be lysed by complement. In yet another embodiment, the compositions according to at least some embodiments of the present invention do not activate complement.

The immunostimulatory VSTM5 therapeutic agent and/or a pharmaceutical composition comprising same according to at least some embodiments of the present invention can also be administered together with complement. Thus, according to at least some embodiments of the present invention there are compositions, comprising human antibodies, multispecific or bispecific molecules and serum or complement. These compositions are advantageous in that the complement is located in close proximity to the human antibodies, multispecific or bispecific molecules. Alternatively, the human antibodies, multispecific or bispecific molecules according to at least some embodiments of the present invention and the complement or serum can be administered separately.

According to an additional aspect of the present invention the immunostimulatory VSTM5 therapeutic agents can be used to prevent pathologic inhibition of T cell activity, such as that directed against cancer cells.

According to an additional aspect of the present invention the immunostimulatory VSTM5 therapeutic agents can be used to inhibit T cell activation, as can be manifested for example by T cell proliferation and cytokine secretion.

Thus, according to an additional aspect of the present invention there is provided a method of treating cancer as recited herein, and/or for promoting immune stimulation mediated by the VSTM5 polypeptide in a subject by administering to a subject in need thereof an effective amount of any one of the immunostimulatory VSTM5 therapeutic agents.

According to at least some embodiments, immune cells, preferably T cells can be contacted in vivo or ex vivo with the immunostimulatory VSTM5 therapeutic agents to modulate immune responses. The T cells contacted with the VSTM5 therapeutic agents can be any cell which expresses the T cell receptor, including α/β and γ/δ T cell receptors. T-cells include all cells which express CD3, including T-cell subsets which also express CD4 and CD8. T-cells include both naive and memory cells and effector cells such as CTLs. T-cells also include cells such as Th1, Tc1, Th2, Tc2, Th3, Th17, Th22, Treg, and Tr1 cells. T-cells also include NKT-cells and similar unique classes of the T-cell lineage.

VSTM5 blockade may also be combined with standard cancer treatments. VSTM5 blockade may be effectively combined with chemotherapeutic regimes. In these instances, it may be possible to reduce the dose of chemotherapeutic reagent administered. An example of such a combination is VSTM5 therapeutic agent in combination with Temsirolimus for the treatment of late stage renal cell cancer. Another example of such a combination is a VSTM5 therapeutic agent in combination with interleukin-2 (IL-2) for the treatment of late stage renal cell cancer, as well as combination with Ipilimumab or BMS-936558. The scientific rationale behind the combined use of VSTM5 blockade and chemotherapy is that cell death, that is a consequence of the cytotoxic action of most chemotherapeutic compounds, should result in increased levels of tumor antigen in the antigen presentation pathway. Other combination therapies that may result in synergy with VSTM5 blockade through cell death are radiotherapy, cryotherapy, surgery, and hormone deprivation. Other additional combination therapies with additional immunomodulatory molecules will synergistically contribute to the stimulation of the immune system to eradicate the cancer. Each of these protocols creates a source of tumor antigen in the host. Angiogenesis inhibitors may also be combined with VSTM5 blockade Inhibition of angiogenesis leads to tumor cell death which may feed tumor antigen into host antigen presentation pathways.

VSTM5 blocking therapeutic agents according to at least some embodiments of the invention can also be used in combination with bispecific antibodies that target Fcα or Fcγ receptor-expressing effectors cells to tumor cells (see, e.g., U.S. Pat. Nos. 5,922,845 and 5,837,243). Bispecific antibodies can be used to target two separate antigens. For example anti-Fc receptor/anti-tumor antigen (e.g., Her-2/neu) bispecific antibodies have been used to target macrophages to sites of tumor. This targeting may more effectively activate tumor specific responses. The T cell arm of these responses would be augmented by the use of VSTM5 blockade. Alternatively, antigen may be delivered directly to DCs by the use of bispecific antibodies which bind to tumor antigen and a dendritic cell specific cell surface marker.

Tumors evade host immune surveillance by a large variety of mechanisms. Many of these mechanisms may be overcome by the inactivation of proteins which are expressed by the tumors and which are immunosuppressive. These include among others TGF-β (Kehrl, J. et al. J. Exp. Med. 163: 1037-1050 (1986)), IL-10 (Howard, M. & O'Garra, A. Immunology Today 13: 198-200 (1992)), and Fas ligand (Hahne, M. et al. Science 274: 1363-1365 (1996)). Antibodies to each of these entities may be used in combination with VSTM5 therapeutic agents to counteract the effects of the immunosuppressive agent and favor tumor immune responses by the host.

Other antibodies which may be used to activate host immune responsiveness can be used in combination with immunostimulatory VSTM5 therapeutic agents according to at least some embodiments of the invention. These include molecules on the surface of dendritic cells which activate DC function and antigen presentation. Anti-CD40 antibodies are able to substitute effectively for T cell helper activity (Ridge, J. et al. Nature 393: 474-478 (1998)) and can be used in conjunction with VSTM5 antibodies (Ito, N. et al. Immunobiology 201 (5) 527-40 (2000)). Activating antibodies to T cell costimulatory molecules such as OX-40 (Weinberg, A. et al. Immunol 164: 2160-2169(2000)), 4-1BB (Melero, I. et al. Nature Medicine 3: 682-685 (1997), and ICOS (Hutloff, A. et al. Nature 397: 262-266 (1999)) as well as antibodies which block the activity of negative costimulatory molecules such as CTLA-4 (e.g., U.S. Pat. No. 5,811,097, implimumab) or BTLA (Watanabe, N. et al. Nat Immunol 4:670-9 (2003)), B7-H4 (Sica, G L et al. Immunity 18:849-6 (2003) 1) PD-1 (may also provide for increased levels of T cell activation. Bone marrow transplantation is currently being used to treat a variety of tumors of hematopoietic origin. While graft versus host disease is a consequence of this treatment, therapeutic benefit may be obtained from graft vs tumor responses. VSTM5 blockade can be used to increase the effectiveness of the donor engrafted tumor specific T cells.

There are also several experimental treatment protocols that involve ex vivo activation and expansion of antigen specific T cells and adoptive transfer of these cells into recipients in order to antigen-specific T cells against tumor (Greenberg, R. & Riddell, S. Science 285: 546-51(1999)). These methods may also be used to activate T cell responses to infectious agents such as CMV. Ex vivo activation in the presence of VSTM5 therapeutic agents may be expected to increase the frequency and activity of the adoptively transferred T cells.

Optionally, VSTM5 therapeutic agents according to at least some embodiments of the invention, can be combined with an immunogenic agent, such as cancerous cells, purified tumor antigens (including recombinant proteins, peptides, and carbohydrate molecules), cells, and cells transfected with genes encoding immune stimulating cytokines (He et al J Immunol. 173:4919-28 (2004). Non-limiting examples of tumor vaccines that can be used include peptides of MUC1 for treatment of colon cancer, peptides of MUC-1/CEA/TRICOM for the treatment of ovary cancer, or tumor cells transfected to express the cytokine GM-CSF (discussed further below).

In humans, some tumors have been shown to be immunogenic such as RCC. It is anticipated that by raising the threshold of T cell activation by VSTM5 blockade, we may expect to activate tumor responses in the host.

VSTM5 blockade is likely to be most effective when combined with a vaccination protocol. Many experimental strategies for vaccination against tumors have been devised (see Rosenberg, S., Development of Cancer Vaccines, ASCO Educational Book Spring: 60-62 (2000); Logothetis, C., ASCO Educational Book Spring: 300-302 (2000); Khayat, D. ASCO Educational Book Spring: 414-428; Foon, K. ASCO Educational Book Spring: 730-738 (2000); see also Restifo, N. and Sznol, M., Cancer Vaccines, Ch. 61, pp. 3023-3043 in DeVita, V. et al. (eds). Cancer: Principles and Practice of Oncology 5^{th} Edition (1997)). In one of these strategies, a vaccine is prepared using autologous or allogeneic tumor cells. These cellular vaccines have been shown to be most effective when the tumor cells are transduced to express GM-CSF. GM-CSF has been shown to be a potent activator of antigen presentation for tumor vaccination (Dranoff et al. Proc Natl. Acad. Sci U.S.A. 90: 3539-43 1993)).

The study of gene expression and large scale gene expression patterns in various tumors has led to the definition of so-called tumor specific antigens (Rosenberg, S A Immunity 10: 281-7 (1999)). In many cases, these tumor specific antigens are differentiation antigens expressed in the tumors and in the cell from which the tumor arose, for example melanocyte antigens gp100, MAGE antigens, and Trp-2. More importantly, many of these antigens can be shown to be the targets of tumor specific T cells found in the host. VSTM5 blockade may be used in conjunction with a collection of recombinant proteins and/or peptides expressed in a tumor in order to generate an immune response to these proteins. These proteins are normally viewed by the immune system as self-antigens and are therefore tolerant to them. The tumor antigen may also include the protein telomerase, which is required for the synthesis of telomeres of chromosomes and which is expressed in more than 85% of human cancers and in only a limited number of somatic tissues (Kim, N et al. Science 266: 2011-2013(1994)). (These somatic tissues may be protected from immune attack by various means). Tumor antigen may also be “neo-antigens” expressed in cancer cells because of somatic mutations that alter protein sequence or create fusion proteins between two unrelated sequences (i.e., bcr-abl in the Philadelphia chromosome), or idiotype from B cell tumors.

Other tumor vaccines may include the proteins from viruses implicated in human cancers such a Human Papilloma Viruses (HPV), Hepatitis Viruses (HBV and HCV) and Kaposi's Herpes Sarcoma Virus (KHSV). Another form of tumor specific antigen which may be used in conjunction with VSTM5 blockade is purified heat shock proteins (HSP) isolated from the tumor tissue itself. These heat shock proteins contain fragments of proteins from the tumor cells and these HSPs are highly efficient at delivery to antigen presenting cells for eliciting tumor immunity (Suot, R & Srivastava, P Science 269:1585-1588 (1995); Tamura, Y. et al. Science 278:117-120 (1997)).

Dendritic cells (DC) are potent antigen presenting cells that can be used to prime antigen-specific responses. DC's can be produced ex vivo and loaded with various protein and peptide antigens as well as tumor cell extracts (Nestle, F. et al. Nature Medicine 4: 328-332 (1998)). DCs may also be transduced by genetic means to express these tumor antigens as well. DCs have also been fused directly to tumor cells for the purposes of immunization (Kugler, A. et al. Nature Medicine 6:332-336(2000)). As a method of vaccination, DC immunization may be effectively combined with VSTM5 blockade to activate more potent anti-tumor responses.

Use of the VSTM5 Therapeutic Agents According to at Least Some Embodiments of the Invention as Adjuvant for Cancer Vaccination:

Immunization against tumor-associated antigens (TAAs) is a promising approach for cancer therapy and prevention, but it faces several challenges and limitations, such as tolerance mechanisms associated with self-antigens expressed by the tumor cells. Costimulatory molecules such as B7.1 (CD80) and B7.2 (CD86) have improved the efficacy of gene-based and cell-based vaccines in animal models and are under investigation as adjuvant in clinical trials. This adjuvant activity can be achieved either by enhancing the costimulatory signal or by blocking inhibitory signal that is transmitted by negative costimulators expressed by tumor cells (Neighbors et al., J Immunother.; 31(7):644-55(2008)).

According to at least some embodiments of the invention, any one of the VSTM5 therapeutic agents can be used as adjuvant for cancer vaccination. According to at least some embodiments, the invention provides methods for improving immunization against TAAs, comprising administering to a patient an effective amount of any one of VSTM5 therapeutic agents.

Use of the VSTM5 therapeutic agents according to at least some embodiments of the invention for Immunoenhancement

1. Treatment of Cancer

The therapeutic agents provided herein are generally useful in vivo and ex vivo as immune response-stimulating therapeutics. In general, the disclosed therapeutic agent compositions are useful for treating a subject having or being predisposed to any disease or disorder to which the subject's immune system mounts an immune response. The ability of therapeutic agents to modulate VSTM5 immune signals enables a more robust immune response to be possible. The therapeutic agents according to at least some embodiments of the invention are useful to stimulate or enhance immune responses involving immune cells, such as T cells.

The VSTM5 therapeutic agents according to at least some embodiments of the invention are useful for stimulating or enhancing an immune response in an individual with cancer by administering to a subject an amount of a therapeutic agent effective to stimulate T cells in the subject alone or in combination with other actives, e.g., other immunomodulators.

2. Use of the VSTM5 Therapeutic Agents in Vaccines

The VSTM5 therapeutic agents according to at least some embodiments of the invention are administered alone or in combination with any other suitable treatment. In one embodiment the therapeutic agents can be administered in conjunction with, or as a component of a vaccine composition as described above. The therapeutic agents according to at least some embodiments of the invention can be administered prior to, concurrently with, or after the administration of a vaccine. In one embodiment the therapeutic agents is administered at the same time as administration of a vaccine.

Use of Immunoinhibitory VSTM5 Therapeutic Agents and Pharmaceutical Compositions for Treatment of Immune-Related and Autoimmune Diseases

According to at least some embodiments, VSTM5 therapeutic agents and/or a pharmaceutical composition comprising same, as described herein, which function as VSTM5 agonizing therapeutic agents, may optionally be used for treating an immune system related disease. In some instances the immune system related condition comprises an immune related condition, including but not limited to autoimmune, inflammatory or allergic diseases such as recited herein, transplant rejection and graft versus host disease.

In some instances the immune condition is selected from autoimmune disease, inflammatory disease, allergic disease, transplant rejection, undesired gene or cell therapy immune responses, or graft versus host disease.

In some embodiments the treatment is combined with another moiety useful for treating immune related condition. Non limiting examples thereof include immunosuppressants such as corticosteroids, cyclosporin, cyclophosphamide, prednisone, azathioprine, methotrexate, rapamycin, tacrolimus, leflunomide or an analog thereof; mizoribine; mycophenolic acid; mycophenolate mofetil; 15-deoxyspergualine or an analog thereof; biological agents such as TNF-α blockers or antagonists, or any other biological agent targeting any inflammatory cytokine, nonsteroidal antiinflammatory drugs/Cox-2 inhibitors, hydroxychloroquine, sulphasalazopryine, gold salts, etanercept, infliximab, mycophenolate mofetil, basiliximab, atacicept, rituximab, cytoxan, interferon β-1a, interferon β-1b, glatiramer acetate, mitoxantrone hydrochloride, anakinra and/or other biologics and/or intravenous immunoglobulin (IVIG), interferons such as IFN-β1a (REBIF®. AVONEX® and CINNOVEX®) and IFN-β1b (BETASERON®); EXTAVIA®, BETAFERON®, ZIFERON®); glatiramer acetate (COPAXONE®), a polypeptide; natalizumab (TYSABRI®), mitoxantrone (NOVANTRONE®), a cytotoxic agent, a calcineurin inhibitor, e.g. Cyclosporin A or FK506; an immunosuppressive macrolide, e.g. Rapamycin or a derivative thereof; e.g. 40-O-(2-hydroxy)ethyl-rapamycin, a lymphocyte homing agent, e.g. FTY720 or an analog thereof, corticosteroids; cyclophosphamide; azathioprene; methotrexate; leflunomide or an analog thereof; mizoribine; mycophenolic acid; mycophenolate mofetil; 15-deoxyspergualine or an analog thereof; immunosuppressive monoclonal antibodies, e.g., monoclonal antibodies to leukocyte receptors, e.g., MHC, CD2, CD3, CD4, CD11a/CD18, CD7, CD25, CD27, B7, CD40, CD45, CD58, CD137, ICOS, CD150 (SLAM), OX40, 4-1BB or their ligands; or other immunomodulatory compounds, e.g. CTLA4-Ig (abatacept, ORENCIA®, belatacept), CD28-Ig, B7-H4-Ig, or other costimulatory agents, or adhesion molecule inhibitors, e.g. mAbs or low molecular weight inhibitors including LFA-1 antagonists, Selectin antagonists and VLA-4 antagonists, or another immunomodulatory agent.

In particular, treatment of multiple sclerosis using VSTM5 immunoinhibitory proteins according to the invention may e.g., be combined with, any therapeutic agent or method suitable for treating multiple sclerosis. Non-limiting examples of such known therapeutic agent or method for treating multiple sclerosis include interferon class, IFN-β-1a (REBIF®. AVONEX® and CINNOVEX®) and IFN-β-1b (BETASERON®, EXTAVIA®, BETAFERON®, ZIFERON®); glatiramer acetate (COPAXONE®), a polypeptide; natalizumab (TYSABRI®); and mitoxantrone (NOVANTRONE®), a cytotoxic agent, Fampridine (AMPYRA®). Other drugs include corticosteroids, methotrexate, cyclophosphamide, azathioprine, and intravenous immunoglobulin (IVIG), inosine, Ocrelizumab (R1594), Mylinax (Caldribine®), alemtuzumab (Campath®), daclizumab (Zenapax®), Panaclar/dimethyl fumarate (BG-12), Teriflunomide (HMR1726), fingolimod (FTY720), laquinimod (ABR216062), as well as Hematopoietic stem cell transplantation, NeuroVax®, Rituximab (Rituxan®) BCG vaccine, low dose naltrexone, helminthic therapy, angioplasty, venous stents, and alternative therapy, such as vitamin D, polyunsaturated fats, medical marijuana.

Similarly, treatment of rheumatoid arthritis, using VSTM5 immunoinhibitory proteins according to the invention may be combined with, for example, any therapeutic agent or method suitable for treating rheumatoid arthritis. Non-limiting examples of such known therapeutic agents or methods for treating rheumatoid arthritis include glucocorticoids, nonsteroidal anti-inflammatory drug (NSAID) such as salicylates, or cyclooxygenase-2 inhibitors, ibuprofen and naproxen, diclofenac, indomethacin, etodolac Disease-modifying antirheumatic drugs (DMARDs)—Oral DMARDs: Auranofin (Ridaura®), Azathioprine (Imuran®), Cyclosporine (Sandimmune®, Gengraf, Neoral, generic), D-Penicillamine (Cuprimine), Hydroxychloroquine (Plaquenil®), IM gold Gold sodium thiomalate (Myochrysine®) Aurothioglucose (Solganal®), Leflunomide (Arava®), Methotrexate (Rheumatrex®), Minocycline (Minocin®), Staphylococcal protein A immunoadsorption (Prosorba column), Sulfasalazine (Azulfidine®). Biologic DMARDs: TNF-α blockers including Adalimumab (Humira®) Etanercept (Enbrel®), Infliximab (Remicade®), golimumab (Simponi®), certolizumab pegol (Cimzia®), and other biological DMARDs, such as Anakinra (Kineret®), Rituximab (Rituxan®), Tocilizumab (Actemra®), CD28 inhibitor including Abatacept (Orencia®) and Belatacept.

Thus, treatment of IBD, using the agents according to at least some embodiments of the present invention may be combined with, for example, any known therapeutic agent or method for treating IBD. Non-limiting examples of such known therapeutic agents or methods for treating IBD include immunosuppression to control the symptom, such as prednisone, Mesalazine (including Asacol®, Pentasa®, Lialda®, Aspiro®, azathioprine (Imuran®), methotrexate, or 6-mercaptopurine, steroids, Ondansetron®, TNF-α blockers (including infliximab, adalimumab golimumab, certolizumab pegol), Orencia® (abatacept), ustekinumab (Stelara®), Briakinumab (ABT-874), Certolizumab pegol (Cimzia®), ITF2357 (Givinostat®), Natalizumab (Tysabri®), Firategrast® (SB-683699), Remicade® (infliximab), vedolizumab (MLN0002), other drugs including GSK1605786 CCX282-B (Traficet-EN®), AJM300, Stelara® (ustekinumab), Semapimod® (CNI-1493) tasocitinib (CP-690550), LMW Heparin MMX, Budesonide MMX, Simponi® (golimumab), MultiStem®, Gardasil® HPV vaccine, Epaxal® (virosomal hepatitis A vaccine), surgery, such as bowel resection, strictureplasty or a temporary or permanent colostomy or ileostomy; antifungal drugs such as nystatin (a broad spectrum gut antifungal) and either itraconazole (Sporanox) or fluconazole (Diflucan); alternative medicine, prebiotics and probiotics, cannabis, Helminthic therapy or ova of the Trichuris suis helminth

Thus, treatment of psoriasis, using the agents according to at least some embodiments of the present invention may be combined with, for example, any known therapeutic agent or method for treating psoriasis. Non-limiting examples of such known therapeutics for treating psoriasis include topical agents, typically used for mild disease, phototherapy for moderate disease, and systemic agents for severe disease. Non-limiting examples of topical agents: bath solutions and moisturizers, mineral oil, and petroleum jelly; ointment and creams containing coal tar, dithranol (anthralin), corticosteroids like desoximetasone (Topicort), Betamethasone, fluocinonide, vitamin D3 analogues (for example, calcipotriol), and retinoids. Non-limiting examples of phototherapy: sunlight; wavelengths of 311-313 nm, psoralen and ultraviolet A phototherapy (PUVA). Non-limiting examples of systemic agents: biologics, such as interleukin antagonists, TNF-α blockers including antibodies such as infliximab (Remicade®), adalimumab (Humira®), golimumab, certolizumab pegol, and recombinant TNF-α decoy receptor, etanercept (Enbrel®); drugs that target T cells, such as efalizumab (Xannelim®/Raptiva®), alefacept (Ameviv®), dendritic cells such Efalizumab; monoclonal antibodies (MAbs) targeting cytokines, including anti-IL-12/IL-23 (ustekinumab (Stelara®)) and anti-Interleukin-17; Briakinumab® (ABT-874); small molecules, including but not limited to ISA247; immunosuppressants, such as methotrexate, cyclosporine; vitamin A and retinoids (synthetic forms of vitamin A); and alternative therapy, such as changes in diet and lifestyle, fasting periods, low energy diets and vegetarian diets, diets supplemented with fish oil rich in vitamin A and vitamin D (such as cod liver oil), fish oils rich in the two omega-3 fatty acids eicosapentaenoic acid (EPA) and docosahexaenoic acid (DHA) and contain vitamin E, ichthyotherapy, hypnotherapy, and cannabis.

Thus, treatment of type 1 diabetes, using the agents according to at least some embodiments of the present invention may be combined with, for example, any known therapeutic agent or method for treating type ldiabetes. Non-limiting examples of such known therapeutics for treating type 1 diabetes include insulin, insulin analogs, islet transplantation, stem cell therapy including PROCHYMAL®, non-insulin therapies such as il-1β inhibitors including Anakinra (Kineret®), Abatacept (Orencia®), Diamyd, alefacept (Ameviv®), Otelixizumab, DiaPep277 (Hsp60 derived peptide), a 1-Antitrypsin, Prednisone, azathioprine, and Cyclosporin, E1-INT (an injectable islet neogenesis therapy comprising an epidermal growth factor analog and a gastrin analog), statins including Zocor®, Simlup®, Simcard®, Simvacor®, and Sitagliptin® (dipeptidyl peptidase (DPP-4) inhibitor), anti-CD3 mAb (e.g., Teplizumab®); CTLA4-Ig (abatacept), anti-IL-1β (Canakinumab), Anti-CD20 mAb (e.g, rituximab) and combinations thereof.

Thus, treatment of uveitis, using the agents according to at least some embodiments of the present invention may be combined with, for example, any known therapeutic agent or method for treating uveitis. Non-limiting examples of such known therapeutics for treating uveitis include corticosteroids, topical cycloplegics, such as atropine or homatropine, or injection of PSTTA (posterior subtenon triamcinolone acetate), antimetabolite medications, such as methotrexate, TNF-α blockers (including infliximab, adalimumab, etanercept, golimumab, and certolizumab pegol).

Thus, treatment for Sjögren's syndrome, using the agents according to at least some embodiments of the present invention may be combined with, for example, any known therapeutic agent or method for treating for Sjögren's syndrome. Non-limiting examples of such known therapeutics for treating for Sjögren's syndrome include Cyclosporine, pilocarpine (Salagen®) and cevimeline (Evoxac®), Hydroxychloroquine (Plaquenil), cortisone (prednisone and others) and/or azathioprine (Imuran®) or cyclophosphamide (Cytoxan®), Dexamethasone, Thalidomide, Dehydroepiandrosterone, NGX267, Rebamipide®, FID 114657, Etanercept®, Raptiva®, Belimumab, MabThera® (rituximab); Anakinra®, intravenous immune globulin (IVIG), Allogeneic Mesenchymal Stem Cells (AIIoMSC®), and Automatic neuro-electrostimulation by “Saliwell Crown”.

Thus, treatment for systemic lupus erythematosus, using the agents according to at least some embodiments of the present invention may be combined with, for example, any known therapeutic agent or method for treating for systemic lupus erythematosus. Non-limiting examples of such known therapeutics for treating for systemic lupus erythematosus include corticosteroids and Disease-modifying antirheumatic drugs (DMARDs), commonly anti-malarial drugs such as plaquenil and immunosuppressants (e.g. methotrexate and azathioprine) Hydroxychloroquine, cytotoxic drugs (e.g., cyclophosphamide and mycophenolate), Hydroxychloroquine (HCQ), Benlysta (belimumab), nonsteroidal anti-inflammatory drugs, Prednisone, Cellcept®, Prograf®, Atacicept®, Lupuzor®, Intravenous Immunoglobulins (IVIGs), CellCept® (mycophenolate mofetil), Orencia®, CTLA4-IgG4m (RG2077), rituximab, Ocrelizumab, Epratuzumab, CNTO 136, Sifalimumab (MEDI-545), A-623 (formerly AMG 623), AMG 557, Rontalizumab, paquinimod (ABR-215757), LY2127399, CEP-33457, Dehydroepiandrosterone, Levothyroxine, abetimus sodium (LIP 394), Memantine®, Opiates, Rapamycin®, renal transplantation, stem cell transplantation and combinations of any of the foregoing.

The immunoinhibitory VSTM5 therapeutic agents and/or a pharmaceutical composition comprising same, as recited herein, according to at least some embodiments of the invention, may be administered as the sole active ingredient or together with other drugs in immunomodulating regimens or other anti-inflammatory agents e.g. for the treatment or prevention of allo- or xenograft acute or chronic rejection or inflammatory or autoimmune disorders, or to induce tolerance.

Use of Immunostimulatory VSTM5 Therapeutic Agents and Pharmaceutical Compositions for the Treatment of Infectious Disease

According to at least some embodiments, immunostimulatory VSTM5 therapeutic agents and/or a pharmaceutical compositions as described herein, which function as VSTM5 blocking therapeutic agents, may optionally be used for treating infectious disease.

Chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection as a result of persistent exposure to foreign antigen, giving rise to T cell exhaustion. Exhausted T cells express high levels of multiple co-inhibitory receptors such as CTLA-4, PD-1, and LAGS (Crawford et al., Curr Opin Immunol. 21:179-186(2009); Kaufmann et al., J Immunol 182:5891-5897(2009); Sharpe et al., Nat Immunol 8:239-245(2007)). PD-1 overexpression by exhausted T cells was observed clinically in patients suffering from chronic viral infections including HIV, HCV and HBV (Crawford et al., Curr Opin Immunol 21:179-186 (2009); Kaufmann et al., J Immunol 182:5891-5897(2009); Sharpe et al., Nat Immunol 8:239-245) (2007). There has been some investigation into this pathway in additional pathogens, including other viruses, bacteria, and parasites (Hofmeyer et al., J Biomed Biotechnol. Vol 2011, Art. ID 451694, Bhadra et al., Proc Natl. Acad Sci. USA 108(22):9196-201 (2011)). For example, the PD-1 pathway was shown to be involved in controlling bacterial infection using a sepsis model induced by the standard cecal ligation and puncture method. The absence of PD-1 in knockout mice protected from sepsis-induced death in this model (Huang et al., Proc Natl. Acad Sci. USA 106; 6303-6308(2009)).

T cell exhaustion can be reversed by blocking co-inhibitory pathways such as PD-1 or CTLA-4 (Rivas et al., J Immunol. 183:4284-91 (2009); Golden-Mason et al., J Virol. 83:9122-30 (2009); Hofmeyer et al., J Biomed Biotechnol. Vol 2011, Art. ID 451694), thus allowing restoration of anti-viral immune function. The therapeutic potential of co-inhibition blockade for treating viral infection was extensively studied by blocking the PD-1/PD-L1 pathway, which was shown to be efficacious in several animal models of infection including acute and chronic simian immunodeficiency virus (SIV) infection in rhesus macaques (Valu et al., Nature 2009; 458:206-210) and in mouse models of chronic viral infection, such as lymphocytic choriomeningitis virus (LCMV) (Barber et al., Nature. 439:682-7 (2006)), and Theiler's murine encephalomyelitis virus (TMEV) model in SJL/J mice (Duncan and Miller PLoS One. 6:e18548 (2011)). In these models PD-1/PD-L1 blockade improved anti-viral responses and promoted clearance of the persisting viruses. In addition, PD-1/PD-L1 blockade increased the humoral immunity manifested as elevated production of specific anti-virus antibodies in the plasma, which in combination with the improved cellular responses leads to decrease in plasma viral loads and increased survival.

As used herein the term “infectious disorder and/or disease” and/or “infection”, used interchangeably, includes any disorder, disease and/or condition caused by presence and/or growth of pathogenic biological agent in an individual host organism. As used herein the term “infection” comprises the disorder, disease and/or condition as above, exhibiting clinically evident illness (i.e., characteristic medical signs and/or symptoms of disease) and/or which is asymtomatic for much or all of it course. As used herein the term “infection” also comprises disorder, disease and/or condition caused by persistence of foreign antigen that lead to exhaustion T cell phenotype characterized by impaired functionality which is manifested as reduced proliferation and cytokine production. As used herein the term “infectious disorder and/or disease” and/or “infection”, further includes any of the below listed infectious disorders, diseases and/or conditions, caused by a bacterial infection, viral infection, fungal infection and/or parasite infection.

As used herein the term “viral infection” comprises any infection caused by a virus, optionally including but not limited to Retroviridae (e.g., human immunodeficiency viruses, such as HIV-1 or HIV-2, acquired immune deficiency (AIDS) also referred to as HTLV-III, LAV or HTLV-III/LAV, or HIV-III; and other isolates, such as HIV-LP; Picornaviridae (e.g., polio viruses, hepatitis A virus; enteroviruses, human coxsackie viruses, rhinoviruses, echoviruses); Calciviridae (e.g., strains that cause gastroenteritis); Togaviridae (e.g., equine encephalitis viruses, rubella viruses); Flaviridae (e.g., dengue viruses, encephalitis viruses, yellow fever viruses); Coronaviridae (e.g., coronaviruses); Rhabdoviridae (e.g., vesicular stomatitis viruses, rabies viruses); Filoviridae (e.g., ebola viruses); Paramyxoviridae (e.g., parainfluenza viruses, mumps virus, measles virus, respiratory syncytial virus); Orthomyxoviridae (e.g., influenza viruses); Bungaviridae (e.g., Hantaan viruses, bunga viruses, phleboviruses and Nairo viruses); Arena viridae (hemorrhagic fever virus); Reoviridae (e.g., reoviruses, orbiviruses and rotaviruses); Birnaviridae; Hepadnaviridae (Hepatitis B virus); Parvoviridae (parvoviruses); Papovaviridae (papilloma viruses, polyoma viruses); Adenoviridae (most adenoviruses); Herperviridae (herpes simplex virus (HSV) 1 and 2, varicella zoster virus, cytomegalovirus (CMV), herpes viruses); Poxviridae (variola virsues, vaccinia viruses, pox viruses); and Iridoviridae (e.g., African swine fever virus); and unclassified viruses (e.g., the etiological agents of Spongiform encephalopathies, the agent of delta hepatitides (thought to be a defective satellite of hepatitis B virus), the agents of non-A, non-B hepatitis (class 1—internally transmitted; class 2—parenterally transmitted (i.e., Hepatitis C); Norwalk and related viruses, and astroviruses) as well as Severe acute respiratory syndrome virus and respiratory syncytial virus (RSV).

As used herein the term “fungal infection” comprises any infection caused by a fungi, optionally including but not limited to Cryptococcus neoformans, Histoplasma capsulatum, Coccidioides immitis, Blastomyces dermatitidis, Chlamydia trachomatis, and Candida albicans.

As used herein the term “parasite infection” comprises any infection caused by a parasite, optionally including but not limited to protozoa, such as Amebae, Flagellates, Plasmodium falciparum, Toxoplasma gondii, Ciliates, Coccidia, Microsporidia, Sporozoa; helminthes, Nematodes (Roundworms), Cestodes (Tapeworms), Trematodes (Flukes), Arthropods, and aberrant proteins known as prions.

An infectious disorder and/or disease caused by bacteria may optionally comprise one or more of Sepsis, septic shock, sinusitis, skin infections, pneumonia, bronchitis, meningitis, Bacterial vaginosis, Urinary tract infection (UCI), Bacterial gastroenteritis, Impetigo and erysipelas, Erysipelas, Cellulitis, anthrax, whooping cough, lyme disease, Brucellosis, enteritis, acute enteritis, Tetanus, diphtheria, Pseudomembranous colitis, Gas gangrene, Acute food poisoning, Anaerobic cellulitis, Nosocomial infections, Diarrhea, Meningitis in infants, Traveller's diarrhea, Hemorrhagic colitis, Hemolytic-uremic syndrome, Tularemia, Peptic ulcer, Gastric and Duodenal ulcers, Legionnaire's Disease, Pontiac fever, Leptospirosis, Listeriosis, Leprosy (Hansen's disease), Tuberculosis, Gonorrhea, Ophthalmia neonatorum, Septic arthritis, Meningococcal disease including meningitis, Waterhouse-Friderichsen syndrome, Pseudomonas infection, Rocky mountain spotted fever, Typhoid fever type salmonellosis, Salmonellosis with gastroenteritis and enterocolitis, Bacillary dysentery/Shigellosis, Coagulase-positive staphylococcal infections: Localized skin infections including Diffuse skin infection (Impetigo), Deep localized infections, Acute infective endocarditis, Septicemia, Necrotizing pneumonia, Toxinoses such as Toxic shock syndrome and Staphylococcal food poisoning, Cystitis, Endometritis, Otitis media, Streptococcal pharyngitis, Scarlet fever, Rheumatic fever, Puerperal fever, Necrotizing fasciitis, Cholera, Plague (including Bubonic plague and Pneumonic plague), as well as any infection caused by a bacteria selected from but not limited to Helicobacter pyloris, Boreliai burgdorferi, Legionella pneumophilia, Mycobacteria sps (e.g., M. tuberculosis, M. avium, M. Intracellulare, M. kansaii, and M gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, and Streptococcus bovis, Streptococcus (anaerobic sps.), Streptococcus pneumoniae, pathogenic Campylobacter sp., Enterococcus sp Haemophilus influenzae, Bacillus antracis, corynebacterium diphtherias, corynebacterium sp., Erysipelothrix rhusiopathiae, Clostridium perfringens, Clostridium tetani, Enterobacter aerogenes, Klebsiella pneumoniae, Pasteurella multocida, Bacteroides sp., Fusobacterium nucleatum, Streptobacillus moniliformis, Treponema palladium, Treponema pertenue, Leptospira, and Actinomyces israeli.

Non limiting examples of infectious disorder and/or disease caused by virus is selected from the group consisting of but not limited to acquired immune deficiency (AIDS), West Nile encephalitis, coronavirus infection, rhinovirus infection, influenza, dengue, hemorrhagic fever; an otological infection; severe acute respiratory syndrome (SARS), acute febrile pharyngitis, pharyngoconjunctival fever, epidemic keratoconjunctivitis, infantile gastroenteritis, infectious mononucleosis, Burkitt lymphoma, acute hepatitis, chronic hepatitis, hepatic cirrhosis, hepatocellular carcinoma, primary HSV-1 infection, (gingivostomatitis in children, tonsillitis & pharyngitis in adults, keratoconjunctivitis), latent HSV-1 infection (herpes labialis, cold sores), aseptic meningitis, Cytomegalovirus infection, Cytomegalic inclusion disease, Kaposi sarcoma, Castleman disease, primary effusion lymphoma, influenza, measles, encephalitis, postinfectious encephalomyelitis, Mumps, hyperplastic epithelial lesions (common, flat, plantar and anogenital warts, laryngeal papillomas, epidermodysplasia verruciformis), croup, pneumonia, bronchiolitis, Poliomyelitis, Rabies, bronchiolitis, pneumonia, German measles, congenital rubella, Hemorrhagic Fever, Chickenpox, Dengue, Ebola infection, Echovirus infection, EBV infection, Fifth Disease, Filovirus, Flavivirus, Hand, foot & mouth disease, Herpes Zoster Virus (Shingles), Human Papilloma Virus Associated Epidermal Lesions, Lassa Fever, Lymphocytic choriomeningitis, Parainfluenza Virus Infection, Paramyxovirus, Parvovirus B19 Infection, Picornavirus, Poxviruses infection, Rotavirus diarrhea, Rubella, Rubeola, Varicella, and Variola infection.

An infectious disorder and/or disease caused by fungi optionally includes but is not limited to Allergic bronchopulmonary aspergillosis, Aspergilloma, Aspergillosis, Basidiobolomycosis, Blastomycosis, Candidiasis, Chronic pulmonary aspergillosis, Chytridiomycosis, Coccidioidomycosis, Conidiobolomycosis, Covered smut (barley), Cryptococcosis, Dermatophyte, Dermatophytid, Dermatophytosis, Endothrix, Entomopathogenic fungus, Epizootic lymphangitis, Epizootic ulcerative syndrome, Esophageal candidiasis, Exothrix, Fungemia, Histoplasmosis, Lobomycosis, Massospora cicadina, Mycosis, Mycosphaerella fragariae, Myringomycosis, Paracoccidioidomycosis, Pathogenic fungi, Penicilliosis, Thousand cankers disease, Tinea, Zeaspora, Zygomycosis. Non limiting examples of infectious disorder and/or disease caused by parasites is selected from the group consisting of but not limited to Acanthamoeba, Amoebiasis, Ascariasis, Ancylostomiasis, Anisakiasis, Babesiosis, Balantidiasis, Baylisascariasis, Blastocystosis, Candiru, Chagas disease, Clonorchiasis, Cochliomyia, Coccidia, Chinese Liver Fluke Cryptosporidiosis, Dientamoebiasis, Diphyllobothriasis, Dioctophyme renalis infection, Dracunculiasis, Echinococcosis, Elephantiasis, Enterobiasis, Fascioliasis, Fasciolopsiasis, Filariasis, Giardiasis, Gnathostomiasis, Hymenolepiasis, Halzoun Syndrome, Isosporiasis, Katayama fever, Leishmaniasis, lymphatic filariasis, Malaria, Metagonimiasis, Myiasis, Onchocerciasis, Pediculosis, Primary amoebic meningoencephalitis, Parasitic pneumonia, Paragonimiasis, Scabies, Schistosomiasis, Sleeping sickness, Strongyloidiasis, Sparganosis, Rhinosporidiosis, River blindness, Taeniasis (cause of Cysticercosis), Toxocariasis, Toxoplasmosis, Trichinosis, Trichomoniasis, Trichuriasis, Trypanosomiasis, and Tapeworm infection.

A preferred example of infectious disease is a disease caused by any of hepatitis B, hepatitis C, infectious mononucleosis, EBV, cytomegalovirus, AIDS, HIV-1, HIV-2, tuberculosis, malaria and schistosomiasis.

According to at least some embodiments of the present invention, there is provided use of a combination of the therapeutic agents and/or a pharmaceutical composition comprising same, as recited herein, and a known therapeutic agent effective for treating infection.

The immunostimulatory VSTM5 therapeutic agents and/or a pharmaceutical composition comprising same, as recited herein, can be administered in combination with one or more additional therapeutic agents used for treatment of bacterial infections, including, but not limited to, antibiotics including aminoglycosides, carbapenems, cephalosporins, macrolides, lincosamides, nitrofurans, penicillins, polypeptides, quinolones, sulfonamides, tetracyclines, drugs against mycobacteria including but not limited to Clofazimine, Cycloserine, Cycloserine, Rifabutin, Rifapentine, Streptomycin and other antibacterial drugs such as Chloramphenicol, Fosfomycin, Metronidazole, Mupirocin, and Tinidazole.

The immunostimulatory VSTM5 therapeutic agents and/or a pharmaceutical composition comprising same, as recited herein, can be administered in combination with one or more additional therapeutic agents used for treatment of viral infections, including, but not limited to, antiviral drugs such as oseltamivir (Tamiflu®) and zanamivir (Relenza®) Arbidol—adamantane derivatives (Amantadine®, Rimantadine®)—neuraminidase inhibitors (Oseltamivir®, Laninamivir®, Peramivir®, Zanamivir®), nucleotide analog reverse transcriptase inhibitor including Purine analogue guanine (Aciclovir#/Valacyclovir®, Ganciclovir®/Valganciclovir®, Penciclovir®/Famciclovir®) and adenine (Vidarabine®), Pyrimidine analogue, uridine (Idoxuridine®, Trifluridine®, Edoxudine), thymine (Brivudine®), cytosine (Cytarabine®); Foscarnet; Nucleoside analogues/NARTIs: Entecavir®, Lamivudine®, Telbivudine®, Clevudine®; Nucleotide analogues/NtRTIs: Adefovir®, Tenofovir®; Nucleic acid inhibitors such as Cidofovir®; Interferon α-2b, Peginterferon α-2a; Ribavirin#/Taribavirin®, antiretroviral drugs including zidovudine, lamivudineabacavir, lopinavir, ritonavir, tenofovir/emtricitabine, efavirenz each of them alone or a various combinations, gp41 (Enfuvirtide®), Raltegravir®, protease inhibitors such as Fosamprenavir®, Lopinavir® and Atazanavir®, Methisazone®, Docosanol®, Fomivirsen®, Tromantadine® and combinations thereof.

The immunostimulatory VSTM5 therapeutic agents and/or a pharmaceutical composition comprising same, as recited herein, can be administered in combination with one or more additional therapeutic agents used for treatment of fungal infections, including, but not limited to, antifungal drugs of the polyene antifungals, imidazole, triazole, and thiazole antifungals, allylamines, echinocandins or other anti-fungal drugs.

Use of Immunostimulatory VSTM5 Therapeutic Agents and Pharmaceutical Compositions for Treatment of Sepsis

According to at least some embodiments, immunostimulatory VSTM5 therapeutic agents and/or a pharmaceutical compositions as described herein, which function as VSTM5 blocking therapeutic agents, may optionally be used for treating sepsis. Sepsis is a potentially life-threatening complication of an infection. Sepsis represents a complex clinical syndrome that develops when the initial host response against an infection becomes inappropriately amplified and dysregulated, becoming harmful to the host. The initial hyperinflammatory phase (‘cytokine storm’) in sepsis is followed by a state of immunosuppression (Hotchkiss et al. Lancet Infect. Dis. 13:260-268 (2013)). This latter phase of impaired immunity, also referred to as ‘immunoparalysis’, is manifested in failure to clear the primary infection, reactivation of viruses such as HSV and cytomegalovirus, and development of new, secondary infections, often with organisms that are not particularly virulent to the immunocompetent patient. The vast majority of septic patients today survive their initial hyperinflammatory insult only to end up in the intensive care unit with sepsis-induced multi-organ dysfunction over the ensuing days to weeks. Sepsis-induced immunosuppression is increasingly recognized as the overriding immune dysfunction in these vulnerable patients. The impaired pathogen clearance after primary infection and/or susceptibility to secondary infections contribute to the high rates of morbidity and mortality associated with sepsis.

Upregulation of inhibitory proteins has lately emerged as one of the critical mechanisms underlying the immunosuppression in sepsis. The PD-1/PDL-1 pathway, for example, appears to be a determining factor of the outcome of sepsis, regulating the delicate balance between effectiveness and damage by the antimicrobial immune response. During sepsis in an experimental model, peritoneal macrophages and blood monocytes markedly increased PD-1 levels, which was associated with the development of cellular dysfunction (Huang et al 2009 PNAS 106:6303-6308). Similarly, in patients with septic shock the expression of PD-1 on peripheral T cells and of PDL-1 on monocytes was dramatically upregulated (Zhang et al 2011 Crit. Care 15:R70). Recent animal studies have shown that blockade of the PD-1/PDL-1 pathway by anti-PD1 or anti-PDL1 antibodies improved survival in sepsis (Brahmamdam et al J. Leukoc. Biol. 88:233-240 (2010); Zhang et al Critical Care 14:R220 (2010); Chang et al Critical Care 17:R85 (2013). Similarly, blockade of CTLA-4 with anti-CTLA4 antibodies improved survival in sepsis (Inoue et al Shock 36:38-44(2011); Chang et al Critical Care 17:R85(2013)). Taken together, these findings suggest that blockade of inhibitory proteins, including negative costimulatory molecules, is a potential therapeutic approach to prevent the detrimental effects of sepsis (Goyert and Silver J Leukoc Biol. (2):225-6) (2010)).

As used herein, the term “sepsis” or “sepsis related condition” encompasses Sepsis, Severe sepsis, Septic shock, Systemic inflammatory response syndrome (SIRS), Bacteremia, Septicemia, Toxemia, Septic syndrome.

According to at least some embodiments of the present invention, there is provided use of a combination of the immunostimulatory VSTM5 therapeutic agents and/or a pharmaceutical composition comprising same, as recited herein, and a known therapeutic agent effective for treating sepsis.

The restoration of the delicate balance that normally exists between the active and suppressor arms of the immune system in sepsis patients may depend on the precise nature of the imbalance, i.e. the pathogenic organism responsible for the infection, its location, the amount of time passed since onset of infection, and other individual parameters. Thus, the correct choice of tools may well depend on the specific immune status or deficit of each individual patient, and may require combination of different drugs.

According to at least some embodiments of the present invention, there is provided use of a combination of the immunostimulatory VSTM5 therapeutic agents and/or a pharmaceutical composition comprising same, as recited herein, can be combined with standard of care or novel treatments for sepsis, with therapies that block the cytokine storm in the initial hyperinflammatory phase of sepsis, and/or with therapies that have immunostimulatory effect in order to overcome the sepsis-induced immunosuppression phase.

Combination with standard of care treatments for sepsis, as recommended by the International Guidelines for Management of Severe sepsis and Septic shock (Dellinger et al 2013 Intensive Care Med 39:165-228), some of which are described below.
Broad spectrum antibiotics having activity against all likely pathogens (bacterial and/or fungal—treatment starts when sepsis is diagnosed, but specific pathogen is not identified)—example Cefotaxime (Claforan®), Ticarcillin® and clavulanate (Timentin®), Piperacillin and tazobactam (Zosyn®), Imipenem and cilastatin (Primaxin®), Meropenem (Merrem®), Clindamycin (Cleocin®), Metronidazole (Flagyl), Ceftriaxone (Rocephin®), Ciprofloxacin (Cipro®), Cefepime (Maxipime®), Levofloxacin (Levaquin®), Vancomycin or any combination of the listed drugs.
Vasopressors: example Norepinephrine, Dopamine, Epinephrine, and vasopressin.
Steroids: for example: Hydrocortisone, Dexamethasone, or Fludrocortisone, intravenous or otherwise
Inotropic therapy: for example Dobutamine for sepsis patients with myocardial dysfunction
Recombinant human activated protein C (rhAPC), such as drotrecogin a (activated) (DrotAA).
β-blockers, which may further reduce local and systemic inflammation.
Metabolic interventions such as pyruvate, succinate or high dose insulin substitutions.

Combination with Novel Potential Therapies for Sepsis:
Selective inhibitors of sPLA2-IIA (such as LY315920NA/S-5920). Mechanism: The Group IIA secretory phospholipase A2 (sPLA2-IIA), released during inflammation, is increased in severe sepsis, and plasma levels are inversely related to survival.
Phospholipid emulsion (such as GR270773). Mechanism: Preclinical and ex vivo studies show that lipoproteins bind and neutralize endotoxin, and experimental animal studies demonstrate protection from septic death when lipoproteins are administered. Endotoxin neutralization correlates with the amount of phospholipid in the lipoprotein particles.
anti-TNF-α antibody: Mechanism: Tumor necrosis factor-α (TNF-α) induces many of the pathophysiological signs and symptoms observed in sepsis
anti-CD14 antibody (such as IC14). Mechanism: Upstream recognition molecules, like CD14, play key roles in the pathogenesis. Bacterial cell wall components bind to CD14 and co-receptors on myeloid cells, resulting in cellular activation and production of proinflammatory mediators. An anti-CD14 monoclonal antibody (IC14) has been shown to decrease lipopolysaccharide-induced responses in animal and human models of endotoxemia.
Inhibitors of Toll-like receptors (TLRs) and their downstream signaling pathways. Mechanism: Infecting microbes display highly conserved macromolecules (e.g., lipopolysaccharides, peptidoglycans) on their surface. When these macromolecules are recognized by pattern-recognition receptors (called Toll-like receptors [TLRs]) on the surface of immune cells, the host's immune response is initiated. This may contribute to the excess systemic inflammatory response that characterizes sepsis Inhibition of several TLRs is being evaluated as a potential therapy for sepsis, in particular TLR4, the receptor for Gram-negative bacteria outer membrane lipopolysaccharide or endotoxin. Various drugs targeting TLR4 expression and pathway have a therapeutic potential in sepsis (Wittebole et al Mediators of Inflammation Vol 10 Article ID 568396(2010)). Among these are antibodies targeting TLR4, soluble TLR4, Statins (such as rosuvastatin, Simvastatin), Ketamine, nicotinic analogues, eritoran (E5564), and resatorvid (TAK242). In addition, antagonists of other TLRs may be used such as chloroquine or an inhibitor of TLR-2 such as a neutralizing antibody (anti-TLR-2).
Lansoprazole through its action on SOCS1 (suppressor of cytokine secretion)
Talactoferrin or Recombinant Human Lactoferrin. Mechanism: Lactoferrin is a glycoprotein with anti-infective and anti-inflammatory properties found in secretions and immune cells. Talactoferrin α, a recombinant form of human lactoferrin, has similar properties and plays an important role in maintaining the gastrointestinal mucosal barrier integrity. Talactoferrin showed efficacy in animal models of sepsis, and in clinical trials in patients with severe sepsis (Guntupalli et al Crit Care Med. 41(3):706-716(2013)).
Milk fat globule EGF factor VIII (MFG-E8)—a bridging molecule between apoptotic cells and phagocytes, which promotes phagocytosis of apoptotic cells.
Agonists of the ‘cholinergic anti-inflammatory pathway’, such as nicotine and analogues. Mechanism: Stimulating the vagus nerve reduces the production of cytokines, or immune system mediators, and blocks inflammation. This nerve “circuitry”, called the “inflammatory reflex”, is carried out through the specific action of acetylcholine, released from the nerve endings, on the β7 subunit of the nicotinic acetylcholine receptor (α7nAChR) expressed on macrophages, a mechanism termed ‘the cholinergic anti-inflammatory pathway’. Activation of this pathway via vagus nerve stimulation or pharmacologic α7 agonists prevents tissue injury in multiple models of systemic inflammation, shock, and sepsis (Matsuda et al J Nippon Med Sch. 79:4-18 (2012); Huston Surg. Infect. 13:187-193(2012)).
Therapeutic agents targeting the inflammasome pathways. Mechanism: The inflammasome pathways greatly contribute to the inflammatory response in sepsis, and critical elements are responsible for driving the transition from localized inflammation to deleterious hyperinflammatory host response (Cinel and Opal Crit. Care Med. 37:291-304 (2009); Matsuda et al JNippon Med Sch. 79:4-18(2012)).
Stem cell therapy. Mechanism: Mesenchymal stem cells (MSCs) exhibit multiple beneficial properties through their capacity to home to injured tissue, activate resident stem cells, secrete paracrine signals to limit systemic and local inflammatory response, beneficially modulate immune cells, promote tissue healing by decreasing apoptosis in threatened tissues and stimulating neoangiogenesis, and exhibit direct antimicrobial activity. These effects are associated with reduced organ dysfunction and improved survival in sepsis animal models, which have provided evidence that MSCs may be useful therapeutic adjuncts (Wannemuehler et al J. Surg. Res. 173:113-26(2012)).
Combination of immunostimulatory VSTM5 therapeutic agent and/or a pharmaceutical composition comprising same with other immunomodulatory agents, such as immunostimulatory antibodies, cytokine therapy, immunomodulatory drugs. Such agents bring about increased immune responsiveness, especially in situations in which immune defenses (whether innate and/or adaptive) have been degraded, such as in sepsis-induced hypoinflammatory and immunosuppressive condition. Reversal of sepsis-induced immunoparalysis by therapeutic agents that augments host immunity may reduce the incidence of secondary infections and improve outcome in patients who have documented immune suppression (Hotchkiss et al Lancet Infect. Dis. 13:260-268(2013); Payen et al Crit Care. 17:118(2013)).
Immunostimulatory antibodies promote immune responses by directly modulating immune functions, i.e. by blocking other inhibitory proteins or by enhancing costimulatory proteins. Mechanism: Experimental models of sepsis have shown that immunostimulation by antibody blockade of inhibitory proteins, such as PD-1, PDL-1 or CTLA-4 improved survival in sepsis (Brahmamdam et al J. Leukoc. Biol. 88:233-240 (2010); Zhang et al Critical Care 14:R220 (2010); Chang et al Critical Care 17:R85 (2013); Inoue et al Shock 36:38-44 (2011)), pointing to such immunostimulatory agents as potential therapies for preventing the detrimental effects of sepsis-induced immunosuppression (Goyert and Silver J Leukoc Biol. 88(2):225-6(2010)). Immunostimulatory antibodies include: 1) antagonistic antibodies targeting inhibitory immune checkpoints include anti-CTLA4 mAbs (such as ipilimumab, tremelimumab), anti-PD-1 (such as nivolumab BMS-936558/MDX-1106/ONO-4538, AMP224, CT-011, lambrozilumab MK-3475), anti-PDL-1 antagonists (such as BMS-936559/MDX-1105, MEDI4736, RG-7446/MPDL3280A); anti-LAG-3 such as IMP-321), anti-TIM-3, anti-BTLA, anti-B7-H4, anti-B7-H3, and anti-VISTA. 2) Agonistic antibodies enhancing immunostimulatory proteins include anti-CD40 mAbs (such as CP-870,893, lucatumumab, dacetuzumab), anti-CD137 mAbs (such as BMS-663513 urelumab, PF-05082566), anti-OX40 mAbs (such as anti-OX40), anti-GITR mAbs (such as TRX518), anti-CD27 mAbs (such as CDX-1127), and anti-ICOS mAbs.
Cytokines which directly stimulate immune effector cells and enhance immune responses can be used in combination with anti-GEN antibody for sepsis therapy: IL-2, IL-7, IL-12, IL-15, IL-17, IL-18 and/or IL-21, IL-23, IL-27, GM-CSF, IFNα, IFNβ, IFNγ. Mechanism: Cytokine-based therapies embody a direct attempt to stimulate the patient's own immune system. Experimental models of sepsis have shown administration of cytokines, such as IL-7 and IL-15, promote T cell viability and result in improved survival in sepsis (Unsinger et al J. Immunol. 184:3768-3779(2010); Inoue et al J. Immunol. 184:1401-1409 (2010)). IFNγ reverses sepsis-induced immunoparalysis of monocytes in vitro. A study in vivo showed that IFNγ partially reverses immunoparalysis in vivo in humans. IFNγ and granulocyte-macrophage colony-stimulating factor (GM-CSF) restore immune competence of ex vivo stimulated leukocytes of patients with sepsis (Mouktaroudi et al Crit Care. 14: 17 (2010); Leentjens et al Am J Respir Crit Care Med 186:838-845, (2012)).
Immunomodulatory drugs such as thymosin a 1. Mechanism: Thymosin a 1 (Tal) is a naturally occurring thymic peptide which acts as an endogenous regulator of both the innate and adaptive immune systems. It is used worldwide for treating diseases associated with immune dysfunction including viral infections such as hepatitis B and C, certain cancers, and for vaccine enhancement. Further, recent immunomodulatory research has supported the beneficial effect of Tal treatment in septic patients (Wu et al. Critical Care 17:R8 (2013)).

Use of Immunoinhibitory VSTM5 Therapeutic Agents and Pharmaceutical Compositions for Reducing the Undesirable Immune Activation that Follows Gene or Cell Therapy

As used herein the term “gene therapy” encompasses any type of gene or cell therapy, vector-mediated gene therapy, gene transfer, virus-mediated gene transfer.

According to at least some embodiments of the present invention, VSTM5 therapeutic agents and/or pharmaceutical compositions as described herein, which target VSTM5 and have inhibitory activity on immune responses, could be used as therapeutic agents for reducing the undesirable immune activation that follows gene therapy used for treatment of various genetic diseases. Such immunoinhibitory VSTM5 therapeutic agents have VSTM5-like inhibitory activity on immune responses and/or enhance VSTM5 immune inhibitory activity, e.g., by the inhibition of pathogenic T cells and/or NK cells.

Gene therapy products for the treatment of genetic diseases are currently in clinical trials. Recent studies document therapeutic success for several genetic diseases using gene therapy vectors. Gene therapy strategies are characterized by 3 critical elements, the gene to be transferred, the target tissue into which the gene will be introduced, and the vector (gene delivery vehicle) used to facilitate entry of the gene to the target tissue. The vast majority of gene therapy clinical trials have exploited viral vectors as very efficient delivery vehicles, including retroviruses, lentiviruses, adenoviruses, adeno-associated viruses, pseudotype viruses and herpes simplex viruses. However, the interactions between the human immune system and all the components of gene therapy vectors seem to represent one of the major limitations to long-lasting therapeutic efficacy. Human studies have shown that the likelihood of a host immune response to the viral vector is high. Such immune responses to the virus or the transgene product itself, resulting in formation of neutralizing antibodies and/or destruction of transduced cells by cytotoxic cells, can greatly interfere with therapeutic efficacy (Seregin and Amalfitano Viruses 2:2013 (2010); Mingozzi and High Blood 122:23 (2013); Masat et al Discov Med. 15:379(2013)). Therefore, developing strategies to circumvent immune responses and facilitate long-term expression of transgenic therapeutic proteins is one of the main challenges for the success of gene therapy in the clinic.

Factors influencing the immune response against transgenic proteins encoded by viral vectors include route of administration, vector dose, immunogenicity of the transgenic protein, inflammatory status of the host and capsid serotype. These factors are thought to influence immunogenicity by triggering innate immunity, cytokine production, APC maturation, antigen presentation and, ultimately, priming of naïve T lymphocytes to functional effectors (Mingozzi and High Blood 122:23 (2013)). Therefore, the idea to dampen immune activation by interfering with these very mechanisms has logically emerged with the aim to induce a short-term immunosuppression, avoid the early immune priming that follows vector administration and promote long-term tolerance.

As a strategy to inhibit the undesirable immune activation that follows gene therapy, particularly after multiple injections, immunomodulation treatment by targeting of two non-redundant checkpoints of the immune response at the time of vector delivery was tested in animal models. Studies of vector-mediated immune responses upon adenoviral vector instilled into the lung in mice or monkeys showed that transient treatment with an anti-CD40L antibody lead to suppression of adenovirus-induced immune responses; consequently, the animals could be re-administered with adenovirus vectors. Short treatment with this Ab resulted in long-term effects on immune functions and prolonged inhibition of the adenovirus-specific humoral response well beyond the time when the Ab effects were no longer significant, pointing to the therapeutic potential in blockade of this costimulatory pathway as an immunomodulatory regimen to enable administration of gene transfer vectors (Scaria et al., Gene Ther. 4: 611 (1997); and Chirmule et al J. Virol. 74: 3345(2000)). Other studies showed that co-administration of CTLA4-Ig and an anti-CD40L Ab around the time of primary vector administration decreased immune responses to the vector, prolonged long term adenovirus-mediated gene expression and enabled secondary adenovirus-mediated gene transfer even after the immunosuppressive effects of these agents were no longer present, indicating that it may be possible to obtain persistence as well as secondary adenoviral-mediated gene transfer with transient immunosuppressive therapies (Kay et al Proc. Natl. Acad. Sci. U.S.A. 94:4686 (1997)). In another study, similar administration of CTLA4-Ig and an anti-CD40L Ab abrogated the formation of neutralizing Abs against the vector, and enabled gene transfer expression, provided the treatment was administered during each gene transfer injection (Lorain et al 2008 Molecular Therapy 16:541). Furthermore, administration of CTLA4-Ig to mice, even as single administration, resulted in suppression of immune responses and prolonged transgene expression at early time points (Adriouch et al Front. Microbiol. 2:199 (2011)). However, CTLA4-Ig alone was not sufficient to permanently wipe out the immune responses against the transgene product. Combined treatment targeting two immune checkpoints with CTLA4-Ig and PD-L1 or PDL-2 resulted in synergistic improvement of transgene tolerance at later time points, by probably targeting two non-redundant mechanisms of immunomodulation, resulting in long term transgene persistence and expression (Adriouch et al Front. Microbiol. 2:199 (2011)).

According to at least some embodiments of the present invention, nucleic acid sequences encoding soluble VSTM5 proteins and/or a fusion protein as described herein; alone or in combination with another immunomodulatory agent or in combination with any of the strategies and approaches tested to overcome the limitation of immune responses to gene therapy, could be used for reducing the undesirable immune activation that follows gene therapy.

Current approaches include exclusion of patients with antibodies to the delivery vector, administration of high vector doses, use of empty capsids to adsorb anti-vector antibodies allowing for subsequent vector transduction, repeated plasma exchange (plasmapheresis) cycles to adsorb immunoglobulins and reduce the anti-vector antibody titer.

Novel approaches attempting to overcome these limitations can be divided into two broad categories: selective modification of the Ad vector itself and pre-emptive immune modulation of the host (Seregin and Amalfitano 2010 Viruses 2:2013). The first category comprises several innovative strategies including: (1) Ad-capsid-display of specific inhibitors or ligands; (2) covalent modifications of the entire Ad vector capsid moiety; (3) the use of tissue specific promoters and local administration routes; (4) the use of genome modified Ads; and (5) the development of chimeric or alternative serotype Ads.

The second category of methods includes the use of immunosuppressive drugs or specific compounds to block important immune pathways, which are known to be induced by viral vectors Immunosuppressive agents have been tested in preclinical studies and shown efficacy in prevention or eradication of immune responses to the transfer vector and transgene product. These include general immunosuppressive agents such as cyclosporine A; cyclophosphamide; FK506; glucocorticoids or steroids such as dexamethasone; TLR9 blockade such as the TLR9 antagonist oligonucleotide ODN-2088; TNF-α blockade with anti-TNF-α antibodies or TNFR-Ig antibody, Erk and other signaling inhibitors such as U0126. In the clinical setting, administration of glucocorticoids has been successfully used to blunt T cell responses directed against the viral capsid upon liver gene transfer of adenovirus-associated virus (AAV) vector expressing human factor IX transgene to severe hemophilia B patients (Nathwani et al N. Engl. J. Med. 365:2357 (2011)).

In contrast to the previous approaches that utilize drugs that tend to “globally” and non-specifically immunosuppress the host, more selective immunosuppressive approaches have been developed. These include the use of agents which provide blockade of positive co-stimulatory interactions, such as between CD40 and CD154, ICOS and ICOSL, CD28 and CD80 or CD86 (including CTLA4-Ig), NKG2D and NKG2D ligands, LFA-1 and ICAM, LFA-3 and CD2, 4-1BB and 4-1BBL, OX40 and OX40L, GITR and GITRL and agents that stimulate negative costimulatory receptors such as CTLA-4, PD-1, BTLA, LAG-3, TIM-1, TIM-3, KIRs, and the receptors for B7-H4 and B7-H3. Some of these have been utilized in preclinical or clinical transplantation studies (Pilat et al Sem. Immunol. 23:293 (2011)).

Use of the VSTM5 Agents and/or Pharmaceutical Compositions for Adoptive Immunotherapy:

One of the cardinal features of some models of tolerance is that once the tolerance state has been established, it can be perpetuated to naive recipients by the adoptive transfer of donor-specific regulatory cells. Such adoptive transfer studies have also addressed the capacity of T-cell subpopulations and non-T cells to transfer tolerance. Such tolerance can be induced by blocking costimulation or upon engagement of a co-inhibitory B7 with its counter receptor. This approach, that has been successfully applied in animals and is evaluated in clinical trials in humans, (Scalapino K J and Daikh D I. PLoS One, 4(6):e6031 (2009); Riley et al., Immunity 30(5): 656-665 (2009)) provides a promising treatment option for autoimmune disorders and transplantation. According to at least some embodiments of the invention, VSTM5 secreted or soluble form or ECD and/or variants, and/or orthologs, and/or fusions and/or conjugates thereof, are used for adoptive immunotherapy. Thus, according to at least some embodiments, the invention provides methods for in vivo or ex vivo tolerance induction, comprising administering effective amount of VSTM5 secreted or soluble form or ECD and/or variants, and/or orthologs, and/or fusions and/or conjugates thereof, to a patient or to leukocytes isolated from the patient, in order to induce differentiation of tolerogenic regulatory cells; followed by ex-vivo enrichment and expansion of said cells and reinfusion of the tolerogenic regulatory cells to said patient.

Alternatively, immune responses can be enhanced in a patient by removing immune cells from the patient, contacting immune cells in vitro with an agent that inhibits VSTM5 activity, and/or which inhibits the interaction of VSTM5 with its natural binding partners, and reintroducing the in vitro stimulated immune cells into the patient. In another embodiment, a method of modulating immune responses involves isolating immune cells from a patient, transfecting them with a nucleic acid molecule encoding a form of VSTM5, such that the cells express all or a portion of the VSTM5 polypeptide according to various embodiments of the present invention on their surface, and reintroducing the transfected cells into the patient. The transfected cells have the capacity to modulate immune responses in the patient.

Pharmaceutical Compositions

In another aspect, the present invention provides a composition, e.g., a pharmaceutical composition, containing one or a combination of the VSTM5 therapeutic agent, according to at least some embodiments of the invention. Thus, the present invention features a pharmaceutical composition comprising a therapeutically effective amount of a therapeutic agent according to at least some embodiments of the present invention.

The pharmaceutical composition according to at least some embodiments of the present invention is further preferably used for the treatment of cancer, wherein the cancer may be non-metastatic, invasive or metastatic, treatment of immune related disorder, infectious disorder and/or sepsis.

A composition is said to be a “pharmaceutically acceptable carrier” if its administration can be tolerated by a recipient patient. As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion).

Such compositions include sterile water, buffered saline (e.g., Tris-HCl, acetate, phosphate), pH and ionic strength and optionally additives such as detergents and solubilizing agents (e.g., Polysorbate 20®, Polysorbate 80®), antioxidants (e.g, ascorbic acid, sodium metabisulfite), preservatives (e.g, Thimersol®, benzyl alcohol) and bulking substances (e.g., lactose, mannitol). Non-aqueous solvents or vehicles may also be used as detailed below.

Examples of suitable aqueous and nonaqueous carriers that may be employed in the pharmaceutical compositions according to at least some embodiments of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. Depending on the route of administration, the active compound, i.e., soluble polypeptide, fusion protein or conjugate containing the ectodomain of the VSTM5 antigen, may be coated in a material to protect the compound from the action of acids and other natural conditions that may inactivate the compound. The pharmaceutical compounds according to at least some embodiments of the invention may include one or more pharmaceutically acceptable salts. A “pharmaceutically acceptable salt” refers to a salt that retains the desired biological activity of the parent compound and does not impart any undesired toxicological effects (see e.g., Berge, S. M., et al. J. Pharm. Sci. 66: 1-19. (1977)). Examples of such salts include acid addition salts and base addition salts. Acid addition salts include those derived from nontoxic inorganic acids, such as hydrochloric, nitric, phosphoric, sulfuric, hydrobromic, hydriodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

A pharmaceutical composition according to at least some embodiments of the invention also may include a pharmaceutically acceptable anti-oxidant. Examples of pharmaceutically acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, α-tocopherol, and the like; and (3) metal chelating agents, such as citric acid, ethylenediamine tetraacetic acid (EDTA), sorbitol, tartaric acid, phosphoric acid, and the like.

These compositions may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions according to at least some embodiments of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin. Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

Sterile injectable solutions can be prepared by incorporating the active compound in the required amount in an appropriate solvent with one or a combination of ingredients enumerated above, as required, followed by sterilization microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about I percent to about 30 percent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms according to at least some embodiments of the invention are dictated by and directly dependent on (a) the unique characteristics of the active compound and the particular therapeutic effect to be achieved, and (b) the limitations inherent in the art of compounding such an active compound for the treatment of sensitivity in individuals.

For fusion proteins as described herein, optionally a similar dosage regimen is followed; alternatively, the fusion proteins may optionally be administered in an amount between 0.0001 to 100 mg/kg weight of the patient/day, preferably between 0.001 to 10.0 mg/kg/day, according to any suitable timing regimen. A therapeutic composition according to at least some embodiments of the invention can be administered, for example, three times a day, twice a day, once a day, three times weekly, twice weekly or once weekly, once every two weeks or 3, 4, 5, 6, 7 or 8 weeks. Moreover, the composition can be administered over a short or long period of time (e.g., 1 week, 1 month, 1 year, 5 years).

Alternatively, therapeutic agent can be administered as a sustained release formulation, in which case less frequent administration is required. Dosage and frequency vary depending on the half-life of the therapeutic agent in the patient. The half-life for fusion proteins may vary widely. The dosage and frequency of administration can vary depending on whether the treatment is prophylactic or therapeutic. In prophylactic applications, a relatively low dosage is administered at relatively infrequent intervals over a long period of time. Some patients continue to receive treatment for the rest of their lives. In therapeutic applications, a relatively high dosage at relatively short intervals is sometimes required until progression of the disease is reduced or terminated, and preferably until the patient shows partial or complete amelioration of symptoms of disease. Thereafter, the patient can be administered a prophylactic regime.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions of the present invention employed, or the ester, salt or amide thereof, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A “therapeutically effective dosage” of VSTM5 soluble protein or VSTM5 ectodomain or fusion protein containing same, according to at least some embodiments of the invention preferably results in a decrease in severity of disease symptoms, an increase in frequency and duration of disease symptom-free periods, an increase in lifespan, disease remission, or a prevention or reduction of impairment or disability due to the disease affliction. For example, for the treatment of VSTM5 positive tumors, a “therapeutically effective dosage” preferably inhibits cell growth or tumor growth by at least about 20%, more preferably by at least about 40%, even more preferably by at least about 60%, and still more preferably by at least about 80% relative to untreated subjects. The ability of a compound to inhibit tumor growth can be evaluated in an animal model system predictive of efficacy in human tumors. Alternatively, this property of a composition can be evaluated by examining the ability of the compound to inhibit, such inhibition in vitro by assays known to the skilled practitioner. A therapeutically effective amount of a therapeutic compound can decrease tumor size, or otherwise ameliorate symptoms in a subject.

One of ordinary skill in the art would be able to determine a therapeutically effective amount based on such factors as the subject's size, the severity of the subject's symptoms, and the particular composition or route of administration selected.

A composition of the present invention can be administered via one or more routes of administration using one or more of a variety of methods known in the art. As will be appreciated by the skilled artisan, the route and/or mode of administration will vary depending upon the desired results. Preferred routes of administration for therapeutic agents according to at least some embodiments of the invention include intravascular delivery (e.g., injection or infusion), intravenous, intramuscular, intradermal, intraperitoneal, subcutaneous, spinal, oral, enteral, rectal, pulmonary (e.g., inhalation), nasal, topical (including transdermal, buccal and sublingual), intravesical, intravitreal, intraperitoneal, vaginal, brain delivery (e.g. intra-cerebroventricular, intra-cerebral, and convection enhanced diffusion), CNS delivery (e.g., intrathecal, perispinal, and intra-spinal) or parenteral (including subcutaneous, intramuscular, intravenous and intradermal), transmucosal (e.g., sublingual administration), administration or administration via an implant, or other parenteral routes of administration, for example by injection or infusion, or other delivery routes and/or forms of administration known in the art. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. In a specific embodiment, a protein, a therapeutic agent or a pharmaceutical composition according to at least some embodiments of the present invention can be administered intraperitoneally or intravenously. Alternatively, a VSTM5 therapeutic agent can be administered via a non-parenteral route, such as a topical, epidermal or mucosal route of administration, for example, intranasally, orally, vaginally, rectally, sublingually or topically.

The active compounds can be prepared with carriers that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, J. R. Robinson, ed., Marcel Dekker, Inc., New York, 1978.

Therapeutic compositions can be administered with medical devices known in the art. For example, in a preferred embodiment, a therapeutic composition according to at least some embodiments of the invention can be administered with a needles hypodermic injection device, such as the devices disclosed in U.S. Pat. Nos. 5,399,163; 5,383,851; 5,312,335; 5,064,413; 4,941,880; 4,790,824; or 4,596,556. Examples of well-known implants and modules useful in the present invention include: U.S. Pat. No. 4,487,603, which discloses an implantable micro-infusion pump for dispensing medication at a controlled rate; U.S. Pat. No. 4,486,194, which discloses a therapeutic device for administering medicaments through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. These patents are incorporated herein by reference. Many other such implants, delivery systems, and modules are known to those skilled in the art.

In certain embodiments, VSTM5 soluble proteins, ectodomains, and/or fusion proteins, can be formulated to ensure proper distribution in vivo. For example, the blood-brain barrier (BBB) excludes many highly hydrophilic compounds. To ensure that the therapeutic compounds according to at least some embodiments of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs, thus enhance targeted drug delivery (see, e.g., V. V. Ranade J. Clin. Pharmacol. 29:685 (1989)). Exemplary targeting moieties include folate or biotin (see, e.g., U.S. Pat. No. 5,416,016 to Low et al.); mannosides (Umezawa et al., Biochem. Biophys. Res. Commun. 153:1038 (1988)); antibodies (P. G. Bloeman et al. FEBS Lett. 357:140 (1995); M. Owais et al. Antimicrob. Agents Chemother. 39:180 (1995)); surfactant protein A receptor (Briscoe et al.) Am. J Physiol. 1233:134 (1995)); p120 (Schreier et al. J. Biol. Chem. 269:9090) (1994); see also K. Keinanen; M. L. Laukkanen FEBS Lett. 346:123 (1994); and Killion and Fidler Immunomethods 4:273 (1994).

Formulations for Parenteral Administration

In a further embodiment, compositions disclosed herein, including those containing peptides and polypeptides, are administered in an aqueous solution, by parenteral injection. The formulation may also be in the form of a suspension or emulsion. In general, pharmaceutical compositions are provided including effective amounts of a peptide or polypeptide, and optionally include pharmaceutically acceptable diluents, preservatives, solubilizers, emulsifiers, adjuvants and/or carriers. Such compositions optionally include one or more for the following: diluents, sterile water, buffered saline of various buffer content (e.g., Tris-HCl, acetate, phosphate), pH and ionic strength; and additives such as detergents and solubilizing agents (e.g., TWEEN 20® (polysorbate-20), TWEEN 80® (polysorbate-80)), anti-oxidants (e.g., water soluble antioxidants such as ascorbic acid, sodium metabisulfite, cysteine hydrochloride, sodium bisulfate, sodium metabisulfite, sodium sulfite; oil-soluble antioxidants, such as ascorbyl palmitate, butylated hydroxyanisole (BHA), butylated hydroxytoluene (BHT), lecithin, propyl gallate, α-tocopherol; and metal chelating agents, such as citric acid, ethylenediamine tetraacetic acid (EDTA), sorbitol, tartaric acid, phosphoric acid), and preservatives (e.g., Thimersol, benzyl alcohol) and bulking substances (e.g., lactose, mannitol). Examples of non-aqueous solvents or vehicles are ethanol, propylene glycol, polyethylene glycol, vegetable oils, such as olive oil and corn oil, gelatin, and injectable organic esters such as ethyl oleate. The formulations may be freeze dried (lyophilized) or vacuum dried and redissolved/resuspended immediately before use. The formulation may be sterilized by, for example, filtration through a bacteria retaining filter, by incorporating sterilizing agents into the compositions, by irradiating the compositions, or by heating the compositions.

Formulations for Topical Administration

VSTM5 polypeptides, fragments, fusion polypeptides, nucleic acids, and vectors disclosed herein can be applied topically. Topical administration does not work well for most peptide formulations, although it can be effective especially if applied to the lungs, nasal, oral (sublingual, buccal), vaginal, or rectal mucosa.

Compositions can be delivered to the lungs while inhaling and traverse across the lung epithelial lining to the blood stream when delivered either as an aerosol or spray dried particles having an aerodynamic diameter of less than about 5 microns. A wide range of mechanical devices designed for pulmonary delivery of therapeutic products can be used, including but not limited to nebulizers, metered dose inhalers, and powder inhalers, all of which are familiar to those skilled in the art. Some specific examples of commercially available devices are the Ultravent nebulizer (Mallinckrodt Inc., St. Louis, Mo.); the Acorn II nebulizer (Marquest Medical Products, Englewood, Colo.); the Ventolin metered dose inhaler (Glaxo Inc., Research Triangle Park, N.C.); and the Spinhaler powder inhaler (Fisons Corp., Bedford, Mass.). Nektar, Alkermes and Mannkind all have inhalable insulin powder preparations approved or in clinical trials where the technology could be applied to the formulations described herein.

Formulations for administration to the mucosa will typically be spray dried drug particles, which may be incorporated into a tablet, gel, capsule, suspension or emulsion. Standard pharmaceutical excipients are available from any formulator. Oral formulations may be in the form of chewing gum, gel strips, tablets or lozenges. Transdermal formulations may also be prepared. These will typically be ointments, lotions, sprays, or patches, all of which can be prepared using standard technology. Transdermal formulations will require the inclusion of penetration enhancers.

Controlled Delivery Polymeric Matrices

VSTM5 polypeptides, fragments, fusion polypeptides, nucleic acids, and vectors disclosed herein may also be administered in controlled release formulations. Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of polypeptides or nucleic acids encoding the polypeptides, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be crosslinked with multivalent ions or polymers.

The matrices can be formed by solvent evaporation, spray drying, solvent extraction and other methods known to those skilled in the art. Bioerodible microspheres can be prepared using any of the methods developed for making microspheres for drug delivery, for example, as described by Mathiowitz and Langer, J. Controlled Release, 5:13-22 (1987); Mathiowitz, et al., Reactive Polymers, 6:275-283 (1987); and Mathiowitz, et al., J. Appl Polymer Sci, 35:755-774 (1988).

The devices can be formulated for local release to treat the area of implantation or injection—which will typically deliver a dosage that is much less than the dosage for treatment of an entire body—or systemic delivery. These can be implanted or injected subcutaneously, into the muscle, fat, or swallowed.

Diagnostic Uses of VSTM5 Polypeptides

Soluble VSTM5 polypeptides according to at least some embodiments of the present invention may also be modified with a label capable of providing a detectable signal, either directly or indirectly, including, but not limited to, radioisotopes and fluorescent compounds. Such labeled polypeptides can be used for various uses, including but not limited to, diagnosis, prognosis, prediction, screening, early diagnosis, determination of progression, therapy selection and treatment monitoring of disease, disorder and/or an indicative condition, as detailed herein.

As used herein the term “diagnosis” refers to the process of identifying or aiding in the identification of a medical condition or disease by its signs, symptoms, and in particular from the results of various diagnostic procedures, including e.g., using labeled VSTM5 polypeptides according to at least some embodiments of the present invention, as described herein. Furthermore, as used herein the term “diagnosis” encompasses screening for a disease, detecting a presence or a severity of a disease, providing prognosis of a disease, monitoring disease progression or relapse, as well as assessment of treatment efficacy and/or relapse of a disease, disorder or condition, as well as selecting a therapy and/or a treatment for a disease, optimization of a given therapy for a disease, monitoring the treatment of a disease, and/or predicting the suitability of a therapy for specific patients or subpopulations or determining the appropriate dosing of a therapeutic product in patients or subpopulations. The diagnostic procedure can be performed in vivo or in vitro.

According to at least some embodiments, the present invention provides a method for imaging an organ or tissue, the method comprising: (a) administering to a subject in need of such imaging, a labeled polypeptide; and (b) detecting the labeled polypeptide to determine where the labeled polypeptide is concentrated in the subject. When used in imaging applications, the labeled polypeptides according to at least some embodiments of the present invention typically have an imaging agent covalently or monovalently attached thereto. Suitable imaging agents include, but are not limited to, radionuclides, detectable tags, fluorophores, fluorescent proteins, enzymatic proteins, and the like. One of skill in the art will be familiar with other methods for attaching imaging agents to polypeptides. For example, the imaging agent can be attached via site-specific conjugation, e.g., covalent attachment of the imaging agent to a peptide linker such as a polyarginine moiety having five to seven arginines present at the carboxyl-terminus of and Fc fusion molecule. The imaging agent can also be directly attached via non-site specific conjugation, e.g., covalent attachment of the imaging agent to primary amine groups present in the polypeptide. One of skill in the art will appreciate that an imaging agent can also be bound to a protein via noncovalent interactions (e.g., ionic bonds, hydrophobic interactions, hydrogen bonds, Van der Waals forces, dipole-dipole bonds, etc.).

In certain instances, the polypeptide is radiolabeled with a radionuclide by directly attaching the radionuclide to the polypeptide. In certain other instances, the radionuclide is bound to a chelating agent or chelating agent-linker attached to the polypeptide. Suitable radionuclides for direct conjugation include, without limitation, ¹⁸F, ¹²⁴I, ¹²⁵I, ¹³¹I, and mixtures thereof. Suitable radionuclides for use with a chelating agent include, without limitation ⁴⁷Sc, ⁴⁶Cu, ⁶⁷Cu, ⁸⁹Sr, ⁸⁶Y, ⁸⁷Y, ⁹⁰Y, ¹⁰⁵Rh, ¹¹¹Ag, ¹¹¹In, ¹⁴⁹P, ¹¹⁷mSn, ¹⁴⁹Pm, ¹⁵³Sm, ¹⁶⁶Ho, ¹⁷⁷Lu, ¹⁸⁶Re, ¹⁸⁸Re, ²¹¹At, ²¹²Bi and mixtures thereof.

Preferably, the radionuclide bound to a chelating agent is ⁶⁴Cu, ⁹⁹Y, ¹¹¹In, or mixtures thereof. Suitable chelating agents include, but are not limited to, DOTA, BAD, TETA, DTPA, EDTA, NTA, HDTA, their phosphonate analogs, and mixtures thereof. One of skill in the art will be familiar with methods for attaching radionuclides, chelating agents, and chelating agent-linkers to polypeptides of the present invention. In particular, attachment can be conveniently accomplished using, for example, commercially available bifunctional linking groups (generally heterobifunctional linking groups) that can be attached to a functional group present in a non-interfering position on the polypeptide and then further linked to a radionuclide, chelating agent, or chelating agent-linker.

Non-limiting examples of fluorophores or fluorescent dyes suitable for use as imaging agents include Alexa Fluor® dyes (Invitrogen Corp.; Carlsbad, Calif.), fluorescein, fluorescein isothiocyanate (FITC), Oregon Green™; rhodamine, Texas red, tetrarhodamine isothiocynate (TRITC), CyDye™ fluors (e.g., Cy2, Cy3, Cy5), and the like.

Examples of fluorescent proteins suitable for use as imaging agents include, but are not limited to, green fluorescent protein, red fluorescent protein (e.g., DsRed), yellow fluorescent protein, cyan fluorescent protein, blue fluorescent protein, and variants thereof (see, e.g., U.S. Pat. Nos. 6,403,374, 6,800,733, and 7,157,566). Specific examples of GFP variants include, but are not limited to, enhanced GFP (EGFP), destabilized EGFP, the GFP variants described in Doan et al., Mol. Microbiol., 55:1767-1781 (2005), the GFP variant described in Crameri et al., Nat. Biotechnol., 14:315-319 (1996), cerulean fluorescent proteins described in Rizzo et al., Nat. Biotechnol, 22:445 (2004) and Tsien, Annu. Rev. Biochem., 67:509 (1998), and the yellow fluorescent protein described in Nagal et al., Nat. Biotechnol., 20:87-90 (2002). DsRed variants are described in, e.g., Shaner et al., Nat. Biotechnol., 22:1567-1572 (2004), and include mStrawberry, mCherry, mOrange, mBanana, mHoneydew, and mTangerine. Additional DsRed variants are described in, e.g., Wang et al., Proc. Natl. Acad. Sci. U.S.A., 101:16745-16749 (2004) and include mRaspberry and mPlum. Further examples of DsRed variants include mRFPmars described in Fischer et al., FEBS Lett., 577:227-232 (2004) and mRFPruby described in Fischer et al., FEBS Lett., 580:2495-2502 (2006).

In other embodiments, the imaging agent that is bound to a polypeptide according to at least some embodiments of the present invention comprises a detectable tag such as, for example, biotin, avidin, streptavidin, or neutravidin. In further embodiments, the imaging agent comprises an enzymatic protein including, but not limited to, luciferase, chloramphenicol acetyltransferase, β-galactosidase, β-glucuronidase, horseradish peroxidase, xylanase, alkaline phosphatase, and the like.

Any device or method known in the art for detecting the radioactive emissions of radionuclides in a subject is suitable for use in the present invention. For example, methods such as Single Photon Emission Computerized Tomography (SPECT), which detects the radiation from a single photon γ-emitting radionuclide using a rotating γ camera, and radionuclide scintigraphy, which obtains an image or series of sequential images of the distribution of a radionuclide in tissues, organs, or body systems using a scintillation gamma camera, may be used for detecting the radiation emitted from a radiolabeled polypeptide of the present invention. Positron emission tomography (PET) is another suitable technique for detecting radiation in a subject. Miniature and flexible radiation detectors intended for medical use are produced by Intra-Medical LLC (Santa Monica, Calif.). Magnetic Resonance Imaging (MRI) or any other imaging technique known to one of skill in the art is also suitable for detecting the radioactive emissions of radionuclides. Regardless of the method or device used, such detection is aimed at determining where the labeled polypeptide is concentrated in a subject, with such concentration being an indicator of disease activity.

Non-invasive fluorescence imaging of animals and humans can also provide in vivo diagnostic information and be used in a wide variety of clinical specialties. For instance, techniques have been developed over the years for simple ocular observations following UV excitation to sophisticated spectroscopic imaging using advanced equipment (see, e.g., Andersson-Engels et al., Phys. Med. Biol., 42:815-824 (1997)). Specific devices or methods known in the art for the in vivo detection of fluorescence, e.g., from fluorophores or fluorescent proteins, include, but are not limited to, in vivo near-infrared fluorescence (see, e.g., Frangioni, Curr. Opin. Chem. Biol., 7:626-634 (2003)), the Maestro™ in vivo fluorescence imaging system (Cambridge Research & Instrumentation, Inc.; Woburn, Mass.), in vivo fluorescence imaging using a flying-spot scanner (see, e.g., Ramanuj am et al., IEEE Transactions on Biomedical Engineering, 48:1034-1041 (2001), and the like.

Other methods or devices for detecting an optical response include, without limitation, visual inspection, CCD cameras, video cameras, photographic film, laser-scanning devices, fluorometers, photodiodes, quantum counters, epifluorescence microscopes, scanning microscopes, flow cytometers, fluorescence microplate readers, or signal amplification using photomultiplier tubes.

In yet another embodiment, immunoconjugates of the invention can be used to target compounds (e.g., therapeutic agents, labels, cytotoxins, radiotoxins immunosuppressants, etc.) to cells which have VSTM5 cell surface receptors by linking such compounds to the antibody. Thus, the invention also provides methods for localizing ex vivo or in vivo cells expressing VSTM5 (e.g., with a detectable label, such as a radioisotope, a fluorescent compound, an enzyme, or an enzyme co-factor). Alternatively, the immunoconjugates can be used to kill cells which have VSTM5 cell surface receptors by targeting cytotoxins or radiotoxins to VSTM5 antigen.

Theranostics:

The term theranostics describes the use of diagnostic testing to diagnose the disease, choose the correct treatment regime according to the results of diagnostic testing and/or monitor the patient response to therapy according to the results of diagnostic testing. Theranostic tests can be used to select patients for treatments that are particularly likely to benefit them and unlikely to produce side-effects. They can also provide an early and objective indication of treatment efficacy in individual patients, so that (if necessary) the treatment can be altered with a minimum of delay. For example: DAKO and Genentech together created HercepTest® and Herceptin® (trastuzumab) for the treatment of breast cancer, the first theranostic test approved simultaneously with a new therapeutic drug. In addition to HercepTest (which is an immunohistochemical test), other theranostic tests are in development which use traditional clinical chemistry, immunoassay, cell-based technologies and nucleic acid tests. PPGx's recently launched TPMT (thiopurine S-methyltransferase) test, which is enabling doctors to identify patients at risk for potentially fatal adverse reactions to 6-mercaptopurine, an agent used in the treatment of leukemia. Also, Nova Molecular pioneered SNP genotyping of the apolipoprotein E gene to predict Alzheimer's disease patients' responses to cholinomimetic therapies and it is now widely used in clinical trials of new drugs for this indication. Thus, the field of theranostics represents the intersection of diagnostic testing information that predicts the response of a patient to a treatment with the selection of the appropriate treatment for that particular patient.

Surrogate Markers:

The VSTM5 protein compositions according to at least some embodiments of the invention can be used as a surrogate marker. A surrogate marker is a marker, that is detectable in a laboratory and/or according to a physical sign or symptom on the patient, and that is used in therapeutic trials as a substitute for a clinically meaningful endpoint. The surrogate marker is a direct measure of how a patient feels, functions, or survives which is expected to predict the effect of the therapy. The need for surrogate markers mainly arises when such markers can be measured earlier, more conveniently, or more frequently than the endpoints of interest in terms of the effect of a treatment on a patient, which are referred to as the clinical endpoints. Ideally, a surrogate marker should be biologically plausible, predictive of disease progression and measurable by standardized assays (including but not limited to traditional clinical chemistry, immunoassay, cell-based technologies, nucleic acid tests and imaging modalities).

VSTM5 protein compositions according to at least some embodiments of the invention which have complement binding sites, such as portions from IgG1, IGg2, or IgG3 or IgM which bind complement, can also be used in the presence of complement. In one embodiment, ex vivo treatment of a population of cells comprising target cells with a binding agent according to at least some embodiments of the invention and appropriate effector cells can be supplemented by the addition of complement or serum containing complement. Phagocytosis of target cells coated with a binding agent according to at least some embodiments of the invention can be improved by binding of complement proteins. In another embodiment target cells coated with the compositions according to at least some embodiments of the invention can also be lysed by complement. In yet another embodiment, the compositions according to at least some embodiments of the invention do not activate complement.

The VSTM5 protein compositions according to at least some embodiments of the invention can also be administered together with complement. Thus, according to at least some embodiments of the invention there are VSTM5 protein compositions, comprising human VSTM5 soluble protein and serum or complement. These compositions are advantageous in that the complement is located in close proximity to the human VSTM5 soluble molecules. Alternatively, the human VSTM5 soluble molecules according to at least some embodiments of the invention and the complement or serum can be administered separately.

The present invention is further illustrated by the following examples. This information and examples is illustrative and should not be construed as further limiting. The contents of all figures and all references, patents and published patent applications cited throughout this application are expressly incorporated herein by reference.

### EXAMPLES

### Example 1

### IHC Analysis of VSTM5 Proteins

In order to evaluate VSTM5 expression in cancer and normal tissues several IHC studies were performed using PIPE (Formalin-Fixed, Paraffin-Embedded) samples or TMAs (Tissue MicroArray) by Asterand (Royston, UK).

### Tissue Details: ‘Multi-Tumor’ TMA

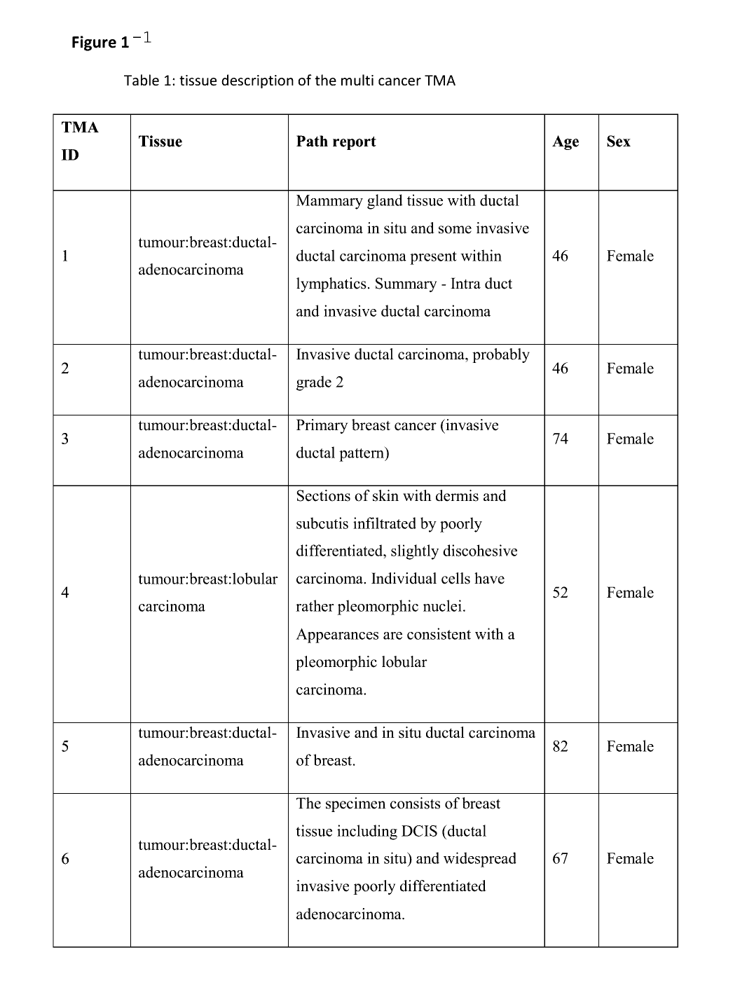

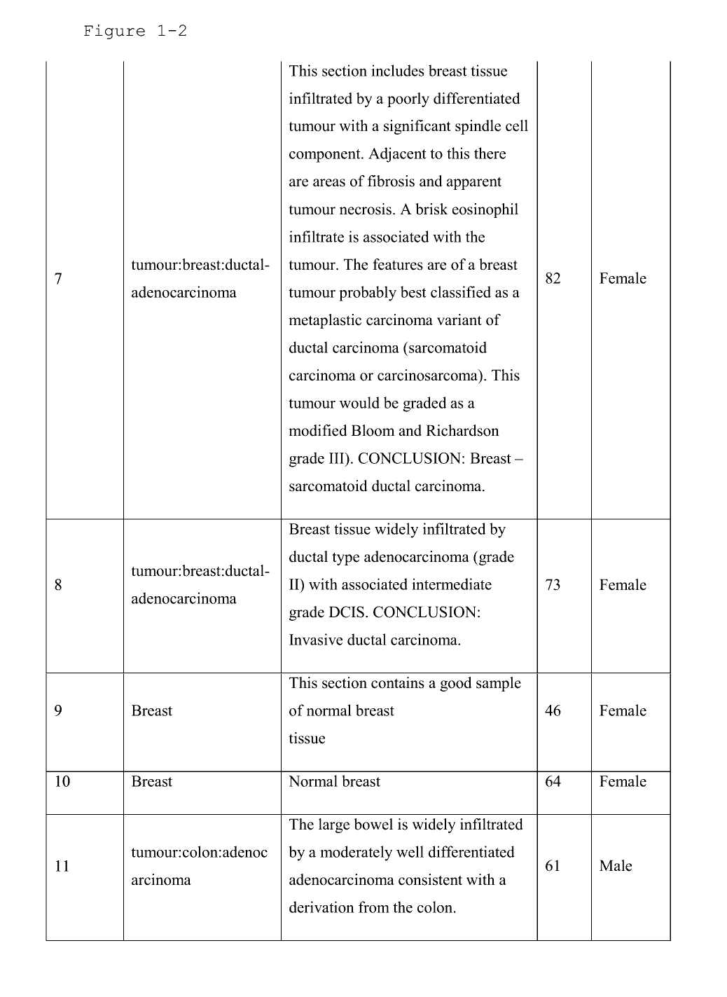

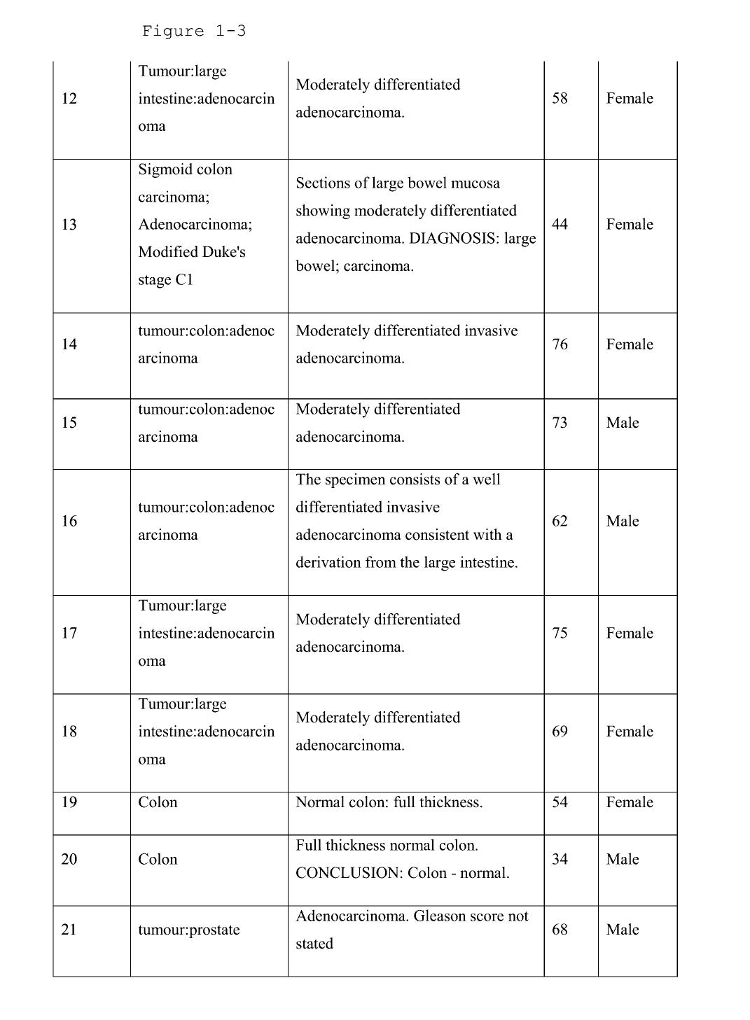

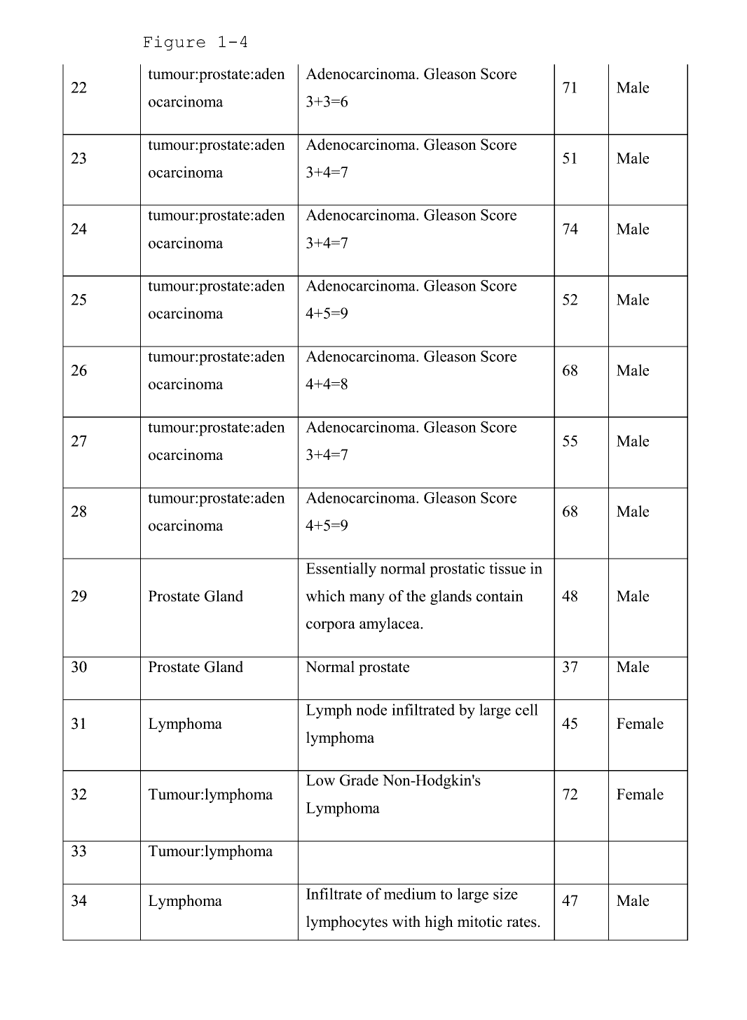

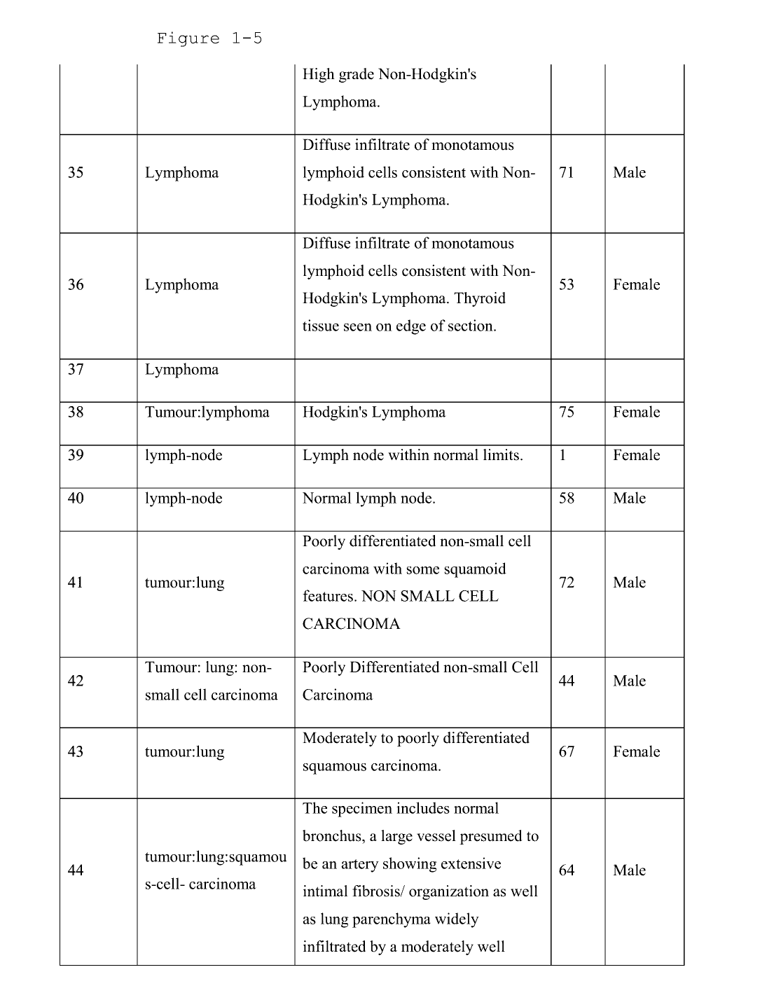

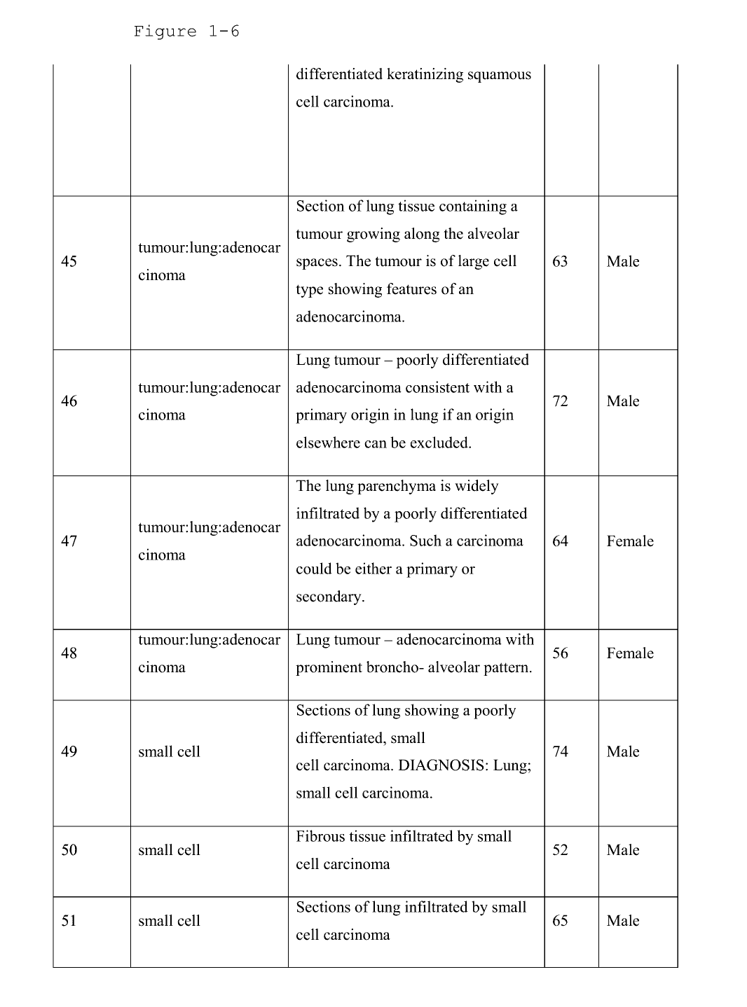

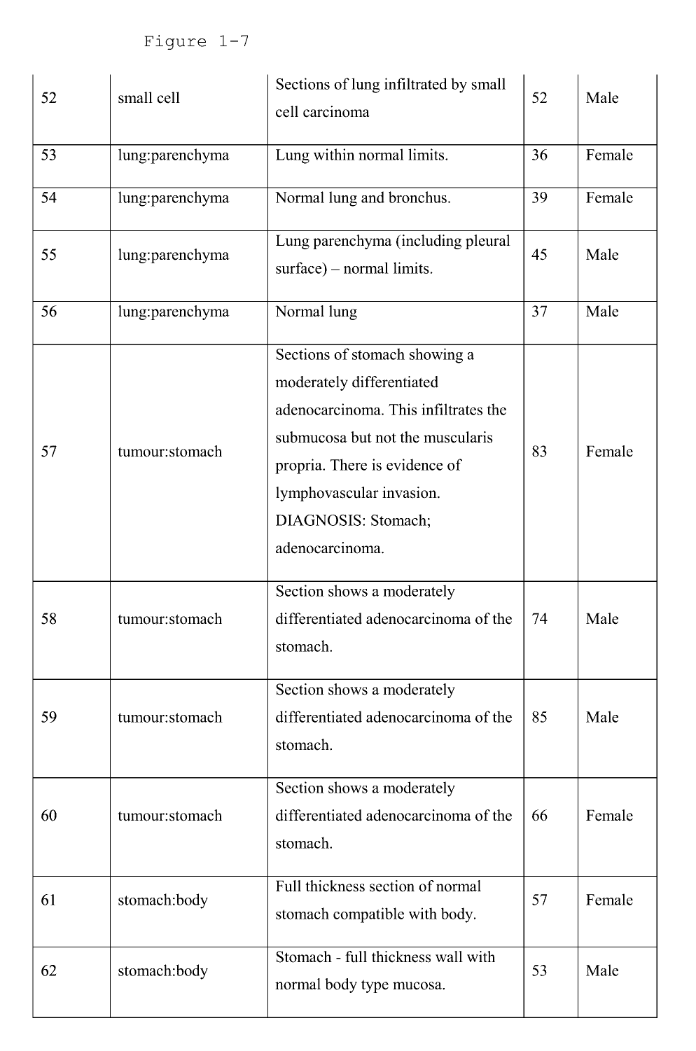

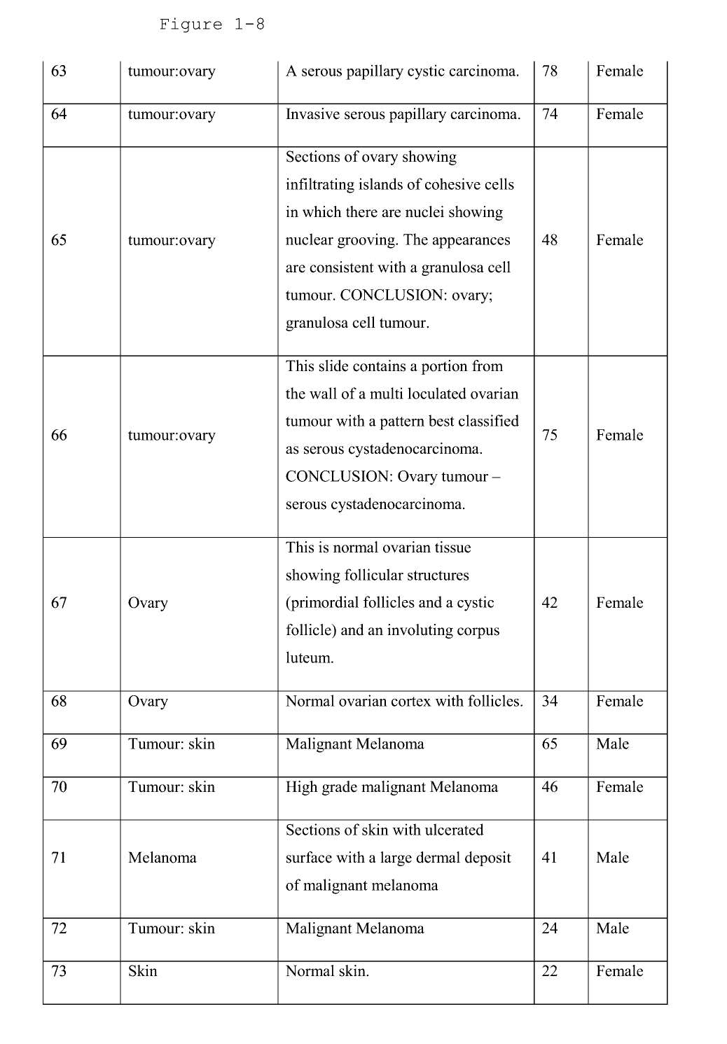

As described in detail in FIG. 1 the TMA comprised 11 tissue types: breast, colon, lymphoid and prostate (8 tumor and 2 normal samples of each), gastric, ovary, brain, kidney, liver and skin (4 tumor and 2 normal samples of each), and lung (8 non-small cell tumor and 4 small cell tumor samples, and 4 normal lung samples).

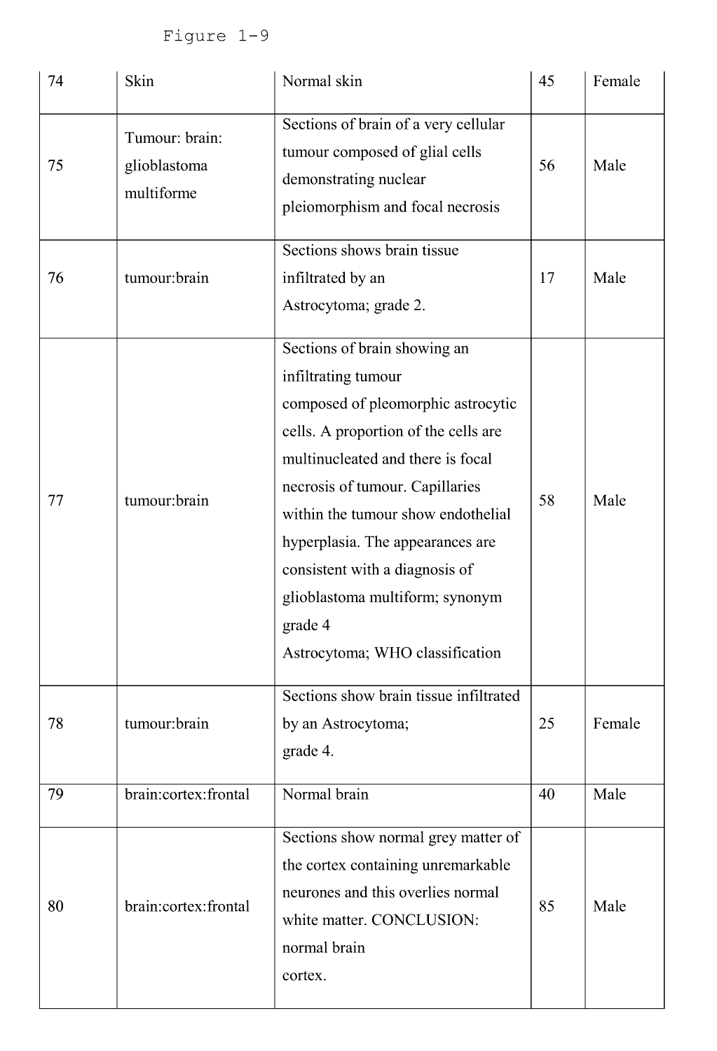

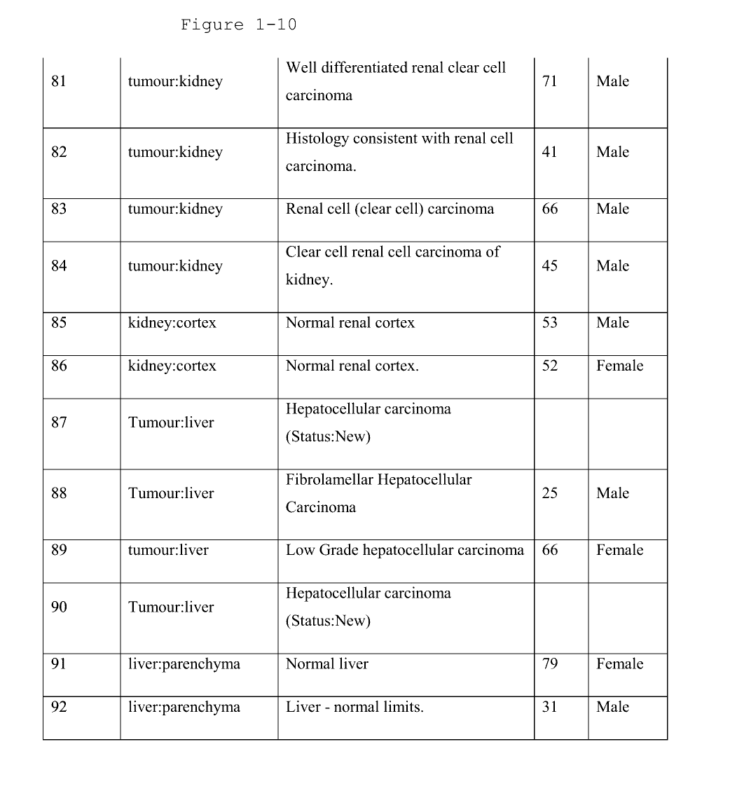

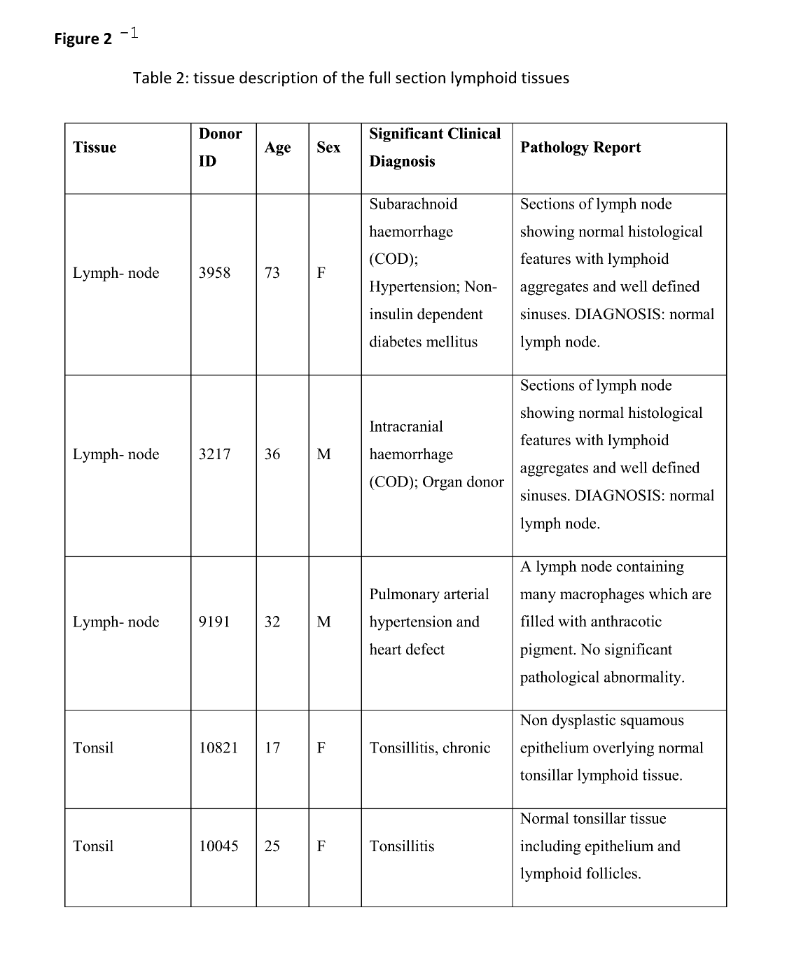

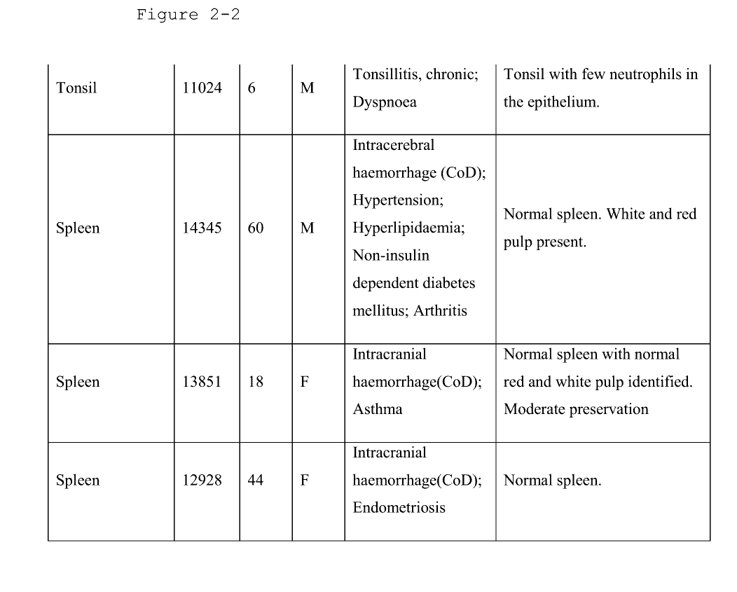

Further additional analysis in normal tissue sections of lymph node (n=3), tonsil (n=3) and spleen (n=3) were included in this study as described in FIG. 2. FIG. 1 and FIG. 2 present full clinical details of the samples used.

FFPE sections (4 μm) of the cell line HEK293T recombinantly expression VSTM5-GFP, the ‘multi-tumor’ TMA and full-face sections of normal lymph node, tonsil and spleen were used.

### Tissue Details: TOP4 TMA

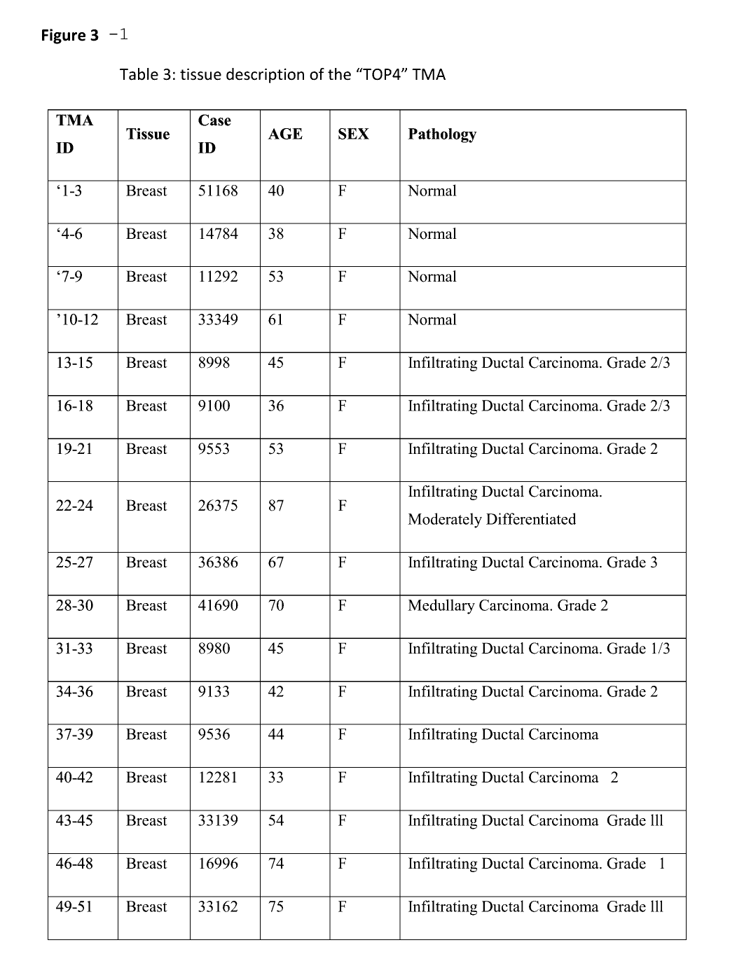

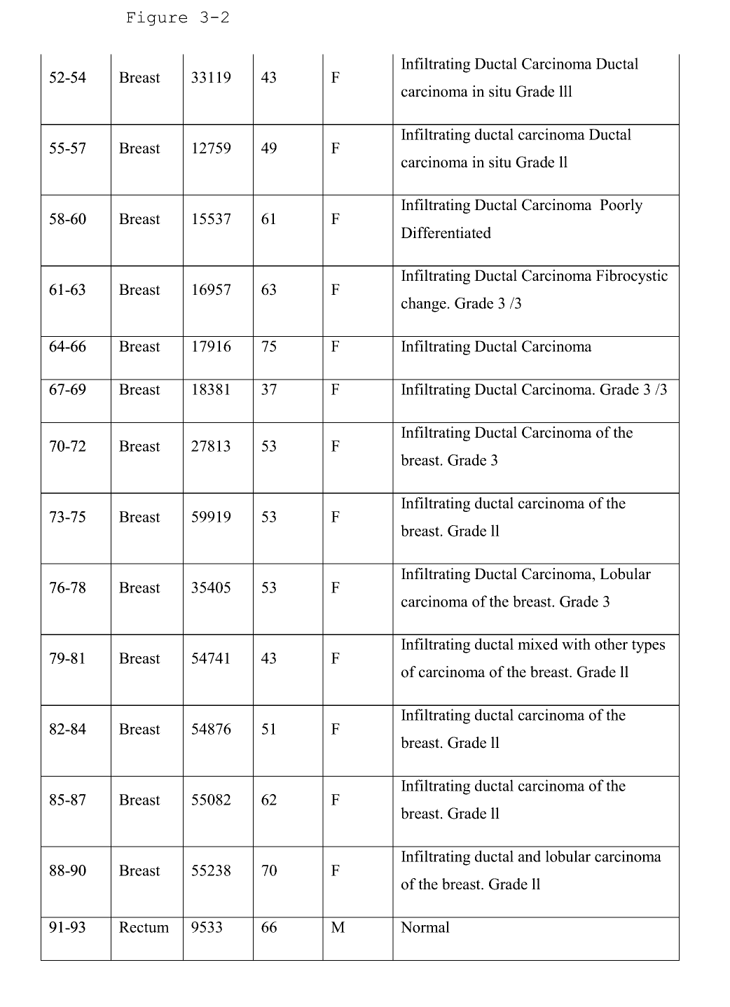

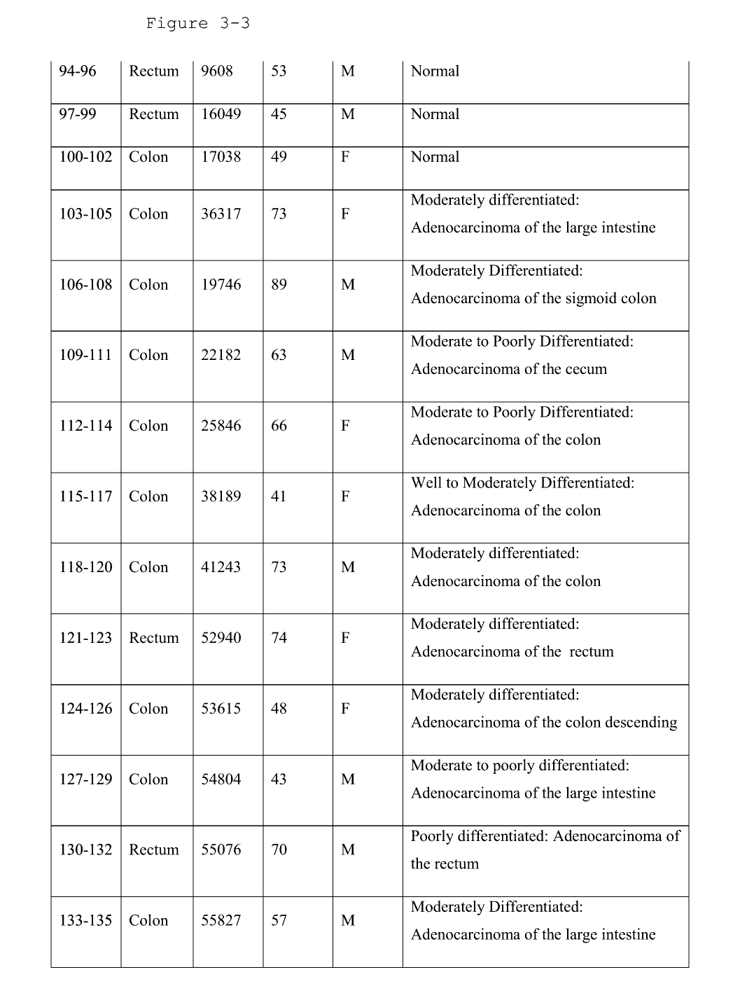

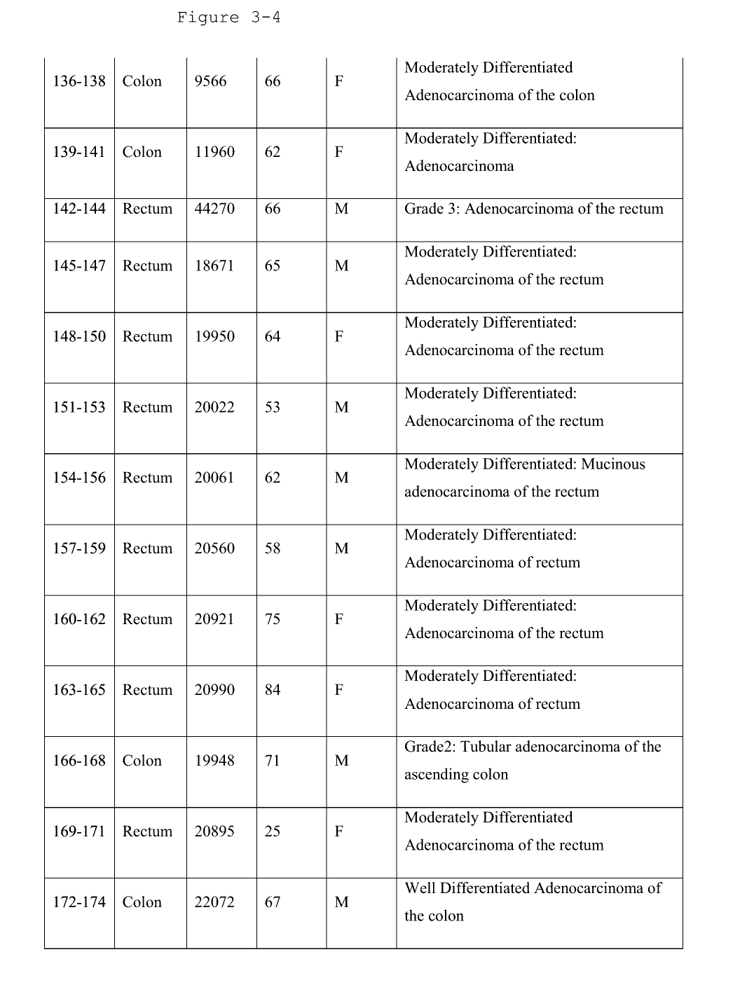

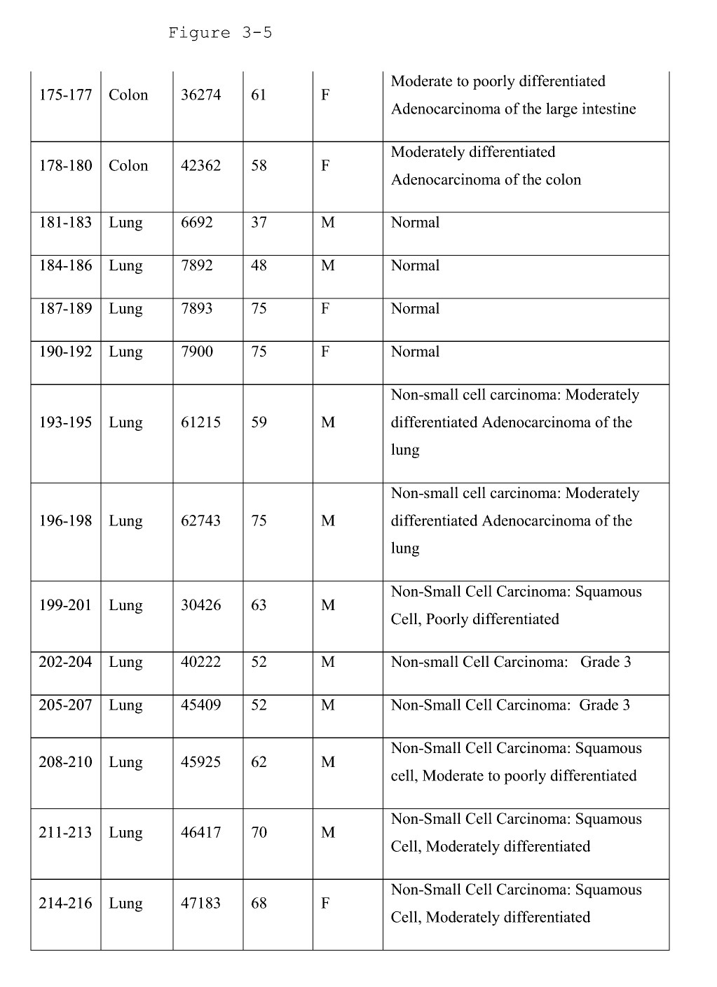

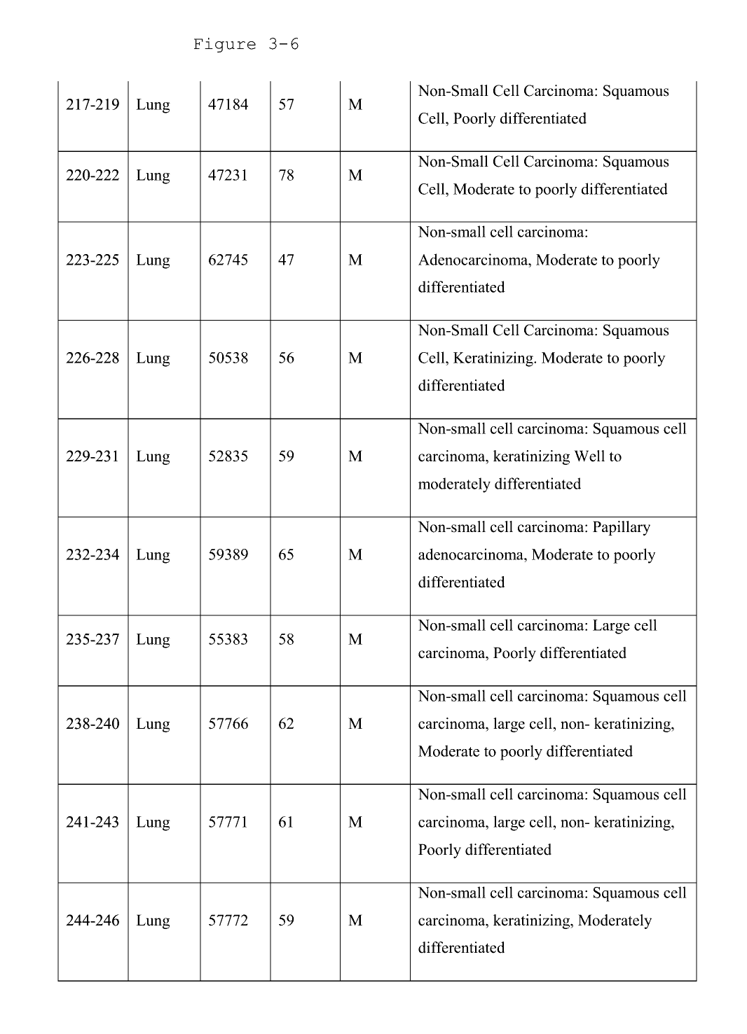

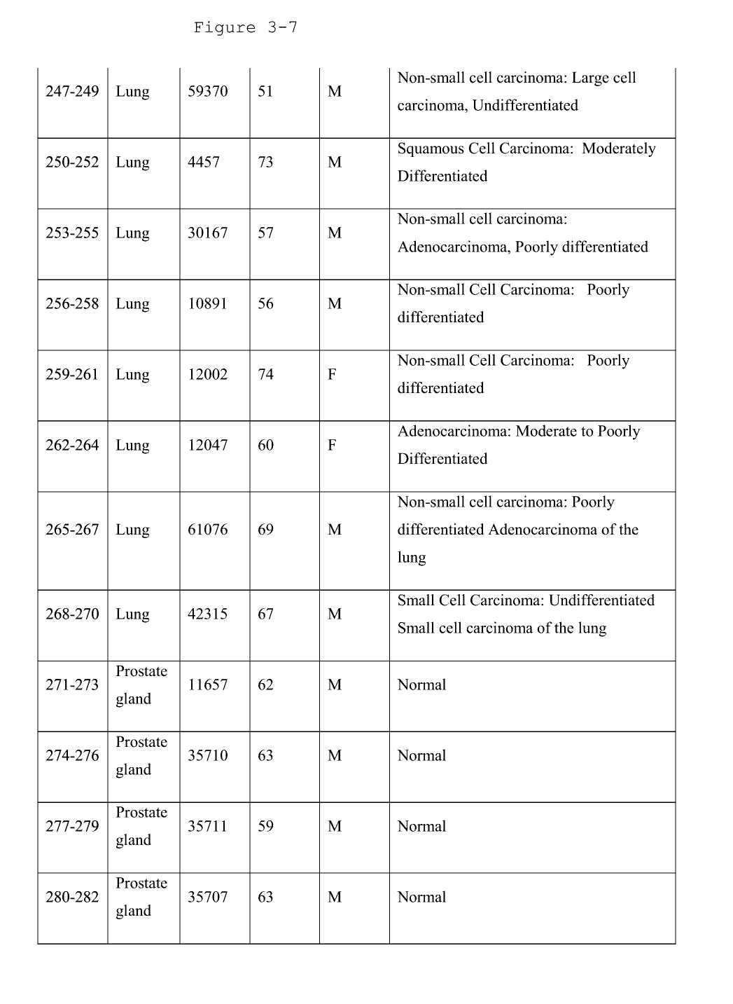

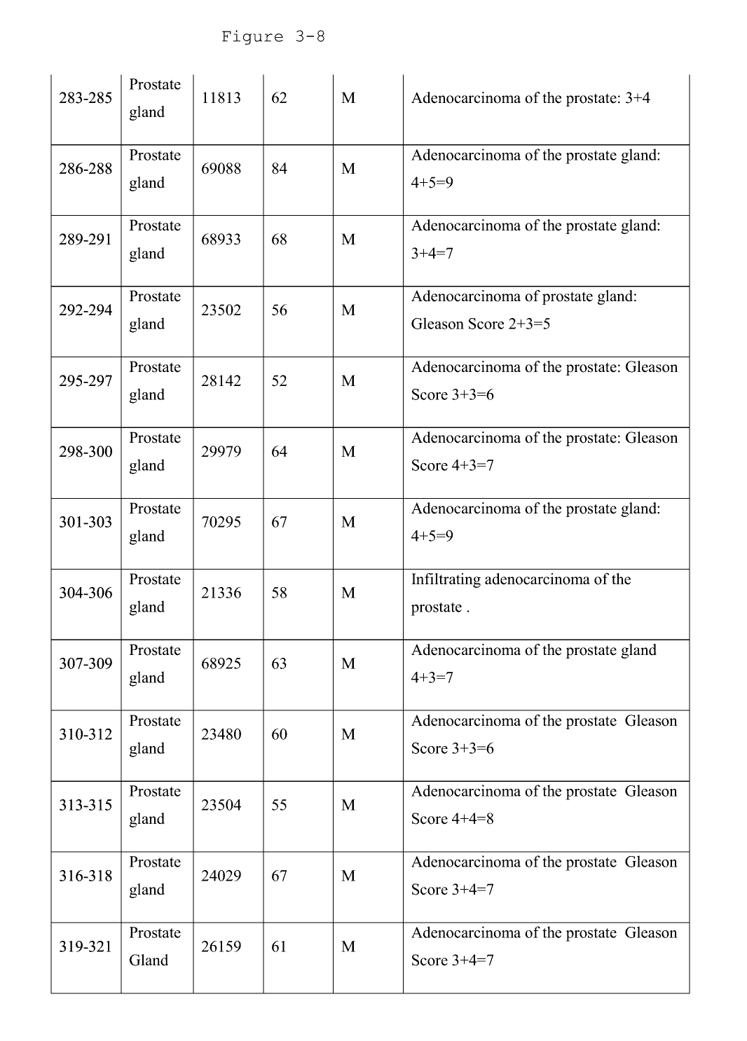

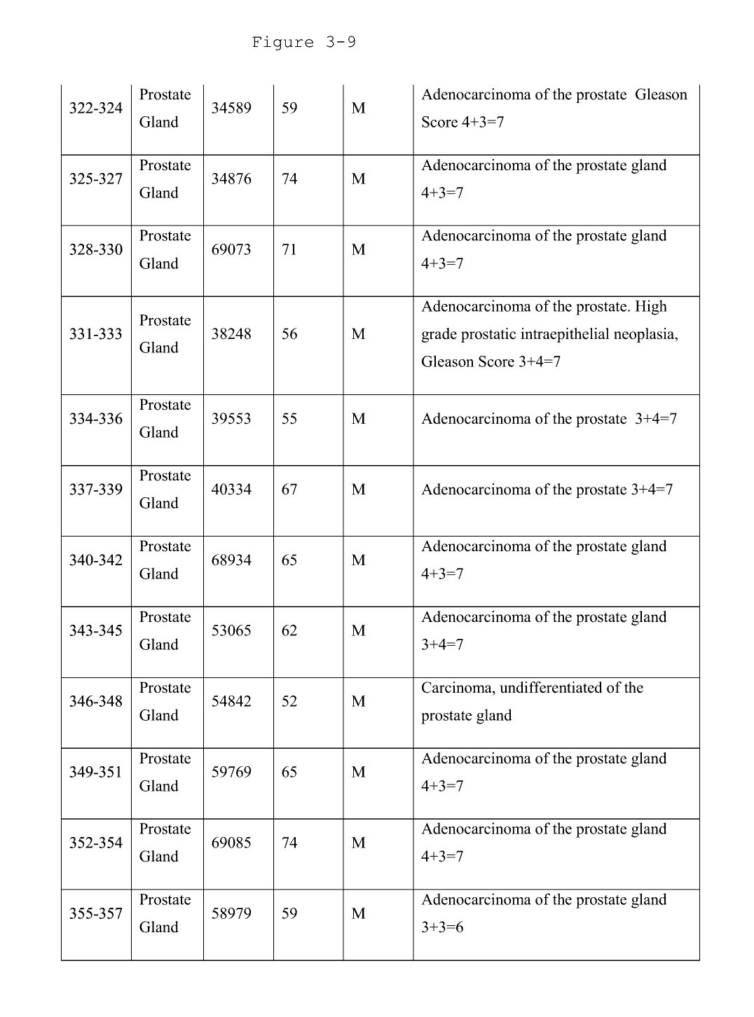

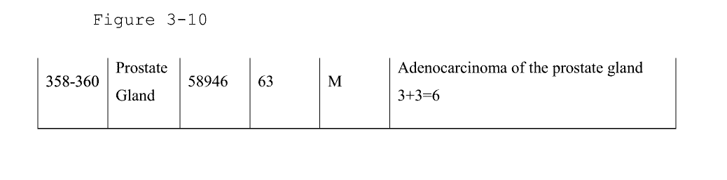

As described in FIG. 3 the ‘Top 4’ TMA is comprised of triplicate tissue cores (0.6 mm diameter) from a total of 120 different donors with an age range of 25-89 years, of which 49 were female and 71 were male. The TMA consisted of cores from 4 tissue types: breast (4 normal and 26 tumors), large intestine (4 normal and 26 tumors), lung (4 normal and 26 tumors) and prostate (4 normal and 26 tumors) FIG. 3 presents full description of the “Top 4” tissue microarray samples used.

### Antigen Retrieval and Staining

The sections were de-paraffinized; antigen retrieved and rehydrated using pH9.0 Flex+3-in-1 antigen retrieval buffers, in PT Link apparatus at 95° C. for 20 min with automatic heating and cooling.

Following antigen retrieval, sections were washed in Flex (TBST) buffer for 2×5 min then loaded into a DAKO Autostainer Plus. The sections were then incubated for 10 min with Flex and Peroxidase Blocking reagent, rinsed twice in 50 mM Tris. HCl, 150 mM NaCl, 0.1% Tween-20, pH 7.6 (TBST), followed by a 10 min incubation with Protein Block reagent (DAKO X0909).

The sections were incubated for 30 min with primary antibody diluted in DAKO Envision Flex antibody diluent (DAKO Cytomation, Cat # K8006). Anti VSTM5 (C110rf90) (HPA029525 Sigma) was applied at 3 μg/ml. Anti Von Willebrand's Factor (vWF) antibody was applied at 1 μg/ml. The negative control sections were incubated with non-immune rabbit IgG antibodies (Dako, CAT #0936) at 3 and 1 μg/ml or in DAKO Envision Flex antibody diluent (‘no primary’ control).

Following incubation with primary antibodies, the sections were then rinsed twice in FLEX buffer, incubated with anti-mouse/rabbit Flex⁺ HRP for 20 min, rinsed twice in FLEX buffer and then incubated with diaminobenzidine (DAB) substrate for 10 min. The chromogenic reaction was stopped by rinsing the slides with distilled water.

Following chromagenesis, the sections were counterstained with haematoxylin, dehydrated in an ascending series of ethanols (90-99-100%), and cleared in three changes of xylene and coverslipped under DePeX.

### Data Analysis

The sections were analyzed for the intensity of the specific staining and a semi-quantitative scoring system was used. The core in the tissue array with the most intense VSTM5-ir was assigned a score of 3⁺ and the intensities of the immunoreactivity in the other cores were scored relative to that of the 3+ core. The percentage of VSTM5-ir tumor was estimated and recorded using the following ranges: 0-25%, 25-50%, 50-75% and 75-100%. Where scoring was too low to quantitate—an assigned ‘+’ was used to denote the presence of staining. The intracellular distribution of the immune-stained cells in the tumor was also recorded.

### Result Summary of the ‘Multi-Tumor’ TMA

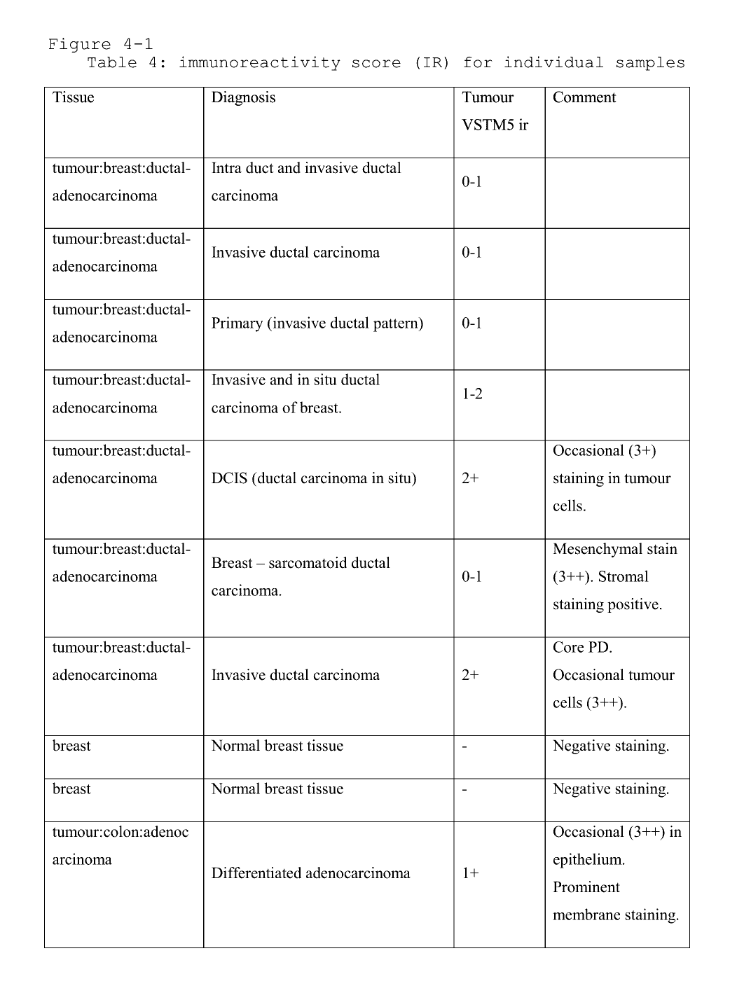

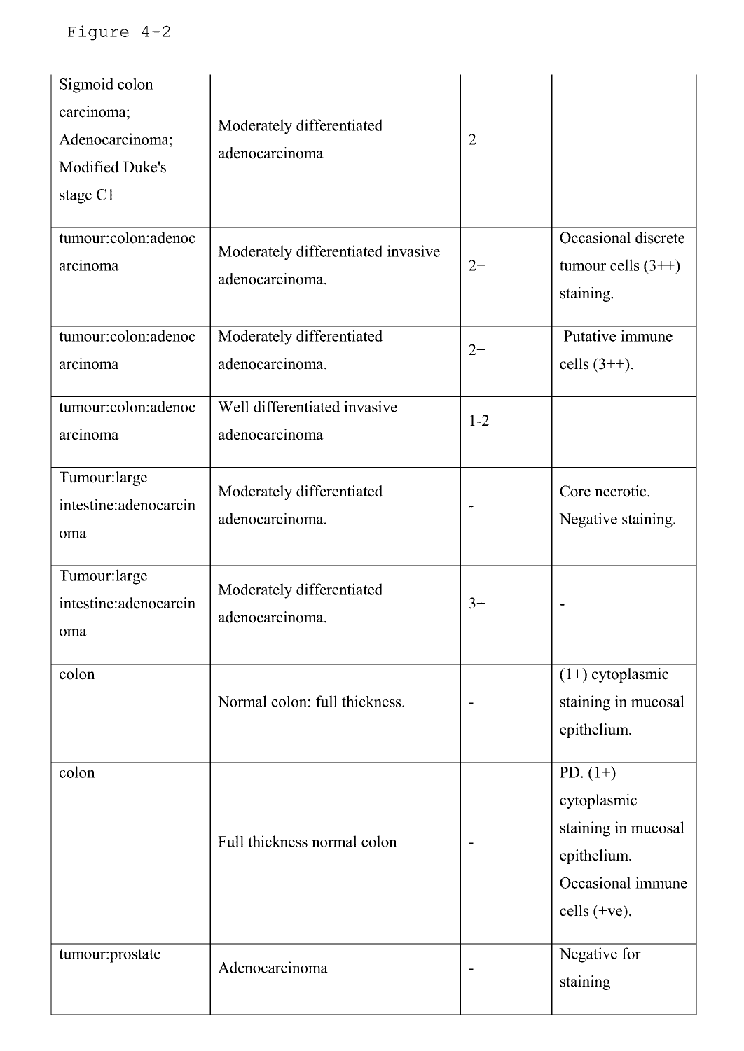

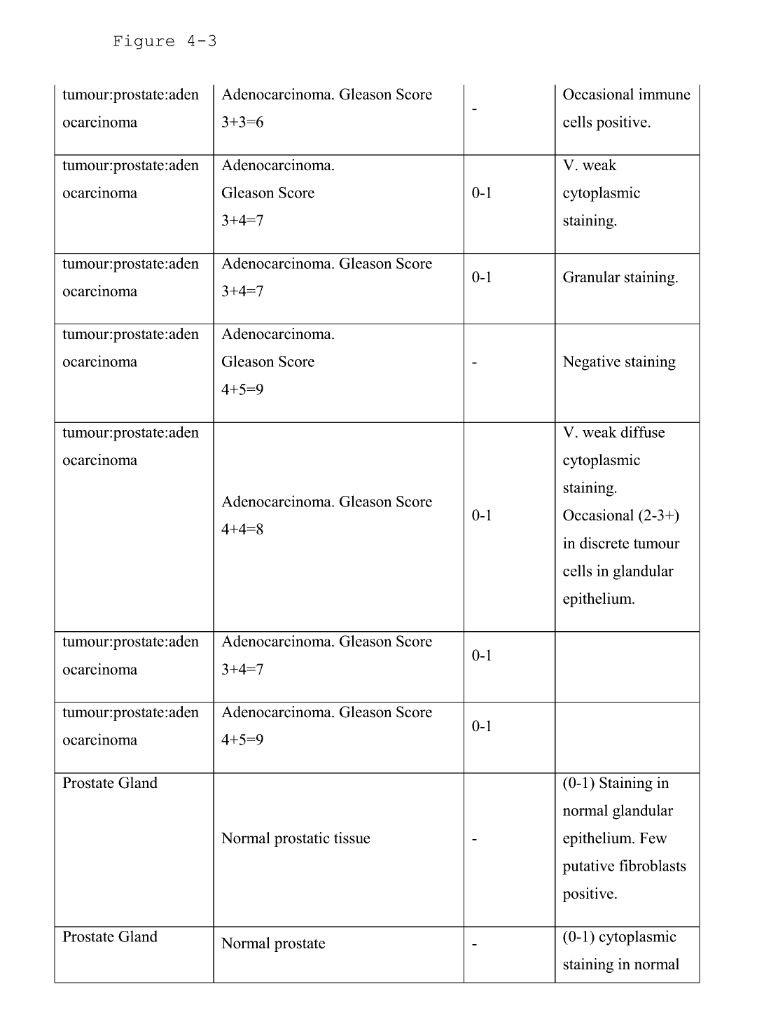

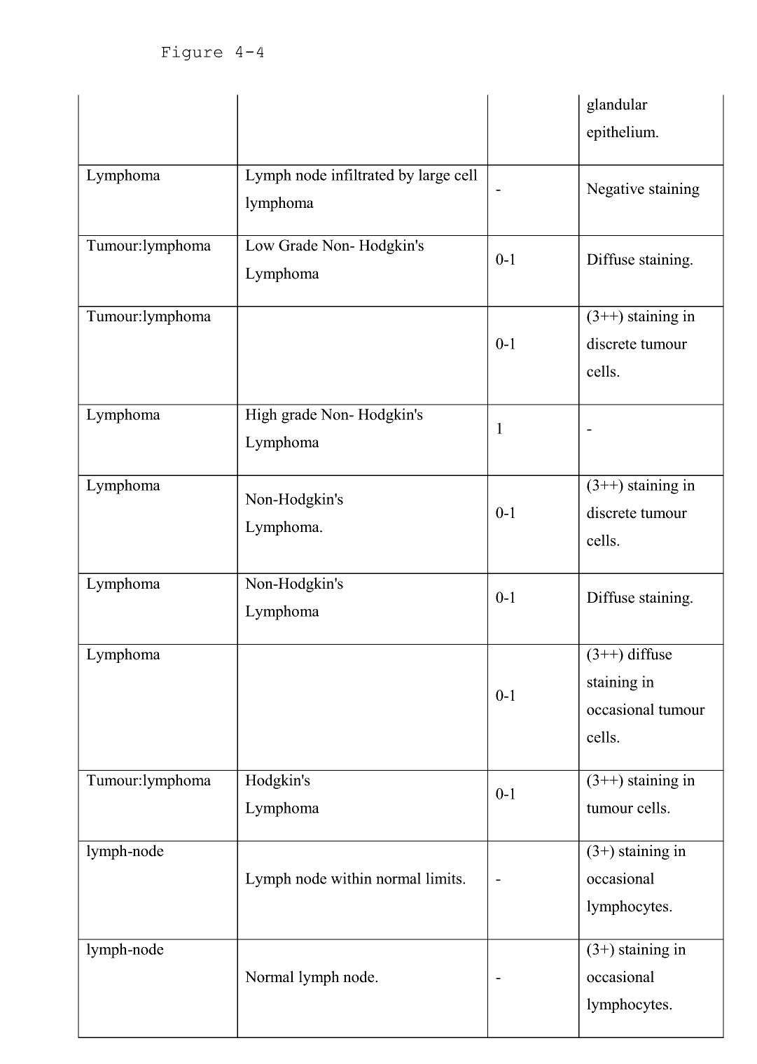

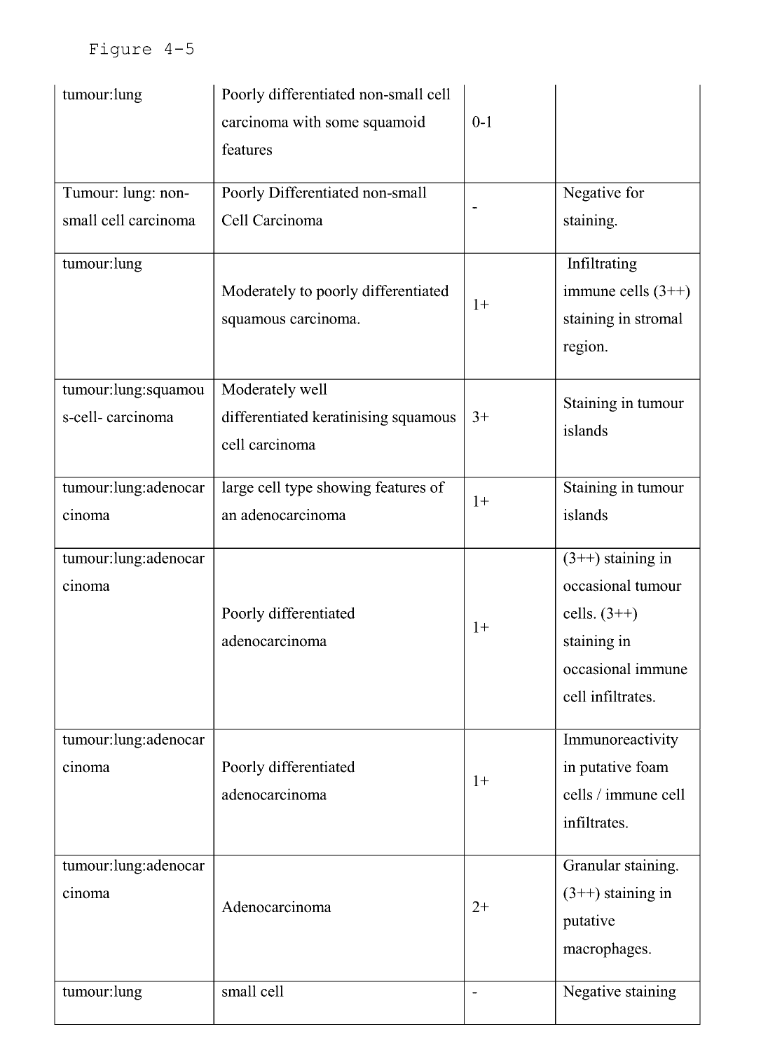

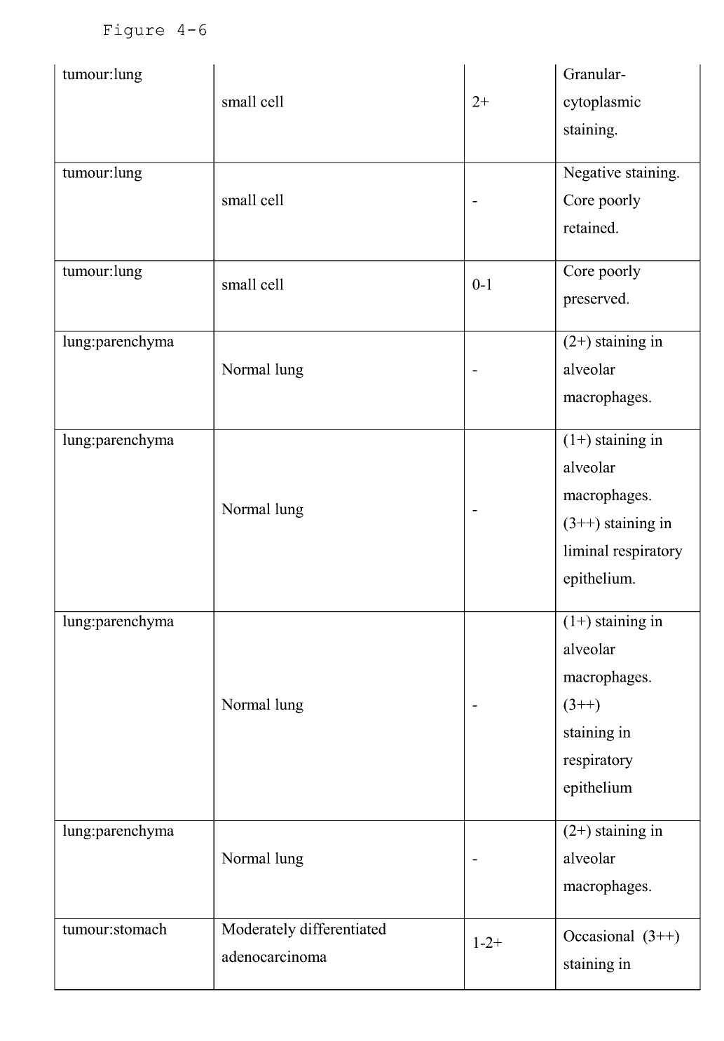

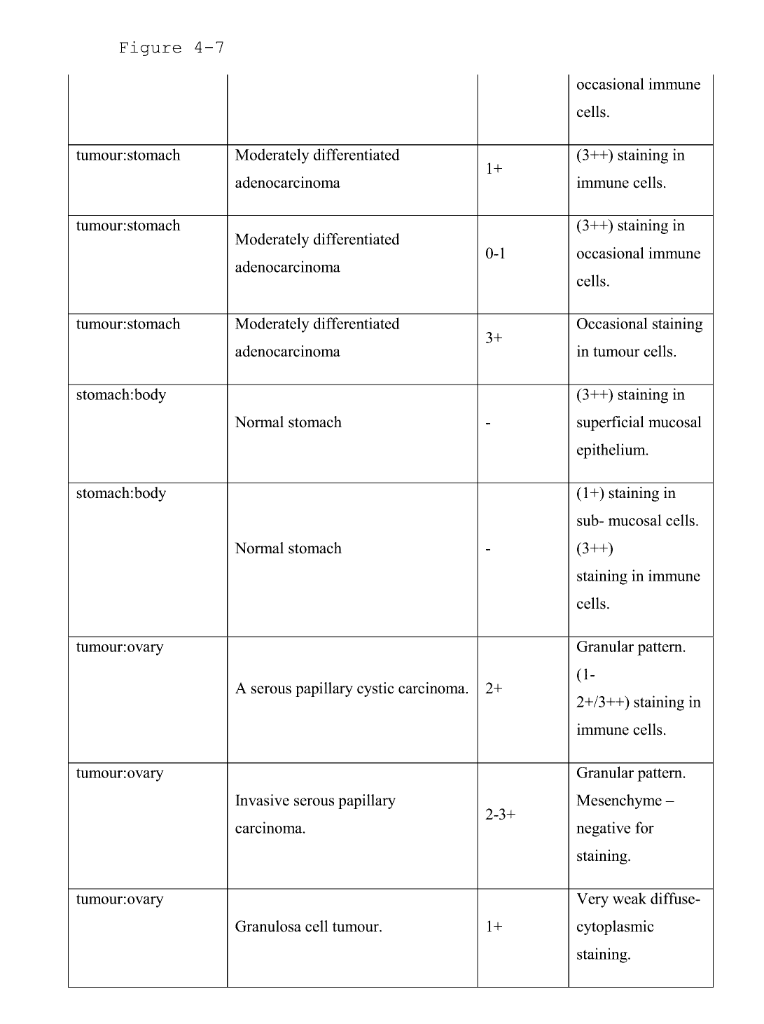

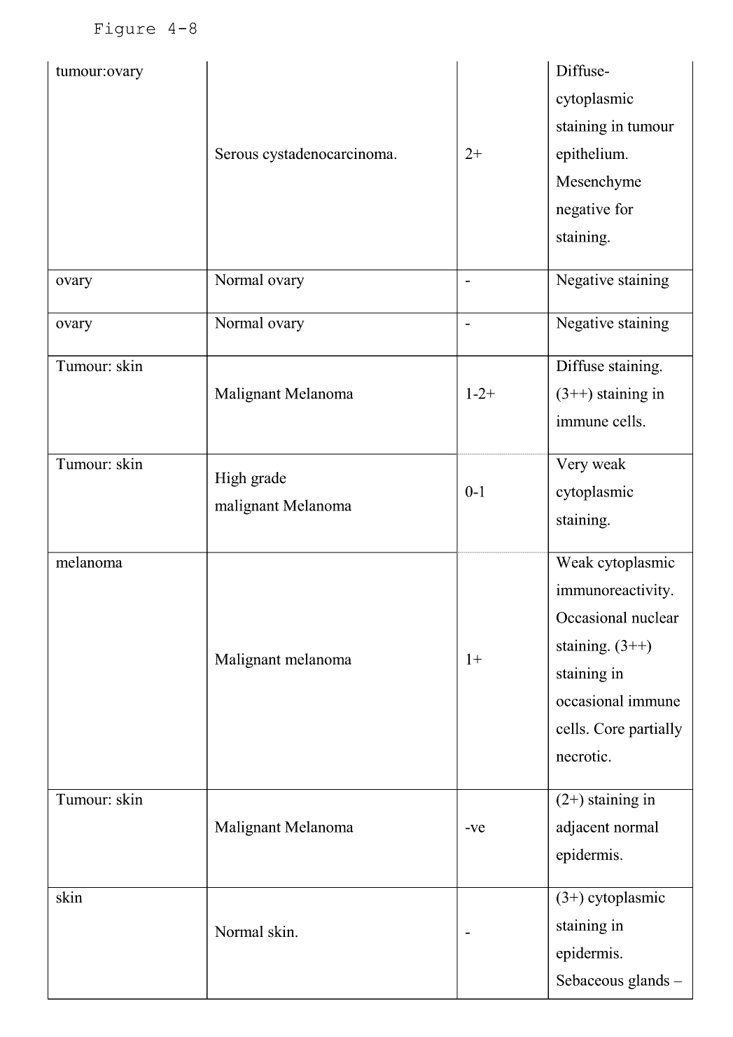

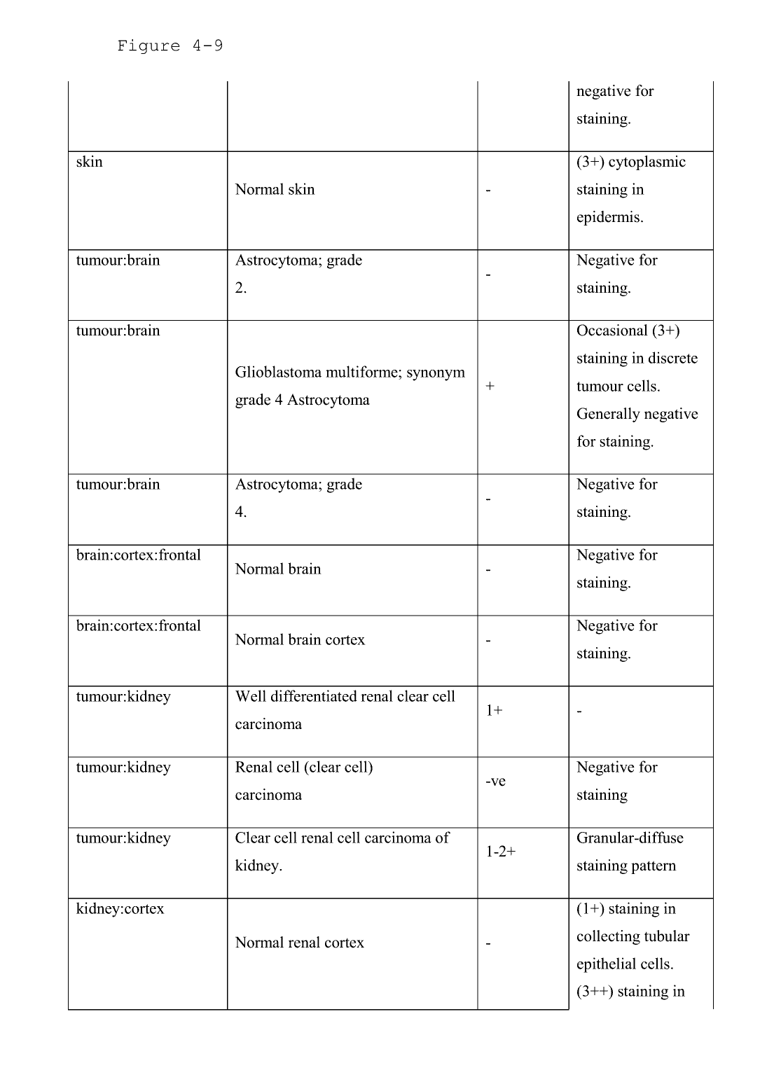

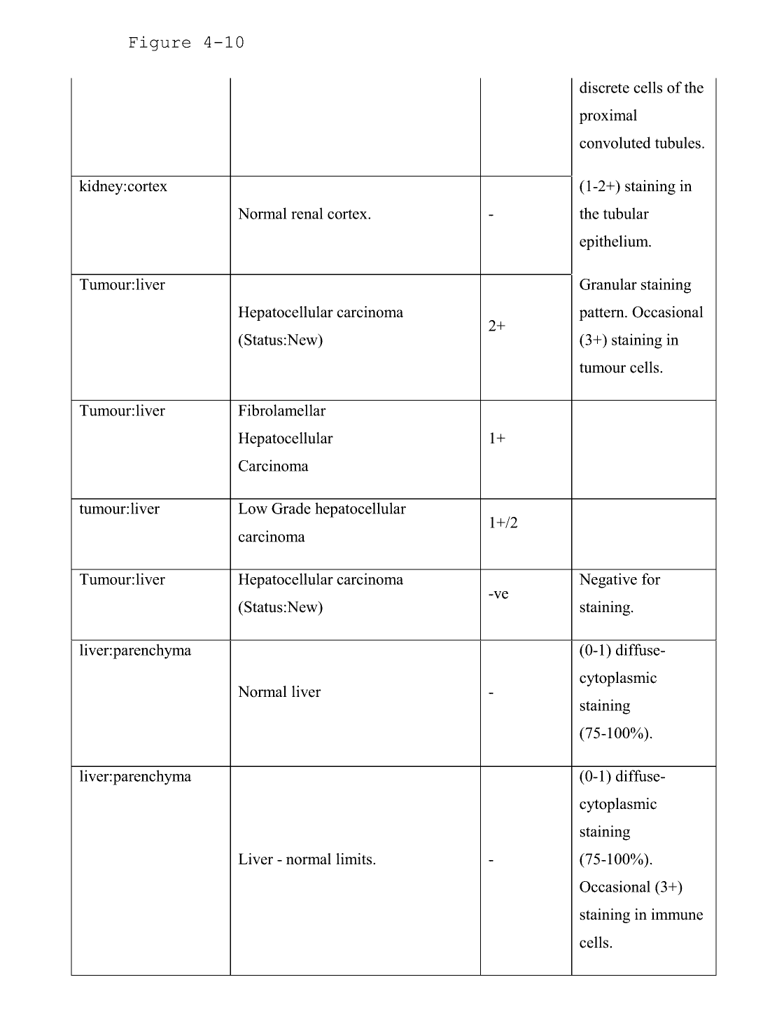

The following observations can be made upon review of the summary of IHC scores of individual samples in FIG. 4:
Within the breast tumor cohort, the majority of staining seen was weak to moderate, with 50-100% of tumor cells immunoreactive. In one sample, occasional immune cells were also observed to show intense staining. In normal breast, no apparent immunoreactivity was observed within the tissue, with exception of a few infiltrating immune cells within the lobular acini regions.
Within the large bowel cohort, staining intensity was seen to be moderate to high within the tumor epithelium, within 75-100% of tumor cells immunoreactive. It was noted that three donor samples were also seen to exhibit intense cytoplasmic-membrane staining in discrete tumor cells. Within the stroma, immunoreactivity was also present and restricted to highly-stained putative immune cells. In the normal tissue set, specific diffuse-cytoplasmic immunoreactivity was seen in the mucosal epithelium and putative immune cells.
In prostate tumors, all but one sample appeared to be immunoreactive in 75-100% of tumor cells. In these cores, the staining pattern and intensity was weak In one sample, a few discrete tumor cells were shown to be highly stained, and where present—other staining observations were seen in immune cell infiltrates. In the normal prostate samples, specific weak-cytoplasmic immunoreactivity was detected in the glandular epithelium, and in putative fibroblasts.
In lymphoma samples, all donors were shown to be immunoreactive in 75-100% of tumor cells. In general, the pattern and intensity appeared to be weak throughout. Occasional tumor cells were also shown to demonstrate intense (3+) staining. In normal lymph node, specific cytoplasmic immunoreactivity was detected in lymphocytes within the cortex and germinal centers.
In the lung tumor set, eleven out of twelve samples demonstrated specific immunoreactivity within 75-100% of tumors. In non-small cell (NSCLC) tumors, weak immunoreactivity was seen in two samples. In two squamous tumor samples, immunoreactivity was seen in tumor islands, scoring (1+) in a moderate-poorly differentiated sample, with highly-stained infiltrating immune cells. In a moderate-well differentiated sample, a (3+) staining was seen. In adenocarcinoma, three samples scoring staining of (1+) is observed in poorly differentiated tumors. In one notable adenocarcinoma sample, 2+ staining was seen in tumors, with occasional highly stained infiltrating immune cells. In one small-cell carcinoma sample, 2+ staining was seen. In the normal lung samples, specific cytoplasmic immunoreactivity was seen in the respiratory epithelium, with highly-stained luminal surfaces, free alveolar macrophages and occasional putative fibroblasts.
In stomach tumors, four moderately differentiated adenocarcinoma samples demonstrated specific immunoreactivity in 75-100% of the tumor cells. The intensity of staining seen varied between donors, ranging from (0-1), (1-2) and (3+) in tumor cells. Highly-intense staining was seen in discrete tumor cells. Infiltrating immune cells were also seen to be immunoreactive, and highly stained in these samples. In normal stomach tissue, apparently specific ‘intense’ membrane-immunoreactivity was seen in the superficial mucosal epithelium, and diffuse-cytoplasmic staining was seen in the sub mucosa, with occasional ‘intense’ staining of discrete cells.
In the ovarian carcinoma cohort, specific immunoreactivity was seen in 75-100% of tumor cells. In the cystadenocarcinoma samples, moderate to intense granular staining was seen in tumor cells—ranging (2+ to 3+). Occasional intense staining of infiltrating immune cells was also noted in these samples.
In one granulosa sample, weak immunoreactivity was seen in tumor cells, staining (1+). In the normal ovarian samples, only one donor, showed specific nuclear-cytoplasmic immunoreactivity in discrete stromal cells. The other ovary sample was wholly negative.
In skin melanoma, weak immunoreactivity was generally seen in the tumor samples. Infiltrating immune cells were also immunoreactive in these samples, and appeared to be intensely stained. In normal skin, apparently intense (3+) cytoplasmic staining was seen in the epidermis from two donors. No other notable immunoreactivity was detected in these samples.
In brain tumor, no apparent immunoreactivity was detected in the majority of donors. In one sample, only a few tumor cells were seen to be immunoreactive. In normal brain, no immunoreactivity was seen.
In the renal carcinomas, two of the three clear-cell type tumors demonstrated immunoreactivity within 75-100% of cells, staining (1+) and (2+) in each core respectively. Other staining features were seen in occasional putative infiltrating immune cells in cores. In the normal kidney samples, a weak diffuse-cytoplasmic staining was generally seen in the collecting tubular epithelium. Intense cytoplasmic staining was also noted in a few cells of the proximal convoluted tubules.
In liver carcinomas, three samples demonstrated weak to moderate immunoreactivity in tumor cells, where 75-100% were stained (1+, 2, 2+) respectively in each core. One particular donor, was seen to have occasional intense (3+) staining in tumor cells. In normal liver samples, a very weak-cytoplasmic blush staining was seen in, and restricted to normal hepatocytes and putative immune cells.
In full-face sections of normal lymph node, tonsil and spleen, it was observed that the majority of samples demonstrated specific staining in immune cells within the germinal centers/paracortex from the three tissue sets. In lymph node, cytoplasmic heterogeneity was seen throughout the tissue samples. In tonsil, specific cytoplasmic staining was seen in the germinal centers and of the paracortex, notable immunoreactivity was also seen in the squamous epithelium of one donor. In spleen samples, specific immunoreactivity was detected in occasional immune cells of the germinal centers, and pulp regions.

### Results Summary of the ‘TOP4’ TMA

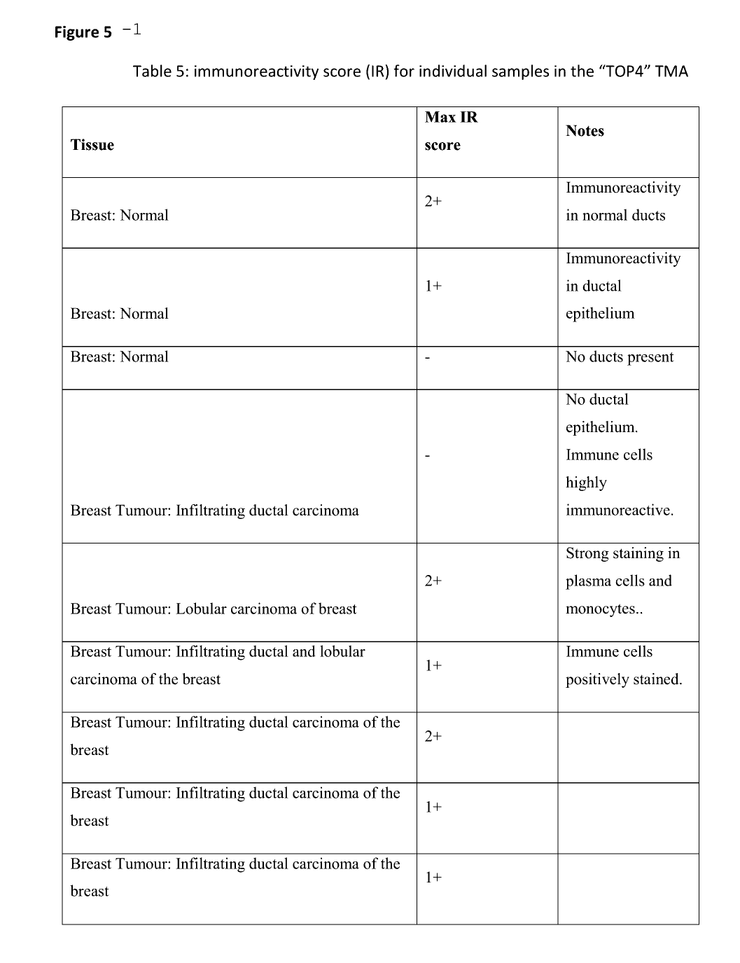

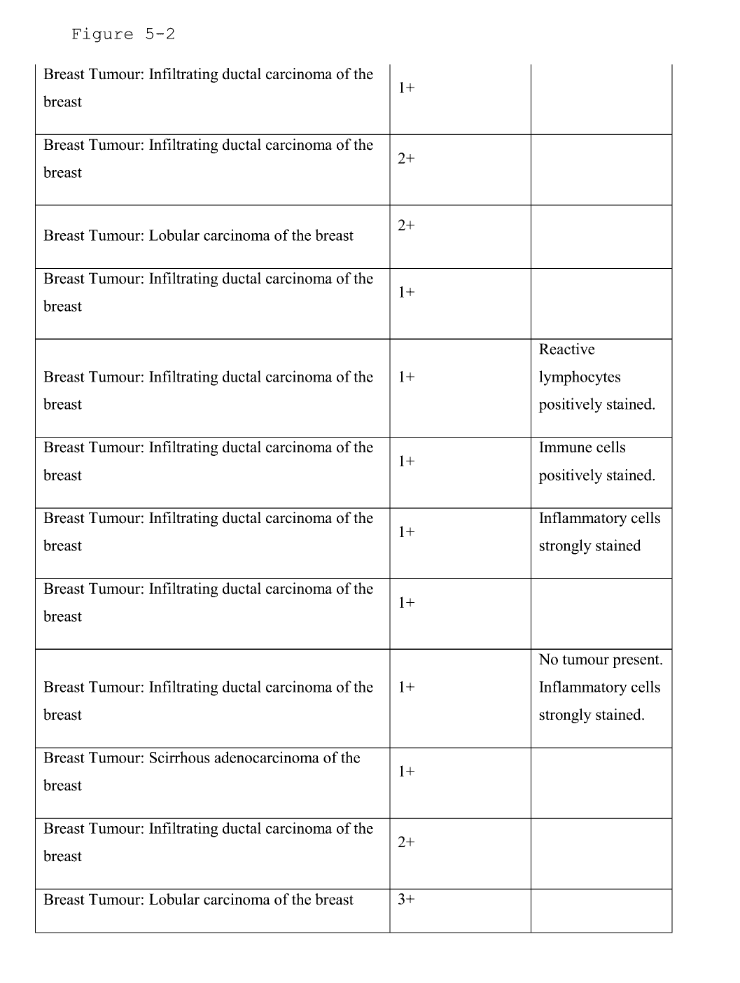

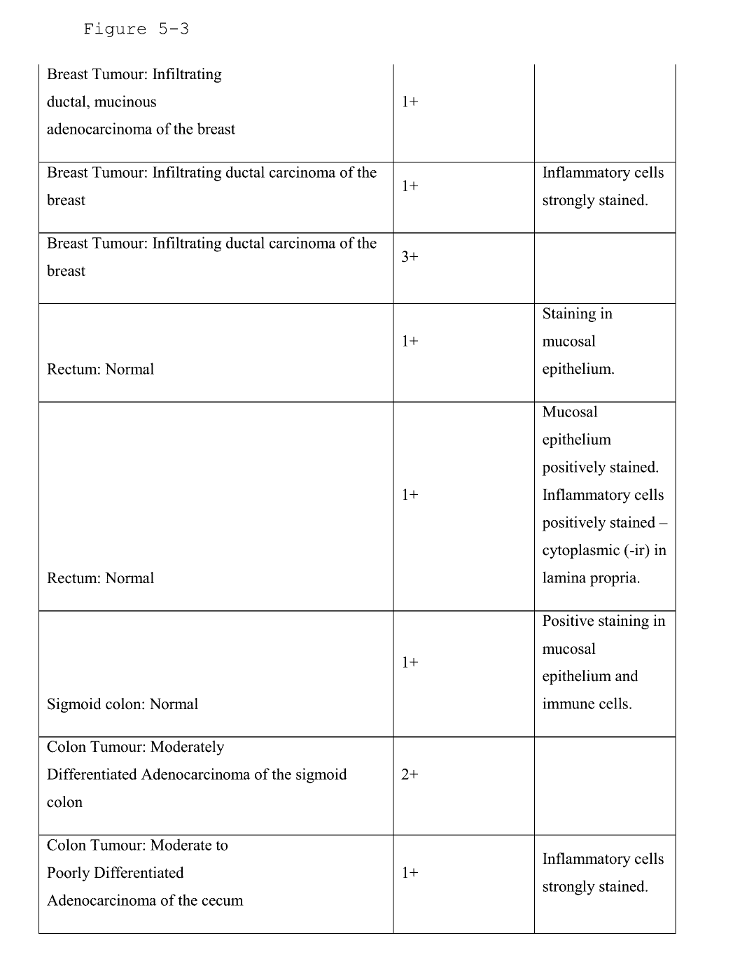

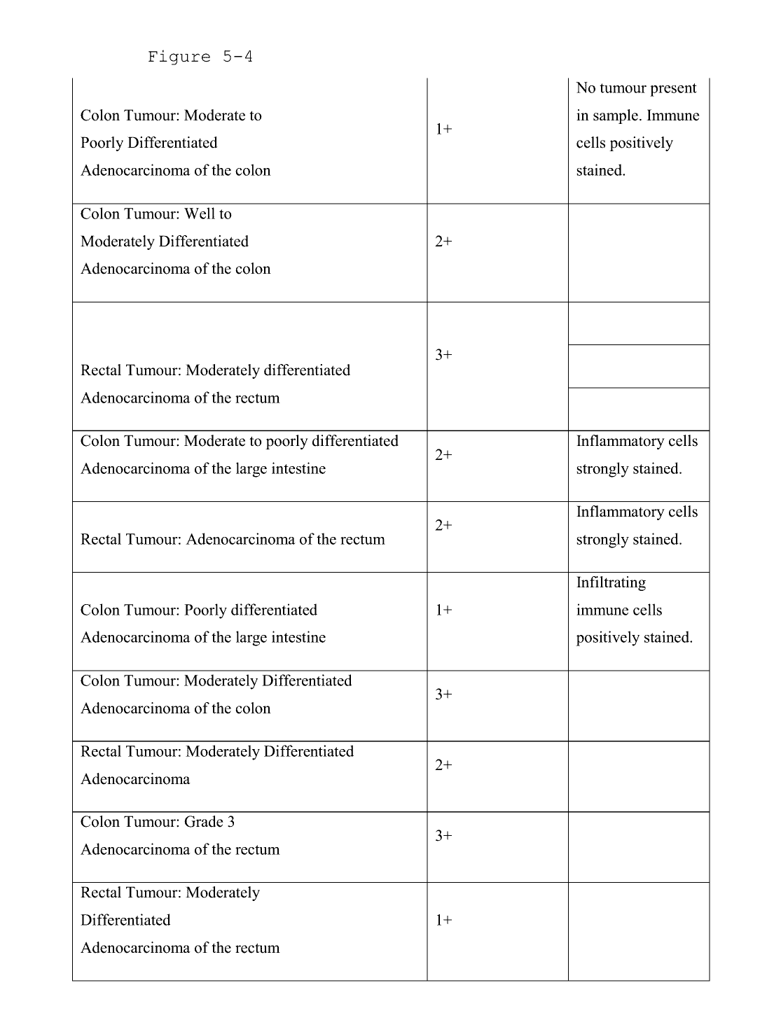

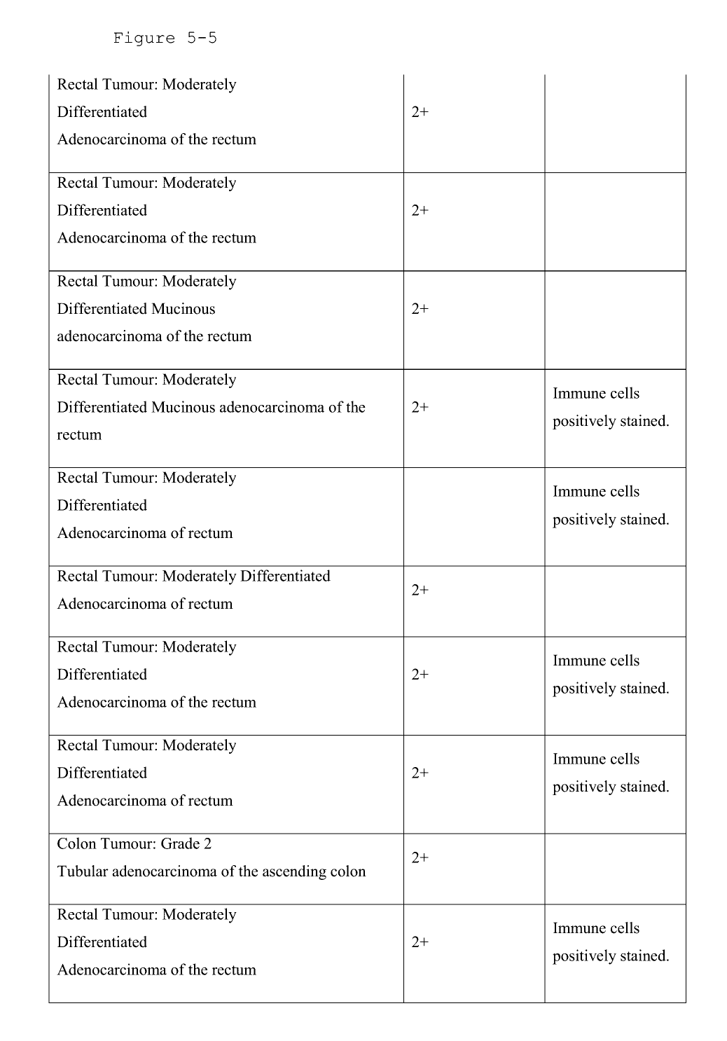

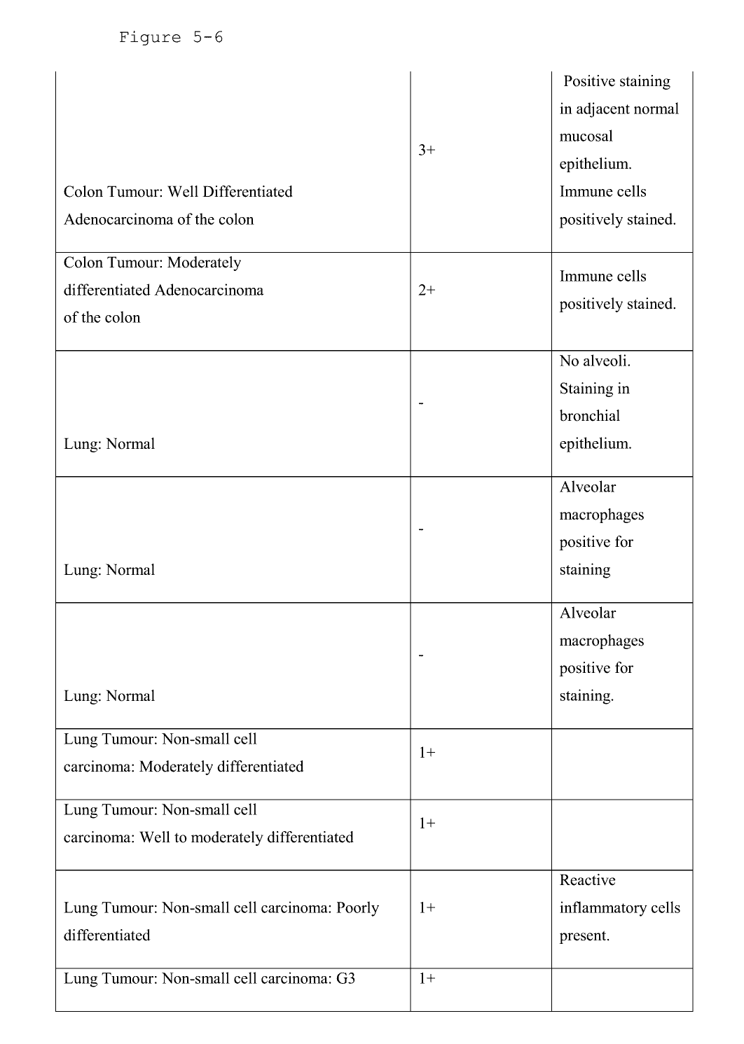

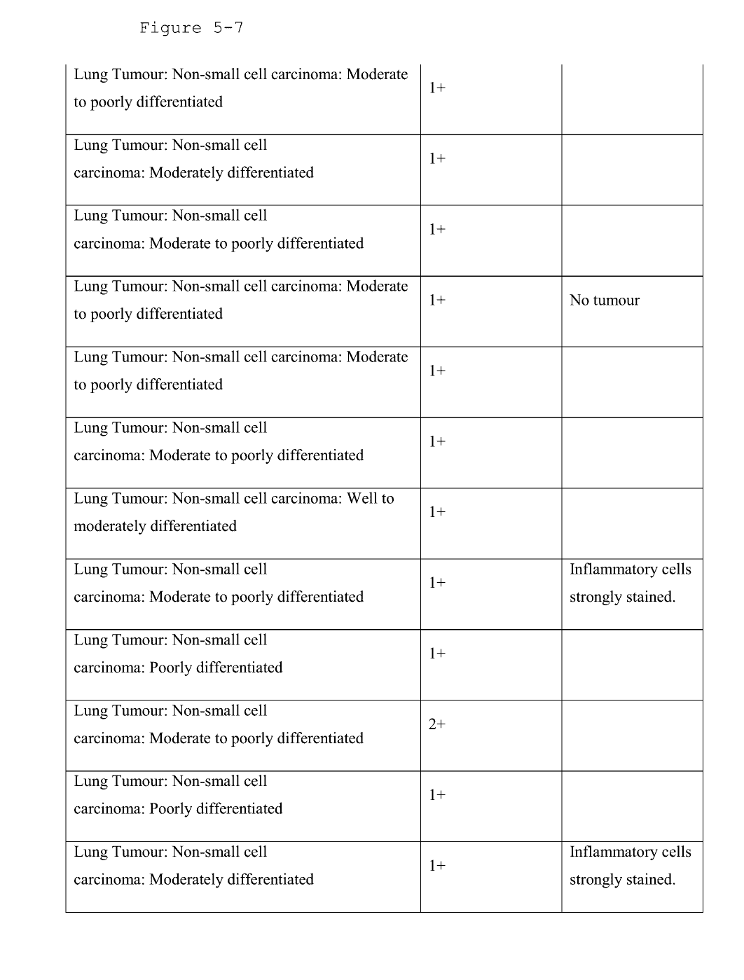

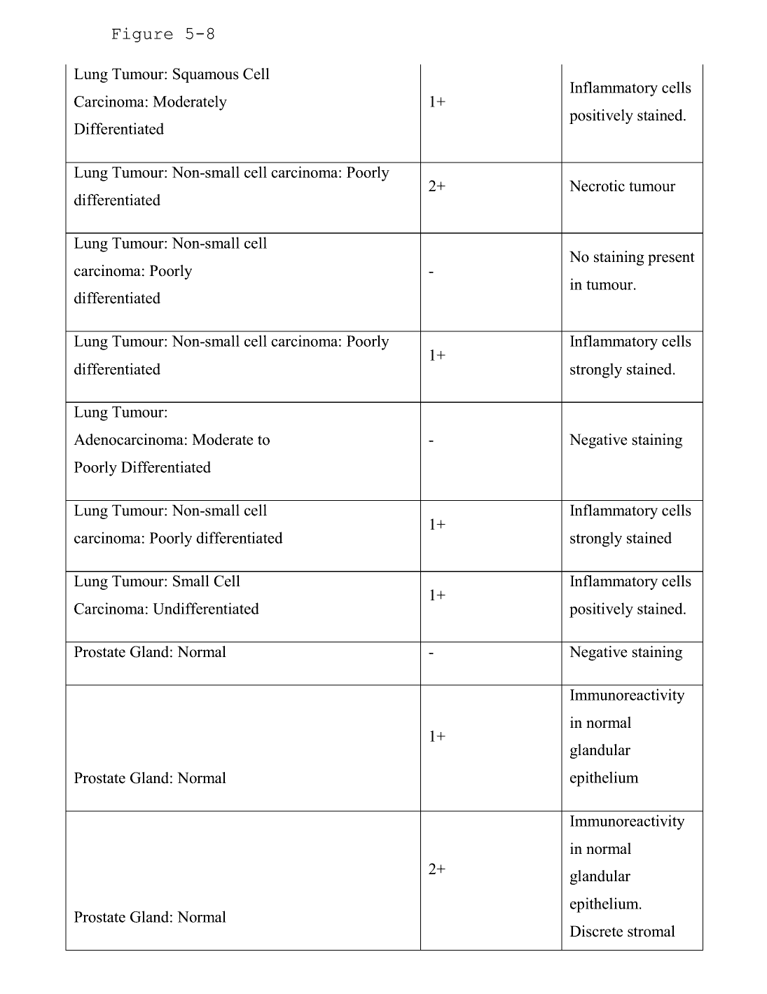

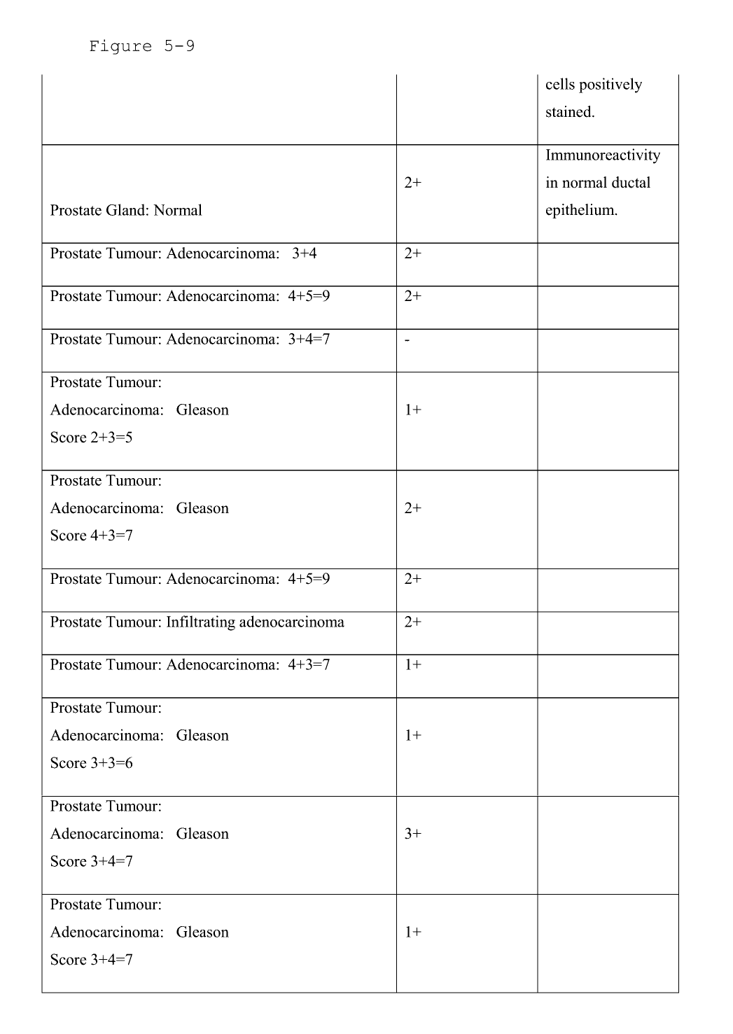

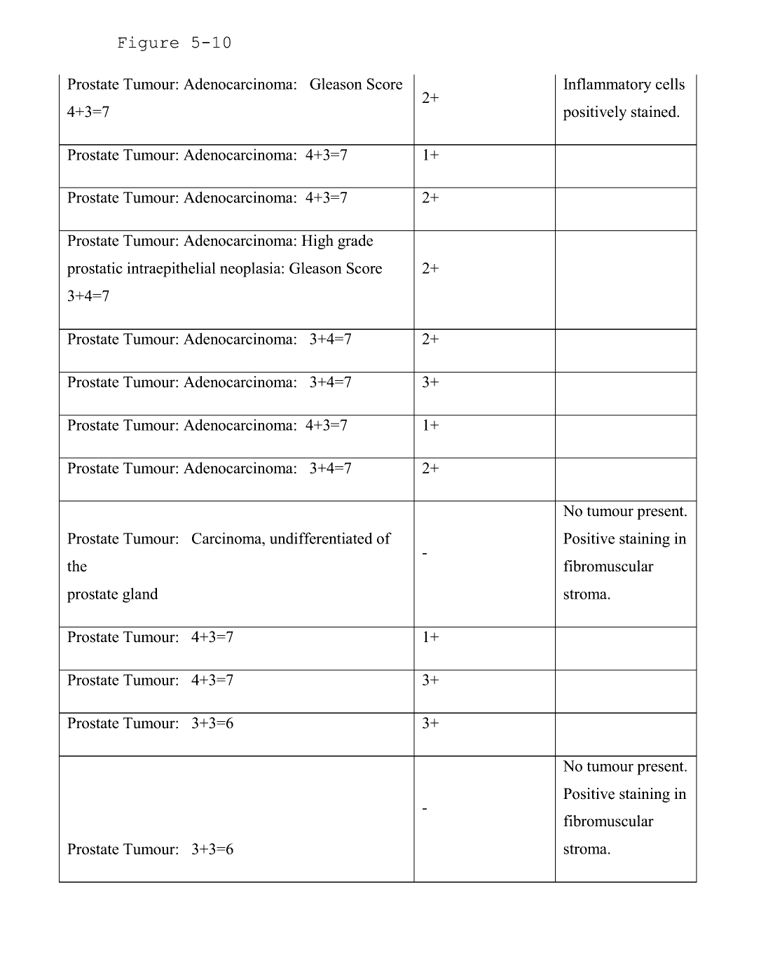

The following observations can be made upon review of the summary of IHC scores of TOP4 TMA samples in FIG. 5:
Within the breast tumor set, the intensity of staining seen was weak to moderate in majority of cases. In this cohort, two samples scored a maximum intensity of 3-3+ within 50-100% of reactive tumors, tumor grades 2/3. Within five of the samples, the staining intensity scored a maximum of 2+, within 50-100% of tumors (grades 2/3). Thirteen other samples scored a lower intensity of +1 of which most tumor samples were 25-100% reactive. Within the stromal regions, infiltrating immune cells were seen highly stained—notably in putative monocytes and plasma cells. The majority of tumors were mainly infiltrating ductal and lobular carcinomas—mixed grades. In normal breast, weak/moderate immunoreactivity was seen within the glandular acini.
Within the large bowel cohort, the adenocarcinoma samples were moderate to well differentiated types. In four samples, an assigned score of 3+ staining was seen within 50-100% of tumor cells of tumor grades 2/3. In fourteen other samples, a score of 2+ was seen in 50-100% of cells from reactive tumor grades 2/3. In the last four samples, a weaker score of 1+ was seen in tumors of moderate-poorly differentiated cell types. Other immunoreactive regions include highly stained infiltrating immune cells. In normal tissue samples, specific immunoreactivity was detected in mucosal epithelium and resident inflammatory cells.
In the lung tumor set, specific immunoreactivity was seen in the majority of tumors investigated, where a weak to moderate staining intensity was noted. The majority of tumors were non-small cell lung carcinomas (NSCLC)—of adenocarcinoma origin, of moderate to poorly differentiated cell types. In these tumors, two samples were assigned a maximum intensity score of 2+, of which 25-100% of tumor cells were immunoreactive. In seventeen other samples, a weaker score of 1+ was seen in 25-100% of tumors. In one sample of small-cell carcinoma immunoreactivity was weak (1+), within 25-50% of tumor cells. Highly immunoreactive infiltrating immune cells were prominent. Pathological scores indicate a heterogeneous pattern of staining of the same tumor type(s).
In the normal lung tissue, immunoreactivity was detected in one sample of bronchial epithelium. No apparent immunoreactivity was detected in other normal lung cores. Occasional free-macrophages were only seen to be immunoreactive.
In the set of prostate tumors, specific staining was seen in most samples, where intensity of staining was weak to moderate in the tumor epithelium. In four samples, a maximum assigned score of (3+) staining was seen in 50-100% of tumors, (Gleason scores 3+3, 4+3 and two—3+4 samples). Eleven other samples had a score of 2+ within 50-100% of tumors. Most of the staining was in tumor islands, with Gleason scores ranging from (3+4), (4+5) and (4+3) respectively. Lastly, six tumor core samples were scored a weaker 1+ staining in 25-100% of tumors. In the normal prostate tissues, a few samples demonstrated weak-moderate cytoplasmic staining in the glandular epithelium. Other notable staining was seen in putative infiltrating immune cells and fibromuscular regions.
Overall, the results from a variety of different human tumor samples, representing different types of tumor tissues and lymphoid tissues indicate that VSTM5 protein was expressed in a large proportion of the tumor types studied. Moreover, in tumor types, including tumors with relatively low immunoreactivity, immune infiltrating cells were positive, further supporting the immune modulatory role of VSTM5 protein.
These results coupled with the functional data in the examples which follow corroborate the fact that immunomodulatory VSTM5 polypeptides and fusion proteins according to the invention should suppress or potentiate the effects of VSTM5 in different human disease conditions, e.g., cancer or infectious disease, wherein the expression of VSTM5 seems to have a suppressive effect on the subject's antitumor immune response.

### Example 2

### Generation and Characterization of VTM5-Expressing Stable Transfectant Cell Pools

As described herein, in these experiments recombinant stable pools of cell lines overexpressing VSTM5 human and mouse proteins were generated. These cell lines are used, in experiments infra for determining the effects of VSTM5 on immunity, as well as for VSTM5 characterization, anti-VSTM5 antibody discovery, and for obtaining cross-species reactive anti-VSTM5 antibodies.

### Materials & Methods

### Expression Constructs

The coding sequence in each of the various expression constructs used in this example was obtained either by full length cloning using RT-PCR derived cDNAs or by gene synthesis, followed by subcloning to mammalian expression vectors.

Full length cDNA of human VSTM5 (SEQ ID NO:4) was obtained by RT-PCR using lung cancer cDNA as a template with gene specific primers, as described in PCT/US2008/075122, owned in common with the present application. The full length cDNA was subsequently cloned into expression vectors to create the constructs described below. All cDNA inserts were digested with specific restriction enzymes and ligated to pIRESpuro3 (pRp3) mammalian expression vector (Clontech, Cat No: 631619) previously digested with the same enzymes.

### Construct Encoding Human VSTM5-Untagged

The full length cDNA of human VSTM5 (SEQ ID NO:4) was cloned in the pIRESpuro3 (pRp3) mammalian expression vector, as described in PCT/US2008/075122, owned in common with the present application.

### Construct Encoding Human VSTM5-EGFP

Full length cDNA of human VSTM5 (SEQ ID NO:4) was cloned in frame to the N terminus of EGFP in EGFP-pIRESpuro3 (Chen et al., Molecular Vision 2002; 8; 372-388) for expression of a VSTM5-EGFP fusion protein. Subcloning was performed by PCR using the above human VSTM5 (SEQ ID NO:4)-untagged expression vector as template.

Construct Encoding the Fusion Protein Human VSTM5 hECD mIgG2a (SEQ ID NO:10)

Cloning of the fusion protein of the extracellular domain (ECD) of human VSTM5 fused to mouse IgG2a Fc, was carried out in two steps: first, cloning of ECD to pIRESpuro3; and second, subcloning of the mouse IgG2a Fc in frame to the C′ terminus of the ECD previously cloned into pIRESpuro3, from step one. Cloning of the ECD was done by PCR using VSTM5 full length sequence as a template, as described in PCT/US2008/075122, owned in common with the present application. The resulting expression constructs were verified by sequence and subsequently used for transfections and stable pool generation as described below.

### Construct Encoding Mouse VSTM5 (SEQ ID NO:11)

Full length cDNA encoding mouse-VSTM5 protein (SEQ ID NO:11) was synthesized, and cloned in pUC57 vector at GeneScript. This cDNA was subsequently cloned in the pIRESpuro3 mammalian expression vector as described below. The resulting expression constructs were verified by sequence and subsequently used for transfections and stable pool generation as described below.

### Generation of Stable Transfectant Pools Expressing Human VSTM5 or VSTM5-EGFP Proteins

The expression constructs described above were used to generate stable pools of VSTM5 expressing HEK293T cells by DNA transfection, followed by establishment of resistant pools of colonies with the specific selection media. Each of the parental cell lines was also transfected with an empty vector, used as negative control.

HEK-293T (ATCC, CRL-11268) cells were transfected with the human VSTM5 and human VSTM5-EGFP pRp3 constructs described above or with the empty vector (pRp3) as negative control, using Fugene6 transfection reagent (Roche, Cat No: 11-988-387). Puromycin resistant colonies were selected for stable pool generation.

### Expression Validation

Whole cell extracts of each cell pool (30 ug of total protein) were analyzed by western blot. As negative control, whole cell extracts of stable cell pools transfected with the empty vector were used. A commercial rabbit anti-VSTM5 polyclonal Ab diluted 1:100 was used (Sigma, Cat. No HPA029525), followed by secondary goat anti-rabbit conjugated to HRP (Jackson, 111-035-003) Ab diluted 1:10000 in blocking solution.

In order to validate the cell surface expression of VSTM5 (SEQ ID NO:132) or VSTM5-EGFP (SEQ ID NO:133) proteins in the recombinant stable pools, 1×10⁵ cells were stained with 10 ug/ml of commercial anti VSTM5 rabbit pAb (Sigma, Cat. No HPA029525) or rabbit IgG control (Sigma, Cat No 15006), followed by donkey anti-rabbit FITC-conjugated secondary Ab diluted 1:400 (Jackson 711-096-152), or by donkey anti-Rabbit PE-conjugated secondary Ab diluted 1:200 (Jackson, 711-116-152), and analyzed by Flow Cytometry (FACS).

### Results

Production of Stable Pool of HEK-293T Cells Over Expressing the Human VSTM5 and VSTM5-EGFP Proteins (SEQ ID NOs:132 and 133, Respectively)

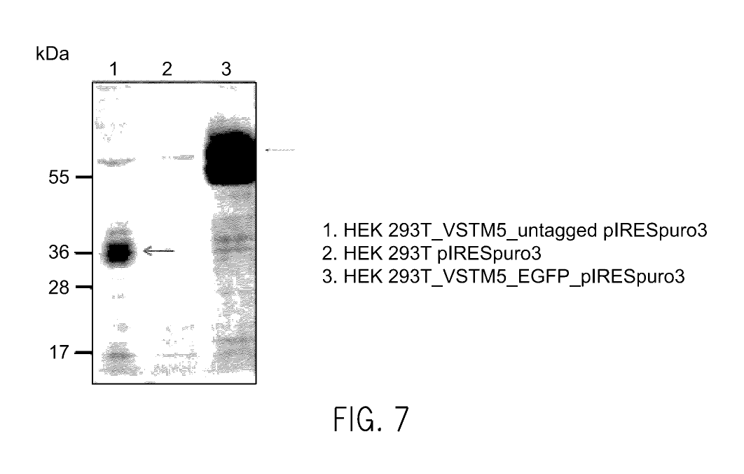

Expression of the VSTM5 proteins (SEQ ID NOs:132 and 133) in stably transfected HEK293T cell pools, whole cell extracts of stable pools expressing VSTM5 or VSTM5-EGFP proteins (SEQ ID NOs:132 or 133, respectively) was analyzed by Western Blot using a commercial rabbit pAb, as described in Materials & Methods in this example. FIG. 7 presents the results of the Western Blot analysis of ectopically expressed human VSTM5 proteins using an anti-VSTM5 antibody. Whole cell extracts (30 ug) of HEK293T cell pools, previously transfected with expression construct encoding human VSTM5 (lane 1), empty vector (lane 2) or with expression construct encoding human VSTM5-EGFP (lane 3), were analyzed by WB using an anti-VSTM5 antibody. The results, shown in FIG. 7, demonstrate a specific band corresponding to the expected protein size of ˜30 kDa or ˜60 kDa in the extracts of HEK293T cell pools expressing human VSTM5 or VSTM5-EGFP (SEQ ID NOs:132 or 133, respectively), but not in the cells transfected with the empty vector.

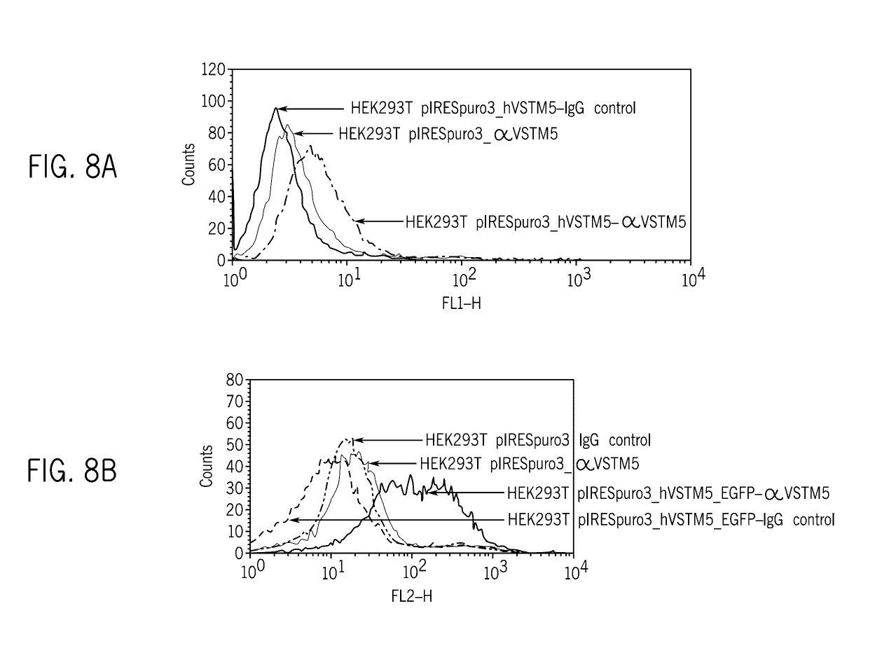

Further, in order to verify cell surface expression of the VSTM5 proteins, HEK293 stably transfected cells over-expressing the human VSTM5 or VSTM5-EGFP proteins (SEQ ID NOs:132 or 133, respectively) were analyzed by flow cytometry (FACS) using anti-VSTM5 pAbs. FIG. 8A-B presents the results of cell surface expression of human VSTM5 (A) and VSTM5-EGFP (B) proteins by FACS analysis. The anti-VSTM5 pAb (10 ug/ml) was used to analyze HEK-293T cells stably expressing the human VSTM5 proteins. Rabbit IgG was used as Isotype control to the pAb. Cells expressing the empty vector (pRp=pIRESpuro3) were used as negative control. Detection was carried out by donkey anti-rabbit FITC or PE-conjugated secondary Ab and analyzed by FACS.

As shown in FIG. 8A and FIG. 8B, binding of the rabbit anti-VSTM5 pAb to cells stably expressing the human VSTM5 or human VSTM5-EGFP proteins was considerably higher than that observed with cells transfected with the empty vector, or cells stained with the rabbit IgG control, indicating specific cell membrane expression of the VSTM5 proteins.

### Example 3

### In-Vitro Testing of the Effect of VSTM5, Expressed on Hek 293T Cells, on the Activation of Jurkat Cells

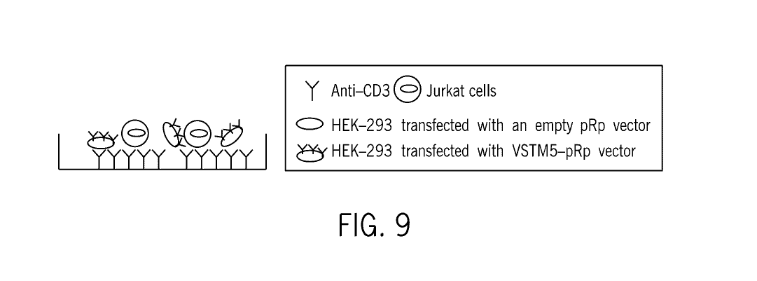

In order to evaluate the effect of the native cell surface expressed VSTM5 protein on T cell activation, a co-culture assay was used. In the assay HEK-293T cells over expressing VSTM5 (the generation of which is described in Example 2) and Jurkat cells (derived from a human T cell leukemia) activated by plate-bound anti-CD3 antibodies were co-cultured. FIG. 9 presents a schematic illustration of the experimental setting of in vitro testing of the effect of VSTM5, expressed on HEK 293T cells, on the activation of Jurkat cells by plate bound anti-CD3.

### Materials and Methods

### Anti-CD3 Mediated Activation of Jurkat T Cells as Measured by CD69 Expression

Anti-CD3 (Clone UCHT1, BD Bioscience; cat#555329) was immobilized at 2 μg/ml on a flat-bottom 96-well plate at 4° C. HEK-293T cell pools stably expressing VSTM5 or transfected with the empty vector, as described in Example 2, were treated with mitomycin C (Sigma, M4287, 0.5 mg/ml, freshly prepared in H₂O): at a final concentration of 50 μg/ml. Cells were incubated with mitomycin C for 1 hour at 37° C. Mitomycin C treated HEK-293T cells were washed, removed by addition of 1 ml of cell dissociation buffer (Gibco; Cat. 13151-014), resuspended in Jurkat cells' growth medium, and diluted to 0.5×10⁶ cells per ml. Cells were serially diluted and seeded at the indicated concentrations in 100 μl per well, in the flat-bottom 96-well plate pre-coated with anti-CD3. After 2 hours to allow HEK-293T cells attachment, 50,000 Jurkat cells (ATCC, clone E6-1, TIB-152) were added to each well at a volume of 100 μl per well. Cells were co-cultured O. N., at 37° C. in a humidified incubator.

For assessment of CD69 upregulation on Jurkat cells (marker of activation), cells were transferred to U-shape plates, and flow cytometry (FACS) analysis of cell surface expression levels of CD69 was carried out using an anti-CD69 Ab (Biolegend, PE-anti human CD69, clone FN50, cat#310906, 10 μg/ml, 2 μl/well) and Fc-blocker (Miltenyi Biotec, human FcR blocking reagent, cat#120000-442, 1 μl/well). Readouts were Mean Fluorescence Intensity (MFI) or percentage of cells expressing CD69 out of total T cells. Jurkat cells were gated according to Forward Scatter (FSC) vs. Side Scatter (SSC). Gating procedure was validated by staining the cells with anti-CD2 antibody (Biolegend; clone RPA-2.10, Cat. 300206) in order to identify the Jurkat T cells.

### Results

### Inhibition of Anti CD3-Mediated Activation of Jurkat T Cells as Measured by CD69 Expression.

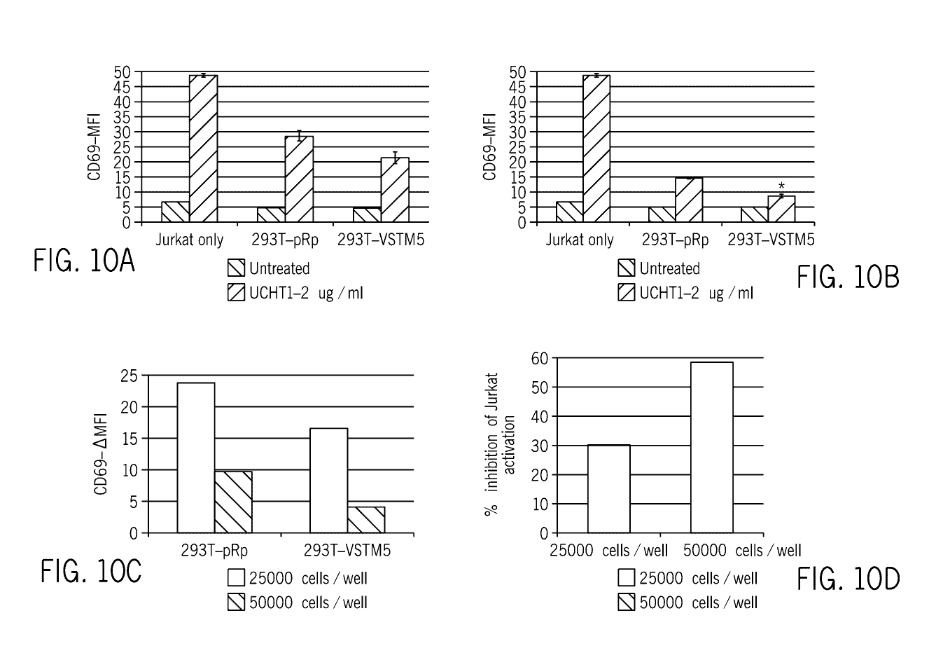

HEK-293T transfectants expressing the VSTM5 protein fused to EGFP (SEQ ID NO:133) were co-cultured with Jurkat T cells activated by plate-bound anti-CD3 antibodies, as described in Materials and Methods herein. HEK-293T cells transfected with the vector only (pRp3) were used as a negative control. Representative results, shown in FIG. 10, indicate that Jurkat T cells stimulated with anti-human CD3 antibodies exhibit reduced activation in the presence of VSTM5-expressing HEK-293T cells, as manifested by reduced upregulation of CD69 (an early marker of T cell activation) in comparison to the effect of HEK-293T cells transfected with the vector only (pRp3). The inhibitory effect of VSTM5 was best detected using 5×10⁴ HEK-293T transfected cells per well.

These results show that VSTM5 expressed on the cell membrane of HEK-293T cells inhibits Jurkat T cell activation, and indicate that the native VSTM5 membrane protein expressed on the cell surface inhibits T cell activation. This inhibition of T cell activation corroborates the therapeutic potential of VSTM5 targeting agents. For example, it suggests that antibodies or VSTM5 polypeptides and fusion proteins according to the invention which agonize VSTM5 may be used as immunoinhibitors for treating T cell-driven autoimmune diseases, such as rheumatoid arthritis, multiple sclerosis, psoriasis and inflammatory bowel disease, as well as other immune related conditions wherein there are pathogenic T cells or wherein reducing undesirable immune activation is desired such as after gene therapy or transplant. In addition, these results also support the therapeutic potential of immunopotentiating VSTM5 targeting agents that reduce the inhibitory activity of VSTM5 (e.g. VSTM5 polypeptides and fusion proteins according to the invention) for treating conditions which would benefit from enhanced immune responses such as immunotherapy and the treatment of cancer, infectious diseases, particularly chronic infections and sepsis.

### Example 4

### In Vitro Testing of the Effect of VSTM5 Expressed on HEK 293T Cells on Activated H9 Cells

In order to evaluate the effect of the native cell surface expressed VSTM5 protein on T cell activation, a co-culture assay was effected using HEK-293T cells over expressing VSTM5 and a H9 cel line (clonal derivative of the Hut 78 cell line derived from a human T cell leukemia) activated by plate-bound anti-CD3 antibodies.

### Materials and Methods

### Anti-CD3 Mediated Activation of H9 Cells as Measured by Human IL-2 Cytokine Secretion in the Supernatant

Day 1:
1. HEK-293T cell pools stably transfected with expression constructs of the pRp3.1 plasmid expressing VSTM5, or with the empty vector pRp3.1, were seeded at a concentration of 7×10⁶ cells per T75 plate and cultured in DMEM medium supplemented with 10% FBS, L-glutamine in a humidified incubator O.N.

Day 2:
1. Anti-CD3 (Clone OKT3, eBioscience; cat#16-0037) diluted in 1×PBS was immobilized on a flat-bottom 96-well plate in 75 μL/well at a concentration of 0.1 and 0.2 μg/ml.
2. Plates were wrapped with parafilm and incubated at 37° C. for 3 hours in humidified incubator.
3. Wells coated with anti-CD3 were washed 3 times with 200 μl of PBS. Fluid was decanted in a sterile environment. After the last wash, the plate was blotted on a sterile absorbent paper to remove any residual fluid.
4. HEK-293T cells, seeded the day before, were treated with mitomycin C (Sigma, cat# M4287): 900 μl of a 0.5 mg/ml solution freshly prepared in H₂O were added directly to 8.1 ml of growth medium, to obtain a final concentration of 50 μg/ml. Cells were incubated with mitomycin C for 1 hour at 37° C.
5. Mitomycin C treated HEK 293T cells were washed 3 times with 10 ml of PBS and detached by addition of 2 ml of cell dissociation buffer (Gibco; cat#13151-014).
6. Detached HEK-293T cells were re-suspended in 8 ml of RPMI supplemented with 10% FBS and L-glutamine (H9 cell growth medium).
7. Cells were counted using a Beckman coulter counter and diluted to 0.5×10⁶ cells per ml.
8. Cells were serially diluted and seeded at the indicated concentrations (50,000 and 75,000 HEK-293T cells) in 100 μl per well of H9 cell growth medium (described above).
9. HEK-293T cells were incubated for 2 hours to allow attachment.
10. 50,000 of the H9 cells (ATCC, HTB-176) were added to each well at a volume of 100 μl per well in H9 cell growth medium (described above).
11. Cells were co-cultured O.N. at 37° C. in a humidified incubator.

Day 3:

Cells were transferred to U-shape plates, centrifuged 5 minutes at 1500 rpm at 4° C. The supernatant was frozen and kept at −20° C. until interleukin 2 (IL-2) immunoassay was performed. (Another early marker of T cell activation (CD69) has been used in the same experiment but the results were inconclusive.)

### Quantitative Determination of Human Interleukin 2 (IL-2) Concentrations in Cell Culture Supernatants (R&D Systems, Quantikine ELISA, Human IL-2, Cat#52050)

In order to assess the response of the T cells, cytokine secretion (hIL-2) was measured by ELISA in culture supernatants, diluted to be in the linear range of the ELISA assay (R&D Systems, Quantikine ELISA, Human IL-2, cat# S2050).
1. All the particles (cells and cell debris) were removed by centrifugation and the supernatants were sealed and kept at −20° C.
2. The reagents, samples and working standards are prepared as directed in assay procedure. The samples are diluted 5 times in PBS.
3. Assay Diluent RD1W is added (100 μl) to each well.
4. The standard (duplicates) and the samples (triplicates) are added to the wells, covered with adhesive strip and incubated 2 hours at room temperature.
5. Each well was aspirated and washed (Wash buffer supplied within kit) 3 times and traces of the wash buffer were removed by blotting the plate against clean paper.
6. Il-2 conjugate is added to each well, covered with adhesive strip and incubated 2 hours at room temperature.
7. Aspiration/wash step (5) is repeated.
8. The substrate solution (provided by the kit) is added to each well and incubated 20 minutes at room temperature.
9. The reaction is stopped by stop solution (provided by the kit).
10. The optical density of each well is determined by using a microplate reader (Biotek, ELx808) set to 450 nm

Results

Inhibition of Anti CD3-Mediated Activation of H9 T Cells as Measured by IL-2 Cytokine Secretion.

HEK-293T transfectants expressing the full length of human VSTM5 protein were co-cultured with H9 T cells activated by plate-bound anti-CD3 antibodies, as described in Mat & Meth. HEK-293T cells transfected with the vector only (pRp3.1) were used as a negative control. The representative results which are shown in FIG. 11 indicate that H9 T cells stimulated with anti-human CD3 antibody exhibit reduced activation in the presence of VSTM5-expressing HEK-293T cells, as evidenced by reduced secretion of IL-2 in the supernatant, in comparison to the effect of HEK-293T cells transfected with the vector only (pRp3.1). The inhibitory effect of VSTM5 is the most prominent when using 50,000 HEK293T transfected cells per well.

Conclusion

These results show that VSTM5 expressed on the cell membrane of HEK-293T cells inhibits H9 T cell activation, and indicate that the native VSTM5 membrane protein expressed on the cell surface has an inhibitory effect on T cell activation.

### Example 5

### In Vitro T Cell Assays to Investigate Immunomodulatory Activities of VSTM5 ECD-Ig on Mouse T Cells

In these experiments the immunomodulatory activities of the recombinant fused protein VSTM5-ECD-Ig was tested on mouse T cells, using a number of in vitro T cell activation readouts. Particularly, in order to evaluate the activity of VSTM5 ECD-Ig protein on T cell activation, a recombinant protein comprising the extracellular domain of human (H) or mouse (M) VSTM5 fused to the Fc of mouse (M) IgG2a (designated VSTM5-hECD-mIg (SEQ ID NO: 131, described in PCT/US2008/075122) or VSTM5-mECD-mIg (SEQ ID NO:8) was produced. The effect of such VSTM5-ECD-Ig on mouse CD4⁺ T cell functions, as manifested by activation markers, cytokine secretion and proliferation was investigated as further described below.

### Materials and Methods

### VSTM5-ECD Fusion Proteins and Control Ig

The effect of VSTM5-hECD-mIg (SEQ ID NO: 131) or VSTM5-mECD-mIg (SEQ ID NO: 8) on mouse CD4⁺ T cells were analyzed. VSTM5-ECD-Ig H:M fusion proteins was produced in-house in HEK-293T cells by culturing stable cell pools for 3-4 days, followed by Protein A purification of cell harvest. VSTM5-mECD-mIg was produced at ExcellGene (Switzeland) by transient transfection in CHO-DG44 cells using Excellgene's proprietary vector system. Cells were cultured for 10 days, followed by Protein A purification of cell harvest. Mouse IgG2a Isotype control (clone C1. 18.4; BioXCell) was used as the control Ig.

### Mouse CD4⁺ T Cell Isolation

CD4⁺CD25− T cells (1 step negative selection) or naïve CD4⁺CD25⁻CD62L⁺ (1 step negative selection, followed by positive selection) T cells were isolated from pools of spleens and lymph nodes of BALB/C by using a T cell isolation Kit (Miltenyi Cat#130-093-227) according to the manufacturer's instructions. The purity obtained was >90%.

### Activation of Mouse CD4⁺ T Cells

Anti-mouse CD3-ε mAb (clone 145-2C11; BD Pharmalgen) alone or together with VSTM5-ECD-Ig or control Ig were co-immobilized overnight at 4° C., at various concentrations, on 96-well flat bottom tissue culture plates (Sigma, Cat. # Z707910). Wells were washed 3 times with PBS and plated with 1-2.5×10⁵ purified CD4⁺CD25⁻ or naïve CD4⁺CD25⁻CD62L⁺ T cells per well and kept in a humidified, 5% CO₂, 37° C. incubator. Culture supernatants were collected at the indicated times post stimulation and analyzed for mouse IFNγ or IL-2 secretion by ELISA kits (R&D Systems). The effect of VSTM5-hECD-mIg (SEQ ID NO: 131) on mouse CD4⁺ T cell proliferation was determined by labeling CD4⁺CD25⁻ T cells with CFSE (0.5 μM; Molecular Probes Cat. #C34554) and analyzing cell division's profiles at 72 h post stimulation. The effect of VSTM5-mECD-mIg (SEQ ID NO: 8) on the expression of the activation marker CD69 on mouse CD4⁺ T cells was analyzed by flow cytometry. Cells were stained 48 h post stimulation with a cocktail of antibodies including PerCP-anti-CD4 (clone G41.5; Biolegend), FITC-anti-CD69 (clone HE 2F3; Biolegend) in the presence of anti-CD16/32 (clone 2.4g2; BD Biosciences) for blocking of Fcγ-receptors. Cells were evaluated using BD FACS Calibur and data analyzed using BD CellQuest Software.

### Results

### VSTM5-ECD-Ig Inhibits Mouse T Cell Activation

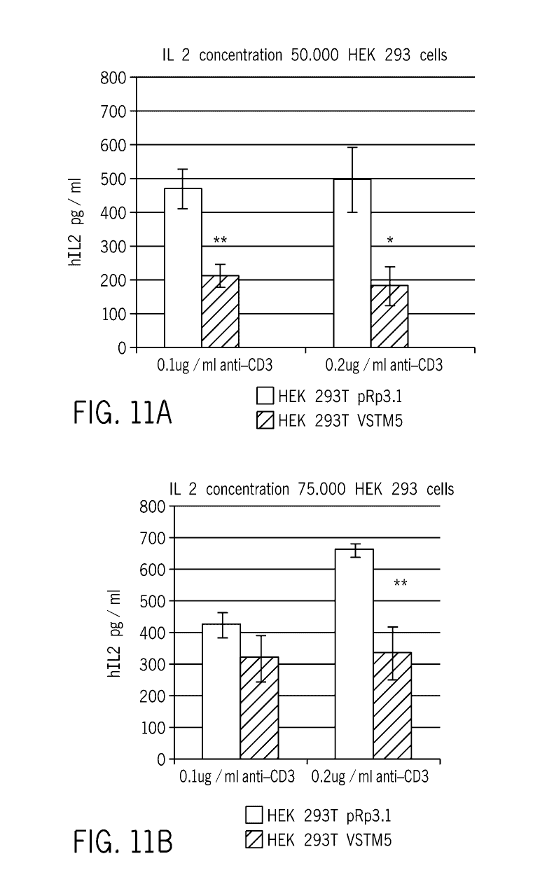

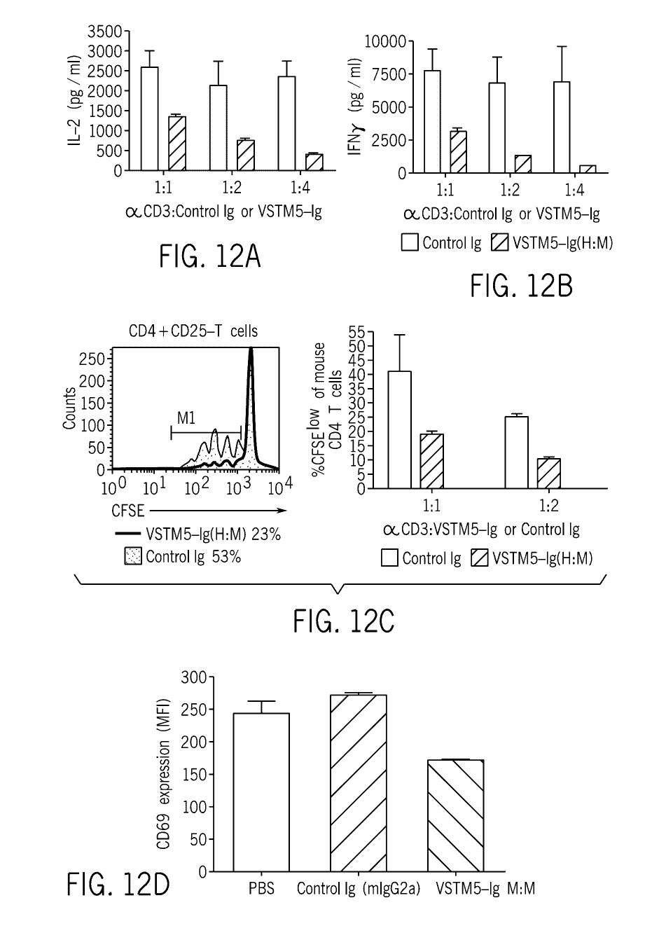

VSTM5-hECD-mIg (SEQ ID NO: 131) and VSTM5-mECD-mIg (SEQ ID NO: 8) were used to evaluate the immunomodulatory role of VSTM5 on mouse T cell responses; and mouse IgG2a was used as negative control. Upon co-immobilization on microplates with anti-CD3, VSTM5-hECD-mIg (SEQ ID NO: 131) suppressed mouse CD4⁺ T cell activation (FIG. 12A-D), as manifested by reduction in TCR-induced cytokine secretion (IFNγ and IL-2; (FIGS. 12A-B)), and in cell division of T cells (FIG. 12C). VSTM5-mECD-mIg (SEQ ID NO: 8) was tested in a similar assay upon co-immobilization with anti-CD3, and was also shown to suppress upregulation of activation marker CD69 in mouse CD4⁺ T cells upon TCR stimulation (FIG. 12D).

The above described functional assays used to evaluate the activity of VSTM5-ECD-Ig on mouse T cells, demonstrate the inhibitory effect of VSTM5-ECD-Ig on mouse T cells activation, manifested by reduced cytokine secretion, proliferation and suppression of activation marker CD69 upregulation. This inhibition of T cell activation, similarly to that observed with the native membrane bound VSTM5 protein, in Example 3, supports the therapeutic potential of VSTM5 therapeutic agents according to the present invention (e.g. VSTM5 polypeptides and fusion proteins according to the invention) in treating T cell-driven autoimmune diseases, such as rheumatoid arthritis, multiple sclerosis, psoriasis and inflammatory bowel disease, as well as for other immune related diseases and/or for reducing the undesirable immune activation that follows gene therapy. In addition, these results also support the therapeutic potential of VSTM5 based therapeutic agents that reduce the inhibitory activity of VSTM5 for treating conditions which should benefit from enhanced immune responses, in particular enhanced CTL immunity and proinflammatory cytokines such as immunotherapy and the treatment of cancer, infectious diseases, particularly chronic infections and sepsis wherein T cell depletion of diseased cells is therapeutically advantageous.

### Example 6

### Effect of VSTM5 Fusion Protein on Human T Cells Activated Using Anti-CD3 and Anti-CD28 in the Presence of Autologous PBMCs

### Materials and Methods

In these experiments the effects of VSTM5 on human T cells which were activated using anti-CD3 and anti-CD28 in the presence of autologous PBMCS was evaluated. In the experiment VSTM5 ECD fused to human IgG1 Fc (VSTM5 hECD-hIg, SEQ ID NO: 130) was produced by ExcellGene in CHO-DG44 cells as described in Example 5 herein. CD4⁺ Human T cell Isolation Kit II was purchased from Miltenyi (Cat. #130-094-131). hIgG1 control (Synagis®) was obtained from Medimmune Inc. Anti-human CD3 Ab (OKT3, Cat#16-0037) and anti-human CD28 Ab (clone CD28.2; Cat#16-0289) were purchased from eBioscience. Dynabeads M-450 Epoxy (Cat. #140.11) were purchased from Invitrogen. Buffy coats of human blood were obtained from LifeSource. Ficoll-Paque Plus (Cat. #17-1440-02), was purchased from GE HealthCare.

Isolation of PBMCs from Buffy Coats Using Ficoll Separation

Total PBMCs were suspended in Ex-Vivo 20 medium, and irradiated at 3000rad. Naïve CD4⁺ T cells were isolated from buffy coats of three healthy human donors' blood using CD4⁺ Human T cell Isolation Kit II (Miltenyi) according to manufacturer's instructions and co-cultured with irradiated autologous PBMCs at a ratio of 1:1 (1.5×10⁵ T cells with 1.5×10⁵ irradiated PBMCs per well). The cultures were activated with anti-CD3 (0.5 ug/ml) and anti-CD28 (0.5 ug/ml) antibodies. VSTM5 hECD-hIg (SEQ ID NO: 130) or hIgG1 control Ig (Synagis®) were added to the culture at the indicated concentrations. After 24 hr in culture, cells were pulsed with H3-thymidine. Cells were harvested after 72 hours in culture.

### Results

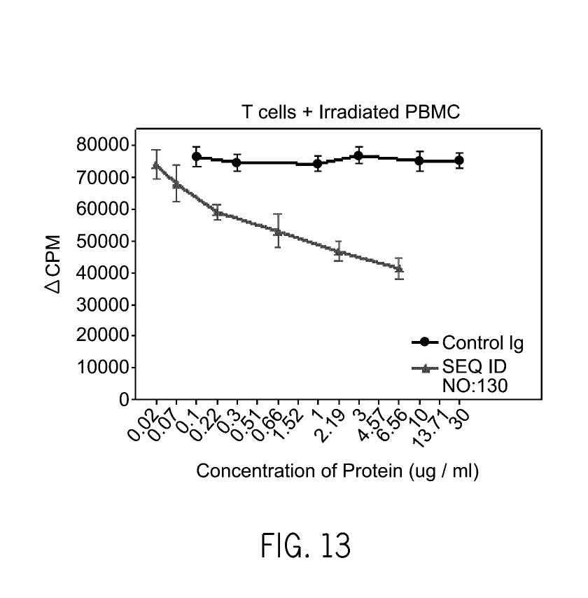

The data from the above assays showed that the addition of VSTM5 hECD-hIg (SEQ ID NO: 130) to cultures of naïve T cells activated by anti-CD3/anti-CD28 in the presence of irradiated autologous PBMCs resulted in a dose dependent inhibition of T cell proliferation, as shown in FIG. 13. This inhibition of T cell activation, similar to that observed with the native membrane bound VSTM5 protein, in Example 3, and to that observed with the VSTM5 ECD-Ig on mouse T cells in Example 5, further supports the therapeutic potential of VSTM5 based therapeutic agents (e.g. VSTM5 polypeptides or VSTM5 fusion proteins according to the invention) for treating T cell-driven autoimmune diseases, such as rheumatoid arthritis, multiple sclerosis, psoriasis and inflammatory bowel disease, as well as for treating other immune related diseases and/or for reducing the undesirable immune activation that follows gene or cell therapy. Essentially, antibodies or VSTM5 polypeptides and fusion proteins that agonize VSTM5 should prevent or reduce the activation of T cells and the production of proinflammatory cytokines involved in the disease pathology of such conditions.

In addition, these results also support a therapeutic potential of VSTM5 targeting agents that reduce the inhibitory activity of VSTM5 (e.g., VSTM5 polypeptides or VSTM5 fusion proteins according to the invention) for treating conditions which will benefit from enhanced immune responses such as immunotherapy of cancer, infectious diseases, particularly chronic infections and sepsis. Essentially, VSTM5 polypeptides, fusion proteins or antibodies that antagonize VSTM5 should promote the activation of T cells and elicit the production of proinflammatory cytokines thereby promoting the depletion of cancerous or infected cells or infectious agents.

### Example 7

### Effect of VSTM5 Fusion Protein Co-Coated on Beads Together with Anti-CD3 and Anti-CD28 on Human T Cell Activation

The effect of VSTM5-Fc fusion protein on T cell activation was evaluated in a bead assay in which VSTM5 hECD-hIg fusion protein (SEQ ID NO: 130) was co-coated on the beads together with anti CD3

### Materials and Methods

### Isolation of Human T Cells:

Buffy coats were obtained from Stanford Blood Bank from healthy human donors. CD3⁺ T cells were isolated from buffy coats using RosetteSep kit (StemCell Technologies) following manufacturer's instructions. Cells were >94% CD3 positive when analyzed with anti-CD45 and anti-CD3 by flow cytometry, and >95% viable after thawing prior to the assay.

### Bead Coating and QC:

Tosyl activated beads (Invitrogen, Cat#14013) at 500×10⁶/ml were coated with anti CD3 mAb and Fc fusion proteins in a two-step protocol: with 50 ug/ml human anti-CD3 clone UTCH1 (R&D systems, Cat# mab 100) in sodium phosphate buffer at 37° C. overnight, followed with 0-320 ug/ml of VSTM5-hECD-hIg fusion protein (SEQ ID NO: 130) for another overnight incubation at 37° C. In the second step, control human F_{c} (Bioxcell, Cat# BE0096) was added together with the Fc fusion protein so that the total amount of protein is 160 ug/ml for the 0, 20, 40, 80 and 160 ug/ml coating condition except for the 320 ug/ml coating condition.

Equal levels of anti-CD3 on the different beads preparations was validated using goat anti-mouse Ab Alexa 647®-conjugated (Jackson ImmunoResearch, Cat#115-606-146). In addition, different levels of VSTM5 hECD-hIg fusion protein corresponding to the above described concentrations were validated using Alexa 647® conjugated anti-VSTM5 mab 53-01.B11 (Lot 20414)

### Bead Assay Setup:

10⁵ human CD3⁺ T cells were cultured with 10⁵ or 2×10⁵ beads co-coated with anti CD3 and various concentrations of VSTM5 hECD-hIg fusion protein (SEQ ID NO: 130) for 5 days in complete IMDM (Gibco, Cat #12440-053) supplemented with 2% AB human serum (Gibco, Cat#34005-100), Glutmax® (Gibco, Cat #35050-061), sodium pyruvate (Gibco, Cat #11360-070), MEM Non-Essential Amino Acids Solution (Gibco, Cat #11140-050), and 2-mercaptoethanol (Gibco, Cat #21985). At the end of 5 day culture, cells were stained with anti-CD25, anti-CD8, and fixable live dead dye to determine CD25 expression levels on each subset of cells. Supernatants were collected and assayed for IFNγ secretion by ELISA (Human INFγ duoset, R&D systems, DY285)

### Results

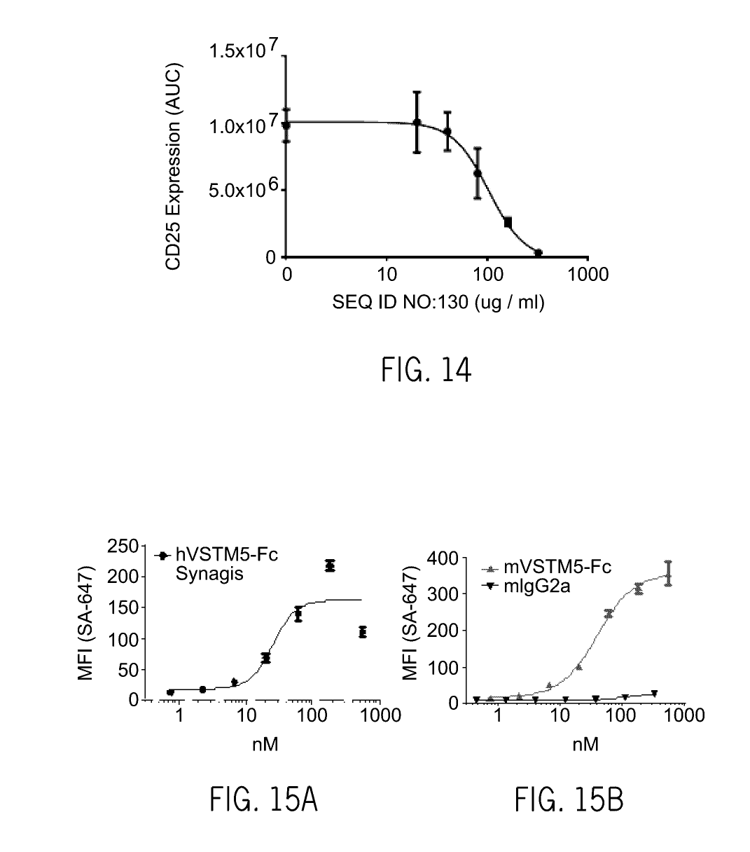

The data obtained from the above-described experiments showed that the stimulation of human T cell with beads co-coated with a constant amount of anti CD3 and different amounts of VSTM5-Fc fusion protein resulted in a dose dependent down-regulation of CD25 expression, as shown in FIG. 14. These results indicate that this VSTM5-Fc fusion protein has an inhibitory effect on human T cell activation and therefore should be useful as a therapeutic agent in indications wherein reduced T cell activity is desired.

### Example 8

### Binding Assays to Investigate the Binding Capacity of VSTM5 ECD-Ig to Ts

Study I: Binding of Human VSTM5 ECD Fused to Human IgG1 Fc to T Cell Lines

### Materials and Methods

### Cell Line:

H9 cells were purchased from ATCC (ATCC cat no: HTB-176) and cultured in complete media (CM): RPMI (Gibco #21870-092) supplemented with 10% FBS (Gibco #16000-044), Glutamine (Gibco #25030-081) and Penstrep® (Gibco #15070-063).

### Fc-Fusion Proteins and Isotype Controls:

Human VSTM5 ECD fused to human IgG1 Fc (also called VSTM5 hECD-hIg) (SEQ ID NO: 130) was produced at GenScript by transient transfection in CHO-3E7 cells. Cells were cultured for 6 days, followed by Protein A purification of cell harvest; mouse VSTM5 ECD fused to mouse IgG2a Fc (also called VSTM5-mECD-mIgG) (SEQ ID NO: 8) was produced at ExcellGene in CHO-DG44 cells as described in Example 5 herein; human IgG1 isotype control (#ET901) was purchased from Eureka Therapeutics, USA; Synagis® (Palivizumab, anti-RSV humanized IgG1 mAb) was purchased from Caligor Rx; biotinylated human IgG-Fc isotype control (#009-060-008) was purchased from Jackson Immunoresearch; mouse IgG2a isotype control, clone MOPC-173 (unlabeled #400224 and biotinylated #400204), was purchased from Biolegend, USA.

### Reagents:

BiotinSP-AffiniPure® Goat anti-human IgG, Fcγ specific (#109-065-098), biotinSP-AffiniPure® goat anti-mouse IgG, Fcγ2a specific (#115-065-206) and streptavidin-AF647 (SA-AF647 #016-600-084) were purchased from Jackson Immunoresearch; Dulbecco's PBS (DPBS, Life Technologies #14190-250); BSA (Sigma Aldrich #A12153); Biotinylation of VSTM5 hECD-hIg was performed using the EZ-Link Sulfo-NHS-LC-Biotin reagent (Pierce #21327) according to manufacturer's instructions.

### Binding Assay:

The binding of VSTM5-ECD-Ig (SEQ ID NOs: 130 or 8) to H9 cells was detected by FACS using either a two-step or three-step detection protocol. H9 cells were harvested after reaching confluency which occurred between 0.6-1.2×10⁶ cells/ml. Cells were plated at 5×10⁴ cells per well in a 96 well v bottom plate, pelleted and the supernatant flicked off. Binding with VSTM5-Fc and its following detection was carried out as described below.

### Two-Step Detection

Biotinylated VSTM5 hECD-hIg or biotinylated human IgG control were titrated in FACS buffer (0.5% BSA/DPBS) by performing an 8 point 3-fold serial dilution ranging from 550 nM to 0.3 nM, and 50 ul added to wells for 45 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

### Three-Step Detection

Unlabeled VSTM5 hECD-hIg, VSTM5 mECD-mIg, human IgG1 isotype control or mouse IgG2a isotype control were titrated in FACS buffer by performing an 8 point 3-fold serial dilution ranging from 550 nM to 0.3 nM, and 50 ul added to wells for 45 minutes at 4° C. Following one wash in FACS buffer, 50 ul of biotin-anti human IgG Fcγ specific or biotin-anti mouse IgG Fcγ2a specific antibody was added for 30 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

### Competition Assay:

Prior to labeling H9 cells with biotinylated VSTM5 hECD-hIg cells were incubated in 50 ul of increasing concentration of unlabeled VSTM5-hECD-hIg or human IgG isotype control for 45 minutes at 4° C. Following centrifugation, supernatant was removed and 50 ul of biotinylated VSTM5 hECD-hIg at a fixed concentration of 44 nM, was added for 45 minutes at 4° C. After one wash in FACS buffer, 50 ul of 1:150 dilution of streptavidin-AF647 made up in FACS buffer was added and incubated in the dark for 20 to 30 minutes at 4° C. Following two washes in FACS buffer, samples were read on a BD Bioscience FACS Calibur with a Cytek HTS or an Accuri Intellicyte HTFC.

### FACS Analysis:

Data was analyzed in FCS Express (DeNovo), exported to Excel and plotted in GraphPad Prism. The data shown in FIG. 15 is representative of two to five experiments.

### Results

### Human and Mouse VSTM5-ECD-Ig Bind H9 Cells

VSTM5 ECD-Ig binding to H9 cells was evaluated using the human or mouse ECD fused to a human or mouse Fc (SEQ ID NOs: 130 or 8), respectively. As shown in FIG. 15A-B, using a 3 step labeling method, both human (FIG. 15A) and mouse (FIG. 15B) VSTM5-Ig were found to bind H9 cells in a dose-dependent manner, while their respective isotype controls showed no binding.

### Binding of VSTM5 ECD-Ig can be Competed Off by Itself

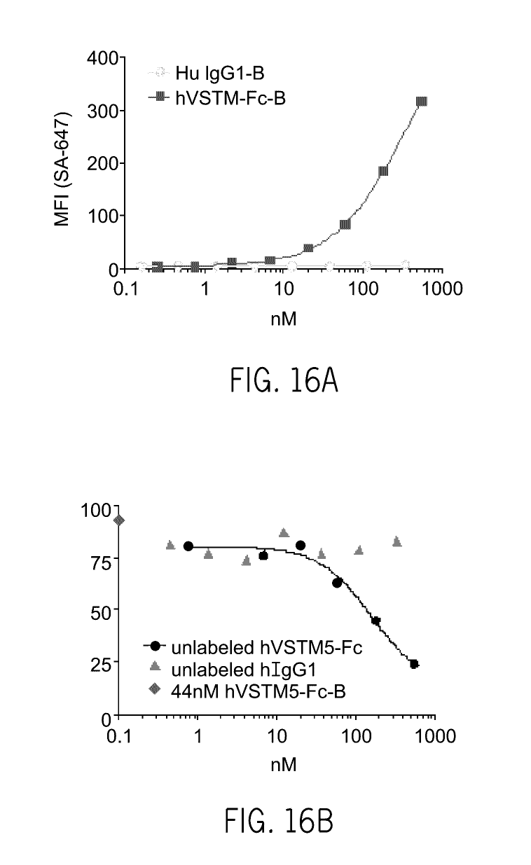

Biotinylated VSTM5 hECD-hIg binds H9 cells, using a two-step detection method (FIG. 16A). At a given concentration of biotinylated VSTM5 hECD-hIg (FIGS. 16A & 16B, diamond), the unlabeled VS™ hECD-hIg reduces binding of the biotinylated VSTM5 hECD-hIg with increasing concentrations, while the isotype control did not (FIG. 16B).

### Review of Results

The results of the above-described assays show that VSTM5-hECD-hIg and VSTM5-mECD-mIg (SEQ ID NOs: 130 or 8, respectively) bind to human H9 T cell line in a dose-dependent manner, indicating that a counterpart receptor of VSTM5 is likely expressed by these cells. These data further indicate that this binding assay can be used to screen for neutralizing anti-VSTM5 blocking monoclonal antibodies or VSTM5 polypeptides and fusion proteins according to the invention.

Study II: Binding of Human VSTM5 ECD Fused to Human IgG1 Fc to Primary Resting and Stimulated Human CD4⁺ or CD8⁺ T Cells

Materials and Methods

### Fc-Fusion Proteins and Isotype Controls:

Human VSTM5 ECD fused to human IgG1 Fc (also called VSTM5 hECD-hIg) (SEQ ID NO: 130) was produced at GenScript by transient transfection in CHO-3E7 cells. Cells were cultured for 6 days, followed by Protein A purification of cell harvest and a second step purification by preparative SEC to remove aggregates; Synagis® (Palivizumab, anti-RSV humanized IgG1 mAb) was purchased from Caligor Rx.

Human primary CD4⁺ or CD8⁺ T cells were enriched from buffy coats of a healthy donors using RosetteSep™ Human T Cell Enrichment Cocktail (Stem Cell Technologies), according to manufacturer's instructions. The purity obtained was >95%. Following enrichment, cells were cryo-preserved (90% FCS, 10% DMSO) until future use.

Frozen human CD4⁺ or CD8⁺ T cells were thawed, washed, and re-suspended at 0.5×10⁶ cells/ml in lymphocyte complete medium (CM) consisting of Iscove's Modified Dulbecco Medium (cat#01-058-1A, Biological Industries) supplemented with 10% (v/v) inactivated fetal calf serum (FBS, Biological Industries), 2 mM 1-glutamine, 100 U/ml penicillin, 100 μg/ml streptomycin, 1 mM sodium pyruvate, and 1% non-essential amino acid. 200 μl of cells suspension was added per well of 96-well plate (Costar Cat#3599) pre-coated with PBS in the absence or presence of 1 ug/ml anti-CD3 (clone UCHT-1; R&D systems) for 4 h, at 37° C. Plates were placed in a humidified, 5% CO₂, 37° C. incubator for 72 hours. Cells were harvested and stained with viability dye (Fixable Viability Stain 450, cat#562247, BD Biosciences), washed with PBS and incubated for 1 hr at room temperature (RT) with B7H1-Ig as a positive control (R&D systems; cat#156-B7-100), human VSTM5 ECD fused to human IgG1 Fc (SEQ ID NO: 130) or control Ig (hIgG1-Synagis, Medimmune) at 100 ug/ml (50 μl/well) in FACS buffer (0.5% BSA, 2 mM EDTA, 0.05% NaN3 in PBS). Cells were washed 3 times and stained with PE-conjugated anti-hIgG (Cat#109-116-098, Jackson laboratory; diluted 1:100) in final volume of 50 μl, 30 min 4° C. Data acquisition was performed with MACSQuant Analyzer 10 (Miltenyi) and data analyzed using FlowJo software (version 10).

Results

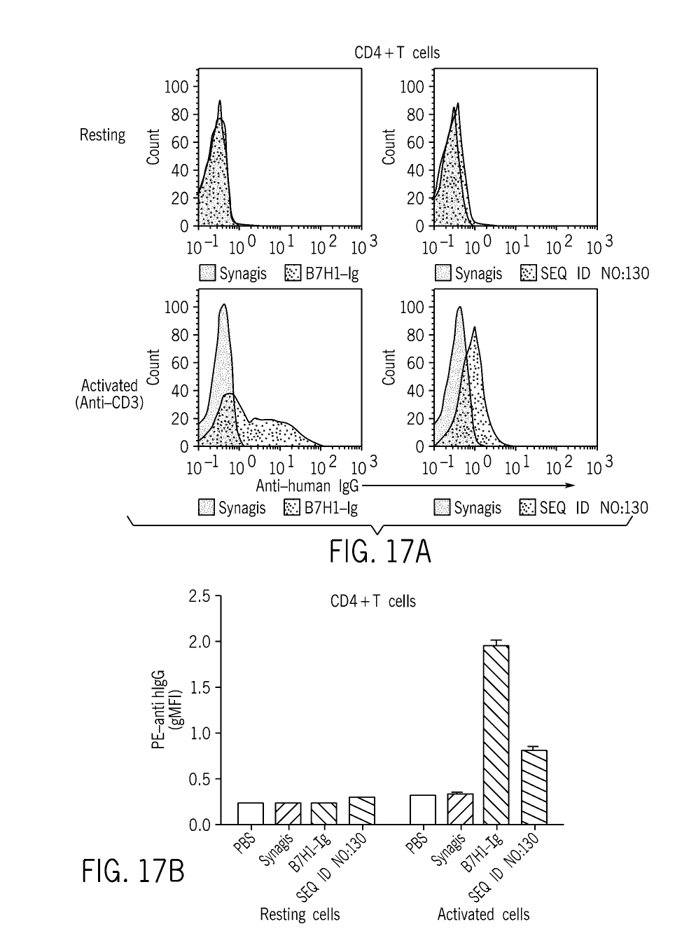

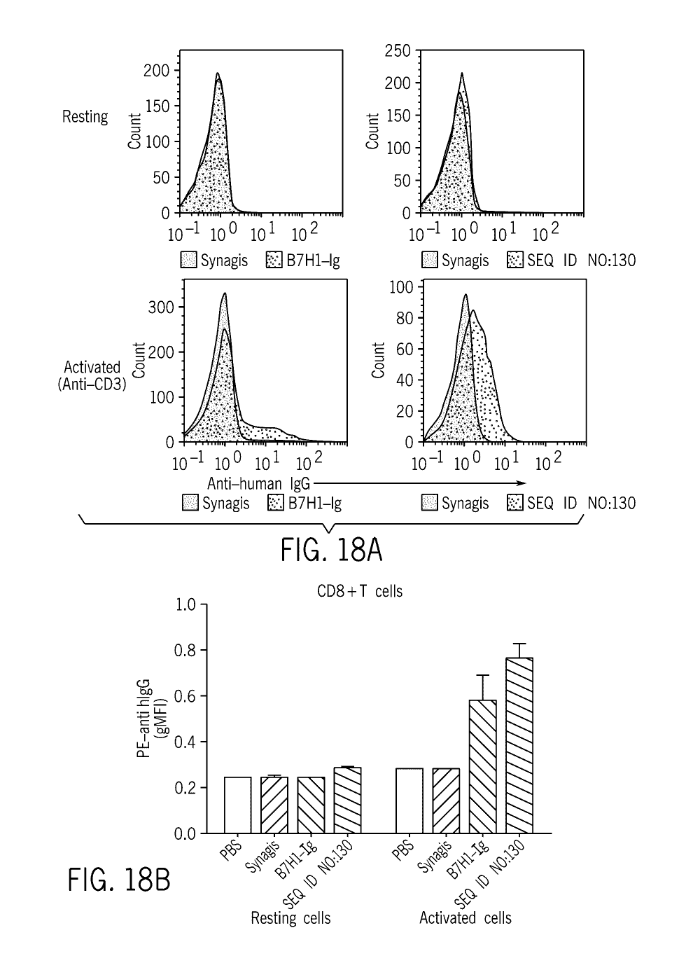

In the above-described experiments showed that isolated human CD4⁺ or CD8⁺ T cells were left untreated (resting) or stimulated with immobilized anti-CD3 (1 ug/ml) for 3 days as described above. Cell activation was validated by evaluating the expression of PD-1 which was observed on activated CD4⁺ T cells, but not on resting CD4⁺ cells (not shown). Results in FIG. 17 show binding of unlabeled human VSTM5 ECD fused to human IgG1 Fc SEQ ID NOs: 130 to anti-CD3 activated, but not resting, human CD4⁺ T cells, with a 2.5 fold increase in the geometric MFI (gMFI) compared to Ig control (FIG. 18; gMFI values: SEQ ID NO: 130: 0.81 vs. control Ig: 0.335). B7H1-Ig was used as a positive control since it is a known ligand of PD-1, which is up-regulated upon T cell activation. As expected, binding of B7H1-Ig to activated, but not to resting CD4⁺ T cells, was observed (FIG. 18; gMFI values, B7H1-Ig 1.9 vs. 0.335 of Ig control). The binding of human VSTM5 ECD fused to human IgG1 Fc (SEQ ID NO: 130) to resting and activated CD8⁺ cells was also evaluated. A similar binding pattern was observed: i.e., human VSTM5 ECD fused to human IgG1 Fc (SEQ ID NO: 130) bound to activated, but not to resting CD8⁺ T cells.

### Review of Results

The results of these experiments demonstrate binding of human VSTM5 ECD fused to human IgG1 Fc to human CD4⁺ and CD8+ T cells, which were pre-activated in a TCR-dependent manner No binding was detected when resting CD4⁺ or CD8⁺ T cells were examined. These data strongly suggest the expression of an inducible counterpart receptor for VSTM5 on activated T cells. These findings are similar to those reported for known B7/CD28 family negative receptors, such as CTLA4 and PD-1, found to be upregulated on T cells following activation.

### Example 9

### In-Vitro Assays to Investigate Immunomodulatory Activities of VSTM5 ECD-Ig on Mouse Induced Tregs

The aim of this example was to investigate the effect of the mouse VSTM5 ECD fused to the Fc of mIgG2a, (VSTM5 mECD-mIg, SEQ ID NO:8) on the induction of mouse iTregs following CD4⁺ T cell activation under iTreg driving conditions.

Materials and Methods

Isolation of Mouse CD4⁺ T Cells

CD4⁺CD25⁻ T cells were negatively isolated from C57BL/6J mouse spleen cell suspensions with CD4⁺ T-cell enrichment isolation kits (Stem Cell Technologies), and further purified by flow cytometry to >99% purity. CD4⁺CD25⁻ T cells were labeled with CFSE (0.5 μM; Molecular Probes Cat. #C34554) according to the manufacturer's instructions.

### Mouse CD11c+ Dendritic Cell Isolation

CD11c⁺ dendritic cells (DCs) were isolated from spleen cell suspensions to >90% purity by magnetic separation using mouse CD11c⁺ selection kits (StemCell Technologies).

Activation of Mouse CD4⁺ T Cells Under iTreg Driving Conditions in the Presence of Antigen Presenting Cells

Anti-mouse CD3-ε mAb (clone 145-2C11; BD Pharmingen) was immobilized overnight at 4° C., at 5 μg/ml, on 96-well flat-bottom tissue culture plates (Sigma, Cat. # Z707910). Wells were washed 3 times with PBS, and plated with 2×10⁴ purified CD11c⁺ dendritic cells at a final volume of 100 μl. VSTM5 mECD-mIg composed of mIgG2a Fc fused to mouse VSTM5 ECD (produced at ExcellGene in CHO-DG44 cells as described in Example 5 herein)(SEQ ID NO:8) was added to a final concentration of 10 μg/ml and kept in a humidified, 5% CO₂, 37° C. incubator for 1 hour. 1×10⁵ CD4⁺ T cells were added to a final volume of 200 μl. Soluble anti-CD28 was added at 1 μg/ml. Afterward IL-2 (5 ng/ml) and TGF-β (3 ng/ml) were added. Cells were maintained in plastic tissue plates at 37° C. in a humidified atmosphere with 5% CO₂, and analyzed by flow cytometry four days later.

Activation of CD4⁺ T Cells in the Presence of iTreg Driving Conditions (without Antigen Presenting Cells)

96-well flat bottom tissue culture plates (Sigma, Cat. # Z707910) were coated with anti-CD3 mAb (2 ug/mL) and VSTM5 mECD-mIg (SEQ ID NO:8) or control Ig control (MOPC-173, Biolegend) at 10 ug/ml. CD4⁺CD25⁻ T cells were thawed and added to wells (0.5×10⁵/well) in the presence of soluble anti-CD28 (1 ug/ml), TGF-β (Cat#7666-MB; R&D systems) and IL-2 (Cat#202-IL; R&D systems) at the indicated concentrations. On Day 5 post-stimulation the percentage of CD4⁺CD25⁺FoxP3⁺ cells was assessed by flow cytometry.

### Results

VSTM5 (SEQ ID NO:8) Enhances Induction of iTregs

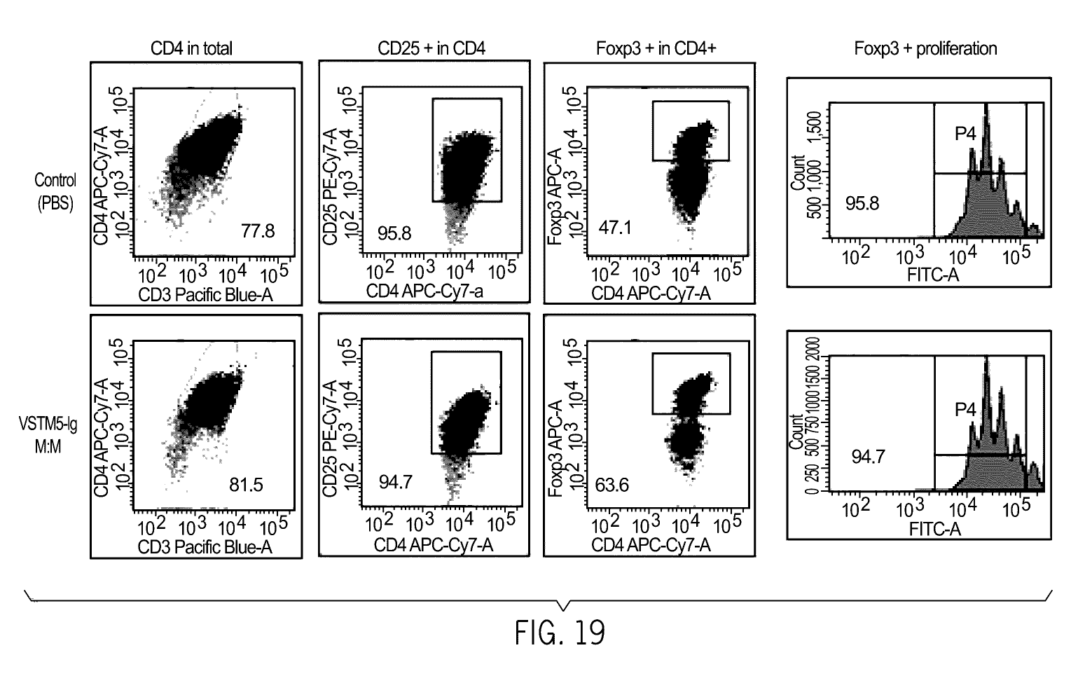

The effect of VSTM5 mECD-mIg (SEQ ID NO: 8) on the induction of mouse iTregs was evaluated following T cell activation under iTreg driving conditions in the presence of antigen presenting cells. The results shown in FIG. 19 indicate that the addition of VSTM5 mECD-mIg (SEQ ID NO: 8), upon activation of CD4⁺ T cells with plate-bound anti-CD3 in the presence of mIL-2 and TGFβ, enhanced the induction of Foxp3⁺ iTregs by ˜50% (from 47.1% of total CD4⁺ T cells in the presence of PBS control, to 63.6% in the presence of VSTM5 mECD-mIg (SEQ ID NO: 8)).

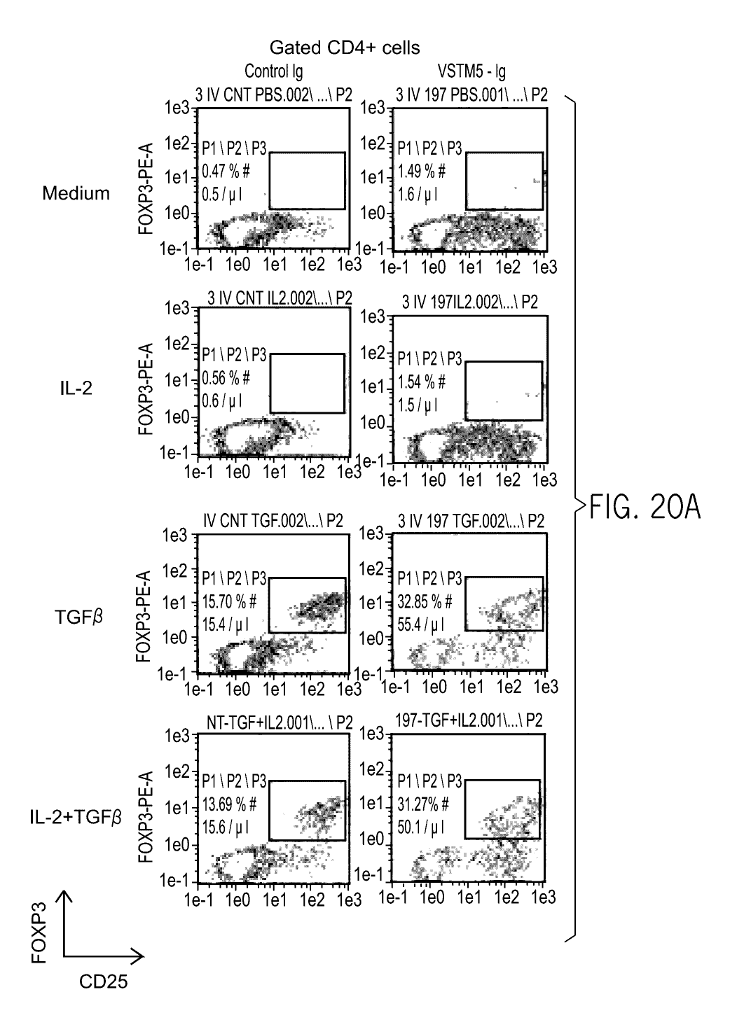

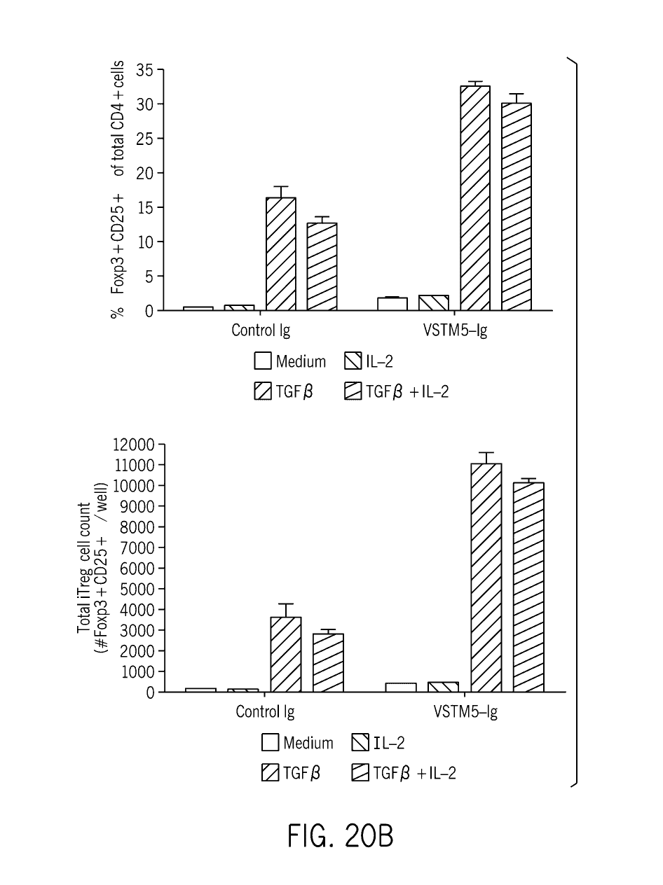

The effect of VSTM5 mECD-mIg (SEQ ID NO: 8) on the induction of mouse iTregs was evaluated following T cell activation under iTreg driving conditions in an experimental set-up in which VSTM5 mECD-mIg (SEQ ID NO: 8) is not presented by antigen presenting cells but rather immobilized to the plate together with the anti-CD3. The effect on iTreg induction was evaluated by two different parameters: the total count of CD25⁺ and FOXP3⁺ cells (given by cell count per microliter), and the percentage CD25⁺ and FOXP3⁺ cells out of the total CD4⁺ cells. The results shown in FIG. 20 indicate that the addition of VSTM5 mECD-mIg (SEQ ID NO: 8), upon activation of CD4⁺ T cells with plate-bound anti-CD3 in the presence of TGFβ with or without mIL-2, enhanced the induction of Foxp3⁺ iTregs by two fold in the percentage CD25⁺ and FOXP3⁺ cells out of the total CD4⁺ cells (from 17% of total CD4⁺ T cells in the presence of Ig control (MOPC-173, Biolegend), to 34% in the presence of VSTM5 mECD-mIg (SEQ ID NO: 8)) and by three-fold in the total count of CD25⁺ and FOXP3⁺ cells (from 3500 Foxp3⁺ iTregs per microliter in the presence of Ig control (MOPC-173, Biolegend), to 11,000 in the presence of VSTM5 mECD-mIg (SEQ ID NO: 8). FIG. 20A presents representative plots of gated CD4⁺ cells. The reported values represent the percentage of CD25⁺Foxp3⁺ cells of the total CD4⁺ cells or total Tregs cell count/μl. FIG. 20B contains plots representing the average percentages or total cell count of iTregs from triplicate cultures for each condition.

### Example 10

### In Vitro Assays to Determine Immunomodulatory Effect of VSTM5 on Human NK Cells

In these experiments the binding potential of human ECD of VSTM5 fused to Fc of human IgG1 (VSTM5 hECD-hIg, SEQ ID NO:130) to NK cells was evaluated in order to assess whether the over expression of human VSTM5 on different human cancer cell lines affects their susceptibility to killing by NK cells.

### Materials and Methods

Isolation of NK Cells from Peripheral Blood Mononuclear Cells

Human NK cells were isolated from PBLs (peripheral blood cells) from one healthy human donor using the human NK cell isolation kit and the autoMACS instrument (Miltenyi Biotec, Auburn, Calif.).

### Generation of Primary NK Cell Clones and Polyclonal NK Cells

Human primary NK cell clones were obtained by seeding purified human primary NK cells at one cell/well in 96-well U-bottomed plates in complete medium supplemented with 10% FCS, 10% leukocyte-conditioned medium and 1 μg/ml PHA. Irradiated feeder cells (2.5×10⁴ allogeneic PBMCs from two donors and 5×10³ RPMI 8866 B cell line in each well) were added. Proliferating clones, as defined by growth at cell densities where growth of cells occurred in less than one third of the wells plated, were expanded in complete medium in 96-well plates. These human activated primary NK cell clones, designated as ‘NK cell clones’ in this report, were cultured in RPMI, 10% human serum supplemented with 1 mM glutamine, 1 mM nonessential amino acids, 1 mM sodium pyruvate, 10000 units penicillin streptomycin and 50 U/ml rhuIL-2. The killing assays were performed using a polyclonal population of NK cells (i.e., after unification of all viable NK cell clones from a certain donor).

### Generation of Human Cell Lines Ectopically Expressing VSTM5

The cDNA encoding VSTM5 was cloned into the pHAGE-DsRED(−)-eGFP(+) lentiviral vector, and transduced to the following human cancer cell lines: HeLa (cervical carcinoma), RKO (colon carcinoma), RPMI-8866 (lymphoblastoid B cell line), BJAB (EBV-negative Burkitt lymphoma). The level of expression of VSTM5 in the various transduced cell lines was evaluated by FACS analysis using a commercial rabbit polyclonal antibody (Sigma, HPA029525). A rabbit IgG (SIGMA 15006) was used as isotype control, and as secondary antibody we used anti-rabbit APC (Jackson, Cat #711-136-152).

Cytotoxicity Assay

The cytotoxic activity of polyclonal NK cells against various human cell lines ectopically expressing VSTM5 was evaluated using S35 release assay, in which effector cells were mixed with 5×10³ [S35] methionine-labeled target cells at different E:T (Effector cells to Target cells) ratios in U-bottomed microtiter plates. Following 5 hours incubation at 37° C., assays were terminated by centrifugation at 1500 rpm for 5 min at 4° C. and 50 μl of the supernatant was collected for liquid scintillation counting. Percent specific lysis was calculated as follows: % lysis=[(cpm experimental well−cpm spontaneous release)/(cpm maximal release—cpm spontaneous release)]×100. Spontaneous release was determined by incubation of the S35-labeled target cells with medium only. Maximal release was determined by solubilizing target cells in 0.1M NaOH. In all presented experiments, the spontaneous release was <25% of maximal release.

### Binding Assay

NK cell clones or polyclonal NK cells were incubated with 5 μg of VSTM5 hECD-hIg (produced at ExcellGene in CHO-DG44 cells as described in Example 5 herein) (SEQ ID NO:130) or isotype control (hIgG1) for 2 hours on ice. Following cell washing, secondary anti-mouse antibody was added and binding was evaluated by flow cytometry.

### Results

VSTM5 hECD-hIg (SEQ ID NO:130) Binds to NK Cells

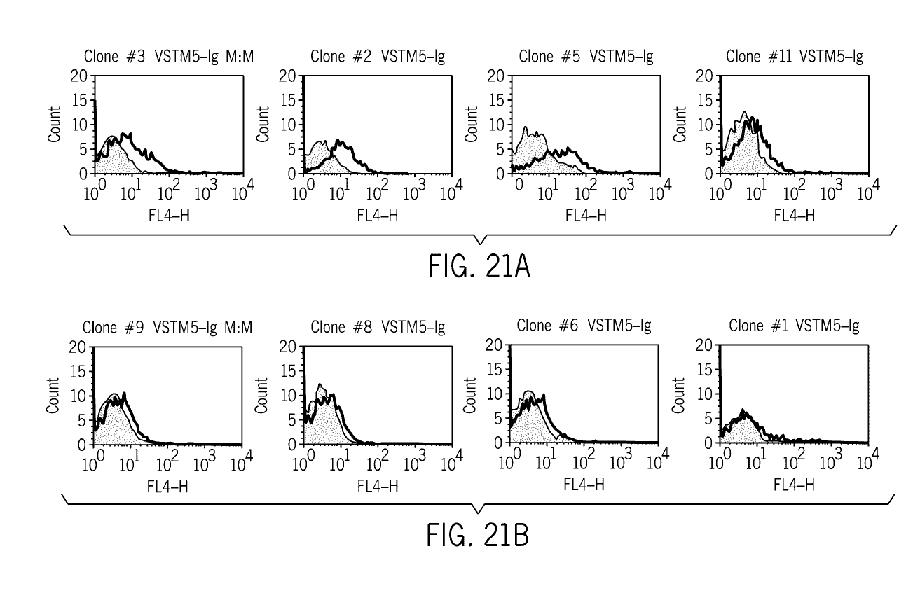

In these experiments, the binding of VSTM5 hECD-hIg (SEQ ID NO:130) to activated primary NK cell clones was evaluated. As shown in FIG. 21A-B, VSTM5 hECD-hIg (SEQ ID NO:130) bound to activated NK cell clones at varying intensities. FIG. 21A presents clones with high binding intensities, and FIG. 21B presents clones with low binding intensities.

### Over Expression of VSTM5 on Human Cells Reduces NK Cytotoxicity.

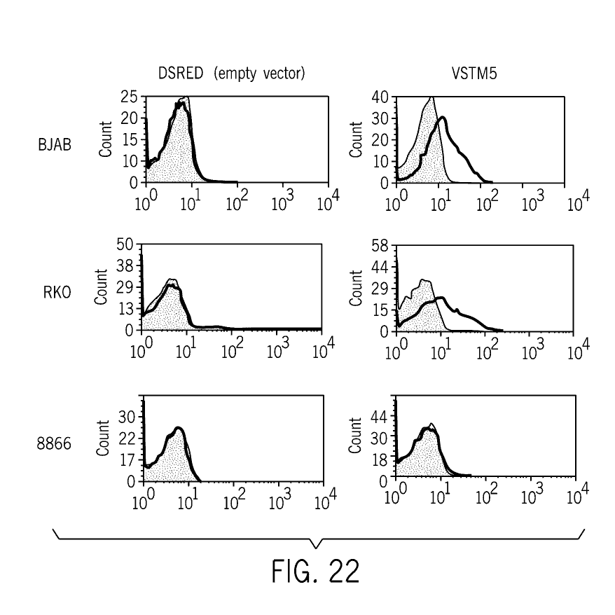

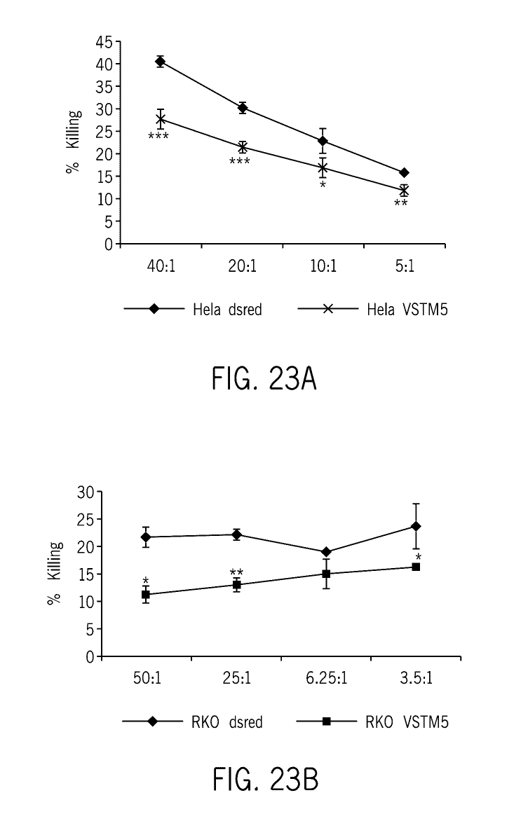

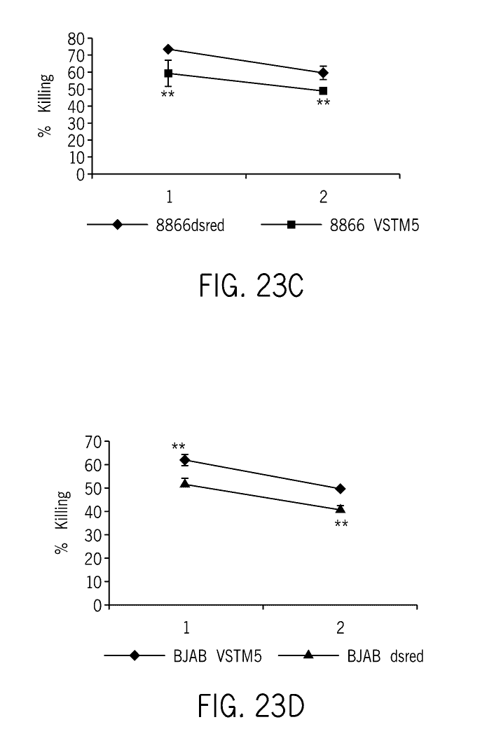

cDNA encoding VSTM5 (SEQ ID NO:6)) was transduced to various human cancer cell lines as described in Materials and Methods. The level of expression of VSTM5 in the various cell lines was evaluated by FACS analysis and shown in FIG. 22. The effect of VSTM5 over expression on the susceptibility to killing by NK cells was assessed. Several experiments were carried out using these cell lines. The results of representative experiments are shown in FIG. 23A-D and indicate that the over expression of VSTM5 on these target cells (Hela—FIG. 23A, RKO—FIG. 23B, 8866—FIG. 23C and BJAB-FIG. 23D) results in a reduction of NK cells killing activity at different E:T ratios.

### Review of Results

Binding of VSTM5 hECD-hIg fusion protein to activated NK cells was detected on several NK cell clones. In co-culture experiments, the over expression of VSTM5 on various human cancer cell lines was shown to reduce the killing activity of NK cells, indicating that the counter receptor of VSTM5 is expressed on NK cells, and that VSTM5 has an inhibitory effect on killing activity of NK cells. Therefore, these results further suggest that VSTM5 fusion proteins as disclosed and claimed herein may be used to modulate the cytotoxicity of NK cells, e.g., to promote the killing of cancerous, infected cells or infectious agents, and to suppress the activity of NK cells when clinically desirable, e.g., in autoimmune or inflammatory conditions.

### Example 11

### In-Vitro Assays to Investigate Immunomodulatory Activities of VSTM5 on Human Cytotoxic T Cells (CTLs)

The experiments described in this example evaluated the effect of ectopic expression of human VSTM5 (SEQ ID NO:132) on different melanoma cell lines on their potential to activate CTLs (Cytotoxic T Lymphocytes) and to serve as targets for killing by these cells.

### Materials and Methods:

### General Design of the Experimental System:

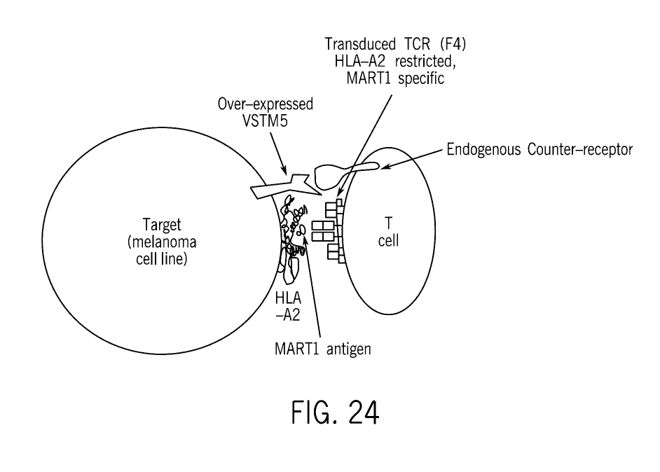

In the experimental system described in FIG. 24, VSTM5 (SEQ ID NO:132) is over expressed on human melanoma cells as target cells, which are then co-cultured with primed human CD8⁺ T cells (CTLs) over expressing a TCR designated F4 which is specific for an antigen derived from the melanoma specific protein MART1, when presented on HLA-A2 (specific class I MHC). (This TCR was used in clinical trials in terminally-ill melanoma patients to specifically confer tumor recognition by autologous lymphocytes from peripheral blood by using a retrovirus encoding the TCR (Morgan et al, 2006 Science, 314:126-129)).

### Expression of VSTM5 (SEQ ID NO:132) in Melanoma Cell Lines:

In order to express VSTM5 (SEQ ID NO:6) on target melanoma cells, the cDNA encoding VSTM5 (SEQ ID NO:6) was amplified using specific primers and cloned into an MSCV-based retroviral vector (pMSGV1). Verification of the cloning was done first using restriction enzyme digestion and subsequently by sequencing. Upon sequence confirmation, large amounts of the retroviral vector (Maxi-prep) were produced for subsequent use.

Three human melanoma cell lines which present the MART-1 antigen in HLA-A2 context (SK-MEL-23, mel-624 and mel-624.38) were transduced with the retroviral constructs encoding VSTM5 (SEQ ID NO:6) or with the empty retroviral vector. A melanoma cell line mel-888 which does not express HLA-A2 served as additional negative control. Transduction was carried out using a retronectin-based protocol; briefly, retroviral supernatant was produced in 293GP cells (a retroviral packaging cell line) following transfection with the retroviral vector and an amphotropic envelop gene (VSV-G). The retroviral supernatant was plated on retronectin-coated plates prior to the transduction to enable the binding of virions to the plate. Then, the melanoma cells were added to the plate for 6 hours. After that, the cells were replenished in a new culture vessel. Transduction efficiency and expression of the protein was determined by staining the transduced tumor cells with a commercial VSTM5-specific polyclonal antibody (Anti-VSTM5, rabbit polyclonal antibody, Sigma, Cat. No. HPA029525). A rabbit IgG (Sigma Cat. No. 15006) was used as isotype control, and as secondary antibody we used APC-conjugated anti-rabbit IgG (Jackson, 711-136-152).

### Transduction of Effector Cells:

In order to obtain effector lymphocytes that express the CD8-dependent MART-1 specific F4 TCR (a MART-126-35-specific TCR that recognizes HLA-A2⁺/MART1⁺ melanoma cells), freshly isolated human PBLs (peripheral blood leukocytes) were stimulated with PHA and cultured for 5-10 days, and subsequently transduced with a retroviral vector encoding both α and β chains from the CD8-dependent MART-1-specific F4 TCR. The transduced lymphocytes were cultured in lymphocyte medium containing 300 IU of IL-2, replenished every 2-3 days. Non-transduced T cells served as negative control.

Cytokine Secretion from F4-TCR Transduced Lymphocytes Upon Co-Culture with VSTM5 (SEQ ID NO:132)-Transduced Melanoma Cells:

Melanoma cells expressing VSTM5 (SEQ ID NO:132) or empty vector were co-cultured for 16-20 hours with F4-TCR transduced lymphocytes. Cytokine secretion (IFN-γ, IL-2 and TNFα) was measured by ELISA, to assess the specific recognition and response of the effector CD8⁺ T cells to the different transduced tumor cell lines. In these assays, 10⁵ effector cells were co-cultured with 105 melanoma target cells for 16 hours. Cytokine secretion was measured in culture supernatants, diluted to be in the linear range of the ELISA assay.

### Killing Assays:

The cytotoxic activity of effector cells (CTLs) against melanoma human cell lines (target cells) ectopically expressing VSTM5 was evaluated by staining for propidium iodide (PI). Effector cells were admixed with CFSE-labeled target cells at optimized E/T (Effector cells to Target cells) ratios in U-bottomed 96 well microtiter plates. Following an overnight incubation at 37° C., cells were stained with PI and read by FACS. The percentage of double positive events (stained for CFSE and PI) out of all CFSE positive events (total melanoma cells) were referred to as melanoma cells undergoing lysis. Non-transduced effector cells were used to obtain the background level of cell lysis not related to T cell specific killing activity.

### Results:

Over Expression of VSTM5 on Human Melanoma Cell Lines

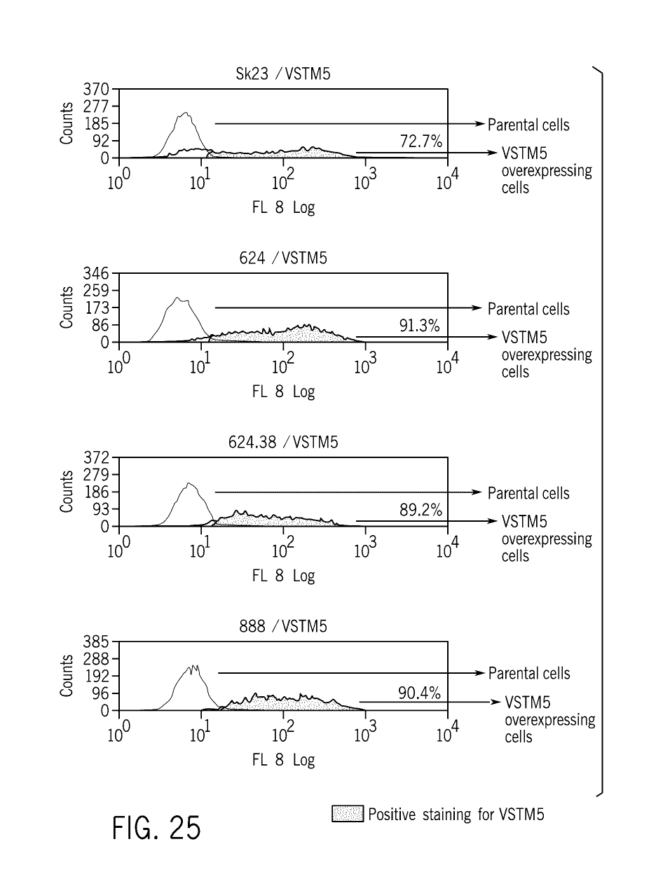

Human melanoma cell lines (SK-MEL-23, mel-624.38, mel-624 and mel-888) were stained with a VSTM5-specific monoclonal antibody. Endogenous expression of VSTM5 was not detected on the surface of these cell lines as shown by flow cytometry (data not shown). Next, these cell lines were transduced with retroviral vector encoding the VSTM5 (SEQ ID NO:6) molecule, as described in Materials & Methods. The levels of VSTM5 expression were assessed by flow cytometry at 48 hrs after transduction, and compared to those of cells transduced with an empty vector. The percent of cells staining positive for the protein ranged between 70-90% for the different cell lines tested (FIG. 25).

Over expression of VSTM5 on human melanoma cells reduces activation-dependent cytokine secretion from F4 transduced CTLs.

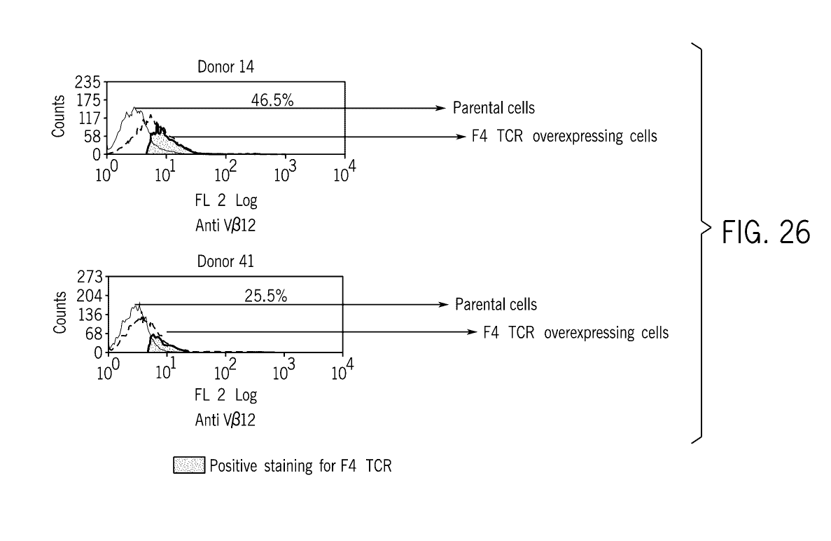

To perform functional assays with human CTLs, primary human lymphocytes were engineered to express the F4 TCR, which recognizes HLA-A2+/MART1+ melanoma cells were used, as described in the Materials & Methods. FIG. 26 shows the level of F4 TCR expression obtained upon transduction of lymphocytes from two representative donors.

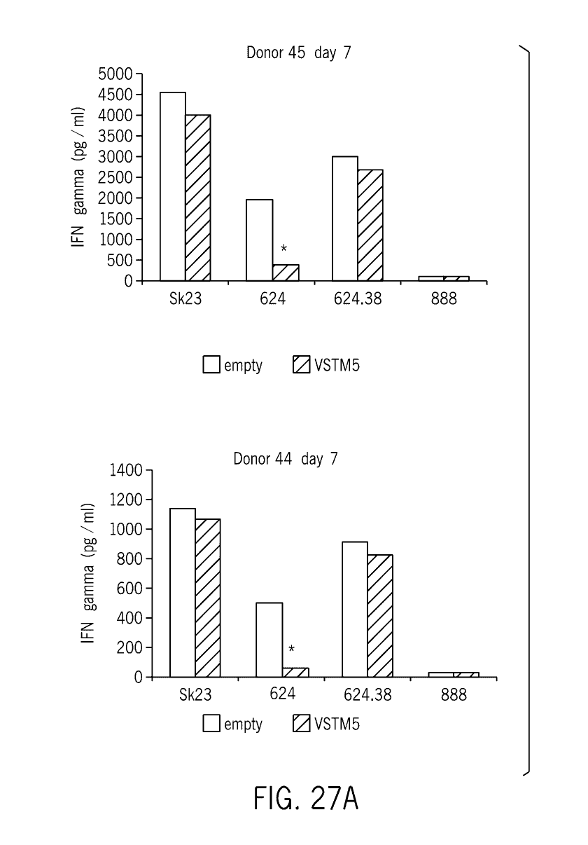

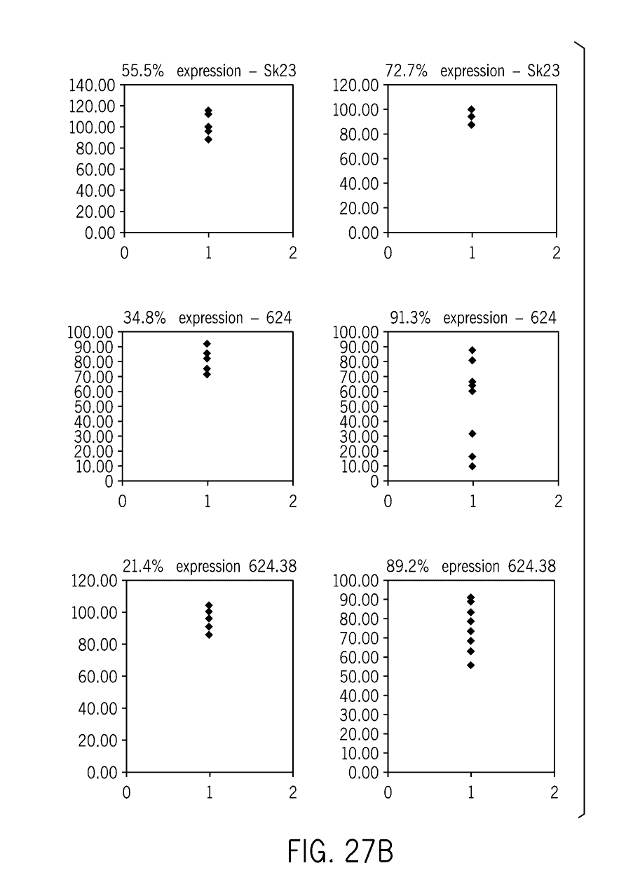

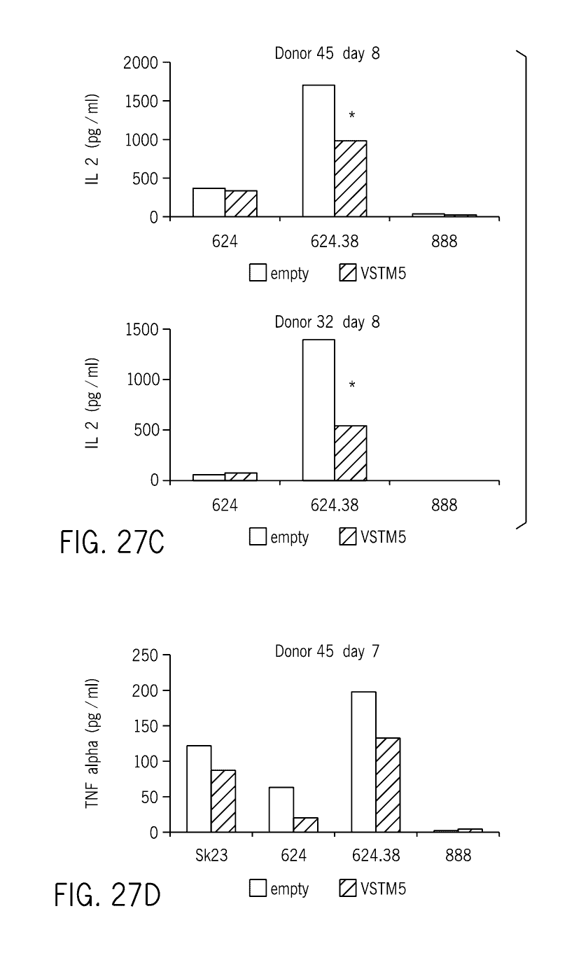

The F4 transduced effector lymphocytes were co-cultured with the melanoma lines expressing VSTM5 (SEQ ID NO:132) or empty vector. The levels of IFNγ, IL-2 and TNFα secretion were assessed at 16-hours of co-culture (FIGS. 27A-27D). In 8 independent experiments using 4 different T-cell donors, a significant reduction (˜30-90%) of IFNγ secretion was observed upon co-culture with VSTM5 expressing mel-624 cell line as compared to co-culture with the same cell line transduced with an empty vector (FIG. 27A and FIG. 27B). By contrast, VSTM5 expressed on SK-mel-23 cells did not appear to have an effect. VSTM5 expressed on mel-624.38 cells, led to a reduction in IFNγ secretion in several experiments (but not in others). As expected, no significant secretion of IFNγ was observed in the presence of mel-888 melanoma cells (which do not express HLA-A2 and thus are not recognized by the F4 TCR), indicating absence of activation of F4-expressing CTLs with these cells.

The secretion of IL-2 or TNFα was also tested in a few experiments. A significant reduction (˜40-60%) of IL-2 secretion from the CTLs upon co-culture with the VSTM5 expressing mel-624.38 cell line was observed, as compared to co-culture with this cell line transfected with empty vector (FIG. 27C). In addition a reduction in TNFα secretion was observed with all three melanoma cell lines expressing VSTM5 (FIG. 27D). However, only the VSTM5 expressing mel-624 cell line showed a statistically significant reduction compared to the empty vector cells. As expected, no significant secretion of IL-2 or TNFα was observed in the presence of mel-888 melanoma cells, indicating absence of activation of F4-expressing CTLs with these cells.

Over Expression of VSTM5 on Mel-624 Human Melanoma Cells Reduces their Susceptibility to Killing by F4 Transduced CTLs.

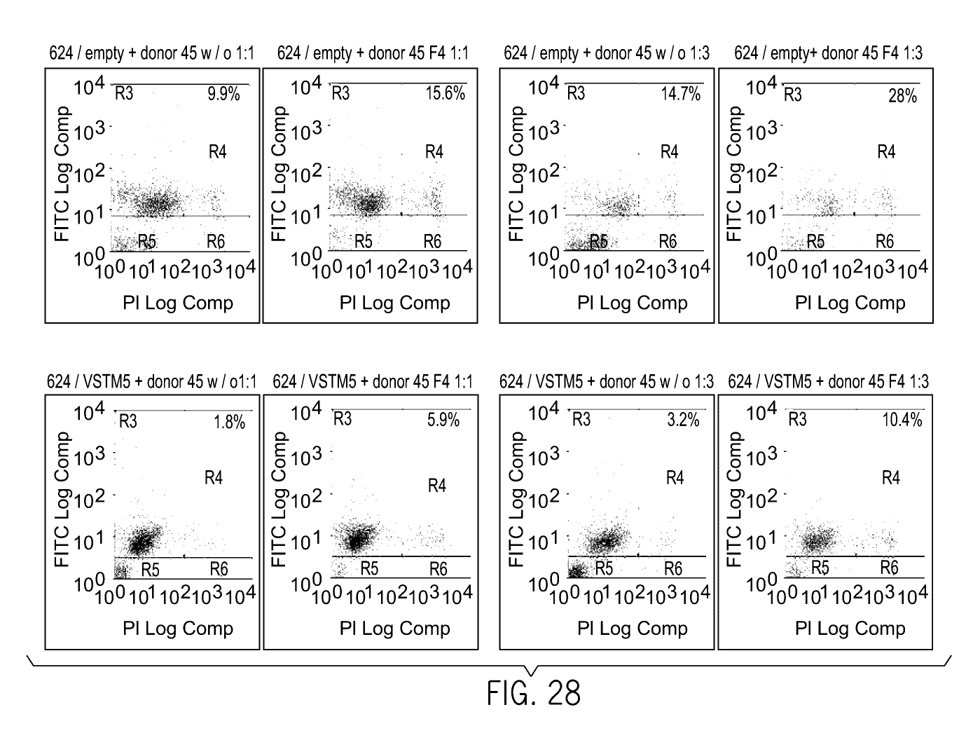

The effect of VSTM5 over expression on the susceptibility to cytotoxicity by effector CTLs was assessed by co-culture of the F4 TCR expressing lymphocytes with CFSE labeled melanoma cells as targets, following by PI staining. Percentage of double positive CFSE Pr cells point to the level of target cells killing. Results are shown in FIG. 28, indicating that over expression of VSTM5 on mel-624 as target cells results in a reduction of CTL killing activity.

### Review of Results

The results presented in this example indicate that VSTM5 overexpression on melanoma cells results in reduced cytokine secretion and killing activity by CTLs, indicating that VSTM5 has an inhibitory effect on CTLs. The observed results suggest that there is a variable effect on the cytotoxic activity of CTLs by VSTM5 on different melanoma cell lines. It is theorized that these differences potentially may be explained by different melanoma lines expressing different repertoires of endogenously expressed co-stimulatory/co-inhibitory proteins or different levels of expression thereof.

### Example 12

### Role of VSTM5 Proteins as Modulators of Cancer Immune Surveillance: In Vivo

### (i) Mouse Cancer Syngeneic Model:

Tumor cells, over expressing VSTM5 proteins or a non-relevant control protein are transplanted to genetically matched mice. Tumor volume (and tumor weight after sacrificing the animals) are then examined to demonstrate delay in the tumor growth (i.e. tumor over expressing VSTM5 grow faster than tumors over expressing the non-relevant control protein). Ex vivo analysis of immune cells from tumor draining lymph nodes is carried out to evaluate the effects of VSTM5 on the ratio of regulatory T cells and effector T cells.

(ii) Treatment of Syngeneic Tumor with Immunostimulatory VSTM5 Therapeutic Agent as Mono-Therapy

Tumor cells are transplanted to genetically identical mice. Tumor bearing mice are injected with different doses of immunostimulatory VSTM5 therapeutic agent. As a result of treatment with immunostimulatory VSTM5 therapeutic agent the rejection of the tumor is increased (i.e. in mice treated with immunostimulatory VSTM5 therapeutic agent tumors grow slower than tumors in mice treated with non-relevant agent). Ex vivo analysis of immune cells from tumor draining lymph nodes is carried out to determine of the ratio of regulatory T cells and effector T cells.

The tumor cells lines tested are from various origins including colon, breast, and ovary carcinomas, melanoma, sarcomas and hematological cancers. Syngeneic models are performed in several mouse strains including BALB/c, C57bl/6 and C3H/Hej. In the first set of experiments the syngeneic transplantable models used are primarily those reported to be predictive for cancer immunotherapy. These include: B16-F10 melanoma (according to the method described in Tihui Fu et al Cancer Res 2011; 71: 5445-5454), MC38 colon cancer (according to the method described in Ngiow S F et al. Cancer Res. 71(10):3540-51(2011)), ID8 ovarian cancer (according to the method described in Krempski et al. J Immunol 186:6905-6913)(2011), MCA105 sarcoma (according to the method described in Wang et al. J. Exp. Med., 208 (3): 577-592)(2011), CT26 colon carcinoma (according to the method described in Ngiow S F et al. Cancer Res. 71(10):3540-51(2011)) and 4T1 mammary carcinoma (according to the method described in Takeda K et al. J Immunol. 184(10):5493-501(2010)) of BALB/c background.

(iii) Establishment of a Syngeneic Tumor and Treatment with Immunostimulatory VSTM5 Therapeutic Agent in Combination with Additional Lines of Treatment.

In this model, tumor cells are transplanted to genetically identical mice. After the establishment of tumors, mice are injected IP with different doses of immunostimulatory VSTM5 therapeutic agent in combination with conventional chemotherapy (e.g. cyclophosphamide, according to the method described in Mkrtichyan et al. Eur. J Immunol. 41, 2977-2986(2011)), in combination with other immune checkpoint blockers (e.g. PD1 and CTLA4, according to the method described in Curran et al.; Proc Natl. Acad Sci USA. 107(9):4275-80(2010)), in combination with other immune-modulators (e.g. anti-IL-18, according to the method described in Terme et al., Cancer Res 71: 5393-5399(2011)), in combination with cancer vaccine (according to the method described in Hurwitz et al., Cancer Res 60:2444-2448(2000)) or in combination with radio-therapy (according to the method described in Verbrugge et al., Cancer Res. 72:3163-3174(2012)).

### (iv) Human Cancer Xenograft Model:

In this model human cancer cell lines, endogenously expressing VSTM5 are transplanted into immune-deficient mice. Tumor volume in mice treated with VSTM5 therapeutic agent are compared with mice treated with non-relevant agent will be assessed. In one arm of the study VSTM5 therapeutic agent are conjugated to a toxin (according to the method described in Luther N et al., Mol Cancer Ther. 9(4):1039-46(2010)). In another arm of the experiment, mice are treated with human IgG1 or mouse IgG2a isotype antibodies against VSTM5 (according to the method described in Holbrook E. Kohrt et al., J Clin Invest. 122(3): 1066-1075(2012)). These antibody isotypes are used to assess antibody-dependent cellular cytotoxicity (ADCC) mediated tumor elimination.

Expression Analysis

Expression of VSTM5 Proteins on Tumor and Immune Cells Isolated from Human Tumor Biopsies

### (i) VSTM5 Expression Validation

The expression of VSTM5 proteins is validated using specific antibodies directed against the VSTM5 proteins and is carried out on separated cell populations from tumors. Various cell populations are freshly isolated from tumor biopsies (e.g., Tumor cells, endothelia, tumor associated macrophages (TAMS) and DCs, B cells and different T cell sub-sets (CD4⁺, CD8⁺ T cells and Tregs) as described in Kryczek I. et al., J. Exp. Med., 203: 871-881 (2006) and Cancer Res. 67: 8900-8905 (2007), to demonstrate expression of VSTM5 in tumor cells and on tumor stroma and immune infiltrate.

### (ii) VSTM5 Binding Assays

Binding assays are performed using human VSTM5 ECD-FC proteins according to the invention and separated cell populations isolated from tumors. Various cell populations from tumor biopsies (e.g. tumor cells, endothelia, tumor associated macrophages (TAMS) and DCs, B cells and different T cells (CD4⁺, CD8⁺ T cells and Tregs) are freshly isolated from tumors as described in J. Exp. Med., 203: 871-881(2006) and Cancer Res. 67:8900-8905(2007), to show expression of the counter receptor for VSTM5 in tumor cells and on tumor stroma and immune cells.

It is anticipated (based on Example 1 results) that VSTM5 will be detected on many of these tumor types and stromal cells and lymphoid or immune cells associated therewith. Based thereon VSTM5 polypeptides and fusion proteins according to the invention should be able to inhibit the growth of tumors that show detectable VSTM5 levels of expression given the suppressive effect of VSTM5 on anti-tumor immunity Inhibiting VSTM5 on these cells will be beneficial in promoting anti-tumor immunity.

Expression of VSTM5 Proteins on Cells Isolated from Draining Lymph Nodes and Spleens of Tumor Bearing Mice

### (i) VSTM5 Expression Validation

The expression of VSTM5 proteins is validated using specific antibodies directed against VSTM5 proteins using epithelial cancer cells as well as on immune cells from tumor draining lymph nodes and is compared to spleen cells of tumor bearing C57 mice, as described in M Rocha et al., Clinical Cancer Research 2:811-820(1996). Three different cancer types are tested: B16 (melanoma), ID8 (ovarian) and MC38 (colon)), in order to evaluate expression of VSTM5 in tumor cells and in immune cells within the tumor draining lymph node.

### (ii) VSTM5 Binding Assays

Binding assays are conducted with mouse VSTM5 ECD-FC proteins and cells isolated from epithelial cancer as well as immune cells from tumor draining lymph nodes versus spleen of tumor bearing C57 mice. These binding assays are carried out as described above, in order to detect the expression of the counter receptor of VSTM5 on tumor cells and on immune cells comprised within in the tumor draining lymph node.

Again it is anticipated (based on Example 1 results) that VSTM5 will be detected on lymphoid cells associated with tumors. These results will further corroborate that VSTM5 polypeptides and fusion proteins according to the invention should be able to inhibit the tumors and prevent metastasis or the spread of tumors to new sites by inhibiting the activity of suppressive immune cells that have infiltrated the lymph nodes and which show detectable VSTM5 levels of expression. Particularly, inhibiting the activity of VSTM5 expressed on these immune cells will be beneficial in promoting anti-tumor immunity.

Expression of VSTM5 Proteins on M2 Polarized Macrophages

### (i) VSTM5 Expression Validation

The expression of VSTM5 proteins is validated using specific antibodies directed against VSTM5 proteins, is done on primary monocytes isolated from peripheral blood, differentiated into macrophages and exposed to “M2 driving stimuli” (e.g. IL-4, IL-10, Glucocorticoids, TGF 13), as described in Biswas S K, Nat. Immunol. 11: 889-896 (2010), to show expression of VSTM5 on M2 differentiated Macrophages.

### ii) VSTM5 Binding Assays

Binding assays are effected using VSTM5 human ECD-FC proteins and primary monocytes isolated from peripheral blood, differentiated into macrophages and exposed to “M2 driving stimuli” (e.g. IL-4, IL-10, glucocorticoids, TGFβ). These assays are carried out as described above in order to detect the expression of the counter receptor for VSTM5 by M2 differentiated Macrophages.

Detection of VSTM5 on these macrophage cells will further corroborate that VSTM5 polypeptides and fusion proteins according to the invention should be able to modulate (enhance or inhibit) the activity of these cells Inhibiting these cells, given their suppressive effects on antitumor immunity will be beneficial in treating cancer, infection or sepsis. Conversely, enhancing the activity of these cells will be advantageous when immunosuppression is desirable such as during autoimmunity, allergy, and inflammatory conditions or to reduce immune responses to transplanted cells, tissue or organ during gene therapy or transplantation, i.e., wherein immune tolerance is desired.

Expression of VSTM5 Proteins on Myeloid Derived Suppressor Cells (MDSCs)

(i) VSTM5 Expression Validation

The expression of VSTM5 proteins is again validated using specific antibodies directed against VSTM5 proteins, in this instance using primary MDSCs isolated from tumor bearing mice, as described in Int. Immunopharmacol. 9(7-8):937-48 (2009).

(ii) VSTM5 Binding Assays

VSTM5 binding assays are carried out with VSTM5 human ECD-Fc proteins as described in PCT/IB2012/051868, and primary MDSCs isolated from tumor bearing mice.

Results

It is anticipated that the expression of VSTM5 will be detected on these suppressor cells in view of VSTM5's enhancing effect on Tregs. Based thereon VSTM5 polypeptides and fusion proteins according to the invention should be able to modulate (enhance or inhibit) the activity of these suppressive cells Inhibiting these cells will be beneficial in treating cancer, infection or sepsis. Conversely, enhancing the activity of these cells will be advantageous where immunosuppression is desirable such as autoimmunity, allergy, and inflammatory conditions or to reduce immune responses to transplanted cells, tissue or organ during gene therapy or transplantation, i.e., therapeutic indications wherein immune tolerance or prolonged immunosuppression is desired.

### Example 13

### Anti-Tumor Effect of Immunostimulatory VSTM5 Therapeutic Agents in Combination with Known Immune Checkpoint Modulators

Inhibitory receptors on immune cells are pivotal regulators of immune escape in cancer. Among these are known immune checkpoints such as CTLA4, PD-1 and LAG-3. Blockade of a single immune checkpoint often leads to enhanced effector T cell infiltration of tumors, but may also lead to compensatory upregulation in these T cells of the other unblocked negative receptors. However, blockade or modulation of more than one inhibitory pathway allows T cells to carry out a more efficient tumor response, and increases the ratio of effector T cells (Teffs) to regulatory T cells (Tregs). Specifically, dual blockade of such inhibitory receptors has been shown to exert synergistic therapeutic effect in animal tumor models (Curran et al PNAS 107: 4275-4280 (2010); Woo et al Cancer Res. 72: 917-927(2011)). Based on these findings, the combination of anti-CTLA-4 and anti-PD-1 blocking antibodies is being tested in clinical trials in patients with metastatic melanoma.

The combination of blocking VSTM5 therapeutic agents and blocking PD-1 agents is tested in the syngeneic cancer MC38 model in the C57Bl/6 background (as described in Woo et al Cancer Res. 72: 917-927 (2011)). Briefly, MC38 cells (2×10⁶) are implanted s.c. C57Bl/6 mice. Mice with palpable tumors are injected i.p. at a dosage of 10 mg/kg anti-VSTM5 mAb and/or anti-PD-1 mAb (4H2). Isotype Control Ab is dosed at 20 mg/kg or added to individual anti-PD-1 or blocking VSTM5 therapeutic agent treatments at 10 mg/kg. Tumor volumes are measured with an electronic caliper, and the effect thereof on tumor growth is calculated. The therapeutic effect, manifested as inhibition of tumor growth, should be enhanced by the combination of the blocking agents against the two targets, PD-1 or VSTM5. The frequency of effector T cells=Teffs (CD8⁺IFNγ⁺) cells and the ratio of Teffs and Tregs are determined in tumor draining lymph nodes and non-draining lymph nodes.

It is anticipated that immunomodulatory VSTM5 fusion proteins and polypeptides according to the invention, based on their modulatory (enhancing or inhibitory) effect on T and NK mediated immunity will elicit at least additive effects and potentially synergistic effects when used in combination with other checkpoint regulatory polypeptides or small molecules such as PD-1 agonists and antagonists which modulate T or NK immunity.

### Example 14

### Anti-Tumor Effect of Immunostimulatory VSTM5 Therapeutic Agents in Combination with Metronomic Therapy with Cyclophosphamide

Cyclophosphamide has been used as a standard alkylating chemotherapeutic agent against certain solid tumors and lymphomas because of its direct cytotoxic effect and its inhibitory activity against actively dividing cells. While high doses of cyclophosphamide may lead to depletion of immune cells, low doses have been shown to enhance immune responses and induce anti-tumor immune-mediated effects, primarily by reducing the number and function of immunosuppressive Treg cells (Brode and Cooke Crit. Rev. Immunol. 28: 109-126(2008)). Metronomic therapy using classical chemotherapies other than cyclophosphamide has also been shown to have immunostimulatory effects, including gemcitabine; platinum based compounds such as oxaliplatin, cisplatin and carboplatin; anthracyclines such as doxorubicin; taxanes such as paclitaxel and docetaxel; and microtubule inhibitors such as vincristine.

Materials & Methods

Combination therapy of cyclophosphamide with other immunotherapies, such as anti-4-1BB activating Ab or anti-PD1 blocking Ab, resulted in synergistic anticancer effects (Kim et al. Mol Cancer Ther 8:469-478 (2009); Mkrtichyan et al. Eur. J. Immunol. 41:2977-2986(2011)). Based thereon, immunostimulatory VSTM5 therapeutic agents are tested in combination with cyclophosphamide in the syngeneic B16 melanoma model in the C57BL/6 background (as described in Kim et al. Mol Cancer Ther 8:469-478(2009)). Briefly, C57BL/6 mice are injected s.c. with 4×10⁵ B16-F10 melanoma cells. A single i.p. injection of cyclophosphamide (150 mg/kg) is administered on the day of tumor implantation, and five injections of 100 μg of the immunostimulatory VSTM5 therapeutic agent, 5 d apart beginning on the day of tumor implantation. To examine the antitumor effects of combination therapy on established tumors, the combination therapy is given beginning either at day 5 or day 10 after tumor cells injection. Tumor volumes are measured with an electronic caliper, and effect on tumor growth is calculated. The therapeutic effect, manifested as inhibition of tumor growth, is enhanced upon combination of cyclophosphamide with the blocking VSTM5 therapeutic agents. The frequency of effector T cells=Teffs (CD8⁺IFNγ⁺) cells and the ratio of Teffs and Tregs are determined in tumor draining lymph nodes and non-draining lymph nodes.

Results

It is anticipated that VSTM5 fusion proteins and polypeptides according to the invention, based on their modulatory effect on T and NK mediated immunity will elicit additive and possibly synergistic results when used to treat tumors when used in combination with chemotherapy by reducing the inhibitory effects of suppressor cells and promoting CTL and NK cytotoxicity and further by making the tumors more susceptible to the cytotoxic effects of chemotherapy agents such as cyclophosphamide

### Example 15

### Anti-Tumor Effect of Immunostimulatory VSTM5 Therapeutic Agents in Combination with Cellular Tumor Vaccines

Therapeutic cancer vaccines enable improved priming of T cells and improved antigen presentation as agents potentiating anti-tumor responses. Among these, are cellular tumor vaccines that use whole cells or cell lysates either as the source of antigens or as the platform in which to deliver the antigens. Dendritic cell (DC)-based vaccines focus on ex vivo antigen delivery to DCs. Other therapeutic cancer vaccines consist of tumor cells genetically modified to secrete immune stimulatory cytokines or growth factors, such as GM-CSF (granulocyte-macrophage colony-stimulating factor) or Flt3-ligand, aim to deliver tumor antigens in vivo in an immune stimulatory context to endogenous DCs.

Several in vivo studies have shown a potent therapeutic effect of immunecheckpoint blockade, such as anti-CTLA-4 antibodies, in poorly immunogenic tumors only when combined with GM-CSF or Flt3-ligand-transduced tumor vaccines, termed Gvax and Fvax, respectively (van Elsas et al J. Exp. Med. 190: 355-366(1999); and Curran and Allison Cancer Res. 69: 7747-7755(2009)), and that the antibody alone was effective only in the most immunogenic tumor models in mice. Furthermore, combination of two immunotherapeutic agents, such as anti-CTLA4 and anti-PD-1 blocking antibodies, is more effective in conjunction with therapeutic cancer vaccine, such as Gvax or Fvax (Curran et al PNAS 107: 4275-4280(2010))

Materials & Methods

The effect of an immunostimulatory VSTM5 fusion protein agent in combination with tumor cell vaccine, is tested using irradiated melanoma cells engineered to secrete GMCSF or Flt3-ligand (GVAX or FVAX respectively) in the presence or absence of anti-PD-1 blocking antibody (as described in Curran et al PNAS 107: 4275-4280(2010)). Briefly, mice are injected in the flank i. d. at day 0 with 5×104 B16-BL6 cells and treated on days 3, 6, and 9 with 106 irradiated (150 Gy) gene-modified B16 cells (expressing GMCSF or Flt3-ligand) in the contralateral flank in combination with intraperitoneal administration of 100 ug of immunostimulatory VSTM5 therapeutic agent, with or without 100 ug of anti-PD-1 blocking antibody (clone RMP1-14) or anti-PDL-1 blocking antibody (9G2). Isotype Ig is used as negative control. Tumor volumes are measured with an electronic caliper, and effect on tumor growth is calculated. It is predicted that the therapeutic effect, as manifested by the inhibition of tumor growth, is will be enhanced by use of the combination of the immunostimulatory VSTM5 therapeutic agent with the gene modified tumor cell vaccine. Anti-PD-1 or anti-PDL-1 blocking antibodies should further enhance this effect. The frequency of effector T cells=Teffs (CD8⁺IFNγ⁺) cells and the ratio of Teffs and Tregs are determined in tumor draining lymph nodes and non-draining lymph nodes.

Results

It is anticipated that VSTM5 fusion proteins and polypeptides according to the invention, based on their modulatory effect on T and NK mediated immunity and on Tregs and MSDCs will elicit additive and possibly synergistic results when used in combination with cancer vaccines by reducing the inhibitory effects of suppressor cells and/or by promoting CTL and NK cytotoxicity and memory responses, thereby allowing the treated subject to elicit a more potent immune response against cancer cells expressing the antigen contained in the cancer vaccine.

### Example 16

### Anti-Tumor Effect of Immunostimulatory VSTM5 Therapeutic Agents in Combination with Radiotherapy

Radiotherapy has long been used as anti-cancer therapy because of its powerful anti-proliferative and death-inducing capacities. However, recent preclinical and clinical data indicate that immunogenic cell death may also be an important consequence of ionizing radiation, and that localized radiotherapy can evoke and/or modulate anti-tumor immune responses (Reits et al. J. Exp. Med. 203:1259-1271(2006)). Preclinical studies have shown enhanced therapeutic effects in combined treatment of radiotherapy and immunotherapy, including blocking antibodies to immune checkpoints such as CTLA4 and PD-1, in the absence or presence of an additional immunotherapy such as activating anti-4-1BB Abs (Demaria et al Clin. Can. Res. 11:728-734 (2005); Verbruge et al Canc. Res. 72:3163-3174(2012)).

Materials & Methods

The combination of blocking VSTM5 therapeutic agent and radiotherapy will be assessed using a syngeneic 4T1 mammary carcinoma cell line in the BALB/c background (as described in Demaria et al. Clin. Can. Res. 11:728-734(2005)). Briefly, 5×10⁴ 4 T1 cells are injected s. c. in the flank of BALB/c mice. Treatment is commenced when tumors reach an average diameter of 5 mm (65 mm³ in volume). Animal groups include treatment with each modality alone (VSTM5 therapeutic agent or radiotherapy) and with the isotype Ig Control, and combination of VSTM5 therapeutic agent with radiotherapy, or of Ig Control with radiotherapy. Radiotherapy is delivered to the primary tumor by one or two fractions (48 hrs interval) of 12Gy. VSTM5 therapeutic agent or Ig control is given i.p. at 200 ug, on days 1, 4 and 7 after radiotherapy. In an additional set of experiments, blocking anti-PD-1 mAb (RMP1-14) and activating anti-4-1BB mAb (3E1) is administered. Tumor volumes are measured with an electronic caliper, and the effect on tumor growth is calculated. It is anticipated that the therapeutic effect, manifested by an inhibition of tumor growth, may be enhanced by the combined use of the blocking VSTM5 therapeutic agent with radiotherapy. Anti-PD-1 blocking antibodies or anti-4-1BB activating Abs, may further enhance this effect. The frequency of effector T cells=Teffs (CD8⁺IFNγ⁺) cells and the ratio of Teffs and Tregs are determined in tumor draining lymph nodes and non-draining lymph nodes.

Results

It is anticipated that VSTM5 fusion proteins and polypeptides according to the invention, based on their modulatory effect on T and NK mediated immunity will elicit additive and possibly synergistic results when used to treat cancer in combination with radiation by reducing the inhibitory effects of suppressor cells and promoting CTL and NK cytotoxicity thereby making the tumors more susceptible to the cytotoxic effects of the therapeutic radiotherapy.

### Example 17

### The Effect of VSTM5-Ig Fusion Protein on Th Differentiation

The effect of VSTM5-Ig fusion protein on Th differentiation using mouse or human CD4⁺ T cells upon activation under specific Th driving conditions is tested in different models.

Study I: Effect of VSTM5 mECD-mIg Fusion Protein on T Cell Differentiation Under Th0, Th1, Th2, and Th17 Driving Conditions In Vitro.

### Materials and Methods

### Reagents

VSTM5 mECD-mIg fusion protein (SEQ ID NO:8), composed of the ECD of mouse VSTM5 fused to the Fc of mouse IgG2a, was produced at ProBioGen (Germany) in CHO-DG44 cells by culturing stable cell pools for 12 days, followed by Protein A purification of cell harvest and preparative SEC purification for aggregate removal. Mouse IgG2a (clone: MOPC-173; Biolegend) was used as control Ig. Mouse CTLA4-Ig, a fusion protein composed of the ECD of mouse CTLA4 fused to the Fc of mouse IgG2a, was used as control and was expected to show no effect on T cell activation in APC-free assays, since it works primarily by antagonizing the interaction between CD28 on T cells and its ligands on APCs (Green et al Immunity 1:501-508 (1994); Wang et al, J. Clin Invest. 108:1771-1780(2001)).

Mouse CD4⁺ T cell isolation

Untouched CD4⁺CD25⁻ CD62L⁺ cells were isolated from pools of spleens of BALB/C mice by using a T cell isolation Kit (Miltenyi Cat#130-093-227) according to the manufacturer's instructions. The purity obtained was >95%.

### Activation of Mouse CD4 T Cells Under Th Promoting Conditions

Fresh mouse CD4⁺CD25⁻CD62L⁺ were stimulated for 5 days with immobilized anti-CD3 (clone 145-2C11, BD Bioscience; 2 μg/ml) in the presence of co-immobilized 10 μg/ml of control Ig, CTLA4-Ig or VSTM5 mECD-mIg fusion protein, and in the presence of soluble anti-CD28 (clone CD28.2, cat#16-0289-85, eBioscience; 1 μg/ml), under Th0, 1, 2, or Th17 driving conditions (as detailed in the table below). On day 5, culture supernatants were collected, and cells were re-stimulated with 50 ng/ml PMA (Sigma) and 1 ug/ml ionomycin (Sigma) for 5 h, in the presence of 2 μM monensin in the last 4 hr in order to block cytokine secretion. Cells were surface stained with APC-anti-CD4 (clone RM4-5; eBiosciences), in the presence of anti-CD16/32 (clone 2.4G2; BD Biosciences) for blocking of Fcγ-receptors. Cells were then fixed and permeabilized using BD Cytofix/Cytoperm kit (cat#5554715; BD Biosciences) according to the manufacturer's protocol, and analyzed for intra-cellular staining of PE-IFNγ (clone XMG1.2; Biolegend), IL-4 (clone 11B11; Biolegend) or IL-17 (clone TC11-18H10.1; Biolegend) using MACSQuant analyzer 9 (Miltenyi). FACS data was analyzed using MACSQuantify™ Software. Data was analyzed Prism4 software. The percentage and total cell numbers expressing each cytokine were assessed by flow cytometry. Culture supernatants were analyzed for IFNγ levels by ELISA (R&D Systems).

### Conditions for Differentiation to Different Th Subtypes:

| Driving | | Final | | Source and |
|---|---|---|---|---|
| Towards | reagent | Concentration | | cat# |
| Th0 | rIL-2 | 20 | ng/ml | R&D systems 402-ML-020) |
| Th1 | rIL-2 | 20 | ng/ml | R&D systems 402-ML-020) |
| | rIL-12 | 20 | ng/ml | Biolegend #505304 |
| | anti-IL-4 | 10 | μg/ml | Biolegend #574302 |
| Th2 | rIL-2 | 20 | ng/ml | R&D systems #402-ML-020 |
| | rIL-4 | 100 | ng/ml | R&D systems #574302 |
| | anti- | 10 | μg/ml | Biolegend #505706 |
| | IFNγ | | | |
| | anti-IL- | 10 | μg/ml | Biolegend #505304 |
| | 12 | | | |
| Th17 | rTGFβ | 1 | ng/ml | R&D systems #7666-MB-005 |
| | rIL-6 | 100 | ng/ml | Biolegend #575702 |
| | anti- | 10 | μg/ml | Biolegend #505706 |
| | IFNγ | | | |
| | anti-IL- | 10 | μg/ml | Biolegend #505304 |
| | 12 | | | |

### Flow Cytometry Analysis

Cells were evaluated using MACSQuant analyzer 9 (Miltenyi) and data analyzed using MACSQuantify™ Software. Data was analyzed using Excel or Prism4 software.

### Results

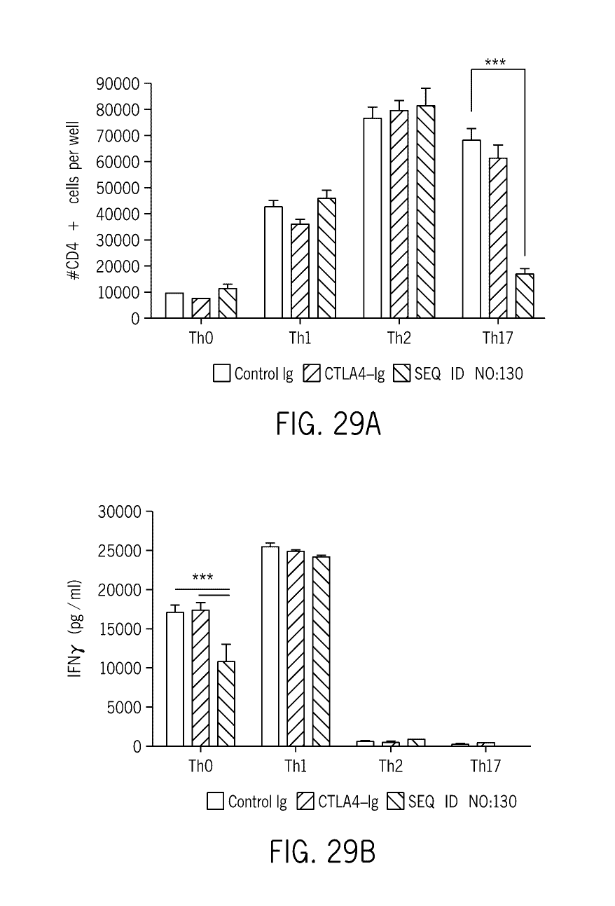

Under Th17-polarized condition, VSTM5 mECD-mIg fusion protein significantly reduced the number of total CD4⁺ cells as assessed by flow cytometry, whereas no effect on cell number under Th1 and Th2 differentiation conditions was observed (FIG. 29A). IFNγ secretion was observed under Th0 and Th1 driving conditions, as expected, while under Th2 and Th17 driving conditions the level of IFNγ secretion was low (FIG. 29B). IFNγ production was significantly reduced in the presence of VSTM5 mECD-mIg fusion protein under Th0 conditions whereas no effect under Th1-polarized condition was observed compared to control Ig or CTLA4Ig (FIG. 29B). This might be due to the specific conditions (i.e. addition of exogenous IL-12) used for the Th1 differentiation, which possibly preclude the detection of the inhibitory effect of VSTM5 mECD-mIg fusion protein. Refinement and calibration of the assay conditions may solve this issue.

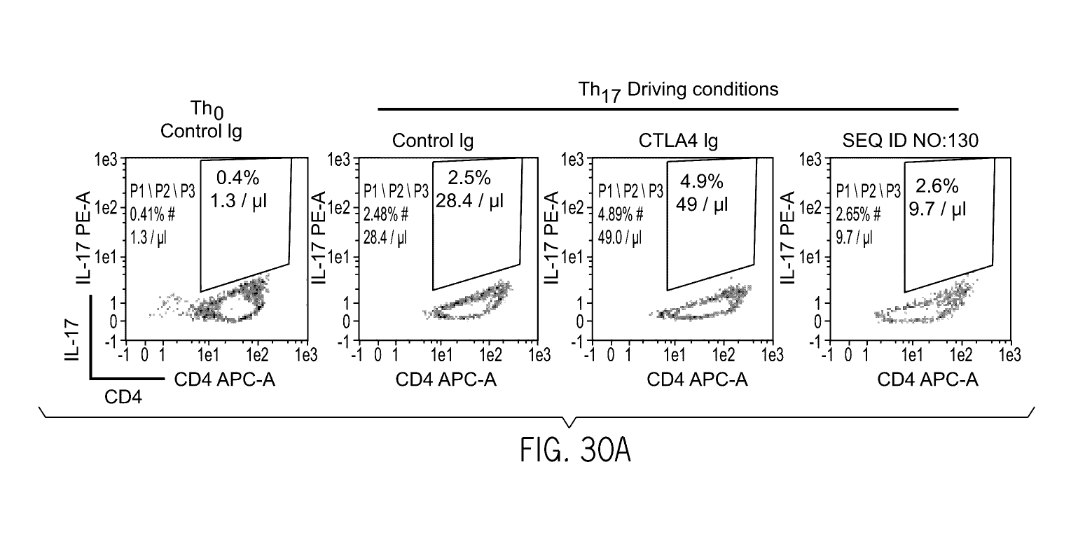

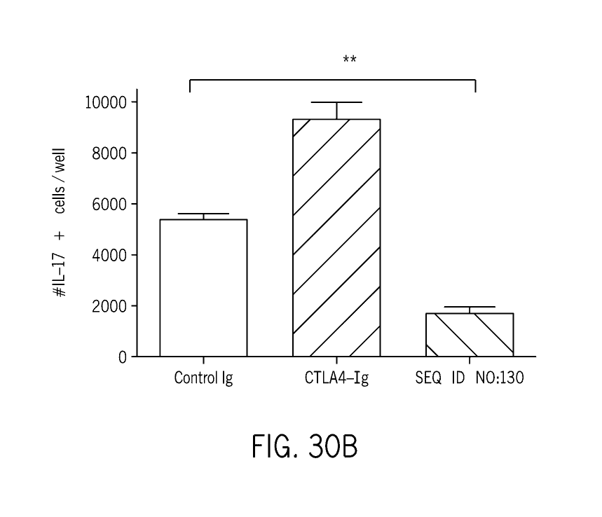

Intracellular staining for IL-17 under Th17 conditions showed ˜5 fold increase in the percent of CD4⁺IL17⁺ cells upon re-stimulation of the cells with PMA/ionophore, compared to Th0 (FIG. 30A). Under these conditions, VSTM5 mECD-mIg fusion protein significantly reduced the total number of CD4⁺IL17⁺ cells compared to the control Ig (FIG. 30B), although the percent of these cells was the same (FIG. 30A).

Intra-cellular staining for IL-4 was undetectable under Th2 driving condition (data not shown), this is not in correlation with the higher number of CD4⁺ cells obtained under Th2 driving conditions (shown in FIG. 29A), suggesting that perhaps a different readout of Th2 differentiation should be analyzed, such as secreted Th2 cytokines.

As expected, the effect of CTLA4-Ig in these experiments was comparable to the negative control Ig. However, under Th17-polarizing conditions it significantly increased the total number of CD4⁺IL17⁺ cells upon re-stimulation (FIG. 30B).

### Conclusion

VSTM5 mECD-mIg (SEQ ID NO:8) fusion protein inhibits Th17 differentiation. In addition, VSTM5 mECD-mIg fusion protein reduced the levels of secreted IFNγ under Th0 but not under Th1 driving conditions. This might be due to the specific conditions (i.e. addition of exogenous IL-12) used for the Th1 differentiation, which do not enable the detection of the inhibitory effect of VSTM5 mECD-mIg fusion protein. The effect of VSTM5 mECD-mIg fusion protein on Th differentiation may be further validated, by repeating these experiments, calibrating the experimental conditions and expanding to additional readouts.

### Study II—Effect of on Th1/Th17/Th2 Differentiation in Mouse Cells Following Antigen-Specific or Polyclonal Activation

In this study, murine T cell are activated either in an antigen-specific manner or by polyclonal activation. Without wishing to be limited by a single hypothesis, the results of these experimental settings, using mouse or human cells, point to an immunomodulatory effect of VSTM5 on T cells, whereby Th1 and Th17 driven responses (secretion of proinflammatory cytokines and cell proliferation under Th1 and Th17 driving conditions) are inhibited, while secretion of anti-inflammatory cytokines (Th2 derived, and IL-10) are promoted.

Materials & Methods

The ability of immunoinhibitory VSTM5 ECD Ig fusion proteins to inhibit CD4⁺ T cell differentiation, cytokine production and proliferation is tested using naïve CD4⁺ T cells isolated from D011.10 mice (transgenic mice in which all of the CD4⁺ T cells express a T cell receptor (TCR) that is specific for OVA323-339 peptide). The rationale for the use of D011.10 CD4⁺ T cells is that both polyclonal (anti-CD3/anti-CD28 mAbs) and peptide-specific CD4⁺ T cell activation may be studied on the same population of CD4⁺ T cells. Experimentally, naïve CD4⁺ cells are activated in the presence of Th0 cell- (IL-2), Th1 cell- (IL-2+IL-12), Th2 cell- (IL-2+IL-4), or Th17 cell- (TGF-β+IL-6+IL-23+anti-IL-2) promoting conditions.

To activate the CD4⁺ T cells, the cells are cultured in the presence of anti-CD3/anti-CD28 coated beads or OVA323-339 peptide plus irradiated BALB/c splenocytes in the presence of immunoinhibitory VSTM5 ECD Ig fusion proteins, Control Ig (negative control), or B7-H4 Ig (positive control). Two side-by-side cultures are set; one culture being pulsed at 24 hours with tritiated-thymidine and harvested at 72 hours while the second plate is harvested at 96 hours for cytokine production. The levels of IL-2, IL-4, IL-5, IL-10, IL-12, IL-17, IFN-γ, and TNF-α are tested via LiquiChip.

Results

Based on the results of the foregoing examples, and the demonstrated immunosuppressive effects of VSTM5, it is anticipated that VSTM5 ECD Ig fusion proteins, based on their agonistic effect on VSTM5, will be demonstrated to inhibit CD4+ T cell differentiation, cytokine production and proliferation.

### Study III: Effect of Immunoinhibitory VSTM5 ECD Ig Fusion Proteins on Th1/Th17/Th2 Differentiation in Human Cells

Materials & Methods

Naïve CD4⁺ T cells are isolated from 5 human donors. Naïve CD4+ cells are activated in the presence of Th0 cell- (IL-2), Th1 cell- (IL-2+IL-12), Th2 cell- (IL-2+IL-4), or Th17 cell- (TGF-β+ IL-1β+ IL-6+ IL-23+ IL-1β+ anti-IL-2) promoting conditions. To activate the CD4⁺ T cells, the cells are cultured in the presence of anti-CD3 mAb/anti-CD28 mAb coated beads in the presence of immunoinhibitory VSTM5 ECD Ig fusion proteins (SEQ ID NO:130) or hIgG1 isotype control. Two side-by-side cultures are set; one culture being pulsed at 24 hours with tritiated-thymidine and harvested at 72 hours while the second plate is harvested at 96 hours for cytokine production. IL-2, IL-4, IL-5, IL-10, IL-12, IL-17, IFN-γ, and TNF-α are tested using LiquiChip.

Results

It is known that one of the mechanisms by which tumors evade immune surveillance is promotion of a Th2/M2 oriented immune response (Biswas S K and Mantovani., Nat Immunol, 11(10):889-96(2010)). Thus, without wishing to be limited by a single hypothesis, a VSTM5 therapeutic agent which suppresses the above demonstrated immunomodulatory effect of VSTM5 (i.e, the promotion of Th2 response and inhibition of Th1 response) should be beneficial for treatment of cancer.

### Example 18

### Assessment of the Effect of VSTM5 Therapeutic Agents on Reversal of the Immunosuppression of Sepsis and Improvement of Survival in an Animal Model of Sepsis

In order to investigate the effect of VSTM5 therapeutic agent on sepsis in mice, the CLP (cecal ligation and puncture) model is used to induce polymicrobial peritonitis (as described by Brahmamdam et al J. Leukoc. Biol. 88:233-240(2010); Zhang et al Critical Care 14:R220(2010); Inoue et al Shock 36:38-44(2011)). CLP is carried out as follows: C57BL/6 mice are anesthetized and a midline abdominal incision is made. The cecum is mobilized, ligated below the ileocecal valve, and punctured twice with a needle. The abdominal wall is closed in two layers and mice are injected subcutaneously with 1 ml of saline within 30 min after surgery for volume resuscitation. Sham-operated mice which did not have their cecum ligated or punctured, serve as control. The VSTM5 therapeutic agent is administered intraperitoneally at different doses (ranging from 10 to 100 ug/mouse) 24 hrs before CLP (for preventive mode) or 1.5 hours after CLP surgery, followed by another injection at 24 hrs (for therapeutic mode). Isotype control or saline is used as negative controls. Survival is followed over the subsequent eight days. The effects of the immunostimulatory VSTM5 therapeutic agent is similarly evaluated on total splenocyte and blood lymphocyte counts, immune cell subtypes and cytokine production at various time points after surgery. The effect on sepsis-induced lymphocyte apoptosis is further evaluated. It is anticipated that treatment with an immunostimulatory VSTM5 therapeutic agent will elicit a beneficial effect on animal survival, and reduce lymphocyte apoptosis and the loss of viable immune cells.

Accordingly, it is anticipated that VSTM5 fusion proteins and polypeptides according to the invention, which inhibit the effects of VSTM5, will promote TH1 immunity and NK mediated cytotoxicity and reverse immunosuppression in sepsis models which should validate the potential use thereof in the treatment of infection or sepsis.

### Example 19

### Assessment of the Effect of VSTM5 Alone or in Combination with CTLA4-Ig or Anti-CD154 (CD40L) Antibody on the Enhanced Persistence of AAV-Mediated Gene Therapy

In order to investigate the effect of VSTM5 protein on AAV-mediated gene transfer, the rAAV-Ova model (as described by Adriouch et al Front. Microbiol. 2:199(2011)) is carried out as follows: C57BL/6 mice are injected with 1011 rAAV-Ova vector genomes in 50 ul PBS in the gastrocnemius muscles. Concomitantly, mice are injected i.p. with different doses of VSTM5 protein, without or with combination therapy with 200 ug CTLA4-Ig or with 200 ug anti-CD40L antibody (MR1). Alternatively, VSTM5 protein is administered via gene transfer with rAAV vectors. Blood samples are collected at day 14 and 40 to analyze the percentage of anti-Ova CD8⁺ T cells, the level of anti-Ova IgG and the presence of soluble Ova in the serum. Quantification of soluble Ova concentration in serum is performed by Ova-specific ELISA. Detection of serum anti-Ova IgG antibodies is performed by ELISA using Ova-coated microtiter plates and biotinylated anti-mouse Abs. CD8⁺ T cells that specifically recognize the Ova peptide are detected using PE-conjugated H-2Kb/Ova pentamers. Transduced gastrocnemius muscles are collected at day 40, and levels of Ova DNA and mRNA are quantified by qPCR and qRT-PCR.

It is anticipated that this experiment will validate that immunoinhibitory VSTM5 fusion proteins and polypeptides according to the invention will enhance the persistence of cell or gene therapy therapeutics, based on their inhibitory effect on T and NK mediated immunity and enhancement of suppressor cell activity. This should alleviate immune responses against gene therapy administered materials and promote the persistence of the administered gene therapeutic in an individual receiving gene or cell therapy thereby enhancing efficacy and safety of such cell or gene by preventing a potentially life-threatening rejection response.

### Example 20

### Characterizing Target Cells for VSTM5 Proteins by Determining their Binding Profile to Immune Cells

Splenocytes from D011.10 mice (transgenic mice in which all of the CD4⁺ T cells express a T cell receptor that is specific for OVA323-339 peptide) are activated in the presence of OVA323-339 peptide, and cells are collected at t=0, 6, 12, 24, 48, 72, 96 and 120 hours to determine which cell type is expressing a receptor for VSTM5 over time. In order to determine the cell types and activation stage in which the putative receptor for VSTM5 is expressed, VSTM5-Ig will be used for co-staining with lineage markers as either CD3, CD4, B220, CD19, CD11b, and CD11c and activation markers as CD137, CD44, CD25, PD1, and GITR.

Splenocytes from pmel-1 mice are activated in the presence of gp10025-33 human peptide (KVPRNQDWL) or gp10025-33 mouse peptide (EGSRNQDWL), and the cells are collected at t=0, 6, 12, 24, 48, 72, 96 and 120 hours following initial activation to determine which cell type is expressing a receptor for VSTM5 over time. Cells are then co-stained with VSTM5 ECD-Ig and immune cells markers such as for example CD3, CD4, CD8, NK1.1, B220, CD19, CD11b, CD11c, GR1, F4/80, CD14, CD40, and CD64 and markers of T cell activation such as for example CD137, CD44, CD25, PD1, and GITR.

Splenocytes from wild type mice such as for example C57Bl/6 or Balb/C are activated in a TCR-dependent manner such as for example by anti-CD3 with or without anti-CD28 Ab, or TCR-independent manner such as for example by PMA/Ionomycin, and the cells are collected at t=0, 6, 12, 24, 48, 72, 96 and 120 hours following initial activation to determine which cell type is expressing a receptor for VSTM5 over time. Cells are then co-stained with VSTM5-Ig and immune cells markers such as for example CD3, CD4, CD8, NK1.1, B220, CD19, CD11b, CD11c, GR1, F4/80, CD14, CD40, and CD64 and markers of T cell activation such as for example CD137, CD44, CD25, PD1, and GITR.

Whole bone marrow cells are obtained from wild type mice such as for example c57BL/6 mice and plated in RPMI supplemented with 10% FCS, antibiotics and differentiation factors such as for example DC differentiation factor GM-CSF (10 ng/ml). The cells are either left non activated (NA) or activated with increasing concentrations of such as for example LPS, CpG and IL-4 for 12, 24 and 72 hrs. Thereafter, the cells are pretreated with Fc-blocking reagent, stained with VSTM5 ECD-Ig or by a relevant isotype control.

### Example 21

### Assessment of the Effect of VSTM5-ECD-Ig Fusion Proteins on the Ability of B Cells to Class-Switch and Secrete Antibody

Resting B cells are isolated from unprimed C57BL/6 mice and activated in vitro in the presence of anti-CD40 plus (i) no exogenous cytokine, (ii) IL-4, or (iii) IFN-γ. The cell cultures receive control Ig (mIgG2a), anti-CD86 mAb (as a positive control for increased Ig production), or any one of VSTM5 ECD fusion proteins, at the time of culture set up, and are cultured for 5 days. The VSTM5 ECD fusion proteins are tested at three concentrations each. At the end of culture, supernatants are tested for the presence of IgM, IgG1, IgG2a, IgG2b, IgG2c, IgG3, IgA, and IgE via ELISA. If there appears to be an alteration in the ability of the B cells to class-switch to one isotype of antibody versus another, then the number of B cells that have class switched is determined via ELISPOT. If there is an alteration in the number of antibody producing cells, then it is determined if there is an alteration in the level of γ1-, γ2a-α-, δ-, ε-, γ-, and μ--sterile transcripts versus the mature transcripts for IgG1 and IgG2a.

### Example 22

### Efficacy of Immunoinhibitory VSTM5 Proteins in Mouse R-EAE Model of Multiple Sclerosis

Study I: Effect of VSTM5 ECD Ig Fusion Proteins in Active R-EAE Model

The therapeutic effect of immunoinhibitory VSTM5 ECD Ig fusion proteins for the treatment of autoimmune diseases is tested in a mouse model of Multiple Sclerosis; Relapsing Remitting Experimental Autoimmune Encephalomyelitis (R-EAE): Female SJL mice 6 weeks old are purchased from Harlan and maintained in the CCM facility for 1 week prior to beginning the experiment. Mice are randomly assigned into groups of 10 animals and primed with 50 μg PLP139-151/CFA on day 0. Mice receive 6 i. p. injections of 100 ug/dose of immunoinhibitory VSTM5 ECD Ig fusion protein, mIgG2a isotype control, or CTLA4-Ig (mouse ECD fused to mouse IgG2a Fc) as positive control. Treatments begin at the time of disease induction (preventive mode) or at onset of disease remission (therapeutic mode) and are given 3 times per week for at least 2 weeks. Mice are scored for disease symptoms on a 0-5 disease score scale: 0, no abnormality; 1, limp tail; 2, limp tail and hind limb weakness; 3, hind limb paralysis; 4, hind limb paralysis and forelimb weakness; and 5, moribund.

It is anticipated that immunoinhibitory VSTM5 fusion proteins and polypeptides according to the invention, based on their inhibitory effect on T and NK mediated immunity and enhancing effect on suppressor cells, will reduce the onset and/or the pathology associated with EAE. As EAE is an accepted model of multiple sclerosis this will further validate the potential use of immunoinhibitory VSTM5 fusion proteins according to the invention to treat multiple sclerosis and other autoimmune conditions.

Study II: Effect of VSTM5 ECD Ig Fusion Proteins in Adoptive Transfer R-EAE Model

Donor and recipient female SJL mice (6 weeks old) are purchased from Harlan and maintained in the CCM facility for 1 week prior to beginning the experiment. Donor mice are primed with PLP139-151/CFA on Day 0. Draining lymph nodes from donor mice are harvested on day 8 post priming, and total lymph node cells are activated ex vivo in the presence of PLP139-151 for 3 days. After culture, cells are stained with PBSE and 10 recipient mice per group receive 5×106 blast cells via i. v. injection.

Mice are scored for disease symptoms on a 0-5 disease score scale: 0, no abnormality; 1, limp tail; 2, limp tail and hind limb weakness; 3, hind limb paralysis; 4, hind limb paralysis and forelimb weakness; and 5, moribund.

At the time of cell transfer or at time of onset of remission, mice are administered i.p. with either Control Ig, immunoinhibitory VSTM5 ECD Ig fusion protein or Enbrel (positive control) (100 ug/dose, 3 times per week for two weeks, each). On day 14, five mice from each treatment group are scarified and the rest are followed for clinical score until day 30. Spleens, LN and CNS are analyzed for difference in total cell counts and trafficking of transferred cells (PBSE⁺).

### Example 23

### Efficacy of Immunoinhibitory VSTM5 Proteins in Mouse CIA Models of Rheumatoid Arthritis

Study I: Therapeutic Effect of VSTM5 ECD Ig Fusion Proteins in CIA Model without Boost

Immunoinhibitory VSTM5 ECD Ig fusion proteins are tested in mouse model of collagen-induced arthritis (CIA) which is a model of rheumatoid arthritis. Male DBA/1 mice are housed in groups of 8-10, and maintained at 21° C.±2° C. on a 12 h light/dark cycle with food and water ad libitum. Arthritis is induced by immunization with type II collagen emulsified in complete Freund's adjuvant. Mice are monitored on a daily basis for signs of arthritis. On the appearance of arthritis (day 1) treatment with immunoinhibitory VSTM5 ECD Ig fusion proteins, mIgG2a isotype control or CTLA4-Ig (mouse ECD fused to mouse IgG2a Fc) as positive control (100 ug/dose, each) is initiated and given 3 times per week for 10 days. Hind footpad swelling is measured (using microcalipers), as well as the number and degree of joint involvement in all four limbs. This yields two measurements, clinical score and footpad thickness that can be used for statistical assessment.

At the end of the treatment period mice are bled and sacrificed. For histological analysis, paws are removed at post mortem, fixed in buffered formalin (10% v/v), then decalcified in EDTA in buffered formalin (5.5% w/v). The tissues are then embedded in paraffin, sectioned and stained with haematoxylin and eosin. The scoring system is as follows: 0=normal; 1=synovitis but cartilage loss and bone erosions absent or limited to discrete foci; 2=synovitis and significant erosions present but normal joint architecture intact; 3=synovitis, extensive erosions, and joint architecture disrupted.

The ability of the treatment of mice with established CIA with immunoinhibitory VSTM5 ECD Ig fusion proteins to result in potent reduction of clinical score, paw swelling and histological damage is tested and compared to the efficacy obtained with CTLA4-Ig.

It is anticipated that immunoinhibitory VSTM5 fusion proteins and polypeptides according to the invention, based on their inhibitory effect on T and NK mediated immunity and enhancing effect on suppressor cells will reduce the onset and/or the pathology associated with collagen induced arthritis (CIA) such as tissue destruction. As CIA is an accepted rheumatoid arthritis animal model this will validate the potential use of immunoinhibitory VSTM5 fusion proteins according to the invention to treat rheumatoid arthritis and potentially other autoimmune conditions.

Study II: Effect of Semi Therapeutic Treatment with VSTM5 ECD Ig Fusion Proteins in CIA Model with Boost

Male DBA/lOlaHsd Mice (Harlan, 12-13 weeks old) are anesthetized with Isoflurane. CIA is induced by two injections of 150 μl of Bovine Type II collagen (Research Inc) in Freund's complete adjuvant (Difco) given on days 0 and 21.

Mice are treated either in a prophylactic, semi-therapeutic or therapeutic manner. For prophylactic treatment, treatment is given starting from day 0. For semi-therapeutic treatment, treatment is given from day 18 excluding animals that develop disease before beginning of treatment. For therapeutic treatment, mice were enrolled into treatment groups on the day of onset for each mouse individually (therapeutic treatment) starting the treatment the same day. Mice are treated IP, Q2D with VSTM5 ECD Ig fusion proteins, mIgG2a isotype control or Enbrel (positive control) starting from day 18 until day 34. During this time clinical scores are given for each of the paws (right front, left front, right rear, left rear) using the following criteria: 0=normal; 1=1 hind or fore paw joint affected or minimal diffuse erythema and swelling; 2=2 hind or fore paw joints affected or mild diffuse erythema and swelling; 3=3 hind or fore paw joints affected or moderate diffuse erythema and swelling; 4=Marked diffuse erythema and swelling, or =4 digit joints affected); 5=Severe diffuse erythema and severe swelling entire paw, and unable to flex digits). Animals are necropsied on study day 34 and joints are collected for histological analysis by H&E stain.

It is anticipated that the results win this CIA model will further corroborate the potential use of immunoinhibitory VSTM5 fusion proteins according to the invention to treat rheumatoid arthritis and potentially other autoimmune conditions.

### Example 24

### Determine Long Term Efficacy of Immunoinhibitory VSTM5 Proteins in Chronic CIA Model

C57BL/6 mice are treated from onset of disease with immunoinhibitory VSTM5 ECD IG fusion proteins, control IgG2a or Enbrel with 3 doses as in previous studies, in groups of 8-10 mice. At day 10, no further treatment is given and the mice are continuously monitored for 20-30 days in order to establish the time taken for the disease to flare again. This assesses the efficacy of immunoinhibitory VSTM5 ECD IG fusion proteins in the chronic CIA model and the duration of its biological effect in rheumatoid arthritis. Long term efficacy is observed in this model. Without being bound by a single hypothesis, a decrease in disease severity is accompanied by decrease in anti-collagen antibody levels as measured for example by ELISA.

It is anticipated that immunoinhibitory VSTM5 fusion proteins and polypeptides according to the invention, based on their inhibitory effect on T and NK mediated immunity and especially their enhancing effect on suppressor cells should result in prolonged inhibition of autoimmunity and inflammation in this chronic collagen induced arthritis (CIA) model. Promoting suppressor cells and inhibiting CTL and NK cytotoxicity and proinflammatory cytokines should promote a prolonged reduction in the disease pathology by inhibiting pathological cytotoxic T and NK cells. As chronic CIA is an accepted rheumatoid arthritis animal model these results will further validate the potential use of immunoinhibitory VSTM5 fusion proteins according to the invention to treat rheumatoid arthritis and potentially other autoimmune conditions,

### Example 25

### Effect on Tolerance Induction by Immunoinhibitory VSTM5 Proteins in Transfer Model of CIA

To further validate the effect of immunoinhibitory VSTM5 ECD IG fusion proteins on immune regulation, the ability of immunoinhibitory VSTM5 ECD IG fusion proteins to induce tolerance in a transfer model of arthritis is analyzed.

In brief, spleen and LN cells from arthritic DBA/1 mice treated for 10 days with immunoinhibitory VSTM5 ECD IG fusion proteins or control Ig2a are removed and injected i. p into T-cell deficient C. B-17 SCID recipients. The mice then receive an injection of 100 μg type II collagen (without CFA), necessary for successful transfer of arthritis. Arthritis is then monitored in the SCID mice; it is determined that the VSTM5 ECD IG fusion proteins treatment confers long-term disease protection. Histology is performed and anti-collagen antibody levels are measured to support this determination.

Again it is anticipated that immunoinhibitory VSTM5 fusion proteins and polypeptides according to the invention, based on their inhibitory effect on T and NK mediated immunity and their enhancing effect on suppressor cells potentially may induce tolerance in this CIA model. Specifically, promoting suppressor cells should promote immune suppression and potentially elicit prolonged immune suppression or tolerance in this CIA model. If these results are observed, as this transfer CIA model is an accepted rheumatoid arthritis animal model, this will validate the potential use of immunoinhibitory VSTM5 fusion proteins according to the invention to treat rheumatoid arthritis and potentially its potential to result in disease remission.

### Example 26

### Assessment of the Effect of VSTM5 ECD-Ig Fusion Proteins in a Viral Infection Model of TMEV

Theiler's murine encephalomyelitis virus (TMEV) is a natural endemic pathogen of mice that causes an induced demyelinating disease (TMEV-IDD) in susceptible strains of mice (SJL/J, H-2KS) that resembles the primary progressive form of MS (Munz et al., Nat Rev Immunol 9:246-58(2009)). TMEV infection results in a life-long persistent virus infection of the CNS leading to development of a chronic T cell-mediated autoimmune demyelinating disease triggered via de novo activation of CD4⁺ T cell responses to endogenous myelin epitopes in the inflamed CNS (i.e., epitope spreading) (Miller et al., Nat Med 3:1133-6(1997); Katz-Levy et al., J Clin Invest 104:599-610(1999)).

SJL mice clear the majority of the virus within 21 days post infection, however a latent viral infection is maintained and infect microglia, astrocytes, and neurons. Disease symptoms are manifested around day 25-30 post infection. The effect of treatment with VSTM5 ECD-Ig fusion proteins on acute and chronic phases of viral infection is studied in the TMEV-IDD model by assessment of viral clearance and disease severity.

Method:

Female SJL/J mice (5-6 weeks) are infected with TMEV by intracranial inoculation in the right cerebral hemisphere of 3×10⁷ plaque forming units (PFU) of the BeAn strain 8386 of TMEV in 30 ul serum-free medium. From day 2 post infection mice are treated with Control Ig, VSTM5 ECD-Ig fusion proteins, at 100 ug/dose each; 3 doses/week for 2 weeks.

Mice are followed for clinical scoring. On day 7 and day 14 post infection (after 3 and 6 treatments respectively) brains and spinal cords are collected from 5 mice in each treatment group for plaque assays. The tissues are weighted so that the ratio of PFU/mg of CNS tissue could be calculated after the plaque assay is completed.

TMEV Plaque Assay:

Brains and spinal cords of mice treated with Control Ig (mouse IgG2a), or with each of VSTM5 ECD-Ig fusion proteins are collected at days 7 and 14 post-infection from non-perfused anesthetized mice. The Brains and spinal cords are weighed, and homogenized. CNS homogenates are serially diluted in DMEM and added to tissue culture-treated plates of confluent BHK-21 cells for 1 h incubation at room temperature, with periodic gentle rocking.

A media/agar solution is mixed 1:1 (volume:volume), added to cells and allowed to solidify at room temperature. The plates are then cultured at 34° C. for 5 days. At the end of culture, 1 ml of formalin is added and incubated at room temperature for 1 h to fix the BHK monolayer. The formalin is poured off into a waste container, and the agar is removed from the plates. Plaques are visualized by staining with crystal violet for 5 min, and plates are gently rinsed with diH2O. To determine PFU/ml homogenate, the number of plaques on each plate is multiplied by the dilution factor of the homogenate and divided by the amount of homogenate added per plate. The PFU/ml is divided by the weight of the tissue to calculate PFU/mg tissue.

It is anticipated that immunoinhibitory VSTM5 fusion proteins and polypeptides according to the invention, based on their inhibitory effect on T and NK mediated immunity and their enhancing effect on suppressor cells potentially will inhibit autoimmunity in this viral model of MS. Promoting suppressor cells should promote immune suppression and potentially elicit prolonged immune suppression or possibly tolerance. If these results are seen as this transfer model is an accepted MS animal model this will validate the potential use of immunoinhibitory VSTM5 fusion proteins according to the invention to treat MS by preventing or alleviating the effects of cytotoxic T cells and autoimmune associated tissue destruction and inflammation.

### Example 27

### Assessment of the Effect of VSTM5 ECD-Ig Fusion Proteins on Primary and Secondary Immune Response to Viral Infection in a Mouse Model of Influenza

To test the effect of VSTM5 ECD-Ig fusion proteins on primary and secondary immune responses to viral infection, BALB/c naïve mice (for primary immune responses) and ‘HA-memory mice’, is used, as well as ‘polyclonal flu-memory mice’ (to assess secondary responses mediated by memory CD4⁺ T cells), which are generated as detailed in Teijaro et al., J Immunol. 182; 5430-5438(2009), and described below.

To obtain ‘HA-memory mice’, first HA-specific memory CD4 T cells are generated, naive CD4 T cells are purified from spleens of HA-TCR mice WALB/c-HA mice which express transgenic T cell receptor (TCR) specific for influenza hemagglutinin (HA) peptide (110-119)] and primed in vitro by culture with 5.0 μg/ml HA peptide and mitomycin C-treated, T-depleted BALB/c splenocytes as APCs for 3 days at 37° C. The resultant activated HA-specific effector cells are transferred into congenic BALB/c (Thy1.1) hosts (5×10⁶ cells/mouse) to yield “HA-memory mice” with a stable population of HA-specific memory CD4⁺ T cells.

To obtain ‘polyclonal-memory mice’, first polyclonal influenza-specific memory CD4⁺ T cells are generated, by infecting BALB/c mice intranasally with a sublethal dose of PR8 influenza, CD4⁺ T cells are isolated 2-4 months postinfection, and the frequency of influenza-specific memory CD4⁺ T cells is determined by ELISPOT. CD4⁺ T cells from previously primed mice are transferred into BALB/c hosts to generate “polyclonal flu-memory” mice with a full complement of endogenous T cells.

Primary and secondary responses to influenza virus are tested by infecting naïve BALB/c mice or BALB/c-HA memory mice and BALB/c ‘polyclonal flu-memory mice’ with sublethal or lethal doses of PR8 influenza virus by intranasal administration.

Mice are treated with VSTM5 ECD-Ig fusion proteins or with mIgG2a control before and following influenza challenge. Weight loss and mortality will be monitored daily. Six days after the challenge, viral content in the bronchoalveolar lavage (BAL) is analyzed by collecting lavage liquid and testing the supernatant for viral content by determining the tissue culture infectious dose 50% (TCID50) in MDCK cells. In addition, lung tissue histopathology is performed.

To test the effect VSTM5 ECD-Ig fusion proteins on T cell expansion BALB/c or BALB/c-HA memory mice or BALB/c ‘polyclonal flu-memory mice’ are infected as above and administered with BrdU (1 mg/dose) on days 3, 4 and 5 post infection. On day 6, spleen and lung are harvested and BrdU incorporation is estimated. Cytokine production by lung memory CD4⁺ T cells during influenza challenge is also studied in HA-specific memory CD4⁺ T cells stimulated in vitro with HA peptide in the presence VSTM5 ECD-Ig fusion proteins or with IgG2a for 18 hours.

It is anticipated that immunostimulatory VSTM5 fusion proteins and polypeptides according to the invention, which inhibit VSTM5 and thereby elicit an enhancing effect on T and NK mediated immunity, and memory responses, and an inhibitory effect on suppressor cells potentially will promote antiviral immunity in this model by inducing CD4⁺ T cell mediated depletion of virally infected cells. If these results are seen this will validate the potential use of immunostimulatory VSTM5 fusion proteins according to the invention to treat infection or sepsis and particularly influenza.

### Example 28

### Assessment of the Effect of VSTM5 ECD-Ig Fusion Proteins on Primary and Secondary CD8⁺ T Cell Response to Viral Infection in a Mouse Model of Influenza

The effect of VSTM5 ECD Ig fusion proteins on primary CD8⁺ T cell responses to influenza virus is studied according to methods as described in the literature (Hendriks et al., J Immunol 175:1665-1676(2005); Bertram et al., J Immunol. 172:981-8(2004)) using C57BL/6 mice infected with influenza A HK×31 by intranasal or intraperitoneal administration. VSTM5 ECD Ig fusion proteins or mIgG2a control are administered during priming. Animal weight loss and mortality is monitored daily. To follow virus-specific CD8⁺ T cells, MHC H-2Db tetramers loaded with the major CD8 T cell epitope, the NP366-374 peptide are used. Virus-specific H-2Db/NP366-374⁺ CD8⁺ T cells in the lung, draining lymph nodes, and spleen are expected to reach a peak around day 8-10 post infection and decline thereafter to only 1.5% virus-specific CD8 T cells (Hendriks et al J Immunol 175:1665-1676 (2005); Bertram et al. J Immunol. 168:3777-85(2002); Bertram et al., J Immunol. 172:981-8(2004). Thus, mice are sacrificed at days 8 and 21 post infection, and virus-specific CD8 T cell numbers is evaluated in the lung, draining lymph nodes and spleen. Viral clearance is assessed. CD8⁺ T cell responses are evaluated in spleen cell suspensions, and include intracellular IFN-γ staining and CTL activity, as previously described (Bertram et al., J Immunol. 172:981-8(2004)) and detailed below.

Cells are surface-stained with FITC-conjugated anti-mouse CD62L, PE-conjugated anti-mouse CD8 to measure CD8⁺ activated T cells (or anti-mouse CD4 to follow CD4⁺ cells). In addition to these Abs, allophycocyanin-labeled tetramers consisting of murine class I MHC molecule H-2Db, β2-microglobulin, and influenza NP peptide, NP366-374 are used to measure influenza-specific CD8 T cells. For intracellular IFN-γ staining, cell suspensions are restimulated in culture medium for 6 h at 37° C. with 1 μM NP366-374 peptide and GolgiStop® (BD PharMingen, San Diego, Calif.). Cells are then harvested, resuspended in PBS/2% FCS/azide, and surface stained with PE-anti-CD8 and FITC-anti-CD62L as described above. After surface staining, cells will be fixed in Cytofix/Cytoperm solution (BD PharMingen) and then stained with allophycocyanin-conjugated antimouse IFN-γ diluted in 1× perm/wash solution (BD PharMingen). Samples are analyzed by Flow Cytometry.

For cytotoxicity assays (CTL responses) splenocytes from influenza-infected mice are incubated for 2 h at 37° C. to remove adherent cells. Serial 3-fold dilutions of effectors are assayed for anti-influenza NP366-374-specific CTL activity against ⁵¹Cr-labeled EL4 cells pulsed with 50 μM NP366-374 peptide for 6 h as described by Bertram et al. J Immunol. 168:3777-85 (2002) and Bertram et al J Immunol. 172:981-8 (2004).

At 3 weeks postinfection, some mice are rechallenged with the serologically distinct influenza A/PR8/34 (PR8), which shares the NP gene with influenza A HK×31, but differs in hemagglutinin and neuraminidase, so that neutralizing Abs do not limit the secondary CTL response. Mice are sacrificed at days 5 & 7 following virus rechallenge, and virus-specific CD8 T cell numbers is evaluated in the lung, draining lymph nodes and spleen as described by Hendriks et al and Bertram et al (Hendriks et al., J Immunol 175; 1665-1676(2005); Bertram et al., J Immunol. 172:981-8(2004)) and detailed above. Secondary CD8⁺ T cell responses, including intracellular IFN-γ staining and CTL activity, are evaluated in spleen cell suspensions of mice at days 5 & 7 following virus rechallenge, as described above.

To determine the effect of VSTM5 ECD-Ig fusion proteins on expansion and accumulation of memory CD8⁺ T cells during the secondary response, adoptive transfer experiments are performed, according to methods previously described (Hendriks et al., J Immunol 175; 1665-1676(2005); Bertram et al., J Immunol. 172:981-8(2004)): mice are immunized with influenza A HK×31. Twenty-one days later, T cells are purified from spleens on mouse T cell enrichment immunocolumns (Cedarlane Laboratories, Hornsby, Ontario, Canada) and labeled with CFSE (alternatively Thy1.1 congenic mice are used as recipients). Equal numbers of tetramer-positive T cells are injected through the tail vein of recipient mice. Mice are rechallenged with influenza virus as described above, and 7 days later splenocytes are evaluated for donor virus-specific CD8⁺ T cells, as detailed above.

It is again anticipated that immunostimulatory VSTM5 fusion proteins and polypeptides according to the invention, which inhibit VSTM5 and thereby elicit an enhancing effect on T and NK mediated immunity, and memory CD8 immune responses, and elicit an inhibitory effect on suppressor cells potentially will promote antiviral protective immunity in this influenza model by inducing memory immune responses against the virus and CD8⁺ T cell mediated depletion of the virus or virally infected cells upon rechallenge in this model. Such results will further validate the potential use of immunostimulatory VSTM5 fusion proteins according to the invention to treat viral infection or sepsis and particularly influenza.

### Example 29

### Assessment of Protein Expression in Exhausted T Cells, and on the Binding and Effect of VSTM5 ECD-Ig Fusion Proteins on Reversing Exhausted T Cell Phenotype

Memory CD8⁺ T-cell differentiation proceeds along distinct pathways after an acute versus a chronic viral infection (Klenerman and Hill Nat Immunol 6, 873-879, (2005)). Memory CD8⁺ T cells generated after an acute viral infection are highly functional and constitute an important component of protective immunity. In contrast, chronic infections are often characterized by varying degrees of functional impairment of virus-specific T-cell responses, and this defect is a principal reason for the inability of the host to eliminate the persisting pathogen. Although functional effector T cells are initially generated during the early stages of infection, they gradually lose function during the course of the chronic infection leading to exhausted phenotype characterized by impaired T cell functionality.

Study I. The Effect of VSTM5 ECD Ig Fusion Proteins on Clearance of Viral Infection and on T Cell Functions During Acute and Chronic Viral Infection.

The effect of VSTM5 ECD Ig fusion proteins on acute and chronic viral infection is evaluated in a mouse model of infection with LCMV (lymphocytic chroriomeningitis virus) according to methodology described by Wherry et al J. Virol. 77: 4911-4927 (2003) and Barber et al Nature (2006), and further detailed below.

Two LCMV strains that can cause either acute or chronic infections in adult mice are used; the Armstrong strain which is cleared within a week, and the clone 13 strain which establishes a persistent infection that can last for months. As these two strains differ in only two amino acids, preserving all known T cell epitopes, it is possible to track the same CD8 T cell responses after an acute or chronic viral infection. In contrast to the highly robust memory CD8 T cells generated after an acute Armstrong infection, LCMV-specific CD8 T cells become exhausted during a persistent clone 13 infection (Wherry et al J. Virol. 77: 4911-4927, 2003; Barber et al., Nature 439:682-7(2006)).

Mice are infected with 2×105 PFU of Armstrong strain of LCMV intraperitoneally to initiate acute infection or 2×106 PFU of C1-13 intravenously to initiate chronic infection. Mice are treated i.p. with VSTM5 ECD Ig fusion proteins or with mIgG2a control, and with specific anti-VSTM5 antibody or an isotype control.

The mice are monitored for numbers of virus specific CD8⁺ T cells in the spleen, using virus-specific MHC tetramer epitopes, such as DbNP396-404 and DbGP33-41 which differ in acute or chronic infections. CD8 T cell functional assays, such as intracellular cytokines levels and CTL activity, are carried out as described by Wherry et al J. Virol. 77: 4911-4927, (2003). Additional assays include production by splenocytes after stimulation with virus specific epitopes; and assessment of viral titers in the serum and in the spleen, liver, lung and kidney (Wherry et al J. Virol. 77: 4911-4927 (2003); Barber et al., Nature, 439:682-7(2006)).

Study II. Assessment of VSTM5 Expression on Exhausted T Cells and Binding of VSTM5 ECD Ig Fusion Proteins to Exhausted T Cells in Order to Evaluate Regulation of these Proteins or their Counterpart Receptors During Exhaustion of T Cells

T cells are isolated from mice with chronic LCMV infection induced with C1-13 strain. The cells are co-stained with fluorescently labeled anti-PD-1 Ab as positive control (PD-1 is highly expressed by exhausted T cells) and biotinylated VSTM5, ECD Ig fusion proteins or biotinylated anti-VSTM5 fusion proteins antibodies, and respective isotype control. Binding is detected by FACS analysis using fluorescently labeled streptavidin.

Immunostimulatory VSTM5 fusion proteins and polypeptides according to the invention, which inhibit VSTM5, should elicit an enhancing effect on T and NK mediated immunity, and memory responses, and moreover elicit an inhibitory effect on suppressor cells. Therefore, it is anticipated that these fusion proteins may inhibit T cell exhaustion in the above-described infection models and thereby provide for longer-lived and more potent antiviral immune responses such as enhanced viral clearance. Such results will further validate the potential use of immunostimulatory VSTM5 fusion proteins according to the invention to treat viral infection and as an adjuvant in therapeutic or prophylactic viral vaccines.

### Example 30

### Assessment of VSTMm5 Protein Expression in Follicular Helper T (Tfh) Cells and the Binding of Ig Fusion Proteins to Tfh Cells

Follicular helper T (Tfh) cells are a subset of CD4⁺ T cells specialized in B cell help (reviewed by Crotty, Annu. Rev. Immunol. 29: 621-663 (2011)). Tfh cells migrate into B cell follicles within lymph nodes, and interact with cognate B cells at the T cell-B cell border and subsequently induce germinal center B cell differentiation and germinal center formation within the follicle (Reviewed by Crotty, Annu. Rev. Immunol. 29: 621-663 (2011)). The requirement of Tfh cells for B cell help and T cell-dependent antibody responses indicates that this cell type is of great importance for protective immunity against various types of infectious agents, as well as for rational vaccine design.

Tfh cells are readily identifiable at the peak of the CD4⁺ T cell response to an acute lymphocytic choriomeningitis virus (LCMV) infection as CXCR5^{hi}SLAM^{lo}BTLA^{hi}PD1hiBcl6⁺ virus-specific CD4⁺ T cells (Choi et al Immunity 34: 932-946(2011)). T cells are isolated from mice with acute LCMV infection induced with 2×10⁵ PFU of Armstrong strain of LCMV administered intraperitoneally. The cells are co-stained with fluorescently labeled antibodies for markers of Tfh (CXCR5, PD1, BTLA, Bcl6) which are highly expressed by Tfh cells, and biotinylated VSTM5 ECD-Ig fusion proteins or biotinylated antibodies specific for VSTM5 and respective isotype controls. Binding of Fc fused protein or antibody is detected by FACS analysis using fluorescently labeled streptavidin.

If antagonistic VSTM5 fusion proteins, based on their suppression of VSTM5, are evidenced to promote the proliferation and activity of follicular helper T (Tfh) cells, this would further validate the use of such VSTM5 proteins in eliciting therapeutic or prophylactic immunity against infectious agents as this T cell subtype is of established importance in eliciting protective immunity against various types of infectious agents.

### Example 31

### Assessment of the Effect of VSTM5 Ig Fusion Proteins on Follicular Helper T (Tfh) Cells Generation and Activity

In order to investigate the effect of VSTM5 ECD Ig fusion proteins on Tfh differentiation and development of B cell immunity in vivo, C57BL/6 are treated with VSTM5 ECD Ig fusion proteins and an isotype control throughout the course of an acute viral infection with Armstrong strain of LCMV (lymphocytic choriomeningitis virus). Tfh differentiation and Bcl6 protein expression is assessed by FACS analysis as described by Eto et al (PLoS One 6: e17739(2011)). Splenocytes are analyzed 8 days following LCMV infection, Tfh generation (CD44^{hi}CXCR5^{hi}SLAM^{lo}) and Bcl6 expression is evaluated by FACS analysis. In addition, the effect of VSTM5 ECD Ig fusion proteins on antigen-specific B cell responses is evaluated as described by Eto et al (PLoS One 6: e17739(2011)), including titers of anti-LCMV IgG in the serum at 8 days following LCMV infection, and quantitation by FACS analysis of plasma cell (CD138⁺IgD⁻) development at 8 days post-infection, gated on CD19⁺ splenocytes.

VSTM5 fusion proteins which block VSTM5, based on their suppression of VSTM5, may promote the proliferation and activity of follicular helper T (Tfh) cells in this assay. This would further validate the use of such VSTM5 proteins in eliciting therapeutic or prophylactic immunity against infectious agents as this T cell subtype is of established importance in protective immunity against various types of infectious agents.

### Example 32

### The Effect of Immunoinhibitory VSTM5 Proteins in the Modulation of Type 1 Diabetes in NOD Mice, CD28-KO NOD, and B7-2-KO NOD

The effect of immunoinhibitory VSTM5 ECD Ig fusion proteins are studied in a widely used mouse model of type 1 diabetes: nonobese diabetic (NOD) mice which develop spontaneous In NOD mice, spontaneous insulitis, the hallmark pathologic lesion, evolves through several characteristic stages that begin with peri-insulitis and end with invading and destructive insulitis and overt diabetes. Peri-insulitis is first observed at 3-4 wk of age, invading insulitis at 8-10 wk, and destructive insulitis appears just before the onset of clinical diabetes, with the earliest cases at 10-12 wk. At 20 wk of age, 70-80% of female NOD mice become diabetic (Ansari et al J. Exp. Med. 198: 63-69(2003)).

Two KO mice: CD-28-KO NOD mice and B7-1/B7-2 double KO NOD mice, —which develop accelerated diabetes (Lenschow et al Immunity 5: 285-293 (1996); Salomon et al Immunity 12: 431-440(2000)), are also used.

Study I: NOD Mice are Treated with Immunoinhibitory VSTM5 ECD-Ig Fusion Proteins

In this 1st study NOD mice are treated with immunoinhibitory VSTM5 ECD-Ig fusion proteins early and late phases during the evolution of diabetes, before or after disease onset, to examine the effects of these compounds on disease pathogenesis and to demonstrate that such treatment reduces disease onset and ameliorates pathogenesis. To study the effect on insulitis, blood glucose levels are measured 3 times/week, for up to 25 weeks (Ansari et al J. Exp. Med. 198: 63-69(2003)).

Mechanism of disease modification and mode of action is studied by experimental evaluation of individual immune cell types: pancreas, pancreatic LNs and spleen will be harvested to obtain Tregs, Th subtypes and CD8⁺ T cells, DCs and B cells. Effect on cytokines secretion from cells isolated from pancreas, pancreatic LN and spleen is analyzed, focused on IFNγ, IL-17, IL-4, IL-10 and TGFβ. Upon effect of the tested compounds, the mechanism of disease modification is studied by examination of individual immune cell types (including Tregs, Th subtypes and CD8⁺ T cells, DCs and B cells); cytokines (IFNγ, IL-17, IL-4, IL-10 and TGFβ) and histology. Histological analysis of the pancreas is carried out to compare the onset of insulitis, and the lymphocyte infiltration.

Study II—the Effect of Immunoinhibitory VSTM5 Ig Fusion Proteins in the Modulation of Type 1 Diabetes in Adoptive Transfer Model

To further investigate the mode of action of the Ig fusion proteins, an adoptive transfer model of diabetes is used. T cells from diabetic or prediabetic NOD donors are transferred to NOD SCID recipient mice. These mice are monitored for development of diabetes. The urine glucose and blood glucose, and assess histology of the pancreas, and T cell responses are monitored as described in the previous example.

Study III: The Effect of Immunoinhibitory VSTM5 Ig Fusion Proteins on the Induction of Diabetes in NOD Mice

Diabetes is also induced by the transfer of activated CD4⁺CD62L+CD25⁻ BDC2.5 T cells (transgenic for TCR recognizing islet specific peptide 1040-p31 activated by incubation with 1040-p31) to NOD recipients. Mice are treated with immunoinhibitory VSTM5 ECD Ig fusion proteins, control mIgG2a or positive control. Treatments begin 1 day following transfer. Mice are followed for glucose levels 10-28 days post transfer (Bour-Jordan et al., J Clin Invest. 114(7):979-87(2004)).

Seven days post treatment pancreas, spleen, pancreatic LN and peripheral lymph node cells are extracted and examined for different immune cell populations. In addition, recall responses are measured by testing ex vivo proliferation and cytokine secretion in response to p31 peptide. It is anticipated that agonistic VSTM5 fusion proteins, based on their immunosuppressive properties may prevent or reduce disease onset or the severity thereof in the above-described accepted animal models of type 1 diabetes.

### Example 33

### The Effect of Immunoinhibitory VSTM5 ECD Ig Fusion Proteins in Lupus Mouse Models

Study I: The Lupus-Prone Mouse Model

In the first lupus animal model, (NZB×NZW)F1 (B/W) mice are used which are prone to developing lupus. Cyclophosphamide (CTX) is the primary drug used for diffuse proliferative glomerulonephritis in patients with renal lupus, Daikh and Wofsy reported (J Immunol, 166(5):2913-6 (2001)) that combination treatment with CTX and CTLA4-Ig was more effective than either agent alone in reducing renal disease and prolonging survival of NZB/NZW F1 lupus mice with advanced nephritis. In this experiment, treatments with VSTM5 ECD Ig fusion proteins and CTX either alone or in combination are tested.

Blood samples are collected 3 days before the protein treatment and then every other week during and after treatments for plasma anti-dsDNA autoantibody analysis by ELISA. Glomerulonephritis is evaluated by histological analysis of kidneys. Proteinuria is measured by testing fresh urine samples using urinalysis dipsticks.

Study II: The NZM2410-Derived B6, Sle1, Sle2, Sle3 Mouse Model of SLE

VSTM5 ECD Ig fusion proteins are tested in a second lupus model to determine their ability to ameliorate the symptoms of lupus nephritis. In this study the NZM2410-derived B6. Sle1Sle2 Sle3 mouse model of SLE is used. NZM2410 is a recombinant inbred strain produced from NZB and NZW that develops a highly penetrant lupus-like disease with an earlier onset of disease (Blenman et al Lab. Invest. 86: 1136-1148(2006)). The effect of immunoinhibitory VSTM5 ECD Ig fusion proteins is studied in this model by detecting changes in proteinuria and autoantibodies as described in the previous lupus model.

Study III: Induced Lupus Model

This model is based on chronic graft-vs-host (cGVH) disease induced by the transfer of Ia-incompatible spleen cells from one normal mouse strain (such as B6. C—H2(bm12)/KhEg (bm12)) to another (such as C57BL/6), which causes an autoimmune syndrome resembling systemic lupus erythematosus (SLE), including anti-double-stranded DNA (anti-dsDNA) autoantibodies and immune complex-type proliferative glomerulonephritis (Appleby et al Clin. Exp. Immunol. 78: 449-453(1989)); Eisenberg and Choudhury Methods Mol. Med. 102:273-284(2004)).

Lupus is induced in this model following injection of spleen cells from bm12 mice into C57BL/6 recipients. The effect of immunoinhibitory VSTM5 ECD Ig fusion proteins is studied in this model by assessment of proteinuria and autoantibodies as described above. T cell and responses B cell responses are also evaluated.

Study IV: The MRL/lpr Lupus Prone Mouse Model

The MRL/lpr lupus prone mouse model is used in this 4th study. The effect of immunoinhibitory VSTM5 ECD Ig fusion proteins is again studied in this model by assessment of proteinuria and autoantibodies as described above.

It is anticipated that the administration of an immunoinhibitory VSTM5 fusion protein in these models of lupus will inhibit glomerulonephritis and result in reduced proteinuria in urine samples. These results will further corroborate the potential use thereof in treating lupus, a chronic autoimmune disease which unless managed, may impair or even destroy kidney function.

### Example 34

### Effect of Immunoinhibitory VSTM5 Proteins in the Control of Intestinal Inflammation

Study I: Effect of VSTM5 ECD Ig Fusion Proteins in Adoptive Transfer Mouse Model of Colitis

An adoptive transfer mouse model of colitis in mice is used, whereby the transfer of CD45RB^{high}-CD4⁺ naïve T cells from BALB/c mice to syngeneic SCID mice leads to the development of an IBD-like syndrome by 6-10 wks after T cell reconstitution, similar to human Crohn's disease.

SCID mice are reconstituted by i.p. injection of syngeneic CD45RB^{high}-CD4⁺ T cells either alone or cotransferred with syngeneic CD45RB^{low}CD4⁺ or CD25⁺CD4⁺ cells (4×10⁵/mouse of each cell population) (Liu et al., J Immunol. 2001; 167(3): 1830-8(2001)). Colitic SCID mice, reconstituted with syngeneic CD45RBhighCD4+ T cells from spleen of normal mice, are treated i.p., with immunoinhibitory VSTM5 ECD Ig fusion proteins or Ig isotype control, twice a week starting at the beginning of T cell transfer up to 8 wk. All mice are monitored weekly for weight, soft stool or diarrhea, and rectal prolapse. All mice are sacrificed 8 wk after T cell transfer or when they exhibit a loss of 20% of original body weight. Colonic tissues are collected for histologic and cytologic examinations.

Study II: Effect of VSTM5 ECD Ig Fusion Proteins in DSS-Induced Colitis

Rag-1 KO mice of B6 backgrounds (Jackson Laboratory) receive either 2% DSS-containing drinking water or regular drinking water Immunoinhibitory VSTM5 ECD Ig fusion proteins, CTLA-4-Fc (positive control) or mIgG2a isotype control are administered intraperitoneally on days 0, 3 and 6. Body weight is followed until study termination. On day 9 mice are sacrificed and the colon length is measured. Colon sections (4 μm) are subjected to histological analysis by H&E-stain.

It is anticipated that the administration of an immunoinhibitory VSTM5 fusion protein according to the invention will inhibit bowel dysfunction in this IBD model based on their inhibitory effect on CD4⁺ pathologic T cells. Such results will corroborate the potential use of VSTM5 polypeptides and fusion proteins according to the invention in treating inflammatory bowel disease.

### Example 35

### The Effect of Immunoinhibitory VSTM5 Proteins in Mouse Model of Psoriasis

Study I: Establishment of Psoriasis SCID Xenograft Model.

Human psoriasis plaques are transplanted on to the SCID mice. Shave biopsies (2.5_2.5 cm) are taken from patients with generalized plaque psoriasis involving 5-10% of the total skin that did not receive any systemic treatment for psoriasis or phototherapy for 6 months and did not receive any topical preparations other than emollients for 6 weeks. The biopsies are obtained from active plaques located on the thigh or arm. Each piece of biopsy is divided into four equal parts of approximately 1 cm2 size. Each piece is transplanted to a separate mouse.

Under general anesthesia, a graft bed of approximately 1 cm² is created on the Under general anesthesia, a graft bed of approximately 1 cm² is created on the shaved area of the back of a 7- to 8-week-old CB 17 SCID mouse by removing a full-thickness skin sample, keeping the vessel plexus intact on the fascia covering the underlying back muscles. The partial thickness human skin obtained by shave biopsy is then orthotopically transferred onto the graft bed. Nexaband, a liquid veterinary bandage (Veterinary Products Laboratories, Phoenix, Ariz.) is used to attach the human skin to the mouse skin and an antibiotic ointment (bacitracin) is applied. Mice are treated intraperitoneally three times per week for 4 weeks with immunoinhibitory VSTM5 ECD Ig fusion proteins, isotype control or CTLA4-Ig (positive control).

Punch biopsies (2 mm) are obtained on day 0 (before treatment) and day 28 (after treatment) of the study period. Biopsies are snap frozen and cryosections for histopathological and immunohistochemical studies. Therapeutic efficacy is determined by comparing pre- and post-treatment data: (i) rete peg lengths to determine the effect on epidermal thickness and (ii) the level of lymphomononuclear cell infiltrates to determine the effect on inflammatory cellular infiltrates. (Raychaudhuri et al., J Invest Dermatol.; 128(8):1969-76(2008); and Boehncke et al., Arch Dermatol Res 291:104-6 (1999)).

It is anticipated that the administration of an immunoinhibitory VSTM5 fusion protein according to the invention will have a beneficial effect in ameliorating psoriasis in this animal model thereby validating its potential use in treating psoriasis.

Study II: The Effect of Immunoinhibitory VSTM5 in Psoriasis Induced in Healthy Human Skin Graft

Healthy human skin pieces with a width of 0.4 mm and surface area of 1.5×1.5 cm are provided from residual skin of routine plastic surgery procedures from the Plastic Surgery Department of the Rambam Medical Center, Israel. In addition, 20 mL blood samples are taken from psoriatic patients.

Normal healthy human donor skin is transplanted on the back of beige-SCID mice as previously described (Nickoloff et al., Amer J Pathol. 146(3):580-8(1995); Wrone-Smith, J Clin. Invest. 98(8):1878-87(1996); Gilhar et al., J Invest. Dermatol. 133:844-7 (2002) Kalish et al., J Invest. Dermatol. 129(3) 773-6 (2009) and the mice are divided into treatment groups.

Peripheral Blood Mononuclear Cells (PBMCs) isolated from psoriatic patients' blood is cultured in the presence of IL-2 [Prospec, 100 U/mL of RPMI 1640 media, 10% human AB serum (Sigma, St. Louis, Mo.), 1% glutamine, 1% antibiotics (media components; Biological Industries, Kibbutz Beit Haemeck, Israel)] for 14 days, as previously described Gilhar et al. J Invest. Dermatol. 133:844-7 (2002).

Four weeks following skin engraftment, each mouse is injected intra-dermally (i. d.) into the grafted skin lesion, with 1×10⁷ activated allogeneic enriched PBMCs from psoriatic patients. Cells from different psoriasis patients are equally distributed between treatment groups so that each patient is represented in each treatment group.

On the day of PBMCs injection into the skin grafts, mice are divided randomly into treatment groups (n=9-10 mice per group) and administration of all drugs begins. All tested compounds including the vehicle are given intradermally (intralesional), three times a week, except for dexamethasone which is given topically for 14 consecutive days.

Twenty eight days after starting the injections and treatment, skin is harvested and analyzed for the following psoriatic parameters by histology: epidermal thickening (acanthosis), suprapapillary epidermal thinning (papillomatosis), hyperkeratosis, parakeratosis, orthokeratosis, agranulosis—hypogranulosis, appearance of neutrophils in the upper spinous layer or in the corneal cell layer—Munro microabscess, regular elongation of rete ridges, angiogenesis, edema and dilated tortuous blood vessels in the dermal papilla and mononuclear cell infiltrate in the papillary dermis. Epidermal thickness is measured with an ocular micrometer at a minimum of 50 points along the epidermis selected to represent points of maximal and minimal thickness. HLA-DR, ICAM-1, Ki-67 and immune infiltrates (anti-CD3 stain) are evaluated by immunohistochemistry.

It is anticipated that the subject VSTM5 fusion protein will inhibit immune response to the graft and inhibit signs of psoriasis in this model.

Study III: The Effect of Immunoinhibitory VSTM5 in Immiquimode-Induced Psoriasis

This model enables the monitoring of psoriasis-like skin inflammation in mice. Psoriasis is induced by imiquimod. The first signs of the disease are observed within 2-3 days as redness and plaque type psoriasis, and from this point on, the disease progresses to the entire back of the animal.

Female BALB/c mice at 8 to 11 wk of age are anesthetized with isoflurane and their backs are shaved. Commercially available imiquimod cream (5%) (Aldara; 3M Pharmaceuticals) is topically applied once daily starting on day 1 to the shaved back and to the right ear for 5-10 consecutive days. Mice are treated with immunoinhibitory VSTM5 ECD Ig fusion proteins, control mIgG2a or positive control from day of disease induction (day 1).

Erythema, scaling, and thickening are scored independently on a scale from 0 to 4: 0, none; 1, slight; 2, moderate; 3, marked; 4, very marked. The level of erythema is scored using a scoring table with red taints. The cumulative score (erythema plus scaling plus thickening) serves as a measure of the severity of inflammation (scale 0-12). Right ear thickness is measured using a digital caliper before psoriasis induction on study day 1 (baseline for ear volume parameter) until study termination.

At the end of the study, 4-6 hours after the final IMQ treatment, the mice are euthanized using pentobarbital sodium injection. One tissue from the back (diseased or healthy by base of tail) is collected into vials containing 4% formalin for histology. The organs is cut at 5 μm sections using paraffin embedding methodology and stained with H&E.

It is anticipated that the subject immunoinhibitory VSTM5 fusion protein will inhibit NK cells and thereby inhibit signs of psoriasis in this model.

Study IV: Effect of Immunoinhibitory VSTM5 in Psoriasis Model Involving Adoptive Transfer of CD45RB^{hi} CD4⁺ T Cells in SCID Mice

Immunocompromised mice are injected intravenously (i. v.) with 0.3×10⁶ CD4⁺ CD45RB′ cells. On the day following the adoptive transfer of cells, mice are injected intraperitoneally (i.p.) with 10 μg of Staphylococcal enterotoxin B (Davenport et al., Int. Immunopharmacol. 2(5):653-72(2002)). Recipient mice are treated with immunoinhibitory VSTM5 ECD-Ig fusion proteins, isotype control or CTLA4-Ig (positive control). Mice are evaluated once a week for 8 weeks for weight loss and presence of skin lesions.

It is anticipated that the administration of an immunoinhibitory VSTM5 fusion protein according to the invention will have a beneficial effect in ameliorating psoriasis in this animal model thereby validating its potential use in treating psoriasis.

### Example 36

### The Effect of Immunoinhibitory VSTM5 Proteins in Modulating Transplant Rejection

Study I: Allogeneic Islet Transplant Diabetes Model

The effect of VSTM5 in a model of allogeneic islet transplantation in diabetic mice. To test the effect of immunoinhibitory VSTM5 ECD Ig fusion proteins on transplant rejection, a model of allogeneic islet transplantation is used. Diabetes is induced in C57BL/6 mice by treatment with streptozotocin. Seven days later, the mice are transplanted under the kidney capsule with pancreatic islets which are isolated from BALB/c donor mice. Recipient mice are treated with immunoinhibitory VSTM5 ECD Ig fusion proteins or with mIgG2a as a negative control. Tolerance with ECDI-fixed donor splenocytes is used as the positive control for successful modulation islet graft rejection. Recipient mice are monitored for blood glucose levels as a measure of graft acceptance/rejection (Luo et al., PNAS, 105(38): 14527-14532(2008)).

Study II: Effect of VSTM5 in the Hya-Model of Skin Graft Rejection.

In humans and certain strains of laboratory mice, male tissue is recognized as non-self and destroyed by the female immune system via recognition of histocompatibility-Y chromosome encoded antigens (Hya). Male tissue destruction is thought to be accomplished by cytotoxic T lymphocytes in a helper-dependent manner Therefore, in order to test the effect of immunoinhibitory VSTM5 ECD Ig fused proteins on transplantation, the Hya model system is used, in which female C57BL/6 mice receive tail skin grafts from male C57BL/6 donors.

In this study, female C57BL/6 mice are engrafted with orthotopic split-thickness tail skin from age matched male C57BL/6 mice. The mice are treated with immunoinhibitory VSTM5 ECD Ig fusion proteins, isotype control mIgG2a. Immunodominant Hya-encoded CD4 epitope (Dby) attached to female splenic leukocytes (Dby-SP) serve as positive control for successful modulation of graft rejection (Martin et al., J Immunol. 185(6): 3326-3336(2010)). Skin grafts are scored daily for edema, pigment loss and hair loss. Rejection is defined as complete hair loss and more than 80% pigment loss.

In addition, T cell recall responses of cells isolated from spleens and draining lymph nodes at different time points are studied in response to CD4 specific epitope (Dby), CD8 epitopes (Uty and Smcy) or irrelevant peptide (OVA 323-339) while anti CD3 stimulation is used as positive control for proliferation and cytokine secretion.

Study III: Graft Rejection Murine Model

The effect of immunoinhibitory VSTM5 ECD Ig fusion proteins on graft rejection is studied in a murine model of syngeneic bone marrow cells transplantation using the Hya model system described above. Male hematopoietic cells expressing the CD45.1 marker are transplanted to female host mice which express the CD45.2 congenic marker. Female hosts are treated with VSTM5 ECD Ig fusion proteins or with isotype control mIgG2a. The female hosts are followed over time and the presence of CD45.1⁺ cells is monitored.

On week 8 post transplantation a blood sample and spleens are analyzed for CD45.2 T cells subpopulations by FACS analysis for CD4, CD8, CD44, CD25, FoxP3, Helios and Nrp-1 and B cell populations including Breg cells using by FACS analysis for CD19, CD24, CD38 and intracellular IL-10.

It is anticipated that the administration of an immunoinhibitory VSTM5 fusion protein according to the invention will have a beneficial effect in preventing rejection responses in these 3 transplant animal models thereby further validating the potential use of immunoinhibitory VSTM5 polypeptides and fusion proteins in treating or preventing rejection responses to transplanted cells, tissues or organs and potentially for promoting tolerance to the transplanted cells, tissue or organ.

### Example 37

### The Effect of VSTM5 ECD Ig Fusion Proteins on Humoral Immunity

The effect of VSTM5 ECD Ig fusion proteins on induction of humoral response by immunization of mice with NP-KLH⁺ each one of the following adjuvants: CFA, LPS, Alum or Ficoll is tested.

Variant A: C57/BL/6 mice are immunized on day 0 by s.c. injection of 50 μg of NP-KLH (4-hydroxy-3-nitrophenyl acetyl-keyhole limpet hemocyanin) plus either CFA, LPS, Alum or Ficoll. Mice are treated intraperitoneally with VSTM5 ECD Ig fusion protein, mIgG2a isotype control or CTLA4-Ig, each at 100 or 300 ug/dose, on days −1, 1, 3 and 5. Blood sample is taken for analysis of serum anti KLH antibodies on day 0 (before immunization with KLH) and then on days 7, 14 and 21.

Anti-Antigen/NP-specific antibodies of isotypes IgM, IgG1, IgG2c, IgG2b, IgG3, IgA and IgE are evaluated using ELISA.

Variant B: In another study, mice are immunized with 50 micrograms of NP-KLH plus either CFA, LPS, Alum® or Ficoll® on day 0 and boosted again on day 28. Treatments are given as above, on days 27, 29, 31, 33 and blood samples are collected on days 0, 35 and 42 for analysis of isotype switch by ELISA for IgM, IgG1, 2c, 2b, 3, IgA and IgE antigen/NP-specific antibodies.

Histological analysis for germinal center formation is carried out by PNA staining on day 14 or 35 for variants 1 and 2 respectively. Plasma cell formation is evaluated by FACS analysis using CD138 staining Other general spleen cell populations (T cells, B cells, monocytes, neutrophils, etc.) are analyzed using specific population markers. In addition, intracellular evaluation of IgG and IgM is carried out by ELISpot.

Based on VSTM5's immunosuppressive properties and inhibitory effect on T cell activation, it is anticipated that the subject VSTM5 fusion protein may inhibit antibody immune responses in this humoral immunity model.

### Example 38

### The Effect of VSTM5 ECD Ig Fusion Proteins on Human CD4⁺ T Cell Responses from Healthy Donors and MS Patients

Total PBMCs are isolated from healthy donors and from RR-MS patients, and cells are activated in vitro in the presence of medium only, anti-CD3, MBP84-99 peptide, or TT peptide in the presence of Control Ig or VSTM5 ECD Ig fusion proteins. Proliferative response as well as the level of cytokines secreted in culture are evaluated.

Based on the immunoinhibitory effects of the VSTM5 fusion protein, it is anticipated that immunoinhibitory VSTM5 fusion protein according to the invention will inhibit CD4⁺ T cell responses of the T cells derived from MS patients. Given the well-known involvement of CD4⁺ T cells in MS disease pathology, this will further corroborate the potential use of VSTM5 polypeptides and fusion proteins according to the invention for treating MS.

### Example 39

### The Effect of VSTM5 ECD Ig Fusion Proteins in Graft Versus Host Disease

In this model, semiallogeneic (partially MHC-mismatched) C57BL/6 (B6,H-2b) naive donor T cells and T cell-depleted BM are adoptively transferred to lethally irradiated C57BL/6 3 DBA/2 F1 hybrid mice (BDF1, H-2bxd). BDF1, H-2bxd mice (n=5 per group) are irradiated using a Gammacell 40 irradiator (Cesium source, 0.50 cGy/min dose rate; Atomic Energy of Canada) 12 h before adoptive transfer. C57BL/6 bone marrow (BM) is isolated from femurs and tibias by flushing bones with RPMI 1640 supplemented with 10% Fetalclone III (Hyclone/Thermo Scientifc) and penicillin-streptomycin (Roche) and disaggregated through a 100-mm mesh screen (BD Biosciences) with a rubber syringe plunger. Red blood cells (RBCs) are lysed in ACK buffer, and T cells are depleted with CD90.2 (thy1.2) MACS beads according to protocol (Miltenyi Biotec). T cells are isolated from total lymph node (LN) cells and enriched using pan T cell MACS negative selection beads according to protocol (Miltenyi Biotec). BD/F1 mice are adoptively transferred with 5×106 T cell-depleted BM plus 3×106 pan T cells by tail vein injection and treated with VSTM5 ECD Ig fusion proteins or Ig control from the day of injection or in a preventive mode, begging 1 wk before adoptive cell transfer. Mice are followed for chimerism of circulating blood cells once weekly until day 30 by FACS analysis of circulating donor neutrophils, CD4⁺ T cells, and CD8⁺ T cells. Numbers of infiltrating T cells in all GVHD target tissues including spleen, liver, small intestine, lung, and kidney are examined by IHC analysis. In addition, mice are followed visually for coat sheen, mobility, activity, and appetite.

Based on the immunoinhibitory effects of the VSTM5 fusion protein, it is anticipated that immunoinhibitory VSTM5 fusion protein according to the invention will result in chimerism of circulating blood cells and/or reduced infiltration of autoreactive T cells to GVHD target tissues.

The invention has been described and various embodiments provided relating to manufacture and selection of desired VSTM5 proteins for use as therapeutics and diagnostic methods wherein the disease or condition is associated with VSTM5. Different embodiments may optionally be combined herein in any suitable manner, beyond those explicit combinations and subcombinations shown herein. The invention is now further described by the claims which follow.

1. An isolated polypeptide comprising a fragment of a VSTM5 ECD, wherein said fragment consists essentially of or consists of an amino acid sequence as set forth in any one of SEQ ID NOs: 1, 12-110, 151-156, or a variant thereof that possesses at least 95% sequence identity therewith.

2. An isolated polypeptide comprising at least two VSTM5 ECD polypeptide fragments, wherein said fragments are the same or different and are selected from the amino acid sequences set forth in any one of SEQ ID NOs: 1, 12-110, 151-156, or a variant thereof that possesses at least 95% sequence identity therewith, but wherein a sequence of said isolated polypeptide has less than 95% sequence identity with an amino acid sequence as set forth in any one of SEQ ID NOs: 1, 12-110, 151-156.

3. The isolated polypeptide of claim 2, which comprises 2-10 of said VSTM5 ECD polypeptide fragments.

4. An isolated polypeptide according to claim 3, wherein said fragments are intervened by a heterologous linker, wherein said linker is not a fragment of a VSTM5 polypeptide.

5. (canceled)

6. (canceled)

7. (canceled)

8. (canceled)

9. (canceled)

10. (canceled)

11. (canceled)

12. A fusion protein comprising the isolated polypeptide of claim 1, or SEQ ID NOs 2 or 3, joined to a heterologous polypeptide and/or half-life extending moiety, with the proviso that said heterologous polypeptide or said half-life extending moiety is not a fragment of a VSTM5 polypeptide.

13. The fusion protein according to claim 12, wherein said isolated polypeptide and said heterologous molecule are intervened by a heterologous linker, with the proviso that said linker does not comprise a polypeptide that is a fragment of a VSTM5 polypeptide.

14. (canceled)

15. (canceled)

16. (canceled)

17. (canceled)

18. (canceled)

19. (canceled)

20. (canceled)

21. The fusion protein of claim 13, a comprising or further comprising a half-life extending moiety.

22. The fusion protein according to claim 21, wherein the half-life extending moiety comprises polyethylene glycol (PEG), monomethoxy PEG (mPEG), an XTEN molecule, an rPEG molecule, an adnectin, a serum albumin, human serum albumin, immunoglobulin constant region or fragment thereof, or acyl group.

23. The fusion protein according to claim 22, wherein the addition of said heterologous polypeptide, half-life extending moiety, or other heterologous molecule increases the in vivo half-life of said fusion protein by at least about 2-fold, at least about 3-fold, at least about 4-fold, at least about 5-fold, at least about 10-fold, or more, as compared to the identical molecule without such said heterologous polypeptide, half-life extending moiety, or other heterologous molecule.

24. The fusion protein according to claim 22 which comprises an immunoglobulin molecule or a fragment thereof.

25. The fusion protein according to claim 24, wherein at least one of the heterologous polypeptides is a human or non-human immunoglobulin Fc polypeptide or fragment that comprises heavy and/or light chain C_{H2} and C_{H3} domains.

26. (canceled)

27. (canceled)

28. (canceled)

29. (canceled)

30. The fusion protein of claim 25, wherein said immunoglobulin molecule or a fragment thereof comprises a hinge region.

31. The fusion protein of claim 30, wherein said hinge region is an intact hinge region.

32. The fusion protein of claim 25, wherein said immunoglobulin molecule or a fragment thereof does not feature a hinge region.

33. (canceled)

34. (canceled)

35. (canceled)

36. The fusion protein of claim 24 comprising an immunoglobulin heavy chain constant region derived from an immunoglobulin isotype selected from the group consisting of an IgG1, IgG2, IgG3, IgG4, IgM, IgE, IgA and IgD.

37-129. (canceled)
